(19) United States
(12) Patent Application Publication
(10) Pub. No.: US 2026/0159603 A1
(43) Pub. Date: Jun. 11, 2026

(54) CD5-BINDING POLYPEPTIDES, COMPOSITIONS COMPRISING THE SAME, AND METHODS FOR USE THEREOF

(71) Applicant: Beam Therapeutics Inc., Cambridge, MA (US)

(72) Inventor: Shawn Jennings, Cambridge, MA (US)

(73) Assignee: Beam Therapeutics Inc., Cambridge, MA (US)

(21) Appl. No.: 19/468,887

(22) Filed: Feb. 3, 2026

Related U.S. Application Data
(63) Continuation of application No. PCT/US2024/042423, filed on Aug. 15, 2024.
(60) Provisional application No. 63/520,065, filed on Aug. 16, 2023.
(60) Provisional application No. 63/592,339, filed on Oct. 23, 2023.

(51) Int. Cl. C07K 16/28

(52) U.S. Cl. CPC C07K 16/2896; C07K 2317/569; C07K 2317/92

(57) ABSTRACT

As described below, the present disclosure features polypeptides capable of binding a cluster of differentiation 5 (CD5) antigen and polynucleotides encoding said CD5-binding polypeptides, compositions comprising the same, and methods for use thereof. The disclosure also features lipid nanoparticles comprising the CD5-binding polypeptides and methods for use thereof for delivery of a polynucleotide (e.g., a polynucleotide encoding a chimeric antigen receptor) to a T cell in vivo.

### CROSS REFERENCE TO RELATED APPLICATIONS

This application is a continuation under 35 U.S.C. § 111 (a) of PCT International Patent Application No. PCT/US2024/042423, filed Aug. 15, 2024, designating the United States and published in English, which claims priority to U.S. Provisional Application No. 63/520,065, filed Aug. 16, 2023, and U.S. Provisional Application No. 63/592,339, filed Oct. 23, 2023, the entire contents of each of which are hereby incorporated by reference in its entirety.

### SEQUENCE LISTING

This application contains a Sequence Listing which has been submitted electronically in XML format and is hereby incorporated by reference in its entirety. The Sequence Listing XML file, created on Aug. 14, 2024, is named 180802-055703PCT_SL.xml and is 1,228,906 bytes in size.

### BACKGROUND

Autologous and allogeneic immunotherapies are neoplasia treatment approaches in which immune cells expressing chimeric antigen receptors are administered to a subject. To generate an immune cell that expresses a chimeric antigen receptor (CAR), an immune cell of a subject (autologous) or from a donor separate from the subject receiving treatment (allogeneic) is genetically modified to express the chimeric antigen receptor. The cells may be genetically modified within the subject or modified in vitro and subsequently administered to the subject. The resulting cell expresses the chimeric antigen receptor on its cell surface (e.g., CAR-T cell) and the chimeric antigen receptor binds to an antigen expressed by a pathogenic cell in the subject, such as a neoplastic cell. This interaction with the antigen activates the CAR-T cell, which then kills the neoplastic cell. There are various challenges to be overcome when administering an autologous or allogeneic immunotherapy to a subject. For example, autologous cell therapies traditionally have disadvantages associated with having to usually obtain the starting material from the patient to be treated, including long manufacturing times and the requirement that the patient cells are suitable despite previous therapies or disease state. Further, for allogeneic cell therapy, graft-versus-host disease (GVHD) and host rejection of CAR-T cells provide additional challenges. Thus, there is a significant need for improved methods and compositions for use in autologous and allogeneic immunotherapies.

### SUMMARY

As described below, the present disclosure features polypeptides capable of binding a cluster of differentiation 5 (CD5) antigen and polynucleotides encoding said CD5-binding polypeptides, compositions comprising the same, and methods for use thereof. The disclosure also features lipid nanoparticles comprising the CD5-binding polypeptides and methods for use thereof for delivery of a polynucleotide (e.g., a polynucleotide encoding a chimeric antigen receptor) to a T cell in vivo.

In one aspect, the disclosure provides a VHH antibody or an antigen binding fragment thereof that specifically binds to a cluster of differentiation 5 (CD5) polypeptide. The VHH antibody contains three Complementarity Determining Regions (CDRs): CDR1, CDR2 and CDR3, that are structurally positioned between four camelid VHH framework (FR) regions (FR1-FR4) as follows: FR1-CDR1-FR2-CDR2-FR3-CDR3-FR4, where: a) CDR1 is selected from one or more of: NYAAG (SEQ ID NO: 478), SYTMG (SEQ ID NO: 479), TYTMG (SEQ ID NO: 480), SYAMG (SEQ ID NO: 481), TYNMG (SEQ ID NO: 482), AYAMG (SEQ ID NO: 483), SSGMG (SEQ ID NO: 484), VDATT (SEQ ID NO: 485), INVIG (SEQ ID NO: 505), SSFMS (SEQ ID NO: 506), TNVMG (SEQ ID NO: 507), TNNMG (SEQ ID NO: 508), TNNMA (SEQ ID NO: 509), RVAMN (SEQ ID NO: 510), RVGMN (SEQ ID NO: 511), FVGWG (SEQ ID NO: 512), FIGWG (SEQ ID NO: 513), MYSMS (SEQ ID NO: 514), and TYGMG (SEQ ID NO: 515); b) CDR2 is selected from one or more of RISRSGGRTDYADSVKG (SEQ ID NO: 486), AISWSAGRTYYADSMKG (SEQ ID NO: 487), VISWSGGRTYYADSVKG (SEQ ID NO: 488), AIDLYGRATRYANSVKG (SEQ ID NO: 489), AINLEGYATRYANSVKG (SEQ ID NO: 615), AIDLYGRATRYANSVRG (SEQ ID NO: 616), AINWNGDTALRWNGFATRYADSVKG (SEQ ID NO: 490), SMDWSGGSTYYGDSVKG (SEQ ID NO: 491), SIDWSGGSTYYGDSVKG (SEQ ID NO: 492), SIDWGGGSTYYGDSVKG (SEQ ID NO: 493), SIDWSGKSTYYGDSVKG (SEQ ID NO: 494), SINWSGGSAYYGDSVKG (SEQ ID NO: 495), SMDWTGGSTYYGDSVKG (SEQ ID NO: 496), IMDIGGVTEYADSVKG (SEQ ID NO: 497), LVNSGGQTHYADSVKG (SEQ ID NO: 516), TIYSDGSTYYADSVKG (SEQ ID NO: 517), TIYSDGSTYYADSMKG (SEQ ID NO: 518), LIRGGGSTHYADSVKG (SEQ ID NO: 519), LIRTGGSTHVADSMKG (SEQ ID NO: 520), TISSDGSRTNYAHSVKG (SEQ ID NO: 522), SISSDGSRTNYAHFVKG (SEQ ID NO: 523), QISTGGLTNYADSVKG (SEQ ID NO: 524), QINTGGLTDVYADSVKG (SEQ ID NO: 617), SISTGARDTAYADSVKG (SEQ ID NO: 526), SISTGARDTSYADSVKG (SEQ ID NO: 618), and VITGSGVGTQYADSVKD (SEQ ID NO: 527); and c) CDR3 is selected from one or more of: ATVWEFTDGADQYDY (SEQ ID NO: 498), DPWTSDSDYDRLTMYDY (SEQ ID NO: 499), DPWTSDSDYERLTMYDY (SEQ ID NO: 500), DTSLPLGVLTESQRLYGA (SEQ ID NO: 501), DTSLPLGVLTKSQRMYGA (SEQ ID NO: 502), DTVVSGSYYLAARAEDYEY (SEQ ID NO: 503), GTSGVAAVNLRGFFS (SEQ ID NO: 504), RGL, RYGIDNY (SEQ ID NO: 528), VTGSI (SEQ ID NO: 529), WLGSPGAMSDY (SEQ ID NO: 530), WTGSPGALSDY (SEQ ID NO: 531), PGNS (SEQ ID NO: 532), PGHP (SEQ ID NO: 533), PGHS (SEQ ID NO: 534), GDLRYGPDGYDY (SEQ ID NO: 535), and GHRPGWAVIRADAYEY (SEQ ID NO: 536).

In another aspect, the disclosure provides a chimeric antigen receptor polypeptide containing the VHH antibody of any aspect of the disclosure, or embodiments thereof, or an antigen-binding fragment thereof.

In another aspect, the disclosure provides an immunoconjugate containing the VHH antibody of any aspect of the disclosure, of embodiments thereof.

In another aspect, the disclosure provides a polynucleotide encoding the VHH antibody of any aspect of the disclosure, or embodiments thereof.

In another aspect, the disclosure provides a vector containing the polynucleotide of any aspect of the disclosure, or embodiments thereof.

In another aspect, the disclosure provides a cell expressing the VHH antibody of any aspect of the disclosure, or embodiments thereof.

In another aspect, the disclosure features a lipid nanoparticle containing the VHH antibody of any aspect of the disclosure, or embodiments thereof.

In another aspect, the disclosure features a composition containing the VHH antibody, the chimeric antigen receptor, the immunoconjugate, the polynucleotide, the vector, the cell, or the lipid nanoparticle of any aspect of the disclosure, or embodiments thereof, and a carrier or excipient.

In another aspect, the disclosure features a method for treating a neoplasia in a subject in need thereof, the method involving administering to the subject the lipid nanoparticle of any aspect of the disclosure, or embodiments thereof.

In any aspect of the disclosure, or embodiments thereof:
a) CDR1 contains the amino acid sequence NYAAG (SEQ ID NO: 478), CDR2 contains the amino acid sequence RISRSGGRTDYADSVKG (SEQ ID NO: 486), and CDR3 contains the amino acid sequence ATVWEFTDGADQYDY (SEQ ID NO: 498);
b) CDR1 contains the amino acid sequence SYTMG (SEQ ID NO: 479), CDR2 contains the amino acid sequence AISWSAGRTYYADSMKG (SEQ ID NO: 487), and CDR3 contains the amino acid sequence DPWTSDSDYDRLTMYDY (SEQ ID NO: 499);
c) CDR1 contains the amino acid sequence TYTMG (SEQ ID NO: 480), CDR2 contains the amino acid sequence AISWSAGRTYYADSMKG (SEQ ID NO: 487), and CDR3 contains the amino acid sequence DPWTSDSDYDRLTMYDY (SEQ ID NO: 499);
d) CDR1 contains the amino acid sequence SYTMG (SEQ ID NO: 479), CDR2 contains the amino acid sequence AISWSAGRTYYADSMKG (SEQ ID NO: 487), and CDR3 contains the amino acid sequence DPWTSDSDYDRLTMYDY (SEQ ID NO: 499);
e) CDR1 contains the amino acid sequence TYTMG (SEQ ID NO: 480), CDR2 contains the amino acid sequence AISWSAGRTYYADSMKG (SEQ ID NO: 487), and CDR3 contains the amino acid sequence DPWTSDSDYDRLTMYDY (SEQ ID NO: 499);
f) CDR1 contains the amino acid sequence TYTMG (SEQ ID NO: 480), CDR2 contains the amino acid sequence AISWSAGRTYYADSMKG (SEQ ID NO: 487), and CDR3 contains the amino acid sequence DPWTSDSDYDRLTMYDY (SEQ ID NO: 499);
g) CDR1 contains the amino acid sequence SYTMG (SEQ ID NO: 479), CDR2 contains the amino acid sequence AISWSAGRTYYADSMKG (SEQ ID NO: 487), and CDR3 contains the amino acid sequence DPWTSDSDYDRLTMYDY (SEQ ID NO: 499);
h) CDR1 contains the amino acid sequence TYTMG (SEQ ID NO: 480), CDR2 contains the amino acid sequence AISWSAGRTYYADSMKG (SEQ ID NO: 487), and CDR3 contains the amino acid sequence DPWTSDSDYDRLTMYDY (SEQ ID NO: 499);
i) CDR1 contains the amino acid sequence TYTMG (SEQ ID NO: 480), CDR2 contains the amino acid sequence AISWSAGRTYYADSMKG (SEQ ID NO: 487), and CDR3 contains the amino acid sequence DPWTSDSDYDRLTMYDY (SEQ ID NO: 499);
j) CDR1 contains the amino acid sequence SYAMG (SEQ ID NO: 481), CDR2 contains the amino acid sequence VISWSGGRTYYADSVKG (SEQ ID NO: 488), and CDR3 contains the amino acid sequence DPWTSDSDYERLTMYDY (SEQ ID NO: 500);
k) CDR1 contains the amino acid sequence SYAMG (SEQ ID NO: 481), CDR2 contains the amino acid sequence VISWSGGRTYYADSVKG (SEQ ID NO: 488), and CDR3 contains the amino acid sequence DPWTSDSDYERLTMYDY (SEQ ID NO: 500);
l) CDR1 contains the amino acid sequence SYAMG, CDR2 contains the amino acid sequence VISWSGGRTYYADSVKG (SEQ ID NO: 488), and CDR3 contains the amino acid sequence DPWTSDSDYERLTMYDY (SEQ ID NO: 500);
m) CDR1 contains the amino acid sequence SYAMG, CDR2 contains the amino acid sequence VISWSGGRTYYADSVKG (SEQ ID NO: 488), and CDR3 contains the amino acid sequence DPWTSDSDYERLTMYDY (SEQ ID NO: 500);
n) CDR1 contains the amino acid sequence TYNMG (SEQ ID NO: 482), CDR2 contains the amino acid sequence AIDLYGRATRYANSVKG (SEQ ID NO: 489), and CDR3 contains the amino acid sequence DTSLPLGVLTESQRLYGA (SEQ ID NO: 501);
o) CDR1 contains the amino acid sequence TYNMG (SEQ ID NO: 482), CDR2 contains the amino acid sequence AINLEGYATRYANSVKG (SEQ ID NO: 615), and CDR3 contains the amino acid sequence DTSLPLGVLTESQRLYGA (SEQ ID NO: 501);
p) CDR1 contains the amino acid sequence TYNMG (SEQ ID NO: 482), CDR2 contains the amino acid sequence AIDLYGRATRYANSVKG (SEQ ID NO: 489), and CDR3 contains the amino acid sequence DTSLPLGVLTESQRLYGA (SEQ ID NO: 501);
q) CDR1 contains the amino acid sequence TYNMG (SEQ ID NO: 482), CDR2 contains the amino acid sequence AIDLYGRATRYANSVRG (SEQ ID NO: 616), and CDR3 contains the amino acid sequence DTSLPLGVLTESQRLYGA (SEQ ID NO: 501);
r) CDR1 contains the amino acid sequence TYNMG (SEQ ID NO: 482), CDR2 contains the amino acid sequence AIDLYGRATRYANSVKG (SEQ ID NO: 489), and CDR3 contains the amino acid sequence DTSLPLGVLTESQRLYGA (SEQ ID NO: 501);
s) CDR1 contains the amino acid sequence TYNMG (SEQ ID NO: 482), CDR2 contains the amino acid sequence AIDLYGRATRYANSVKG (SEQ ID NO: 489), and CDR3 contains the amino acid sequence DTSLPLGVLTKSQRMYGA (SEQ ID NO: 502);
t) CDR1 contains the amino acid sequence AYAMG, CDR2 contains the amino acid sequence AINWNGDTALRWNGFATRYADSVKG (SEQ ID NO: 490), and CDR3 contains the amino acid sequence DTVVSGSYYLAARAEDYEY (SEQ ID NO: 503);
u) CDR1 contains the amino acid sequence AYAMG, CDR2 contains the amino acid sequence AINWNGDTALRWNGFATRYADSVKG (SEQ ID NO: 490), and CDR3 contains the amino acid sequence DTVVSGSYYLAARAEDYEY (SEQ ID NO: 503);
v) CDR1 contains the amino acid sequence AYAMG, CDR2 contains the amino acid sequence AINWNGDTALRWNGFATRYADSVKG (SEQ ID NO: 490), and CDR3 contains the amino acid sequence DTVVSGSYYLAARAEDYEY (SEQ ID NO: 503);
w) CDR1 contains the amino acid sequence AYAMG, CDR2 contains the amino acid sequence AINWNGDTALRWNGFATRYADSVKG (SEQ ID NO: 490), and CDR3 contains the amino acid sequence DTVVSGSYYLAARAEDYEY (SEQ ID NO: 503);
x) CDR1 contains the amino acid sequence SSGMG (SEQ ID NO: 484), CDR2 contains the amino acid sequence SMDWSGGSTYYGDSVKG (SEQ ID NO: 491), and CDR3 contains the amino acid sequence GTSGVAAVNLRGFFS (SEQ ID NO: 504);
y) CDR1 contains the amino acid sequence SSGMG (SEQ ID NO: 484), CDR2 contains the amino acid sequence SIDWSGGSTYYGDSVKG (SEQ ID NO: 492), and CDR3 contains the amino acid sequence GTSGVAAVNLRGFFS (SEQ ID NO: 504);
z) CDR1 contains the amino acid sequence SSGMG (SEQ ID NO: 484), CDR2 contains the amino acid sequence SMDWSGGSTYYGDSVKG (SEQ ID NO: 491), and CDR3 contains the amino acid sequence GTSGVAAVNLRGFFS (SEQ ID NO: 504);
aa) CDR1 contains the amino acid sequence SSGMG (SEQ ID NO: 484), CDR2 contains the amino acid sequence SIDWGGGSTYYGDSVKG (SEQ ID NO: 493), and CDR3 contains the amino acid sequence GTSGVAAVNLRGFFS (SEQ ID NO: 504);
ab) CDR1 contains the amino acid sequence SSGMG (SEQ ID NO: 484), CDR2 contains the amino acid sequence SIDWSGGSTYYGDSVKG (SEQ ID NO: 492), and CDR3 contains the amino acid sequence GTSGVAAVNLRGFFS (SEQ ID NO: 504);
ac) CDR1 contains the amino acid sequence SSGMG (SEQ ID NO: 484), CDR2 contains the amino acid sequence SIDWSGKSTYYGDSVKG (SEQ ID NO: 494), and CDR3 contains the amino acid sequence GTSGVAAVNLRGFFS (SEQ ID NO: 504);
ad) CDR1 contains the amino acid sequence SSGMG (SEQ ID NO: 484), CDR2 contains the amino acid sequence SINWSGGSAYYGDSVKG (SEQ ID NO: 495), and CDR3 contains the amino acid sequence GTSGVAAVNLRGFFS (SEQ ID NO: 504);
ae) CDR1 contains the amino acid sequence SSGMG (SEQ ID NO: 484), CDR2 contains the amino acid sequence SMDWSGGSTYYGDSVKG (SEQ ID NO: 491), and CDR3 contains the amino acid sequence GTSGVAAVNLRGFFS (SEQ ID NO: 504);
af) CDR1 contains the amino acid sequence SSGMG (SEQ ID NO: 484), CDR2 contains the amino acid sequence SMDWTGGSTYYGDSVKG (SEQ ID NO: 496), and CDR3 contains the amino acid sequence GTSGVAAVNLRGFFS (SEQ ID NO: 504);
ag) CDR1 contains the amino acid sequence VDATT (SEQ ID NO: 485), CDR2 contains the amino acid sequence IMDIGGVTEYADSVKG (SEQ ID NO): 497), and CDR3 contains the amino acid sequence RGL;
ah) CDR1 contains the amino acid sequence INVIG (SEQ ID NO: 505), CDR2 contains the amino acid sequence LVNSGGQTHYADSVKG (SEQ ID NO: 516), and CDR3 contains the amino acid sequence RYGIDNY (SEQ ID NO: 528);
ai) CDR1 contains the amino acid sequence SSFMS (SEQ ID NO: 506), CDR2 contains the amino acid sequence TIYSDGSTYYADSVKG (SEQ ID NO: 517), and CDR3 contains the amino acid sequence VTGSI (SEQ ID NO: 529);
aj) CDR1 contains the amino acid sequence SSFMS (SEQ ID NO: 506), CDR2 contains the amino acid sequence TIYSDGSTYYADSMKG (SEQ ID NO: 518), and CDR3 contains the amino acid sequence VTGSI (SEQ ID NO: 529);
ak) CDR1 contains the amino acid sequence TNVMG (SEQ ID NO: 507), CDR2 contains the amino acid sequence LIRGGGSTHYADSVKG (SEQ ID NO: 519), and CDR3 contains the amino acid sequence WLGSPGAMSDY (SEQ ID NO: 530);
al) CDR1 contains the amino acid sequence TNNMG (SEQ ID NO: 508), CDR2 contains the amino acid sequence LIRTGGSTHVADSMKG (SEQ ID NO: 520), and CDR3 contains the amino acid sequence WTGSPGALSDY (SEQ ID NO: 531);
am) CDR1 contains the amino acid sequence TNNMA (SEQ ID NO: 509), CDR2 contains the amino acid sequence LIRTGGSTHVADSMKG (SEQ ID NO: 520), and CDR3 contains the amino acid sequence WTGSPGALSDY (SEQ ID NO: 531);
an) CDR1 contains the amino acid sequence RVAMN (SEQ ID NO: 510), CDR2 contains the amino acid sequence TISSDGSRTNYAHSVKG (SEQ ID NO: 522), and CDR3 contains the amino acid sequence PGNS (SEQ ID NO: 532);
ao) CDR1 contains the amino acid sequence RVGMN (SEQ ID NO: 511), CDR2 contains the amino acid sequence SISSDGSRTNYAHFVKG (SEQ ID NO: 523), and CDR3 contains the amino acid sequence PGNS (SEQ ID NO: 532);
ap) CDR1 contains the amino acid sequence FVGWG (SEQ ID NO: 512), CDR2 contains the amino acid sequence QISTGGLTNYADSVKG (SEQ ID NO: 524), and CDR3 contains the amino acid sequence PGHP (SEQ ID NO: 533);
aq) CDR1 contains the amino acid sequence FIGWG (SEQ ID NO: 513), CDR2 contains the amino acid sequence QINTGGLTDYADSVKG (SEQ ID NO: 525), and CDR3 contains the amino acid sequence PGHS (SEQ ID NO: 534);
ar) CDR1 contains the amino acid sequence MYSMS (SEQ ID NO: 514), CDR2 contains the amino acid sequence SISTGARDTAYADSVKG (SEQ ID NO: 526), and CDR3 contains the amino acid sequence GDLRYGPDGYDY (SEQ ID NO: 535);
as) CDR1 contains the amino acid sequence MYSMS (SEQ ID NO: 514), CDR2 contains the amino acid sequence SISTGARDTAYADSVKG (SEQ ID NO: 526), and CDR3 contains the amino acid sequence GDLRYGPDGYDY (SEQ ID NO: 535);
at) CDR1 contains the amino acid sequence MYSMS (SEQ ID NO: 514), CDR2 contains the amino acid sequence SISTGARDTSYADSVKG (SEQ ID NO: 618), and CDR3 contains the amino acid sequence GDLRYGPDGYDY (SEQ ID NO: 535); or
au) CDR1 contains the amino acid sequence TYGMG (SEQ ID NO: 515), CDR2 contains the amino acid sequence VITGSGVGTQYADSVKD (SEQ ID NO: 527), and CDR3 contains the amino acid sequence GHRPGWAVIRADAYEY (SEQ ID NO: 536).

In any aspect of the disclosure, or embodiments thereof:
a) FR1 contains the following amino acid sequence: X₁X₂QLX₃ESGGX₄VQX₅GX₆SX₇RLX₈CX₉X₁₀SGX₁₁X₁₂X₁₃X₁₄ (SEQ ID NO: 604), where X₁ is E or Q; X₂ is L or V; X₃ is V or Q; X₄ is L or S; X₅ is P or A; X₆ is A, E, or G; X₇ is L, R, or V; X₈ is A or S; X₉ is A or V; X₁₀ is A, T, or V; X₁₁ is A, D, F, G, I, P, R, or S; X₁₂ is A, D, I, N, P, S, T, or V; X₁₃ is A, F, null, S, or V; and X₁₄ is I, L, null, or S;
b) FR2 contains the amino acid sequence: WX₁₅RX₁₆APGX₁₇X₁₈X₁₉X₂₀X₂₁VX₂₂ (SEQ ID NO: 605), where X₁₅ is F, V, or Y; X₁₆ is H or Q; X₁₇ is E or K; X₁₈ A, D, E, G, R, or Q; X₁₉ is L or R; X₂₀ is D or E; X₂₁ is F, L, V, or W; and X₂₂ is A or S;
c) FR3 contains the amino acid sequence: RFX₂₃X₂₄SRX₂₅X₂₆X₂₇X₂₈X₂₉X₃₀X₃₁X₃₂LX₃₃MX₃₄X₃₅LX₃₆X₃₇EDTAX₃₈YYCX₃₉X₄₀ (SEQ ID NO: 606), where X₂₃ is A, I, or T; X₂₄ is I or V; X₂₅ is D, E, or V; X₂₆ is H, I, or N; X₂₇ is A or T; X₂₈ is D or K; X₂₉ is K, M, N, R, S, or T; X₃₀ is A, M, or T; X₃₁ is A, L, or V; X₃₂ is F, H, N, or Y; X₃₃ is H or Q; X₃₄ is N or S; X₃₅ is G, N, S, or T; X₃₆ is K or R; X₃₇ is A, F, L, P, or V; X₃₈ is V or E; X₃₉ is A, H, N, or V; and X₄₀ is A, E, F, G, I, N, R, T, or V; and/or
d) FR4 contains the amino acid sequence: X₄₀GX₄₁GTX₄₂VX₄₃VX₄₄S (SEQ ID NO: 607), where X₄₀ is R or W; X₄₁ is Q, E, or P; X₄₂ is L or Q; X₄₃ is S or T; and X₄₄ is S or V.

In any aspect of the disclosure, or embodiments thereof:
a) FR1 contains an amino acid sequence selected from one or more of:

```
(SEQ ID NO: 537)
QVQLVESGGGLVQPGGSLRLSCAASGRTF,
  (SEQ ID NO: 538)
EVQLVESGGGLVQAGGSLRLSCAASGRTFG,
  (SEQ ID NO: 543)
QVQLQESGGGLVQAGGSLRLSCAASGRTFG,
  (SEQ ID NO: 619)
QVQLVESGGGLVQAGGSLRLSCAASGRTFG,
  (SEQ ID NO: 544)
EVQLVESGGGLVQAGGSLRLSCAASGGTVS,
(SEQ ID NO: 545)
EVQLVESGGGLVQAGGSRRLSCAASGGTVS,
  (SEQ ID NO: 546)
QVQLVESGGGLVQAGGSLRLSCAASGGTVS,
(SEQ ID NO: 548)
EVQLVESGGGLVQAGASLRLSCAASGRT,
  (SEQ ID NO: 549)
QVQLQESGGGLVQAGASLRLSCAASGRA,
(SEQ ID NO: 550)
QVQLQESGGGLVQAGASLRLSCAASGRT,
(SEQ ID NO: 551)
QVQLVESGGGLVQAGASLRLSCAASGRT,
(SEQ ID NO: 554)
QVQLQESGGGSVQAGGSLRLSCAASGRAFS,
(SEQ ID NO: 559)
EVQLVESGGGLVQAGGSLRLSCAASGPAFS,
(SEQ ID NO: 560)
QVQLQESGGGLVQAGGSLRLSCAASGPAFS,
(SEQ ID NO: 561)
QVQLVESGGGLVQAGGSLRLACAASGAAFS,
(SEQ ID NO: 562)
QVQLVESGGGLVQAGGSLRLSCAASGPAFS,
(SEQ ID NO: 569)
QLQLVESGGGLVQPGGSLRLSCAASGSDFL,
(SEQ ID NO: 573)
QVQLQESGGGLVQAGGSLRLSCATSGITSS,
(SEQ ID NO: 577)
EVQLVESGGGLVQPGGSLRLSCAASGFPFS,
(SEQ ID NO: 578)
QVQLVESGGGLVQPGGSLRLSCAASGENFS,
(SEQ ID NO: 584)
QVQLVESGGGLVQPGGSVRLSCATSGSIFS,
(SEQ ID NO: 587)
EVQLVESGGGLVQPGGSLRLSCAASGSVVS,
(SEQ ID NO: 588)
QVQLVESGGGLVQPGGSLRLSCAASGSDAS,
(SEQ ID NO: 590)
QLQLVESGGGLVQPGESLRLSCAASGFSFS,
(SEQ ID NO: 594)
QLQLVESGGGLVQPGESLRLSCVVSGDIFS,
(SEQ ID NO: 597)
QVQLVESGGGLVQPGESLRLSCVVSGDIFS,
(SEQ ID NO: 599)
QVQLVESGGGLVQPGGSLRLSCAASGFTFS,
and
(SEQ ID NO: 602)
QVQLVESGGGLVQPGGSLRLSCVASGGTFS;
```

b) FR2 contains an amino acid sequence selected from one or more of:

```
(SEQ ID NO: 539)
WFRQAPGKEREFVA,
  (SEQ ID NO: 620)
WFRQAPGKGREFVA,
  (SEQ ID NO: 621)
WFRQAPGREREFVA,
  (SEQ ID NO: 552)
WFRHAPGKDREFVA,
(SEQ ID NO: 553)
WFRHAPGEDREFVA,
(SEQ ID NO: 563)
WFRQAPGKARDFVA,
  (SEQ ID NO: 567)
WFRQAPGKAREFVA,
  (SEQ ID NO: 570)
WFRQAPGNQREFVA,
  (SEQ ID NO: 574)
WYRQAPGKQRELVA,
  (SEQ ID NO: 579)
WVRQAPGKGLEWVS,
  (SEQ ID NO: 580)
WVRQAPGKEVEWVS,
  (SEQ ID NO: 585)
WYRQAPGKEREFVA,
  (SEQ ID NO: 591)
WYRQAPGKERELVA,
  (SEQ ID NO: 595)
WYRQAPGKQREVVA,
and
  (SEQ ID NO: 600)
WVRQAPGKRLEWVS;
```

c) FR3 contains an amino acid sequence selected from one or more of:

(SEQ ID NO: 540)
RFTISRDNAKSTVYLQMNSLRPEDTAVYYCAE,
(SEQ ID NO: 541)
RFTISRDNAKNTVYLQMNSLKPEDTAVYYCAA,
(SEQ ID NO: 547)
RFTISRDNAKNTVNLQMNSLKPEDTAVYYCAA,
(SEQ ID NO: 555)
RFIISRVNAKNTVNLQMNSLKPEDTAVYYCAA,
(SEQ ID NO: 556)
RFTISRVIAKNTVNLQMNSLKPEDTAVYYCAA,
(SEQ ID NO: 558)
RFTISRVNAKNTVNLQMNSLKPEDTAVYYCAA,
(SEQ ID NO: 564)
RFTVSRDNAKNTVHLQMNSLRPEDTAVYYCAR,
(SEQ ID NO: 565)
RFTVSRDNAKNAVHLQMNSLRPEDTAVYYCAR,
(SEQ ID NO: 568)
RFTVSRDNAKMTVHLQMNSLRPEDTAVYYCAR,
(SEQ ID NO: 571)
RFTISRDHTKNTVYLQMNSLKVEDTAVYYCNT,
(SEQ ID NO: 575)
RFTISRDNAKNTVFLQMNSLKPEDTAEYYCHG,
(SEQ ID NO: 581)
RFTISRDNAKKTAYLQMNSLKAEDTAVYYCAT,
(SEQ ID NO: 582)
RFTISRDNAKNTVYLQMSNLKAEDTAVYYCAT,
(SEQ ID NO: 586)
RFIISRENAKTTVYLQMNGLKPEDTAVYYCVI,
(SEQ ID NO: 589)
RFTISRENAKNTVYLQMNSLKPEDTAVYYCVI,
(SEQ ID NO: 592)
RFTISRENAKNMVYLQMNSLKLEDTAVYYCNV,
(SEQ ID NO: 593)
RFTISRDNVKNMVYLQMNSLKLEDTAVYYCNV,
(SEQ ID NO: 596)
RFAISRDNAKRTVYLQMNSLKFEDTAVYYCNV,
(SEQ ID NO: 598)
RFTISRDNAKRTVYLQMNSLKFEDTAVYYCNF,
(SEQ ID NO: 601)
RFTISRDNADNTLYLHMNNLKPEDTAVYYCAN,
and
(SEQ ID NO: 603)
RFTISRENAKNTVYLQMNTLKLEDTAVYYCVS;

and/or
d) FR4 contains an amino acid sequence selected from one or more of: WGQGTQVTVSS (SEQ ID NO: 542), WGQGTQVSVSS (SEQ ID NO: 557), WGPGTQVTVSS (SEQ ID NO: 566), WGQGTLVTVSS (SEQ ID NO: 572), WGEGTQVTVSS (SEQ ID NO: 576), RGQGTQVTVSS (SEQ ID NO: 583), and RGQGTQVTVVS (SEQ ID NO: 622).

In any aspect of the disclosure, or embodiments thereof:
a) FR1 contains the amino acid sequence QVQLVESGGGLVQPGGSLRLSCAASGRTFI (SEQ ID NO: 537), FR2 contains the amino acid sequence WFRQAPGKEREFVA (SEQ ID NO: 539), FR3 contains the amino acid sequence RFTISRDNAKSTVYLQMNSLRPEDTAVYYCAE (SEQ ID NO: 540), and FR4 contains the amino acid sequence WGQGTQVTVSS (SEQ ID NO: 542);
b) FR1 contains the amino acid sequence EVOLVESGGGLVQAGGSLRLSCAASGRTFG (SEQ ID NO: 538), FR2 contains the amino acid sequence WFRQAPGKEREFVA (SEQ ID NO: 539), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
c) FR1 contains the amino acid sequence EVOLVESGGGLVQAGGSLRLSCAASGRTFG (SEQ ID NO: 538), FR2 contains the amino acid sequence WFRQAPGKEREFVA (SEQ ID NO: 539), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
d) FR1 contains the amino acid sequence QVQLQESGGGLVQAGGSLRLSCAASGRTFG (SEQ ID NO: 543), FR2 contains the amino acid sequence WFRQAPGKEREFVA (SEQ ID NO: 539), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
e) FR1 contains the amino acid sequence QVQLQESGGGLVQAGGSLRLSCAASGRTFG (SEQ ID NO: 543), FR2 contains the amino acid sequence WFRQAPGKEREFVA (SEQ ID NO: 539), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
f) FR1 contains the amino acid sequence QVQLQESGGGLVQAGGSLRLSCAASGRTFG (SEQ ID NO: 543), FR2 contains the amino acid sequence WFRQAPGKGREFVA (SEQ ID NO: 620), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
g) FR1 contains the amino acid sequence QVQLVESGGGLVQAGGSLRLSCAASGRTFG (SEQ ID NO: 619), FR2 contains the amino acid sequence WFRQAPGKEREFVA (SEQ ID NO: 539), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
h) FR1 contains the amino acid sequence QVQLVESGGGLVQAGGSLRLSCAASGRTFG (SEQ ID NO: 619), FR2 contains the amino acid sequence WFRQAPGKEREFVA (SEQ ID NO: 539), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
i) FR1 contains the amino acid sequence QVQLVESGGGLVQAGGSLRLSCAASGRTFG (SEQ ID NO: 619), FR2 contains the amino acid sequence WFRQAPGREREFVA (SEQ ID NO: 621), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
j) FR1 contains the amino acid sequence EVOLVESGGGLVQAGGSLRLSCAASGGTVS (SEQ ID NO: 544), FR2 contains the amino acid sequence WFRQAPGKEREFVA (SEQ ID NO: 539), FR3 contains the amino acid sequence RFTISRDNAKNTVNLQMNSLKPEDTAVYYCAA (SEQ ID NO: 547), and FR4 contains the amino acid sequence WGQGTQVTVSS (SEQ ID NO: 542);
k) FR1 contains the amino acid sequence EVOLVESGGGLVQAGGSRRLSCAASGGTVS (SEQ ID NO: 545), FR2 contains the amino acid sequence WFRQAPGKEREFVA (SEQ ID NO: 539), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
l) FR1 contains the amino acid sequence QVQLVESGGGLVQAGGSLRLSCAASGGTVS (SEQ ID NO: 546), FR2 contains the amino acid sequence WFRQAPGKEREFVA (SEQ ID NO: 539), FR3 contains the amino acid sequence RFTISRDNAKNTVNLQMNSLKPEDTAVYYCAA (SEQ ID NO: 547), and FR4 contains the amino acid sequence WGQGTQVTVSS (SEQ ID NO: 542);
m) FR1 contains the amino acid sequence QVQLVESGGGLVQAGGSLRLSCAASGGTVS (SEQ ID NO: 546), FR2 contains the amino acid sequence WFRQAPGKEREFVA (SEQ ID NO: 539), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
n) FR1 contains the amino acid sequence EVOLVESGGGLVQAGASLRLSCAASGRT (SEQ ID NO: 548), FR2 contains the amino acid sequence WFRHAPGKDREFVA (SEQ ID NO: 552), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
o) FR1 contains the amino acid sequence QVQLQESGGGLVQAGASLRLSCAASGRA (SEQ ID NO: 549), FR2 contains the amino acid sequence WFRHAPGEDREFVA (SEQ ID NO: 553), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
p) FR1 contains the amino acid sequence QVQLQESGGGLVQAGASLRLSCAASGRT (SEQ ID NO: 550), FR2 contains the amino acid sequence WFRHAPGKDREFVA (SEQ ID NO: 552), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
q) FR1 contains the amino acid sequence QVQLQESGGGLVQAGASLRLSCAASGRT (SEQ ID NO: 550), FR2 contains the amino acid sequence WFRHAPGKDREFVA (SEQ ID NO: 552), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
r) FR1 contains the amino acid sequence QVQLVESGGGLVQAGASLRLSCAASGRT (SEQ ID NO: 551), FR2 contains the amino acid sequence WFRHAPGKDREFVA (SEQ ID NO: 552), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
s) FR1 contains the amino acid sequence QVQLVESGGGLVQAGASLRLSCAASGRT (SEQ ID NO: 551), FR2 contains the amino acid sequence WFRHAPGKDREFVA (SEQ ID NO: 552), FR3 contains the amino acid sequence RFTISRDNAKNTVYLQMNSLKPEDTAVYYCAA (SEQ ID NO: 541), and FR4 contains the amino acid sequence WGQGTQVTVSS (SEQ ID NO: 542);
t) FR1 contains the amino acid sequence QVQLQESGGGSVQAGGSLRLSCAASGRAFS (SEQ ID NO: 554), FR2 contains the amino acid sequence WFRQAPGKEREFVA (SEQ ID NO: 539), FR3 contains the amino acid sequence RFIISRVNAKNTVNLQMNSLKPEDTAVYYCAA (SEQ ID NO: 555), and FR4 contains the amino acid sequence WGQGTQVSVSS (SEQ ID NO: 557);
u) FR1 contains the amino acid sequence QVQLQESGGGSVQAGGSLRLSCAASGRAFS (SEQ ID NO: 554), FR2 contains the amino acid sequence WFRQAPGKEREFVA (SEQ ID NO: 539), FR3 contains the amino acid sequence RFTISRVIAKNTVNLQMNSLKPEDTAVYYCAA (SEQ ID NO: 556), and FR4 contains the amino acid sequence WGQGTQVTVSS (SEQ ID NO: 542);
v) FR1 contains the amino acid sequence QVQLQESGGGSVQAGGSLRLSCAASGRAFS (SEQ ID NO: 554), FR2 contains the amino acid sequence WFRQAPGKEREFVA (SEQ ID NO: 539), FR3 contains the amino acid sequence RFTISRVNAKNTVNLQMNSLKPEDTAVYYCAA (SEQ ID NO: 558), and FR4 contains the amino acid sequence WGQGTQVSVSS (SEQ ID NO: 557);
w) FR1 contains the amino acid sequence QVQLQESGGGSVQAGGSLRLSCAASGRAFS (SEQ ID NO: 554), FR2 contains the amino acid sequence WFRQAPGKEREFVA (SEQ ID NO: 539), FR3 contains the amino acid sequence RFTISRVNAKNTVNLQMNSLKPEDTAVYYCAA (SEQ ID NO: 558), and FR4 contains the amino acid sequence WGQGTQVTVSS (SEQ ID NO: 542);
x) FR1 contains the amino acid sequence EVOLVESGGGLVQAGGSLRLSCAASGPAFS (SEQ ID NO: 559), FR2 contains the amino acid sequence WFRQAPGKARDFVA (SEQ ID NO: 563), FR3 contains the amino acid sequence RFTVSRDNAKNTVHLQMNSLRPEDTAVYYCAR (SEQ ID NO: 564), and FR4 contains the amino acid sequence WGPGTQVTVSS (SEQ ID NO: 566);
y) FR1 contains the amino acid sequence QVQLQESGGGLVQAGGSLRLSCAASGPAFS (SEQ ID NO: 560), FR2 contains the amino acid sequence WFRQAPGKARDFVA (SEQ ID NO: 563), FR3 contains the amino acid sequence RFTVSRDNAKNTVHLQMNSLRPEDTAVYYCAR (SEQ ID NO: 564), and FR4 contains the amino acid sequence WGPGTQVTVSS (SEQ ID NO: 566);
z) FR1 contains the amino acid sequence QVQLVESGGGLVQAGGSLRLACAASGAAFS (SEQ ID NO: 561), FR2 contains the amino acid sequence WFRQAPGKARDFVA (SEQ ID NO: 563), FR3 contains the amino acid sequence RFTVSRDNAKNTVHLQMNSLRPEDTAVYYCAR (SEQ ID NO: 564), and FR4 contains the amino acid sequence WGPGTQVTVSS (SEQ ID NO: 566);
aa) FR1 contains the amino acid sequence QVQLVESGGGLVQAGGSLRLSCAASGPAFS (SEQ ID NO: 562), FR2 contains the amino acid sequence WFRQAPGKARDFVA (SEQ ID NO: 563), FR3 contains the amino acid sequence RFTVSRDNAKNAVHLQMNSLRPEDTAVYYCAR (SEQ ID NO: 565), and FR4 contains the amino acid sequence WGPGTQVTVSS (SEQ ID NO: 566);
ab) FR1 contains the amino acid sequence QVQLVESGGGLVQAGGSLRLSCAASGPAFS (SEQ ID NO: 562), FR2 contains the amino acid sequence WFRQAPGKARDFVA (SEQ ID NO: 563), FR3 contains the amino acid sequence RFTVSRDNAKNTVHLQMNSLRPEDTAVYYCAR (SEQ ID NO: 564), and FR4 contains the amino acid sequence WGPGTQVTVSS (SEQ ID NO: 566);
ac) FR1 contains the amino acid sequence QVQLVESGGGLVQAGGSLRLSCAASGPAFS (SEQ ID NO: 562), FR2 contains the amino acid sequence WFRQAPGKARDFVA (SEQ ID NO: 563), FR3 contains the amino acid sequence RFTVSRDNAKNTVHLQMNSLRPEDTAVYYCAR (SEQ ID NO: 564), and FR4 contains the amino acid sequence WGPGTQVTVSS (SEQ ID NO: 566);
ad) FR1 contains the amino acid sequence QVQLVESGGGLVQAGGSLRLSCAASGPAFS (SEQ ID NO: 562), FR2 contains the amino acid sequence WFRQAPGKARDFVA (SEQ ID NO: 563), FR3 contains the amino acid sequence RFTVSRDNAKNTVHLQMNSLRPEDTAVYYCAR (SEQ ID NO: 564), and FR4 contains the amino acid sequence WGPGTQVTVSS (SEQ ID NO: 566);
ae) FR1 contains the amino acid sequence QVQLVESGGGLVQAGGSLRLSCAASGPAFS (SEQ ID NO: 562), FR2 contains the amino acid sequence WFRQAPGKARDFVA (SEQ ID NO: 563), FR3 contains the amino acid sequence RFTVSRDNAKNTVHLQMNSLRPEDTAVYYCAR (SEQ ID NO: 564), and FR4 contains the amino acid sequence WGPGTQVTVSS (SEQ ID NO: 566);
af) FR1 contains the amino acid sequence QVQLVESGGGLVQAGGSLRLSCAASGPAFS (SEQ ID NO: 562), FR2 contains the amino acid sequence WFRQAPGKAREFVA (SEQ ID NO: 567), FR3 contains the amino acid sequence RFTVSRDNAKMTVHLQMNSLRPEDTAVYYCAR (SEQ ID NO: 568), and FR4 contains the amino acid sequence WGPGTQVTVSS (SEQ ID NO: 566);
ag) FR1 contains the amino acid sequence QLQLVESGGGLVQPGGSLRLSCAASGSDFL (SEQ ID NO: 569), FR2 contains the amino acid sequence WFRQAPGNQREFVA (SEQ ID NO: 570), FR3 contains the amino acid sequence RFTISRDHTKNTVYLQMNSLKVEDTAVYYCNT (SEQ ID NO: 571), and FR4 contains the amino acid sequence WGQGTLVTVSS (SEQ ID NO: 572);
ah) FR1 contains the amino acid sequence QVQLQESGGGLVQAGGSLRLSCATSGITSS (SEQ ID NO: 573), FR2 contains the amino acid sequence WYRQAPGKQRELVA (SEQ ID NO: 574), FR3 contains the amino acid sequence RFTISRDNAKNTVFLQMNSLKPEDTAEYYCHG (SEQ ID NO: 575), and FR4 contains the amino acid sequence WGEGTQVTVSS (SEQ ID NO: 576);
ai) FR1 contains the amino acid sequence EVOLVESGGGLVQPGGSLRLSCAASGFPFS (SEQ ID NO: 577), FR2 contains the amino acid sequence WVRQAPGKGLEWVS (SEQ ID NO: 579), FR3 contains the amino acid sequence RFTISRDNAKKTAYLQMNSLKAEDTAVYYCAT (SEQ ID NO: 581), and FR4 contains the amino acid sequence RGQGTQVTVSS (SEQ ID NO: 583);
aj) FR1 contains the amino acid sequence QVQLVESGGGLVQPGGSLRLSCAASGFNFS (SEQ ID NO: 578), FR2 contains the amino acid sequence WVRQAPGKEVEWVS (SEQ ID NO: 580), FR3 contains the amino acid sequence RFTISRDNAKNTVYLQMSNLKAEDTAVYYCAT (SEQ ID NO: 582), and FR4 contains the amino acid sequence RGQGTQVTVSS (SEQ ID NO: 583);
ak) FR1 contains the amino acid sequence QVQLVESGGGLVQPGGSVRLSCATSGSIFS (SEQ ID NO: 584), FR2 contains the amino acid sequence WYRQAPGKEREFVA (SEQ ID NO: 585), FR3 contains the amino acid sequence RFIISRENAKTTVYLQMNGLKPEDTAVYYCVI (SEQ ID NO: 586), and FR4 contains the amino acid sequence WGQGTQVTVSS (SEQ ID NO: 542);
al) FR1 contains the amino acid sequence EVOLVESGGGLVQPGGSLRLSCAASGSVVS (SEQ ID NO: 587), FR2 contains the amino acid sequence WYRQAPGKEREFVA (SEQ ID NO: 585), FR3 contains the amino acid sequence RFTISRENAKNTVYLQMNSLKPEDTAVYYCVI (SEQ ID NO: 589), and FR4 contains the amino acid sequence WGQGTQVTVSS (SEQ ID NO: 542);
am) FR1 contains the amino acid sequence QVQLVESGGGLVQPGGSLRLSCAASGSDAS (SEQ ID NO: 588), FR2 contains the amino acid sequence WYRQAPGKEREFVA (SEQ ID NO: 585), FR3 contains the amino acid sequence RFTISRENAKNTVYLQMNSLKPEDTAVYYCVI (SEQ ID NO: 589), and FR4 contains the amino acid sequence WGQGTQVTVSS (SEQ ID NO: 542);
an) FR1 contains the amino acid sequence QLQLVESGGGLVQPGESLRLSCAASGFSFS (SEQ ID NO: 590), FR2 contains the amino acid sequence WYRQAPGKERELVA (SEQ ID NO: 591), FR3 contains the amino acid sequence RFTISRENAKNMVYLQMNSLKLEDTAVYYCNV (SEQ ID NO: 592), and FR4 contains the amino acid sequence WGQGTQVTVSS (SEQ ID NO: 542);
ao) FR1 contains the amino acid sequence QLQLVESGGGLVQPGESLRLSCAASGFSFS (SEQ ID NO: 590), FR2 contains the amino acid sequence WYRQAPGKERELVA (SEQ ID NO: 591), FR3 contains the amino acid sequence RFTISRDNVKNMVYLQMNSLKLEDTAVYYCNV (SEQ ID NO: 593), and FR4 contains the amino acid sequence WGQGTQVTVSS (SEQ ID NO: 542);
ap) FR1 contains the amino acid sequence QLQLVESGGGLVQPGESLRLSCVVSGDIFS (SEQ ID NO: 594), FR2 contains the amino acid sequence WYRQAPGKQREVVA (SEQ ID NO: 595), FR3 contains the amino acid sequence RFAISRDNAKRTVYLQMNSLKFEDTAVYYCNV (SEQ ID NO: 596), and FR4 contains the amino acid sequence WGQGTQVTVSS (SEQ ID NO: 542);
aq) FR1 contains the amino acid sequence QVQLVESGGGLVQPGESLRLSCVVSGDIFS (SEQ ID NO: 597), FR2 contains the amino acid sequence WYRQAPGKQRELVA (SEQ ID NO: 574), FR3 contains the amino acid sequence RFTISRDNAKRTVYLQMNSLKFEDTAVYYCNF (SEQ ID NO: 598), and FR4 contains the amino acid sequence WGQGTQVTVSS (SEQ ID NO: 542);
ar) FR1 contains the amino acid sequence QVQLVESGGGLVQPGGSLRLSCAASGFTFS (SEQ ID NO: 599), FR2 contains the amino acid sequence WVRQAPGKRLEWVS (SEQ ID NO: 600), FR3 contains the amino acid sequence RFTISRDNADNTLYLHMNNLKPEDTAVYYCAN (SEQ ID NO: 601), and FR4 contains the amino acid sequence RGQGTQVTVSS (SEQ ID NO: 583);
as) FR1 contains the amino acid sequence QVQLVESGGGLVQPGGSLRLSCAASGFTFS (SEQ ID NO: 599), FR2 contains the amino acid sequence WVRQAPGKRLEWVS (SEQ ID NO: 600), FR3 contains the amino acid sequence RFTISRDNADNTLYLHMNNLKPEDTAVYYCAN (SEQ ID NO: 601), and FR4 contains the amino acid sequence RGQGTQVTVVS (SEQ ID NO: 622);
at) FR1 contains the amino acid sequence QVQLVESGGGLVQPGGSLRLSCAASGFTFS (SEQ ID NO: 599), FR2 contains the amino acid sequence WVRQAPGKRLEWVS (SEQ ID NO: 600), FR3 contains the amino acid sequence RFTISRDNADNTLYLHMNNLKPEDTAVYYCAN (SEQ ID NO: 601), and FR4 contains the amino acid sequence RGQGTQVTVSS (SEQ ID NO: 583); or
au) FR1 contains the amino acid sequence QVQLVESGGGLVQPGGSLRLSCVASGGTFS, FR2 contains the amino acid sequence WFRQAPGKEREFVA (SEQ ID NO: 539), FR3 contains the amino acid sequence RFTISRENAKNTVYLQMNTLKLEDTAVYYCVS (SEQ ID NO: 603), and FR4 contains the amino acid sequence WGQGTQVTVSS (SEQ ID NO: 542).

In any aspect of the disclosure, or embodiments thereof, the VHH antibody contains an amino acid sequence having at least 85% sequence identity to an amino acid sequence selected from one or more of:

```
  (SEQ ID NO: 431)
QVQLVESGGGLVQPGGSLRLSCAASGRTFINYAAGWFRQAPGKEREFVARISRSGGRTDYADSV
KGRFTISRDNAKSTVYLQMNSLRPEDTAVYYCAEATVWEFTDGADQYDYWGQGTQVTVSS;
(SEQ ID NO: 432)
EVQLVESGGGLVQAGGSLRLSCAASGRTFGSYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 433)
EVQLVESGGGLVQAGGSLRLSCAASGRTFGTYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 434)
QVQLQESGGGLVQAGGSLRLSCAASGRTFGSYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 435)
QVQLQESGGGLVQAGGSLRLSCAASGRTFGTYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 436)
QVQLQESGGGLVQAGGSLRLSCAASGRTFGTYTMGWFRQAPGKGREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 437)
QVQLVESGGGLVQAGGSLRLSCAASGRTFGSYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 438)
QVQLVESGGGLVQAGGSLRLSCAASGRTFGTYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 439)
QVQLVESGGGLVQAGGSLRLSCAASGRTFGTYTMGWFRQAPGREREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 440)
EVQLVESGGGLVQAGGSLRLSCAASGGTVSSYAMGWFRQAPGKEREFVAVISWSGGRTYYADSV
KGRFTISRDNAKNTVNLQMNSLKPEDTAVYYCAADPWTSDSDYERLTMYDYWGQGTQVTVSS;
(SEQ ID NO: 441)
EVQLVESGGGLVQAGGSRRLSCAASGGTVSSYAMGWFRQAPGKEREFVAVISWSGGRTYYADSV
KGRFTISRDNAKNTVYLQMNSLKPEDTAVYYCAADPWTSDSDYERLTMYDYWGQGTQVTVSS;
(SEQ ID NO: 442)
QVQLVESGGGLVQAGGSLRLSCAASGGTVSSYAMGWFRQAPGKEREFVAVISWSGGRTYYADSV
KGRFTISRDNAKNTVNLQMNSLKPEDTAVYYCAADPWTSDSDYERLTMYDYWGQGTQVTVSS;
(SEQ ID NO: 443)
QVQLVESGGGLVQAGGSLRLSCAASGGTVSSYAMGWFRQAPGKEREFVAVISWSGGRTYYADSV
KGRFTISRDNAKNTVYLQMNSLKPEDTAVYYCAADPWTSDSDYERLTMYDYWGQGTQVTVSS;
(SEQ ID NO: 444)
EVQLVESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTESQRLYGAWGQGTQVTVSS;
(SEQ ID NO: 445)
QVQLQESGGGLVQAGASLRLSCAASGRATYNMGWFRHAPGEDREFVAAINLEGYATRYANSVKG
RFTISRDNAKNTVYLQMNSLKPEDTAVYYCAADTSLPLGVLTESQRLYGAWGQGTQVTVSS;
(SEQ ID NO: 446)
QVQLQESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTESQRLYGAWGQGTQVTVSS;
(SEQ ID NO: 447)
QVQLQESGGGLVQAGASLRLSCAASGRTTYNMGWFRHAPGKDREFVAAIDLYGRATRYANSVRG
RFTISRDNAKNTVYLQMNSLKPEDTAVYYCAADTSLPLGVLTESQRLYGAWGQGTQVTVSS;
(SEQ ID NO: 448)
QVQLVESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTESQRLYGAWGQGTQVTVSS;
(SEQ ID NO: 449)
QVQLVESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTKSQRMYGAWGQGTQVTVSS;
  (SEQ ID NO: 450)
QVQLQESGGGSVQAGGSLRLSCAASGRAFSAYAMGWFRQAPGKEREFVAAINWNGDTALRWNGF
ATRYADSVKGRFIISRVNAKNTVNLQMNSLKPEDTAVYYCAADTVVSGSYYLAARAEDYEYWGQ
GTQVSVSS;
  (SEQ ID NO: 451)
QVQLQESGGGSVQAGGSLRLSCAASGRAFSAYAMGWFRQAPGKEREFVAAINWNGDTALRWNGF
ATRYADSVKGRFTISRVIAKNTVNLQMNSLKPEDTAVYYCAADTVVSGSYYLAARAEDYEYWGQ
GTQVTVSS;
  (SEQ ID NO: 452)
QVQLQESGGGSVQAGGSLRLSCAASGRAFSAYAMGWFRQAPGKEREFVAAINWNGDTALRWNGF
ATRYADSVKGRFTISRVNAKNTVNLQMNSLKPEDTAVYYCAADTVVSGSYYLAARAEDYEYWGQ
GTQVSVSS;
  (SEQ ID NO: 453)
QVQLQESGGGSVQAGGSLRLSCAASGRAFSAYAMGWFRQAPGKEREFVAAINWNGDTALRWNGF
ATRYADSVKGRFTISRVNAKNTVNLQMNSLKPEDTAVYYCAADTVVSGSYYLAARAEDYEYWGQ
GTQVTVSS;
  (SEQ ID NO: 454)
EVQLVESGGGLVQAGGSLRLSCAASGPAFSSSGMGWFRQAPGKARDFVASMDWSGGSTYYGDSV
KGRFTVSRDNAKNTVHLQMNSLRPEDTAVYYCARGTSGVAAVNLRGFFSWGPGTQVTVSS;
  (SEQ ID NO: 455)
QVQLQESGGGLVQAGGSLRLSCAASGPAFSSSGMGWFRQAPGKARDFVASIDWSGGSTYYGDSV
KGRFTVSRDNAKNTVHLQMNSLRPEDTAVYYCARGTSGVAAVNLRGFFSWGPGTQVTVSS;
  (SEQ ID NO: 456)
QVQLVESGGGLVQAGGSLRLACAASGAAFSSSGMGWFRQAPGKARDFVASMDWSGGSTYYGDSV
KGRFTVSRDNAKNTVHLQMNSLRPEDTAVYYCARGTSGVAAVNLRGFFSWGPGTQVTVSS;
  (SEQ ID NO: 457)
QVQLVESGGGLVQAGGSLRLSCAASGPAFSSSGMGWFRQAPGKARDFVASIDWGGGSTYYGDSV
KGRFTVSRDNAKNAVHLQMNSLRPEDTAVYYCARGTSGVAAVNLRGFFSWGPGTQVTVSS;
  (SEQ ID NO: 458)
QVQLVESGGGLVQAGGSLRLSCAASGPAFSSSGMGWFRQAPGKARDFVASIDWSGGSTYYGDSV
KGRFTVSRDNAKNTVHLQMNSLRPEDTAVYYCARGTSGVAAVNLRGFFSWGPGTQVTVSS;
  (SEQ ID NO: 459)
QVQLVESGGGLVQAGGSLRLSCAASGPAFSSSGMGWFRQAPGKARDFVASIDWSGKSTYYGDSV
KGRFTVSRDNAKNTVHLQMNSLRPEDTAVYYCARGTSGVAAVNLRGFFSWGPGTQVTVSS;
  (SEQ ID NO: 460)
QVQLVESGGGLVQAGGSLRLSCAASGPAFSSSGMGWFRQAPGKARDFVASINWSGGSAYYGDSV
KGRFTVSRDNAKNTVHLQMNSLRPEDTAVYYCARGTSGVAAVNLRGFFSWGPGTQVTVSS;
  (SEQ ID NO: 461)
QVQLVESGGGLVQAGGSLRLSCAASGPAFSSSGMGWFRQAPGKARDFVASMDWSGGSTYYGDSV
KGRFTVSRDNAKNTVHLQMNSLRPEDTAVYYCARGTSGVAAVNLRGFFSWGPGTQVTVSS;
  (SEQ ID NO: 462)
QVQLVESGGGLVQAGGSLRLSCAASGPAFSSSGMGWFRQAPGKAREFVASMDWTGGSTYYGDSV
KGRFTVSRDNAKMTVHLQMNSLRPEDTAVYYCARGTSGVAAVNLRGFFSWGPGTQVTVSS;
  (SEQ ID NO: 463)
QLQLVESGGGLVQPGGSLRLSCAASGSDFLVDATTWFRQAPGNQREFVAIMDIGGVTEYADSVK
GRFTISRDHTKNTVYLQMNSLKVEDTAVYYCNTRGLWGQGTLVTVSS;
  (SEQ ID NO: 464)
QVQLQESGGGLVQAGGSLRLSCATSGITSSINVIGWYRQAPGKQRELVALVNSGGQTHYADSVK
GRFTISRDNAKNTVFLQMNSLKPEDTAEYYCHGRYGIDNYWGEGTQVTVSS;
  (SEQ ID NO: 465)
EVQLVESGGGLVQPGGSLRLSCAASGFPFSSSFMSWVRQAPGKGLEWVSTIYSDGSTYYADSVK
GRFTISRDNAKKTAYLQMNSLKAEDTAVYYCATVTGSIRGQGTQVTVSS;
  (SEQ ID NO: 466)
QVQLVESGGGLVQPGGSLRLSCAASGFNFSSSFMSWVRQAPGKEVEWVSTIYSDGSTYYADSMK
GRFTISRDNAKNTVYLQMSNLKAEDTAVYYCATVTGSIRGQGTQVTVSS;
  (SEQ ID NO: 467)
QVQLVESGGGLVQPGGSVRLSCATSGSIFSTNVMGWYRQAPGKEREFVALIRGGGSTHYADSVK
GRFIISRENAKTTVYLQMNGLKPEDTAVYYCVIWLGSPGAMSDYWGQGTQVTVSS;
(SEQ ID NO: 468)
EVQLVESGGGLVQPGGSLRLSCAASGSVVSTNNMGWYRQAPGKEREFVALIRTGGSTHVADSMK
GRFTISRENAKNTVYLQMNSLKPEDTAVYYCVIWTGSPGALSDYWGQGTQVTVSS;
  (SEQ ID NO: 469)
QVQLVESGGGLVQPGGSLRLSCAASGSDASTNNMAWYRQAPGKEREFVALIRTGGSTHVADSMK
GRFTISRENAKNTVYLQMNSLKPEDTAVYYCVIWTGSPGALSDYWGQGTQVTVSS;
  (SEQ ID NO: 470)
QLQLVESGGGLVQPGESLRLSCAASGFSFSRVAMNWYRQAPGKERELVATISSDGSRTNYAHSV
KGRFTISRENAKNMVYLQMNSLKLEDTAVYYCNVPGNSWGQGTQVTVSS;
  (SEQ ID NO: 471)
QLQLVESGGGLVQPGESLRLSCAASGFSFSRVGMNWYRQAPGKERELVASISSDGSRTNYAHFV
KGRFTISRDNVKNMVYLQMNSLKLEDTAVYYCNVPGNSWGQGTQVTVSS;
  (SEQ ID NO: 472)
QLQLVESGGGLVQPGESLRLSCVVSGDIFSFVGWGWYRQAPGKQREVVAQISTGGLTNYADSVK
GRFAISRDNAKRTVYLQMNSLKFEDTAVYYCNVPGHPWGQGTQVTVSS;
  (SEQ ID NO: 473)
QVQLVESGGGLVQPGESLRLSCVVSGDIFSFIGWGWYRQAPGKQRELVAQINTGGLTDYADSVK
GRFTISRDNAKRTVYLQMNSLKFEDTAVYYCNFPGHSWGQGTQVTVSS;
  (SEQ ID NO: 474)
QVQLVESGGGLVQPGGSLRLSCAASGFTFSMYSMSWVRQAPGKRLEWVSSISTGARDTAYADSV
KGRFTISRDNADNTLYLHMNNLKPEDTAVYYCANGDLRYGPDGYDYRGQGTQVTVSS;
  (SEQ ID NO: 475)
QVQLVESGGGLVQPGGSLRLSCAASGFTFSMYSMSWVRQAPGKRLEWVSSISTGARDTAYADSV
KGRFTISRDNADNTLYLHMNNLKPEDTAVYYCANGDLRYGPDGYDYRGQGTQVTVVS;
  (SEQ ID NO: 476)
QVQLVESGGGLVQPGGSLRLSCAASGFTFSMYSMSWVRQAPGKRLEWVSSISTGARDTSYADSV
KGRFTISRDNADNTLYLHMNNLKPEDTAVYYCANGDLRYGPDGYDYRGQGTQVTVSS;
and
(SEQ ID NO: 477)
QVQLVESGGGLVQPGGSLRLSCVASGGTFSTYGMGWFRQAPGKEREFVAVITGSGVGTQYADSV
KDRFTISRENAKNTVYLQMNTLKLEDTAVYYCVSGHRPGWAVIRADAYEYWGQGTQVTVSS.
```

In any aspect of the disclosure, or embodiments thereof, the VHH antibody contains an amino acid sequence selected from one or more of:

```
  (SEQ ID NO: 431)
QVQLVESGGGLVQPGGSLRLSCAASGRTFINYAAGWFRQAPGKEREFVARISRSGGRTDYADSV
KGRFTISRDNAKSTVYLQMNSLRPEDTAVYYCAEATVWEFTDGADQYDYWGQGTQVTVSS;
(SEQ ID NO: 432)
EVQLVESGGGLVQAGGSLRLSCAASGRTFGSYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 433)
EVQLVESGGGLVQAGGSLRLSCAASGRTFGTYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 434)
QVQLQESGGGLVQAGGSLRLSCAASGRTFGSYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 435)
QVQLQESGGGLVQAGGSLRLSCAASGRTFGTYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 436)
QVQLQESGGGLVQAGGSLRLSCAASGRTFGTYTMGWFRQAPGKGREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 437)
QVQLVESGGGLVQAGGSLRLSCAASGRTFGSYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 438)
QVQLVESGGGLVQAGGSLRLSCAASGRTFGTYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 439)
QVQLVESGGGLVQAGGSLRLSCAASGRTFGTYTMGWFRQAPGREREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 440)
EVQLVESGGGLVQAGGSLRLSCAASGGTVSSYAMGWFRQAPGKEREFVAVISWSGGRTYYADSV
KGRFTISRDNAKNTVNLQMNSLKPEDTAVYYCAADPWTSDSDYERLTMYDYWGQGTQVTVSS;
(SEQ ID NO: 441)
EVQLVESGGGLVQAGGSRRLSCAASGGTVSSYAMGWFRQAPGKEREFVAVISWSGGRTYYADSV
KGRFTISRDNAKNTVYLQMNSLKPEDTAVYYCAADPWTSDSDYERLTMYDYWGQGTQVTVSS;
(SEQ ID NO: 442)
QVQLVESGGGLVQAGGSLRLSCAASGGTVSSYAMGWFRQAPGKEREFVAVISWSGGRTYYADSV
KGRFTISRDNAKNTVNLQMNSLKPEDTAVYYCAADPWTSDSDYERLTMYDYWGQGTQVTVSS;
(SEQ ID NO: 443)
QVQLVESGGGLVQAGGSLRLSCAASGGTVSSYAMGWFRQAPGKEREFVAVISWSGGRTYYADSV
KGRFTISRDNAKNTVYLQMNSLKPEDTAVYYCAADPWTSDSDYERLTMYDYWGQGTQVTVSS;
(SEQ ID NO: 444)
EVQLVESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTESQRLYGAWGQGTQVTVSS;
(SEQ ID NO: 445)
QVQLQESGGGLVQAGASLRLSCAASGRATYNMGWFRHAPGEDREFVAAINLEGYATRYANSVKG
RFTISRDNAKNTVYLQMNSLKPEDTAVYYCAADTSLPLGVLTESQRLYGAWGQGTQVTVSS;
(SEQ ID NO: 446)
QVQLQESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTESQRLYGAWGQGTQVTVSS;
(SEQ ID NO: 447)
QVQLQESGGGLVQAGASLRLSCAASGRTTYNMGWFRHAPGKDREFVAAIDLYGRATRYANSVRG
RFTISRDNAKNTVYLQMNSLKPEDTAVYYCAADTSLPLGVLTESQRLYGAWGQGTQVTVSS;
(SEQ ID NO: 448)
QVQLVESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTESQRLYGAWGQGTQVTVSS;
(SEQ ID NO: 449)
QVQLVESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTKSQRMYGAWGQGTQVTVSS;
  (SEQ ID NO: 450)
QVQLQESGGGSVQAGGSLRLSCAASGRAFSAYAMGWFRQAPGKEREFVAAINWNGDTALRWNGF
ATRYADSVKGRFIISRVNAKNTVNLQMNSLKPEDTAVYYCAADTVVSGSYYLAARAEDYEYWGQ
GTQVSVSS;
  (SEQ ID NO: 451)
QVQLQESGGGSVQAGGSLRLSCAASGRAFSAYAMGWFRQAPGKEREFVAAINWNGDTALRWNGF
ATRYADSVKGRFTISRVIAKNTVNLQMNSLKPEDTAVYYCAADTVVSGSYYLAARAEDYEYWGQ
GTQVTVSS;
  (SEQ ID NO: 452)
QVQLQESGGGSVQAGGSLRLSCAASGRAFSAYAMGWFRQAPGKEREFVAAINWNGDTALRWNGF
ATRYADSVKGRFTISRVNAKNTVNLQMNSLKPEDTAVYYCAADTVVSGSYYLAARAEDYEYWGQ
GTQVSVSS;
  (SEQ ID NO: 453)
QVQLQESGGGSVQAGGSLRLSCAASGRAFSAYAMGWFRQAPGKEREFVAAINWNGDTALRWNGF
ATRYADSVKGRFTISRVNAKNTVNLQMNSLKPEDTAVYYCAADTVVSGSYYLAARAEDYEYWGQ
GTQVTVSS;
  (SEQ ID NO: 454)
EVQLVESGGGLVQAGGSLRLSCAASGPAFSSSGMGWFRQAPGKARDFVASMDWSGGSTYYGDSV
KGRFTVSRDNAKNTVHLQMNSLRPEDTAVYYCARGTSGVAAVNLRGFFSWGPGTQVTVSS;
  (SEQ ID NO: 455)
QVQLQESGGGLVQAGGSLRLSCAASGPAFSSSGMGWFRQAPGKARDFVASIDWSGGSTYYGDSV
KGRFTVSRDNAKNTVHLQMNSLRPEDTAVYYCARGTSGVAAVNLRGFFSWGPGTQVTVSS;
  (SEQ ID NO: 456)
QVQLVESGGGLVQAGGSLRLACAASGAAFSSSGMGWFRQAPGKARDFVASMDWSGGSTYYGDSV
KGRFTVSRDNAKNTVHLQMNSLRPEDTAVYYCARGTSGVAAVNLRGFFSWGPGTQVTVSS;
  (SEQ ID NO: 457)
QVQLVESGGGLVQAGGSLRLSCAASGPAFSSSGMGWFRQAPGKARDFVASIDWGGGSTYYGDSV
KGRFTVSRDNAKNAVHLQMNSLRPEDTAVYYCARGTSGVAAVNLRGFFSWGPGTQVTVSS;
  (SEQ ID NO: 458)
QVQLVESGGGLVQAGGSLRLSCAASGPAFSSSGMGWFRQAPGKARDFVASIDWSGGSTYYGDSV
KGRFTVSRDNAKNTVHLQMNSLRPEDTAVYYCARGTSGVAAVNLRGFFSWGPGTQVTVSS;
  (SEQ ID NO: 459)
QVQLVESGGGLVQAGGSLRLSCAASGPAFSSSGMGWFRQAPGKARDFVASIDWSGKSTYYGDSV
KGRFTVSRDNAKNTVHLQMNSLRPEDTAVYYCARGTSGVAAVNLRGFFSWGPGTQVTVSS;
  (SEQ ID NO: 460)
QVQLVESGGGLVQAGGSLRLSCAASGPAFSSSGMGWFRQAPGKARDFVASINWSGGSAYYGDSV
KGRFTVSRDNAKNTVHLQMNSLRPEDTAVYYCARGTSGVAAVNLRGFFSWGPGTQVTVSS;
  (SEQ ID NO: 461)
QVQLVESGGGLVQAGGSLRLSCAASGPAFSSSGMGWFRQAPGKARDFVASMDWSGGSTYYGDSV
KGRFTVSRDNAKNTVHLQMNSLRPEDTAVYYCARGTSGVAAVNLRGFFSWGPGTQVTVSS;
  (SEQ ID NO: 462)
QVQLVESGGGLVQAGGSLRLSCAASGPAFSSSGMGWFRQAPGKAREFVASMDWTGGSTYYGDSV
KGRFTVSRDNAKMTVHLQMNSLRPEDTAVYYCARGTSGVAAVNLRGFFSWGPGTQVTVSS;
  (SEQ ID NO: 463)
QLQLVESGGGLVQPGGSLRLSCAASGSDFLVDATTWFRQAPGNQREFVAIMDIGGVTEYADSVK
GRFTISRDHTKNTVYLQMNSLKVEDTAVYYCNTRGLWGQGTLVTVSS;
  (SEQ ID NO: 464)
QVQLQESGGGLVQAGGSLRLSCATSGITSSINVIGWYRQAPGKQRELVALVNSGGQTHYADSVK
GRFTISRDNAKNTVFLQMNSLKPEDTAEYYCHGRYGIDNYWGEGTQVTVSS;
  (SEQ ID NO: 465)
EVQLVESGGGLVQPGGSLRLSCAASGFPFSSSFMSWVRQAPGKGLEWVSTIYSDGSTYYADSVK
GRFTISRDNAKKTAYLQMNSLKAEDTAVYYCATVTGSIRGQGTQVTVSS;
  (SEQ ID NO: 466)
QVQLVESGGGLVQPGGSLRLSCAASGFNFSSSFMSWVRQAPGKEVEWVSTIYSDGSTYYADSMK
GRFTISRDNAKNTVYLQMSNLKAEDTAVYYCATVTGSIRGQGTQVTVSS;
  (SEQ ID NO: 467)
QVQLVESGGGLVQPGGSVRLSCATSGSIFSTNVMGWYRQAPGKEREFVALIRGGGSTHYADSVK
GRFIISRENAKTTVYLQMNGLKPEDTAVYYCVIWLGSPGAMSDYWGQGTQVTVSS;
  (SEQ ID NO: 468)
EVQLVESGGGLVQPGGSLRLSCAASGSVVSTNNMGWYRQAPGKEREFVALIRTGGSTHVADSMK
GRFTISRENAKNTVYLQMNSLKPEDTAVYYCVIWTGSPGALSDYWGQGTQVTVSS;
  (SEQ ID NO: 469)
QVQLVESGGGLVQPGGSLRLSCAASGSDASTNNMAWYRQAPGKEREFVALIRTGGSTHVADSMK
GRFTISRENAKNTVYLQMNSLKPEDTAVYYCVIWTGSPGALSDYWGQGTQVTVSS;
  (SEQ ID NO: 470)
QLQLVESGGGLVQPGESLRLSCAASGFSFSRVAMNWYRQAPGKERELVATISSDGSRTNYAHSV
KGRFTISRENAKNMVYLQMNSLKLEDTAVYYCNVPGNSWGQGTQVTVSS;
  (SEQ ID NO: 471)
QLQLVESGGGLVQPGESLRLSCAASGFSFSRVGMNWYRQAPGKERELVASISSDGSRTNYAHFV
KGRFTISRDNVKNMVYLQMNSLKLEDTAVYYCNVPGNSWGQGTQVTVSS;
  (SEQ ID NO: 472)
QLQLVESGGGLVQPGESLRLSCVVSGDIFSFVGWGWYRQAPGKQREVVAQISTGGLTNYADSVK
GRFAISRDNAKRTVYLQMNSLKFEDTAVYYCNVPGHPWGQGTQVTVSS;
  (SEQ ID NO: 473)
QVQLVESGGGLVQPGESLRLSCVVSGDIFSFIGWGWYRQAPGKQRELVAQINTGGLTDYADSVK
GRFTISRDNAKRTVYLQMNSLKFEDTAVYYCNFPGHSWGQGTQVTVSS;
  (SEQ ID NO: 474)
QVQLVESGGGLVQPGGSLRLSCAASGFTFSMYSMSWVRQAPGKRLEWVSSISTGARDTAYADSV
KGRFTISRDNADNTLYLHMNNLKPEDTAVYYCANGDLRYGPDGYDYRGQGTQVTVSS;
  (SEQ ID NO: 475)
QVQLVESGGGLVQPGGSLRLSCAASGFTFSMYSMSWVRQAPGKRLEWVSSISTGARDTAYADSV
KGRFTISRDNADNTLYLHMNNLKPEDTAVYYCANGDLRYGPDGYDYRGQGTQVTVVS;
  (SEQ ID NO: 476)
QVQLVESGGGLVQPGGSLRLSCAASGFTFSMYSMSWVRQAPGKRLEWVSSISTGARDTSYADSV
KGRFTISRDNADNTLYLHMNNLKPEDTAVYYCANGDLRYGPDGYDYRGQGTQVTVSS;
and
(SEQ ID NO: 477)
QVQLVESGGGLVQPGGSLRLSCVASGGTFSTYGMGWFRQAPGKEREFVAVITGSGVGTQYADSV
KDRFTISRENAKNTVYLQMNTLKLEDTAVYYCVSGHRPGWAVIRADAYEYWGQGTQVTVSS.
```

In any aspect of the disclosure, or embodiments thereof, the lipid nanoparticle is conjugated to the VHH antibody. In any aspect of the disclosure, or embodiments thereof, the VHH antibody is covalently bound to a polyethylene glycol (PEG) molecule of the lipid nanoparticle. In any aspect of the disclosure, or embodiments thereof, the VHH antibody is covalently bound to a PEG portion of a PEG-modified lipid of the lipid nanoparticle.

In any aspect of the disclosure, or embodiments thereof, the lipid nanoparticle contains a polynucleotide encoding a chimeric antigen receptor.

In any aspect of the disclosure, or embodiments thereof, the chimeric antigen receptor contains an antigen binding domain capable of binding a marker associated with a neoplasia.

In any aspect of the disclosure, or embodiments thereof, the carrier or excipient is a pharmaceutical carrier or excipient.

In any aspect of the disclosure, or embodiments thereof, the neoplasia is a B cell lymphoma or a T cell lymphoma.

In any aspect of the disclosure, or embodiments thereof, the T cell lymphoma is a T-cell acute lymphoblastic leukemia.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “adenine” or “9H-Purin-6-amine” is meant a purine nucleobase with the molecular formula C₅H₅N₅, having the structure

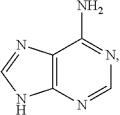

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2(1H)-one” is meant an adenine molecule attached to a ribose sugar via a glycosidic bond, having the structure

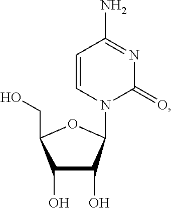

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA) and may be referred to as a “dual deaminase”. Non-limiting examples of dual deaminases include those described in PCT/US22/22050. In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA. In embodiments, the adenosine deaminase variant is selected from those described in PCT/US2020/018192, PCT/US2020/049975, PCT/US2017/045381, PCT/US2021/016827, PCT/US2022/073781, PCT/US24/34189, or PCT/US2020/028568, the full contents of which are each incorporated herein by reference in their entireties for all purposes. Further non-limiting examples of adenosine deaminases include those disclosed or referenced in Rufflow, et al., “Design of highly functional genome editors by modeling of the universe of CRISPR-Cas Sequences,” bioRxiv, posted Apr. 22, 2024, doi: 10.1101/2024.04.22.590591, the disclosure of which is incorporated herein by reference in its entirety for all purposes, which were designed using artificial intelligence. Further exemplary adenosine deaminase amino acid sequences include: TadA-8e (SEQ ID NO: 628), Tad1 (SEQ ID NO: 629), Tad2 (SEQ ID NO: 630), Tad3 (SEQ ID NO: 631), Tad4 (SEQ ID NO: 632), Tad6 (SEQ ID NO: 633), Tad6-SR (SEQ ID NO: 634), TadA9 (SEQ ID NO: 635), TadA20 (SEQ ID NO: 636), Staphylococcus aureus TadA (SEQ ID NO: 637), Bacillus subtilis TadA (SEQ ID NO: 638), Salmonella typhimurium TadA (SEQ ID NO: 639), Shewanella putrefaciens (SEQ ID NO: 640), Haemophilus influenzae F3031 TadA (SEQ ID NO: 641), Caulobacter crescentus TadA (SEQ ID NO: 642), Geobacter sulfurreducens TadA (SEQ ID NO: 643), Streptococcus pyogenes TadA (SEQ ID NO: 644), Aquifex aeolicus TadA (SEQ ID NO: 645), and E. coli TadA deaminase (ecTadA) (SEQ ID NO: 646).

By “adenosine deaminase activity” is meant catalyzing the deamination of adenine or adenosine to guanine in a polynucleotide.

By “Adenosine Base Editor (ABE)” is meant a base editor comprising an adenosine deaminase.

By “Adenosine Base Editor (ABE) polynucleotide” is meant a polynucleotide encoding an ABE.

By “Adenosine Base Editor 8 (ABE8) polypeptide” or “ABE8” is meant a base editor as defined herein comprising an adenosine deaminase or adenosine deaminase variant comprising one or more of the alterations listed in Table 5B, one of the combinations of alterations listed in Table 5B, or an alteration at one or more of the amino acid positions listed in Table 5B, where such alterations are relative to the following reference sequence: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or a corresponding position in another adenosine deaminase. In embodiments, ABE8 comprises alterations at amino acids 82 and/or 166 of SEQ ID NO: 1. In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

“Administering” is referred to herein as providing one or more compositions described herein to a patient or a subject. By way of example and without limitation, composition administration (e.g., injection) can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally. Alternatively, or concurrently, administration can be by the oral route.

By “agent” is meant any small molecule chemical compound, antibody, nucleic acid molecule, or polypeptide, or fragments thereof.

“Allogeneic,” as used herein, refers to cells that are genetically dissimilar and immunologically incompatible. In embodiments, allogeneic cells are administered to a genetically dissimilar and immunologically incompatible subject. In some embodiments, the allogeneic cells comprise modifications improving their persistence in the subject allogeneic to the cells.

By “alteration” is meant a change in the level, structure, or activity of an analyte, gene or polypeptide as detected by standard art known methods, such as those described herein. As used herein, an alteration includes a change (e.g., increase or reduction) in expression levels. In embodiments, the increase or reduction in expression levels is by 10%, 25%, 40%, 50% or greater. In some embodiments, an alteration (e.g., in structure) includes an insertion, deletion, or substitution of a nucleobase or amino acid (e.g., by genetic engineering).

By “ameliorate” is meant reduce, suppress, attenuate, diminish, arrest, or stabilize the development or progression of a disease.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “anionic lipid” is meant a lipid species that carries a net negative charge at a selected pH.

As used herein, the term “antibody” or “antigen-binding domain” refers to an immunoglobulin molecule, a single-domain antibody (sdAb), or a fragment thereof that specifically binds to, or is immunologically reactive with, a particular antigen. Non-limiting examples of antibodies or antigen-binding domains include VHH antibodies, polyclonal, monoclonal, genetically engineered and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)2, Fab, Fv, rIgG, and scFv fragments, as well as engineered antibodies, which include CrossMabs (e.g., CrossMab^{Fabs}, CrossMab^{CH1-CL} and CrossMab^{VH-VL} formats), or fragments thereof. Moreover, unless otherwise indicated, the term “monoclonal antibody” (mAb) is meant to include both intact molecules, as well as antibody fragments (such as, for example, Fab and F(ab′)2 fragments) that are capable of specifically binding to a target protein. Fab and F(ab′)₂ fragments lack the Fc fragment of an intact antibody, clear more rapidly from the circulation of the animal, and may have less non-specific tissue binding than an intact antibody (see Wahl et al., J. Nucl. Med. 24:316, 1983; incorporated herein by reference).

Antibody structure is well known in the art. Briefly, the variable (V) regions or domains of antibody heavy (H) and light (L) chains contain Complementarity-Determining Regions (CDRs), which bind to specific antigens or immunogens (e.g., protein antigens or immunogens). CDRs are situated within framework (FR) sequences of the V regions of the heavy (V_{H}) and light chains (V_{L}) of an antibody. CDRs are the most variable parts of antibodies and are critical components in the diversity of antigen specificities of antibodies produced by B lymphocytes. In general, three CDRs (CDR1, CDR2 and CDR3) are arranged consecutively in a V domain of an antibody. Because a VHH, such as a camelid VHH, is essentially a single chain antibody polypeptide, it contains three CDRs that bind to an antigen or target protein such as CD5 in the context of four framework (FR) regions, as follows: FR1-CDR1-FR2-CDR2-FR3-CDR3-FR4. Because most of the sequence variability associated with immunoglobulins and antigen binding is found in the CDRs, these regions are sometimes referred to as hypervariable regions. Typically, CDR1, CDR2 and CDR3 of VHHs contribute to and/or do not interfere with antigen binding. The CDRs of a number of anti-CD5 VHHs described herein are shown, for example, in Tables 1A and 1B.

By “antigen” is meant an agent to which an antibody or other polypeptide capture molecule specifically binds. In an embodiment, the antigen is a tumor antigen. Exemplary antigens include small molecules, carbohydrates, proteins, and polynucleotides.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1). Representative nucleic acid and protein sequences of base editors include those sequences having about or at least about 85% sequence identity to any base editor sequence provided in the sequence listing, such as those corresponding to SEQ ID NOs: 2-11.

By “BE4 cytidine deaminase (BE4) polypeptide,” is meant a base editor comprising a nucleic acid programmable DNA binding protein (napDNAbp) domain, a cytidine deaminase domain, and two uracil glycosylase inhibitor domains (UGIs). In embodiments, the napDNAbp is a Cas9n (D10A) polypeptide. Non-limiting examples of cytidine deaminase domains include rAPOBEC, ppAPOBEC, RrA3F, AmAPOBEC1, and SsAPOBEC3B.

By “BE4 cytidine deaminase (BE4) polynucleotide,” is meant a polynucleotide encoding a BE4 polypeptide.

By “base editing activity” is meant acting to chemically alter a base within a polynucleotide. In one embodiment, a first base is converted to a second base. In one embodiment, the base editing activity is cytidine deaminase activity, e.g., converting target C·G to T·A. In another embodiment, the base editing activity is adenosine or adenine deaminase activity, e.g., converting A·T to G·C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system.

A “camelid VHH framework region (FR)” refers to the structural FR portions or components of a camelid VHH antibody or binding molecule, namely, FR1, FR2, FR4 and FR4, that positionally and structurally support the three CDR components, namely, CDR1, CDR2 and CDR3 of a VHH polypeptide, as described above. Similar to the FRs in conventional antibody polypeptides, the respective FR regions (FR1, FR2, FR3 and FR4) of the anti-CD5 VHH polypeptides described herein are highly similar in sequence not only among different CD5 binding VHHs but also among camelid VHH polypeptides that bind to other antigens, e.g., unrelated VHH polypeptides. (See, e.g., L. S. Mitchell and L. J. Colwell, 2018, Proteins, 86(7): 697-706 and A. M. Vattekatte et al., March 2020, PeerJ., 6(8): e8408. DOI: 10.7717/peerj.8408). Accordingly, the FR regions FR1, FR2, FR3 and FR4 of different VHHs do not vary significantly in sequence. By way of example, the below FR sequences of the VHH in the above-mentioned publication of Mitchell and Colwell are similar to the FR sequences of other VHHs, including the anti-CD5 VHH polypeptides described herein.

FR1 (SEQ ID NO: 625):

| Position | | | | | | | | | | | | | |
|---|---|---|---|---|---|---|---|---|---|---|---|---|---|
| # | 1 | 2 | 3 | 4 | 5 | 6 | 7 | 8 | 9 | 10 | 11 | 12 | 13 |
| AA | Q | V | Q | | Q | E | S | G | G | G | L | V | Q |
| | | | | | or | | | | | | or | | |
| | | | | | V | | | | | | S | | |

FR1 (continued)

| Position | | | | | | | | | | | | |
|---|---|---|---|---|---|---|---|---|---|---|---|---|
| # | 14 | 15 | 16 | 17 | 18 | 19 | 20 | 21 | 22 | 23 | 24 | 25 |
| AA | A | G | G | S | L | R | L | S | C | A | A | S |
| | or | | | | | | | | | | | |
| | P | | | | | | | | | | | |

FR2 (SEQ ID NO: 626):

| Position | | | | | | | | | | | | | | |
|---|---|---|---|---|---|---|---|---|---|---|---|---|---|---|
| # | 36 | 37 | 38 | 39 | 40 | 41 | 42 | 43 | 44 | 45 | 46 | 47 | 48 | 49 |
| AA | W | F, | R | Q | A | P | G | K | E, | R | E | F, | V | A, |
| | | Y, | | | | | | | C, | or | | G, | | S, |
| | | or | | | | | | | or | L | | L, | | or |
| | | V | | | | | | | G | | | or | | T |
| | | | | | | | | | | | | W | | |

FR3 (SEQ ID NO: 627):

| Position | | | | | | | | | | | | | | | | | | | |
|---|---|---|---|---|---|---|---|---|---|---|---|---|---|---|---|---|---|---|---|
| # | 60 | 61 | 62 | 63 | 64 | 65 | 66 | 67 | 68 | 69 | 70 | 71 | 72 | 73 | 74 | 75 | 76 | 77 | 78 |
| AA | Y | A, | D | S | V | K | G | R | F | T | I | S | R | D | N | A | K | N | T |
| | | Q, | or | | or | | | | | or | or | | or | | or | | or | | |
| | | T, | E | | A | | | | | A | V | | Q | | K | | A | | |
| | | or | | | | | | | | | | | | | | | | | |
| | | V | | | | | | | | | | | | | | | | | |

FR3 (continued):

| Position | | | | | | | | | | | | | | | | | | |
|---|---|---|---|---|---|---|---|---|---|---|---|---|---|---|---|---|---|---|
| # | 79 | 80 | 81 | 82 | 83 | 84 | 85 | 86 | 87 | 88 | 89 | 90 | 91 | 92 | 93 | 94 | 95 | 96 |
| AA | V, | Y | L | Q | M | N | S | L | K | P | E | D | T | A | V, | Y | Y | C |
| | L, | | | | | or | or | | or | | or | | | or | I, | | | |
| | or | | | | | D | N | | R | | D | | | G | T, | | | |
| | M | | | | | | | | | | | | | | or | | | |
| | | | | | | | | | | | | | | | M | | | |

FR4 (SEQ ID NO: 542):

| Position | | | | | | | | | | | |
|---|---|---|---|---|---|---|---|---|---|---|---|
| # | 117 | 118 | 119 | 120 | 121 | 122 | 123 | 124 | 125 | 126 | 127 |
| AA | W | G | Q | G | T | Q | V | T | V | S | S |

It will be appreciated that the amino acid position numbers of the VHH FRs shown above are approximate and may vary to some degree in length or amino acid sequence depending on VHH length and on the start and termination amino acid positions of the VHH CDRs. Thus, substantial similarities exist among the structural FRs of camelid VHHs, independent of antigen binding specificity.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease.

By “chimeric antigen receptor” or “CAR” is meant a synthetic or engineered receptor comprising an extracellular antigen binding domain joined to one or more intracellular signaling domains (e.g., T cell signaling domain) that confers specificity for an antigen onto an immune effector cell (e.g., a T-cell, an NK cell, or a macrophage). In embodiments, the CAR is a SUPRA CAR, an anti-tag CAR, a TCR-CAR, or a TCR-like CAR (see, e.g., Guedan, et al. “Engineering and Design of Chimeric Antigen Receptors,” Methods and Clinical Development, 12:145-156 (2019); Poorebrahim, et al., “TCR-like CARs and TCR-CARs targeting neoepitopes: an emerging potential,” Cancer Gene Therapy, 28:581-589 (2021); and Minutolo, et al. “The Emergence of Universal Immune Receptor T Cell Therapy for Cancer,” Front Oncol., 9:176 (2019), the disclosures of which are incorporated herein by reference in their entireties for all purposes).

By “chimeric antigen receptor (CAR) T cell” or “CAR-T cell” is meant a T cell expressing a CAR that has antigen specificity determined by the antibody-derived targeting domain of the CAR. As used herein, “CAR-T cells” includes T cells, regulatory T cells (TREG), macrophages, or NK cells. As used herein, “CAR-T cells” include cells engineered to express a CAR or a T cell receptor (TCR, sometimes referred to as TCR-CARs or TCR-like CARs). Methods of making CARs (e.g., for treatment of cancer) are publicly available (see, e.g., Park et al., Trends Biotechnol., 29:550-557, 2011; Grupp et al., N Engl J Med., 368:1509-1518, 2013; Han et al., J. Hematol Oncol. 6:47, 2013; Haso et al., (2013) Blood, 121, 1165-1174; Mohseni, et al., (2020) Front. Immunol., 11, art. 1608, doi: 10.3389/fimmu.2020.01608; Eggenhuizen, et al. Int. J. Mol. Sci. (2020), 21:7015, doi: 10.3390/ijms21197015; Poorebrahim, et al., Cancer Gene Ther 28, 581-589 (2021), doi.org/10.1038/s41417-021-00307-7, PCT Pubs. WO2012/079000, WO2013/059593; and U.S. Pub. 2012/0213783, the disclosure of each of which is incorporated herein by reference herein in its entirety).

By “cluster of differentiation 5 (CD5) polypeptide” is meant a protein having at least about 85% amino acid sequence identity to NCBI Accession No. NP_001333385.1 or fragment thereof, having immunomodulatory activity. An exemplary amino acid sequence is provided below.

>NP_001333385.1 T-cell surface glycoprotein CD5
isoform 2 [Homo sapiens]
(SEQ ID NO: 426)
MVCSQSWGRSSKQWEDPSQASKVCQRLNCGVPLSLGPFLVTYTPQSSIICYGQLGSFSNCSHSR
NDMCHSLGLTCLEPQKTTPPTTRPPPTTTPEPTAPPRLQLVAQSGGQHCAGVVEFYSGSLGGTI
SYEAQDKTQDLENFLCNNLQCGSFLKHLPETEAGRAQDPGEPREHQPLPIQWKIQNSSCTSLEH
CFRKIKPQKSGRVLALLCSGFQPKVQSRLVGGSSICEGTVEVRQGAQWAALCDSSSARSSLRWE
EVCREQQCGSVNSYRVLDAGDPTSRGLFCPHQKLSQCHELWERNSYCKKVFVTCQDPNPAGLAA
GTVASIILALVLLVVLLVVCGPLAYKKLVKKFRQKKQRQWIGPTGMNQNMSFHRNHTATVRSHA
ENPTASHVDNEYSQPPRNSHLSAYPALEGALHRSSMQPDNSSDSDYDLHGAQRL.

By “cluster of differentiation 5 (CD5) polynucleotide” is meant a polynucleotide encoding a CD5 polypeptide, as well as the introns, exons, 3′ untranslated regions, 5′ untranslated regions, and regulatory sequences associated with its expression, or fragments thereof. In embodiments, a CD5 polynucleotide is the genomic sequence, cDNA, RNA, or gene associated with and/or required for CD5 expression. An exemplary CD5 nucleic acid sequence is provided below. >NM_001346456.1 Homo sapiens CD5 molecule (CD5), transcript variant 2, mRNA

(SEQ ID NO: 427)
GAGTCTTGCTGATGCTCCCGGCTGAATAAACCCCTTCCTTCTTTAACTTGGTGTCTGAGGGGTT
TTGTCTGTGGCTTGTCCTGCTACATTTCTTGGTTCCCTGACCAGGAAGCAAAGTGATTAACGGA
CAGTTGAGGCAGCCCCTTAGGCAGCTTAGGCCTGCCTTGTGGAGCATCCCCGCGGGGAACTCTG
GCCAGCTTGAGCGACACGGATCCTCAGAGCGCTCCCAGGTAGGCAATTGCCCCAGTGGAATGCC
TCGTCAGAGCAGTGCATGGCAGGCCCCTGTGGAGGATCAACGCAGTGGCTGAACACAGGGAAGG
AACTGGCACTTGGAGTCCGGACAACTGAAACTTGTCGCTTCCTGCCTCGGACGGCTCAGCTGGT
ATGACCCAGATTTCCAGGCAAGGCTCACCCGTTCCAACTCGAAGTGCCAGGGCCAGCTGGAGGT
CTACCTCAAGGACGGATGGCACATGGTTTGCAGCCAGAGCTGGGGCCGGAGCTCCAAGCAGTGG
GAGGACCCCAGTCAAGCGTCAAAAGTCTGCCAGCGGCTGAACTGTGGGGTGCCCTTAAGCCTTG
GCCCCTTCCTTGTCACCTACACACCTCAGAGCTCAATCATCTGCTACGGACAACTGGGCTCCTT
CTCCAACTGCAGCCACAGCAGAAATGACATGTGTCACTCTCTGGGCCTGACCTGCTTAGAACCC
CAGAAGACAACACCTCCAACGACAAGGCCCCCGCCCACCACAACTCCAGAGCCCACAGCTCCTC
CCAGGCTGCAGCTGGTGGCACAGTCTGGCGGCCAGCACTGTGCCGGCGTGGTGGAGTTCTACAG
CGGCAGCCTGGGGGGTACCATCAGCTATGAGGCCCAGGACAAGACCCAGGACCTGGAGAACTTC
CTCTGCAACAACCTCCAGTGTGGCTCCTTCTTGAAGCATCTGCCAGAGACTGAGGCAGGCAGAG
CCCAAGACCCAGGGGAGCCACGGGAACACCAGCCCTTGCCAATCCAATGGAAGATCCAGAACTC
AAGCTGTACCTCCCTGGAGCATTGCTTCAGGAAAATCAAGCCCCAGAAAAGTGGCCGAGTTCTT
GCCCTCCTTTGCTCAGGTTTCCAGCCCAAGGTGCAGAGCCGTCTGGTGGGGGGCAGCAGCATCT
GTGAAGGCACCGTGGAGGTGCGCCAGGGGGCTCAGTGGGCAGCCCTGTGTGACAGCTCTTCAGC
CAGGAGCTCGCTGCGGTGGGAGGAGGTGTGCCGGGAGCAGCAGTGTGGCAGCGTCAACTCCTAT
CGAGTGCTGGACGCTGGTGACCCAACATCCCGGGGGCTCTTCTGTCCCCATCAGAAGCTGTCCC
AGTGCCACGAACTTTGGGAGAGAAATTCCTACTGCAAGAAGGTGTTTGTCACATGCCAGGATCC
AAACCCCGCAGGCCTGGCCGCAGGCACGGTGGCAAGCATCATCCTGGCCCTGGTGCTCCTGGTG
GTGCTGCTGGTCGTGTGCGGCCCCCTTGCCTACAAGAAGCTAGTGAAGAAATTCCGCCAGAAGA
AGCAGCGCCAGTGGATTGGCCCAACGGGAATGAACCAAAACATGTCTTTCCATCGCAACCACAC
GGCAACCGTCCGATCCCATGCTGAGAACCCCACAGCCTCCCACGTGGATAACGAATACAGCCAA
CCTCCCAGGAACTCCCACCTGTCAGCTTATCCAGCTCTGGAAGGGGCTCTGCATCGCTCCTCCA
TGCAGCCTGACAACTCCTCCGACAGTGACTATGATCTGCATGGGGCTCAGAGGCTGTAAAGAAC
TGGGATCCATGAGCAAAAAGCCGAGAGCCAGACCTGTTTGTCCTGAGAAAACTGTCCGCTCTTC
ACTTGAAATCATGTCCCTATTTCTACCCCGGCCAGAACATGGACAGAGGCCAGAAGCCTTCCGG
ACAGGCGCTGCTGCCCCGAGTGGCAGGCCAGCTCACACTCTGCTGCACAACAGCTCGGCCGCCC
CTCCACTTGTGGAAGCTGTGGTGGGCAGAGCCCCAAAACAAGCAGCCTTCCAACTAGAGACTCG
GGGGTGTCTGAAGGGGGCCCCCTTTCCCTGCCCGCTGGGGAGCGGCGTCTCAGTGAAATCGGCT
TTCTCCTCAGACTCTGTCCCTGGTAAGGAGTGACAAGGAAGCTCACAGCTGGGCGAGTGCATTT
TGAATAGTTTTTTGTAAGTAGTGCTTTTCCTCCTTCCTGACAAATCGAGCGCTTTGGCCTCTTC
TGTGCAGCATCCACCCCTGCGGATCCCTCTGGGGAGGACAGGAAGGGGACTCCCGGAGACCTCT
GCAGCCGTGGTGGTCAGAGGCTGCTCACCTGAGCACAAAGACAGCTCTGCACATTCACCGCAGC
TGCCAGCCAGGGGTCTGGGTGGGCACCACCCTGACCCACAGCGTCACCCCACTCCCTCTGTCTT
ATGACTCCCCTCCCCAACCCCCTCATCTAAAGACACCTTCCTTTCCACTGGCTGTCAAGCCCAC
AGGGCACCAGTGCCACCCAGGGCCCGGCACAAAGGGGCGCCTAGTAAACCTTAACCAACTTGGT
TTTTTGCTTCACCCAGCAATTAAAAGTCCCAAGCTGAGGTAGTTTCAGTCCATCACAGTTCATC
TTCTAACCCAAGAGTCAGAGATGGGGCTGGTCATGTTCCTTTGGTTTGAATAACTCCCTTGACG
AAAACAGACTCCTCTAGTACTTGGAGATCTTGGACGTACACCTAATCCCATGGGGCCTCGGCTT
CCTTAACTGCAAGTGAGAAGAGGAGGTCTACCCAGGAGCCTCGGGTCTGATCAAGGGAGAGGCC
AGGCGCAGCTCACTGCGGCGGCTCCCTAAGAAGGTGAAGCAACATGGGAACACATCCTAAGACA
GGTCCTTTCTCCACGCCATTTGATGCTGTATCTCCTGGGAGCACAGGCATCAATGGTCCAAGCC
GCATAATAAGTCTGGAAGAGCAAAAGGGAGTTACTAGGATATGGGGTGGGCTGCTCCCAGAATC
TGCTCAGCTTTCTGCCCCCACCAACACCCTCCAACCAGGCCTTGCCTTCTGAGAGCCCCCGTGG
CCAAGCCCAGGTCACAGATCTTCCCCCGACCATGCTGGGAATCCAGAAACAGGGACCCCATTTG
TCTTCCCATATCTGGTGGAGGTGAGGGGGCTCCTCAAAAGGGAACTGAGAGGCTGCTCTTAGGG
AGGGCAAAGGTTCGGGGGCAGCCAGTGTCTCCCATCAGTGCCTTTTTTAATAAAAGCTCTTTCA
TCTATAGTTTGGCCACCATACAGTGGCCTCAAAGCAACCATGGCCTACTTAAAAACCAAACCAA
AAATAAAGAGTTTAGTTGAGGAGAAAAAAAAAAAAAAAAAAAAAAAAA.

An exemplary CD5 gene sequence is provided at ENSEMBL Accession No. ENSG00000110448.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

Amino acids generally can be grouped into classes according to the following common side-chain properties:
(1) hydrophobic: Norleucine, Met, Ala, Val, Leu, He;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following: TAG, TAA, and TGA.

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and x-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2(1H)-one” is meant a purine nucleobase with the molecular formula C₄H₅N₃O, having the structure

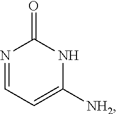

and corresponding to CAS No. 71-30-7.

By “cytidine” is meant a cytosine molecule attached to a ribose sugar via a glycosidic bond, having the structure

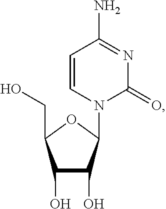

and corresponding to CAS No. 65-46-3. Its molecular formula is C₉H₁₃N₃O₅.

By “Cytidine Base Editor (CBE)” is meant a base editor comprising a cytidine deaminase.

By “Cytidine Base Editor (CBE) polynucleotide” is meant a polynucleotide encoding a CBE.

By “cytidine deaminase” or “cytosine deaminase” is meant a polypeptide or fragment thereof capable of deaminating cytidine or cytosine. In embodiments, the cytidine or cytosine is present in a polynucleotide. In one embodiment, the cytidine deaminase converts cytosine to uracil or 5-methylcytosine to thymine. The terms “cytidine deaminase” and “cytosine deaminase” are used interchangeably throughout the application. Petromyzon marinus cytosine deaminase 1 (PmCDA1) (SEQ ID NO: 13-14), Activation-induced cytidine deaminase (AICDA) (SEQ ID NOs: 15-21), and APOBEC (SEQ ID NOs: 12-61) are exemplary cytidine deaminases. Further exemplary cytidine deaminase (CDA) sequences are provided in the Sequence Listing as SEQ ID NOs: 62-66 and SEQ ID NOs: 67-189. Non-limiting examples of cytidine deaminases include those described in PCT/US20/16288, PCT/US2018/021878, 180802-021804/PCT, PCT/US2018/048969, and PCT/US2016/058344.

By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

The term “deaminase” or “deaminase domain,” as used herein, refers to a protein or fragment thereof that catalyzes a deamination reaction.

The term “detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. In some embodiments, the disease is a cancer (e.g., a hematological cancer or a solid tumor). In some instances, the disease is a disease that can be treated using the modified allogeneic T cells of the disclosure. In one embodiment, the disease is a neoplasia or cancer. In some instances, the disease is a malignancy. In some cases, the disease is a dysplasia, or a non-malignant or benign neoplasia. In some embodiments, the disease is a hematological cancer. By “hematological cancer” is meant a malignancy of immune system cells. In some embodiments, the hematological cancer is leukemia, myeloma, and/or lymphoma. Lymphomas and Leukemias are examples of “liquid cancers” or cancers present in the blood and are derived from the transformation of either a hematopoietic precursor in the bone marrow or a mature hematopoietic cell in the blood. Leukemias can be lymphoid or myeloid, and acute or chronic. In the case of myelomas, the transformed cell is a fully differentiated plasma cell that may be present as a dispersed collection of malignant cells or as a solid mass in the bone marrow. In the case of lymphomas, a transformed lymphocyte in a secondary lymphoid tissue generates a solid mass. Lymphomas are classified either Hodgkin lymphoma (HL) or non-Hodgkin lymphoma (NHL). In some cases, the disease or disorder is an autoimmune disorder, such as arthritis (e.g., rheumatoid arthritis) or systemic lupus erythematosus (SLE). In some embodiments, the disease is a B-cell lymphoma or a T-cell lymphoma (e.g., T-cell acute lymphoblastic leukemia (T-ALL)).

By “dual editing activity” or “dual deaminase activity” is meant having adenosine deaminase and cytidine deaminase activity. In one embodiment, a base editor having dual editing activity has both A→G and C→T activity, wherein the two activities are approximately equal or are within about 10% or 20% of each other. In another embodiment, a dual editor has A→G activity that no more than about 10% or 20% greater than C→T activity. In another embodiment, a dual editor has A→G activity that is no more than about 10% or 20% less than C→T activity. In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. Non-limiting examples of proteins having dual deaminase activity include those described in International Patent Application Publications No. WO 2024/040083 and WO 2022/204574, the disclosures of which are hereby incorporated by reference in their entireties for all purposes.

By “effective amount” is meant the amount of an agent or active compound, that is required to ameliorate the symptoms of a disease relative to an untreated patient or an individual without disease, i.e., a healthy individual, or is the amount of the agent or active compound sufficient to elicit a desired biological response. The effective amount of active compound(s) used to practice embodiments of the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

An “epitope tag” refers to a peptide or amino acid sequence (e.g., an epitope) that is fused, linked, or coupled to a protein, such as a recombinant protein produced by recombinant techniques, and that can be specifically bound by an antibody, e.g., an anti-tag monoclonal antibody or binding molecule that is directed to or generated against the tag peptide or amino acid sequence. In an embodiment, the protein to which an epitope tag is fused, linked, or coupled is an antibody or VHH protein, e.g., a recombinantly produced antibody or VHH protein. In an embodiment, the VHH is an anti-CD5 VHH antibody.

Other molecules may serve as protein, amino acid sequence, or polynucleotide tags that are fused, linked, or coupled to a protein, such as a recombinant protein produced by recombinant techniques, e.g., an anti-CD5 VHH antibody described herein. In an embodiment, the tag can be specifically bound by an antibody, e.g., an anti-tag monoclonal antibody or binding molecule that is directed to or generated against the tag peptide or amino acid sequence. Examples of tags include, without limitation, FLAG tags (peptide sequence DYKDDDDK (SEQ ID NO: 428) recognized by an anti-FLAG antibody), polyHistidine (His) tags (5-10 histidine residues (HHHHHH (SEQ ID NO: 429)) bound by a nickel or cobalt chelate), E-tag, a peptide comprising amino acid sequence GAPVPYPDPLEPR (SEQ ID NO: 430) recognized by an antibody; an immunoglobulin Fc region or portion thereof, e.g., having effector or modulator function (Fc tag).

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment.

A “framework (FR) region” or “FR region” includes amino acid residues that are adjacent to the CDRs in V_{H}, and V_{L} regions, and in VHHs. For example, FR region residues may be present in VHHs as described herein, camelid antibodies (VHHs), human antibodies, rodent-derived antibodies (e.g., murine and rat antibodies), humanized antibodies, primatized antibodies, chimeric antibodies, antibody fragments (e.g., Fab fragments), VHHs, single-chain antibody fragments (e.g., scFv fragments), antibody domains, and bispecific antibodies, among others.

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule. A guide polynucleotide typically contains a “spacer,” which may be about 20 base pairs in length. Shorter or longer spacers may also be used in guide polynucleotides, e.g., 15-, 16-, 17-, 18-, 19-, 21-, 22-, 23-, 24-, or 25-nucleotides in length. In some embodiments, a target sequence is in a gene or on a chromosome, for example, and is complementary to a space. In some embodiments, a degree of complementarity or identity between a spacer sequence and its corresponding target sequence may be about or at least 75%, 80%, 85%, 90%, 95%, 96%, 97%, 98%, 99%, or 100%.

The term “humanized” antibodies refers to forms of non-human (e.g., murine) antibodies, camelid-derived single domain antibody (sdAb) binding molecules, which are comprised of the heavy chain variable (V_{H}) region of heavy-chain-only antibodies (Abs) or VHHs. Humanized antibodies include chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, a humanized antibody or VHH may comprise substantially all of at least one variable domain (or two variable domains in the case of non-VHH antibodies), in which all or substantially all of the CDR regions correspond to those of a non-human immunoglobulin. All or substantially all of the FR regions of a humanized antibody may also be derived from a human immunoglobulin sequence. In the case of non-VHH antibodies, a VHH or a humanized antibody can also comprise at least a portion of an immunoglobulin constant region (Fc), which may be that of a human immunoglobulin consensus sequence. Techniques and protocols for humanizing antibodies (as well as VHHs) are known and practiced in the art, as described, for examples, in Riechmann et al., Nature, 332:323-7, 1988; Kasmiri et al., Methods, 36(1): 25-34, 2005; U.S. Pat. Nos. 5,530,101; 5,585,089; 5,693,761; 5,693,762; and U.S. Pat. No. 6,180,370 to Queen et al; EP239400; WO 1991/09967; U.S. Pat. No. 5,225,539; EP592106; and EP519596, the contents of which are incorporated herein by reference. Humanized antibodies or VHHs are molecularly engineered to contain even more human-like immunoglobulin domains, and incorporate only the CDRs of the VHH or animal-derived monoclonal antibody by carefully examining the sequence of the hyper-variable loops of the V regions of the monoclonal antibody or VHH, and fitting them to the structure of the human antibody chains. This process is routinely and commonly carried out by one having skill in the art. See, e.g., U.S. Pat. No. 6,187,287, the contents of which are incorporated by reference herein.

“Graft versus host disease” (GVHD) refers to a pathological condition where transplanted cells of a donor generate an immune response against cells of the host.

By “heterologous,” or “exogenous” is meant a polynucleotide or polypeptide that 1) has been experimentally incorporated into a polynucleotide or polypeptide sequence to which the polynucleotide or polypeptide is not normally found in nature; and/or 2) has been experimentally placed into a cell that does not normally comprise the polynucleotide or polypeptide. In some embodiments, “heterologous” means that a polynucleotide or polypeptide has been experimentally placed into a non-native context. In some embodiments, a heterologous polynucleotide or polypeptide is derived from a first species or host organism and is incorporated into a polynucleotide or polypeptide derived from a second species or host organism. In some embodiments, the first species or host organism is different from the second species or host organism. In some embodiments the heterologous polynucleotide is DNA. In some embodiments the heterologous polynucleotide is RNA.

“Host versus graft disease” (HVGD) or “host-versus-graft rejection” refers to a pathological condition where the immune system of a host generates an immune response against transplanted cells of an allogeneic donor.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immune cell” is meant a cell of the immune system capable of generating an immune response. Exemplary immune cells include, but are not limited to, T cells, NK cells, B cells, macrophages, hematopoietic stem cells, or precursors thereof. In embodiments, an immune cell is allogeneic to a subject to whom the cell is to be administered. In embodiments, an immune cell is from a donor and is allogeneic to a subject to which the immune cell will be administered after being modified according to the methods provided herein. The disclosure features methods for preparing modified allogeneic immune cells with improved characteristics (e.g., increased persistence in a subject) as well as the cells produced by these methods.

By “immune effector cell” is meant a lymphocyte, once activated, capable of effecting an immune response upon a target cell. In some embodiments, immune effector cells are effector T cells. In some embodiments, the effector T cell is a naïve CD8⁺ T cell, a cytotoxic T cell, a natural killer T (NKT) cell, a natural killer (NK) cell, or a regulatory T (Treg) cell. In some embodiments, immune effector cells are effector NK cells. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell).

By “immunomodulatory activity” is meant increasing, reducing, or sustaining an immune response.

By “increases” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The terms “inhibitor of base repair,” “base repair inhibitor,” “IBR” or their grammatical equivalents refer to a protein that is capable in inhibiting the activity of a nucleic acid repair enzyme, for example a base excision repair enzyme.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In some embodiments, the preparation is at least 75%, at least 90%, or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “kill switch” refers to a polypeptide capable of mediating the killing of a cell when the polypeptide is specifically bound by an agent. In some cases, the agent is a small molecule or monoclonal antibody. In some cases, the agent is Ritixumab. In various embodiments, the kill switch is selected from RQR1, RQR2, RQR8, RQR1G4S, RQR2G4S, RR, G4SRR, G4SRRG4S, G4SRRG4SCD8, G4SRRG4SCD28, G4SRRCD28, and QG4S. In various embodiments, the kill switch is fused to a chimeric antigen receptor. In some embodiments, a kill switch is expressed on the surface of a cell and is not fused to a chimeric antigen receptor.

The term “linker”, as used herein, refers to a molecule that links two moieties. In one embodiment, the term “linker” refers to a covalent linker (e.g., covalent bond) or a non-covalent linker.

By “marker” is meant any agent or clinical parameter having an alteration that is associated with a disease or disorder. In embodiments, the agent is a polypeptide or polynucleotide and the alteration is in expression, level, structure, or activity. In embodiments, the marker is associated with a disease or disorder. In some instances, the disease or disorder is a neoplasia, such as a hematologic cancer (e.g., T-cell acute lymphoblastic leukemia (T-ALL)). Non-limiting examples of markers include B2M, CD2, CD5, CD45, CIITA, HLA-DR, IFNg, PD1, and TCRαβ.

The term “mRNA” refers to a polynucleotide and comprises an open reading frame that can be translated into a polypeptide. An mRNA molecule may serve as a substrate for translation by a ribosome and amino-acylated tRNAs. An mRNA molecule may comprise a phosphate-sugar backbone including ribose residues or analogs thereof, e.g., 2′-methoxy ribose residues. In some embodiments, the sugars of an mRNA phosphate-sugar backbone contain or contain only ribose residues, 2′-methoxy ribose residues, or a combination thereof.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

“Neoplasia” refers to cells or tissues exhibiting abnormal growth or proliferation. The term neoplasia encompasses cancer, liquid, and solid tumors. In some embodiments, the neoplasia is a solid tumor. In other embodiments, the neoplasia is a liquid tumor. In some embodiments, the neoplasia is a hematological cancer. In some embodiments, the hematological cancer is leukemia, myeloma, and/or lymphoma. In some embodiments, the hematological cancer is a B cell cancer. In some embodiments, the B cell cancer is a lymphoma or a leukemia. In some cases, the leukemia comprises a pre-leukemia. In some cases, the leukemia is an acute leukemia. Acute leukemias include, for example, an acute myeloid leukemia (AML). Acute leukemias also include, for example, an acute lymphoid leukemia or an acute lymphocytic leukemia (ALL); ALL includes B-lineage ALL; T-lineage ALL; and T-cell acute lymphocytic leukemia (T-ALL).

By “helper lipid” is meant any neutral, zwitterionic, or anionic lipid.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (2′—e.g., fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi: 10.1038/nbt.4172. In some embodiments, an NLS comprises the amino acid sequence

```
  (SEQ ID NO: 190)
KRTADGSEFESPKKKRKV,
  (SEQ ID NO: 191)
KRPAATKKAGQAKKKK,
  (SEQ ID NO: 192)
KKTELQTTNAENKTKKL,
  (SEQ ID NO: 193)
KRGINDRNFWRGENGRKTR,
  (SEQ ID NO: 194)
RKSGKIAAIVVKRPRK,
  (SEQ ID NO: 195)
PKKKRKV,
  (SEQ ID NO: 196)
MDSLLMNRRKFLYQFKNVRWAKGRRETYLC,
  (SEQ ID NO: 328)
PKKKRKVEGADKRTADGSEFESPKKKRKV,
or
  (SEQ ID NO: 329)
RKSGKIAAIVVKRPRKPKKKRKV.
```

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (I). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpfl, Cas12b/C2cl, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpfl, Cas12b/C2cl, Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422): 88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-231, 232-245, 254-257, 260, and 378. In some embodiments, the napDNAbp is a (CRISPR-associated system) Cas9 endonuclease, for example, Cas9 (Csnl) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9). Further non-limiting examples of nucleic acid programmable DNA binding proteins include those disclosed or referenced in Rufflow, et al., “Design of highly functional genome editors by modeling of the universe of CRISPR-Cas Sequences,” bioRxiv, posted Apr. 22, 2024, doi: 10.1101/2024.04.22.590591, the disclosure of which is incorporated herein by reference in its entirety for all purposes, which were designed using artificial intelligence. In some embodiments, the napDNAbp is OpenCRISPR-1, or a variant thereof (e.g., a variant comprising a D10A amino acid alteration and/or lacking an N-terminal methionine). Further non-limiting examples of nucleic acid programmable DNA binding proteins include those disclosed in International Patent Application No. PCT/US2019/047996.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

By “operably linked” is meant the connection between regulatory elements and one or more polynucleotides (genes) or a coding region. That is, gene expression is typically placed under the control of certain regulatory elements, including constitutive or inducible promoters, tissue-specific regulatory elements, and enhancers. A polynucleotide (gene or genes) or coding region is said to be “operably linked to” or “operatively linked to” or “operably associated with” the regulatory elements, meaning that the polynucleotide (gene or genes) or coding region is controlled or influenced by the regulatory elements. The one or more polynucleotides may be separated by spacers or linkers.

The term “PEG lipid” or “PEG-modified lipid” refers to polyethylene glycol (PEG)-modified lipids.

By “OpenCRISPR-1 polypeptide” is meant a protein with an amino acid sequence having at least about 85% amino acid sequence identity to SEQ ID NO: 647, or a fragment thereof that associates with a nucleic acid, such as a guide nucleic acid or guide polynucleotide, that guides the napDNAbp to a specific nucleic acid sequence. Further details relating to the OpenCRISPR-1 polypeptide are disclosed in Rufflow, et al., “Design of highly functional genome editors by modeling of the universe of CRISPR-Cas Sequences,” bioRxiv, posted Apr. 22, 2024, doi: 10.1101/2024.04.22.590591, the disclosure of which is incorporated herein by reference in its entirety for all purposes.

By “OpenCRISPR-1 polynucleotide” is meant a nucleic acid molecule encoding an OpenCRISPR-1 polypeptide, as well as the introns, exons, 3′ untranslated regions, 5′ untranslated regions, and regulatory sequences associated with its expression, or fragments thereof. In embodiments, an OpenCRISPR-1 polynucleotide is the genomic sequence, cDNA, mRNA, or gene associated with and/or required for OpenCRISPR-1 expression. An exemplary OpenCRISPR-1 nucleotide sequence is provided at SEQ ID NO: 648.

In various embodiments, a guide RNA suitable for use in combination with an OpenCRISPR-1 polypeptide contains a scaffold having at least 85% sequence identity to a nucleotide sequence selected from the following, or fragments thereof capable of binding to an OpenCRISPR-1 polypeptide:

```
  (SEQ ID NO: 649)
GUUUUAGAGCUGUGUUGAAAAACACAGCAAGUUAAAAUAAGGCUUUGUCC
GUAUCCAACUUGAAAAAGUGAGCACCGAUUCGGUGC;
  (SEQ ID NO: 650)
GUUUUAGAGCUGGAAACAGCAAGUUAAAAUAAGGCUUUGUCCGUAUCCAA
CUUGAAAAAGUGAGCACCGAUUCGGUGC;
and
(SEQ ID NO: 651)
GUUUUAGAGCUAGAAAUAGCAAGUUAAAAUAAGGCUAGUCCGUUAUCAAC
UUGAAAAAGUGGCACCGAGUCGGUGC.
```

By “subject” or “patient” is meant a mammal, including, but not limited to, a human or non-human mammal. In embodiments, the mammal is a bovine, equine, canine, ovine, rabbit, rodent, nonhuman primate, or feline. In an embodiment, “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

By “persistence” in the context of an allogeneic transplant is meant the continued survival of a donor cell in a host organism. In some embodiments, allogeneic cell(s) comprising one or more of the edits described herein (e.g., a base edit in a CD5, CD3e, CD3g, B2M, and/or CIITA gene, or regulatory element(s) thereof; or knockdown of a CD5, TCRαβ, B2M, and/or CIITA polypeptide) persist in a subject allogeneic to the cells at higher levels over time post-infusion than corresponding unedited allogeneic control cells. In embodiments, the percentage of edited cells (e.g., T cells, NK cells, or lymphocytes) persisting in a subject at a given time point (e.g., 7 days, 14 days, 1 month, 3 months, 6 months, 9 months, or greater than 1, 2, or 3 years is at least about 10%, 15%, 20%, 25%, 30%, 35%, 40%, 45%, 50%, 60%, 70%, 80%, 90%, 95%, 99%, or 100% greater than the level of unedited control cells at the same time point. A cell(s) modified by methods of the present disclosure are more persistent than a reference unmodified cell(s).

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

The term “recombinant” as used herein in the context of proteins or nucleic acids refers to proteins or nucleic acids that do not occur in nature but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In one embodiment, the reference is a wild-type or healthy cell. In other embodiments and without limitation, a reference is an untreated cell or subject that is not subjected to a test condition, or is subjected to placebo or normal saline, medium, buffer, and/or a control vector that does not harbor a polynucleotide of interest. In some cases, the reference is an unedited or wild type cell (e.g., a T cell). In some cases, a reference is a healthy subject, such as a subject not having a neoplasia. In some embodiments, the reference is a subject not treated according to a method provided herein or not administered a composition provided herein (e.g., a composition comprising a CD5-binding polypeptide of the disclosure). The reference can be a cell that does not express one or more of the polypeptides described herein. The reference can be a subject before administration of a composition provided herein or treated according to a method provided herein and/or the subject before a change in a treatment (e.g., an alteration in dose or agent administered to the subject).

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease-RNA complex. Typically, the bound RNA(s) is referred to as a guide RNA (gRNA). In some embodiments, the RNA-programmable nuclease is the (CRISPR-associated system) Cas9 endonuclease, for example, Cas9 (Csnl) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9).

By “specifically binds” is meant recognizes and binds a polypeptide of the disclosure, but which does not substantially recognize and bind other molecules in a sample. In embodiments, a capture molecule is a VHH domain or a fragment thereof. A VHH domain or fragment thereof that specifically binds to an antigen will bind to the antigen with a K_{D} of less than 100 nM. For example, a VHH domain or fragment thereof that specifically binds to an antigen will bind to the antigen with a K_{D} of up to 100 nM (e.g., between 1 uM and 100 nM). A VHH domain or fragment thereof that does not exhibit specific binding to a particular antigen or epitope thereof will exhibit a K_{D} of greater than 100 nM (e.g., greater than 500 nm, 1 uM, 100 uM, 500 uM, or 1 mM) for that particular antigen or epitope thereof. A variety of immunoassay formats may be used to select a VHH domain or fragment thereof that specifically immunoreactive with a particular protein or carbohydrate. For example, solid-phase ELISA immunoassays are routinely used to select VHH domains or fragments thereof specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99% identical at the amino acid level or nucleic acid level to the sequence used for comparison.

Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “specifically binds” or “selectively binds” is meant a polypeptide (e.g., antibody) that recognizes and binds a molecule (e.g., polypeptide, antigen, ligand), but that does not substantially recognize or bind to other molecules in a sample, for example, a biological sample. For example, two molecules (e.g., an antibody and its ligand) that specifically bind to each other form a complex that is relatively stable under physiologic conditions. Specific binding is characterized by a high affinity and a low to moderate capacity, as distinguished from nonspecific binding which usually has a low affinity with a moderate to high capacity.

The term “targeting molecules” refers to molecules that bind to target cells of interest. In some embodiments, a targeting molecule is an anti-CD5 VHH antibody of the disclosure, or a CD5-binding fragment thereof. In some embodiments, a targeting molecule is an antibody or antigen-binding fragment thereof. In some embodiments, the targeting molecule is a ligand, receptor and/or antibody/antibody fragment. In some cases, a targeting molecule bind specifically to a target cell. A targeting molecule is considered to bind to a target cell if it binds to a cell surface marker (e.g., antigen, ligand, receptor) of the target cell. In some embodiments, targeting molecules bind specifically to particular target cells—that is, they bind to cell surface markers that are present only on the particular target cells. Thus, a targeting molecule is considered to bind specifically to a T cell if it binds a cell surface marker that is expressed only on T cells. In some embodiments, a targeting molecule is an anti-CD5 VHH antibody of the disclosure, or a CD5-binding fragment thereof.

The term “Targeting particles” or “targeted particles” refers to particles that comprise on their surface targeting molecules that bind to cell surface markers on target cells of interest. In some embodiments, the target cells are lymphocytes (e.g., T cells). A targeting particle is considered to comprise a targeting molecule on its surface if the targeting molecule is associated with or interacts with (e.g., is covalently or non-covalently conjugated to/bound to) the surface of the targeting particle.

The term “target site” refers to a nucleotide sequence or nucleobase of interest within a nucleic acid molecule that is modified. In embodiments, the modification is deamination of a base. The deaminase can be a cytidine or an adenine deaminase. The fusion protein or base editing complex comprising a deaminase may comprise a dCas9-adenosine deaminase fusion protein, a Cas12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, reduces the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. In various embodiments, a uracil DNA glycosylase (UGI) prevent base excision repair which changes the U back to a C. In some instances, contacting a cell and/or polynucleotide with a UGI and a base editor prevents base excision repair which changes the U back to a C. An exemplary UGI comprises an amino acid sequence as follows:

>splP14739IUNGI_BPPB2 Uracil-DNA
glycosylase inhibitor
(SEQ ID NO: 231)
MTNLSDIIEKETGKQLVIQESILMLPEEVEEVIGNKPESDILVHTAYDES
TDENVMLLTSDAPEYKPWALVIQDSNGENKIKML.

In some embodiments, the agent inhibiting the uracil-excision repair system is a uracil stabilizing protein (USP). See, e.g., WO 2022015969 A1, incorporated herein by reference.

As used herein, the term “vector” refers to a means of introducing a nucleic acid sequence into a cell, resulting in a transformed cell. Vectors include plasmids, transposons, phages, viruses, liposomes, lipid nanoparticles, and episomes.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “VHH domain” is meant an antigen binding domain of a heavy chain only antibody or an antigen binding fragment thereof.

A “VHH binding molecule” or “VHH antibody,” or simply “VHH,” as referred to herein is, in general, a single domain immunoglobulin molecule (antibody). A VHH (or VHH antibody) corresponds to the heavy chain of a VHH antibody having a single variable domain (or single variable region), e.g., a camelid-derived single variable H (V_{H}) domain antibody. A VHH typically has a molecular weight (MW) of about 12-15 kDa. VHH antibodies lack light chains. These heavy-chain antibody molecules contain a single variable domain (VHH) and, typically, two constant domains (CH₂ and CH₃). See, e.g., Methods in Molecular Biology, “Single Domain Antibodies—Methods and Protocols,” Eds. D. Saerens and S. Muyldermans, Humana Press (Springer), 2012. A cloned (recombinantly produced) and isolated VHH domain is a stable polypeptide harboring the antigen-binding capacity of the original heavy-chain antibody. See, e.g., U.S. Pat. Nos. 5,840,526 and 6,015,695, each of which is incorporated by reference herein in its entirety.

VHHs are efficiently expressed in E. coli, coupled to detection markers, such as a fluorescent marker, or conjugated with enzymes. The small size of VHHs permits their binding to epitopes (antigenic determinants in antigen proteins), e.g., “hidden epitopes” that are not accessible to whole antibodies of much larger size. As a therapeutic, a VHH is capable of efficient penetration and rapid clearance. Its single domain nature allows a VHH to be expressed in a cell without a requirement for supramolecular assembly, as is needed for whole antibodies which are typically tetrameric (two heavy chains and two light chains, having a MW of about 150 kDa). VHHs are also exhibit stability over time and have a longer half-life versus non-VHH antibody molecules, which comprise disulfide bonds that are susceptible to chemical reduction or enzymatic cleavage. Similar to immunoglobulins, VHHs may be modified post-translationally, e.g., to add chemical linkers, detectable moieties, such as fluorescent dyes, enzymes, substrates, chemiluminescent moieties, etc., or specific binding moieties, such as streptavidin, avidin, or biotin, etc., for use in the compositions and methods described herein.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended. This wording indicates that specified elements, features, components, and/or method steps are present, but does not exclude the presence of other elements, features, components, and/or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIGS. 1A and 1B provide plots showing binding of the indicated anti-CD5 VHH antibodies to CD5-expressing Jurkat cells.

FIGS. 2A and 2B provide plots providing representative data showing noncompetitive antibody binding (FIG. 2A) or competitive antibody binding (FIG. 2B). In FIGS. 2A and 2B Ab 1 was UCHT2, which was a control antibody capable of binding CD5, and Ab 2 was a CD5-binding polypeptide of the disclosure.

### DETAILED DESCRIPTION

The present disclosure features polypeptides capable of binding a cluster of differentiation 5 (CD5) antigen and polynucleotides encoding said CD5-binding polypeptides, compositions comprising the same, and methods for use thereof. The disclosure also features lipid nanoparticles comprising the CD5-binding polypeptides and methods for use thereof for delivery of a polynucleotide (e.g., a polynucleotide encoding a chimeric antigen receptor) to a T cell in vivo.

The various aspects of the disclosure are based, at least in part, on the discovery detailed in the Examples provided herein of new VHH antibodies capable of binding to a CD5 antigen.

### VHH Antibodies

In various aspects, the disclosure provides VHH antibodies, also known as “single-domain antibodies (sdAbs),” capable of binding a CD5 antigen, as well as polypeptides containing VHH domains or polynucleotides encoding the same. In embodiments, the CD5 bound by the VHH antibodies is associated with a disease or disorder, such as a neoplasia. In embodiments, the VHH binds an antigen associated with a target cell. In embodiments, the target cell is a neoplastic cell.

VHH domains are derived from sdAbs. Single-domain antibodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, a cloned and isolated VHH domain is a stable polypeptide harboring the full antigen-binding capacity of the original heavy-chain antibody. Single-domain antibodies have a high homology with the VH domains of human antibodies and can be further humanized without any loss of activity. Importantly, Single-domain antibodies have a low immunogenic potential, which has been confirmed in primate studies with sdAb lead compounds.

Single-domain antibodies combine the advantages of conventional antibodies with important features of small molecule drugs. Like conventional antibodies, sdAbs show high target specificity, high affinity for their target, and low inherent toxicity. However, like small molecule drugs they can inhibit enzymes and readily access receptor clefts. Furthermore, sdAbs are stable, can be administered by means other than injection (see, e.g., WO2004041867A2, which is herein incorporated by reference in its entirety) and are easy to manufacture. Other advantages of sdAbs include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Single-domain antibodies are encoded by single genes and are efficiently produced in almost all prokaryotic and eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula, or Pichia) (see, e.g., U.S. Pat. No. 6,838,254, which is herein incorporated by reference in its entirety).

VHHs, such as the anti-CD5 VHHs described herein, have a number of advantages over conventional antibodies and recombinant antibody domains, including (i) they are small monomeric proteins (14 kDa) that express and fold efficiently in recombinant hosts; (ii) they are more stable to extremes of pH and temperature compared with conventional antibodies; (iii) they typically bind conformational epitopes; and (iv) they are amenable to designed multimerization which often leads to higher potencies; and (v) they offer more therapeutic versatility, such as multispecificity, thus supporting their beneficial utility in treating diseases caused by or associated with CD5.

The amino acid sequences of representative anti-CD5 VHH antibodies described herein are provided in Table 1A below. Representative embodiments of the binding regions of the anti-CD5 VHHs include CDRs (CDR1, CDR2 and CDR3) as set forth in the sequences of representative anti-CD5 VHHs are presented in Tables 1A and 1B below. The CDR binding regions are positioned within framework (FR) regions (see Table 1C) of the VHH polypeptide (see Table 1A), which do not vary substantially in sequence between discrete anti-CD5 VHHs and which provide a “structural scaffold” for the CDRs, which bind to CD5. By way of non-limiting example, the binding of CDRs within FRs to a target protein (antigen), e.g., CD5, may be via conformational binding or interaction, electrostatic binding interaction, hydrogen bonding, Van der Waals forces, or hydrophobic bonding, or combinations thereof, as would be appreciated by those having skill in the art.

TABLE 1A
Anti-CD5 VHH antibody amino acid sequences.

| | | | SEQ |
|---|---|---|---|
| Clone | | | ID |
| Number | VHH Name | VHH Amino Acid Sequence | NO: |
| HCDR3 1 | | | |
| 199 | ABTX326 | QVQLVESGGGLVQPGGSLRLSCAASGRTFINYAAGWFRQA | 431 |
| | | PGKEREFVARISRSGGRTDYADSVKGRFTISRDNAKSTVY | |
| | | LQMNSLRPEDTAVYYCAEATVWEFTDGADQYDYWGQGTQV | |
| | | TVSS | |
| HCDR3 12 | | | |
| 662 | | EVQLVESGGGLVQAGGSLRLSCAASGRTFGSYTMGWFRQA | 432 |
| | | PGKEREFVAAISWSAGRTYYADSMKGRFTISRDNAKNTVY | |
| | | LQMNSLKPEDTAVYYCAADPWTSDSDYDRLTMYDYWGQGT | |
| | | QVTVSS | |
| 661 | | EVQLVESGGGLVQAGGSLRLSCAASGRTFGTYTMGWFRQA | 433 |
| | | PGKEREFVAAISWSAGRTYYADSMKGRFTISRDNAKNTVY | |
| | | LQMNSLKPEDTAVYYCAADPWTSDSDYDRLTMYDYWGQGT | |
| | | QVTVSS | |
| 641 | | QVQLQESGGGLVQAGGSLRLSCAASGRTFGSYTMGWFRQA | 434 |
| | | PGKEREFVAAISWSAGRTYYADSMKGRFTISRDNAKNTVY | |
| | | LQMNSLKPEDTAVYYCAADPWTSDSDYDRLTMYDYWGQGT | |
| | | QVTVSS | |
| 636 | | QVQLQESGGGLVQAGGSLRLSCAASGRTFGTYTMGWFRQA | 435 |
| | | PGKEREFVAAISWSAGRTYYADSMKGRFTISRDNAKNTVY | |
| | | LQMNSLKPEDTAVYYCAADPWTSDSDYDRLTMYDYWGQGT | |
| | | QVTVSS | |
| 739 | | QVQLQESGGGLVQAGGSLRLSCAASGRTFGTYTMGWFRQA | 436 |
| | | PGKGREFVAAISWSAGRTYYADSMKGRFTISRDNAKNTVY | |
| | | LQMNSLKPEDTAVYYCAADPWTSDSDYDRLTMYDYWGQGT | |
| | | QVTVSS | |
| 667 | | QVQLVESGGGLVQAGGSLRLSCAASGRTFGSYTMGWFRQA | 437 |
| | | PGKEREFVAAISWSAGRTYYADSMKGRFTISRDNAKNTVY | |
| | | LQMNSLKPEDTAVYYCAADPWTSDSDYDRLTMYDYWGQGT | |
| | | QVTVSS | |
| 657 | | QVQLVESGGGLVQAGGSLRLSCAASGRTFGTYTMGWFRQA | 438 |
| | | PGKEREFVAAI SWSAGRTYYADSMKGRFTISRDNAKNTVY | |
| | | LQMNSLKPEDTAVYYCAADPWTSDSDYDRLTMYDYWGQGT | |
| | | QVTVSS | |
| 502 | ABTX315 | QVQLVESGGGLVQAGGSLRLSCAASGRTFGTYTMGWFRQA | 439 |
| | | PGREREFVAAISWSAGRTYYADSMKGRFTISRDNAKNTVY | |
| | | LQMNSLKPEDTAVYYCAADPWTSDSDYDRLTMYDYWGQGT | |
| | | QVTVSS | |
| HCDR3 13 | | | |
| 727 | | EVQLVESGGGLVQAGGSLRLSCAASGGTVSSYAMGWFRQA | 440 |
| | | PGKEREFVAVISWSGGRTYYADSVKGRFTISRDNAKNTVN | |
| | | LQMNSLKPEDTAVYYCAADPWTSDSDYERLTMYDYWGQGT | |
| | | QVTVSS | |
| 630 | | EVQLVESGGGLVQAGGSRRLSCAASGGTVSSYAMGWFRQA | 441 |
| | | PGKEREFVAVISWSGGRTYYADSVKGRFTISRDNAKNTVY | |
| | | LQMNSLKPEDTAVYYCAADPWTSDSDYERLTMYDYWGQGT | |
| | | QVTVSS | |
| 525 | ABTX316 | QVQLVESGGGLVQAGGSLRLSCAASGGTVSSYAMGWFRQA | 442 |
| | | PGKEREFVAVISWSGGRTYYADSVKGRFTISRDNAKNTVN | |
| | | LQMNSLKPEDTAVYYCAADPWTSDSDYERLTMYDYWGQGT | |
| | | QVTVSS | |
| 728 | | QVQLVESGGGLVQAGGSLRLSCAASGGTVSSYAMGWFRQA | 443 |
| | | PGKEREFVAVISWSGGRTYYADSVKGRFTISRDNAKNTVY | |
| | | LQMNSLKPEDTAVYYCAADPWTSDSDYERLTMYDYWGQGT | |
| | | QVTVSS | |
| HCDR3 15 | | | |
| 133 | | EVQLVESGGGLVQAGASLRLSCAASGRTTYNMGWFRHAPG | 444 |
| | | KDREFVAAIDLYGRATRYANSVKGRFTISRDNAKNTVYLQ | |
| | | MNSLKPEDTAVYYCAADTSLPLGVLTESQRLYGAWGQGTQ | |
| | | VTVSS | |
| 242 | | QVQLQESGGGLVQAGASLRLSCAASGRATYNMGWFRHAPG | 445 |
| | | EDREFVAAINLEGYATRYANSVKGRFTISRDNAKNTVYLQ | |
| | | MNSLKPEDTAVYYCAADTSLPLGVLTESQRLYGAWGQGTQ | |
| | | VTVSS | |
| 218 | | QVQLQESGGGLVQAGASLRLSCAASGRTTYNMGWFRHAPG | 446 |
| | | KDREFVAAIDLYGRATRYANSVKGRFTISRDNAKNTVYLQ | |
| | | MNSLKPEDTAVYYCAADTSLPLGVLTESQRLYGAWGQGTQ | |
| | | VTVSS | |
| 309 | | QVQLQESGGGLVQAGASLRLSCAASGRTTYNMGWFRHAPG | 447 |
| | | KDREFVAAIDLYGRATRYANSVRGRFTISRDNAKNTVYLQ | |
| | | MNSLKPEDTAVYYCAADTSLPLGVLTESQRLYGAWGQGTQ | |
| | | VTVSS | |
| 225 | ABTX331 | QVQLVESGGGLVQAGASLRLSCAASGRTTYNMGWFRHAPG | 448 |
| | | KDREFVAAIDLYGRATRYANSVKGRFTISRDNAKNTVYLQ | |
| | | MNSLKPEDTAVYYCAADTSLPLGVLTESQRLYGAWGQGTQ | |
| | | VTVSS | |
| HCDR3 17 | | | |
| 280 | ABTX317 | QVQLVESGGGLVQAGASLRLSCAASGRTTYNMGWFRHAPG | 449 |
| | | KDREFVAAIDLYGRATRYANSVKGRFTISRDNAKNTVYLQ | |
| | | MNSLKPEDTAVYYCAADTSLPLGVLTKSQRMYGAWGQGTQ | |
| | | VTVSS | |
| HCDR3 28 | | | |
| 294 | | QVQLQESGGGSVQAGGSLRLSCAASGRAFSAYAMGWFRQA | 450 |
| | | PGKEREFVAAINWNGDTALRWNGFATRYADSVKGRFIISR | |
| | | VNAKNTVNLQMNSLKPEDTAVYYCAADTVVSGSYYLAARA | |
| | | EDYEYWGQGTQVSVSS | |
| 333 | ABTX318 | QVQLQESGGGSVQAGGSLRLSCAASGRAFSAYAMGWFRQA | 451 |
| | | PGKEREFVAAINWNGDTALRWNGFATRYADSVKGRFTISR | |
| | | VIAKNTVNLQMNSLKPEDTAVYYCAADTVVSGSYYLAARA | |
| | | EDYEYWGQGTQVTVSS | |
| 253 | | QVQLQESGGGSVQAGGSLRLSCAASGRAFSAYAMGWFRQA | 452 |
| | | PGKEREFVAAINWNGDTALRWNGFATRYADSVKGRFTISR | |
| | | VNAKNTVNLQMNSLKPEDTAVYYCAADTVVSGSYYLAARA | |
| | | EDYEYWGQGTQVSVSS | |
| 86 | | QVQLQESGGGSVQAGGSLRLSCAASGRAFSAYAMGWFRQA | 453 |
| | | PGKEREFVAAINWNGDTALRWNGFATRYADSVKGRFTISR | |
| | | VNAKNTVNLQMNSLKPEDTAVYYCAADTVVSGSYYLAARA | |
| | | EDYEYWGQGTQVTVSS | |
| HCDR3 50 | | | |
| 71 | | EVQLVESGGGLVQAGGSLRLSCAASGPAFSSSGMGWFRQA | 454 |
| | | PGKARDFVASMDWSGGSTYYGDSVKGRFTVSRDNAKNTVH | |
| | | LQMNSLRPEDTAVYYCARGTSGVAAVNLRGFFSWGPGTQV | |
| | | TVSS | |
| 3 | | QVQLQESGGGLVQAGGSLRLSCAASGPAFSSSGMGWFRQA | 455 |
| | | PGKARDFVASIDWSGGSTYYGDSVKGRFTVSRDNAKNTVH | |
| | | LQMNSLRPEDTAVYYCARGTSGVAAVNLRGFFSWGPGTQV | |
| | | TVSS | |
| 15 | | QVQLVESGGGLVQAGGSLRLACAASGAAFSSSGMGWFRQA | 456 |
| | | PGKARDFVASMDWSGGSTYYGDSVKGRFTVSRDNAKNTVH | |
| | | LQMNSLRPEDTAVYYCARGTSGVAAVNLRGFFSWGPGTQV | |
| | | TVSS | |
| 43 | | QVQLVESGGGLVQAGGSLRLSCAASGPAFSSSGMGWFRQA | 457 |
| | | PGKARDFVASIDWGGGSTYYGDSVKGRFTVSRDNAKNAVH | |
| | | LQMNSLRPEDTAVYYCARGTSGVAAVNLRGFFSWGPGTQV | |
| | | TVSS | |
| 5 | | QVQLVESGGGLVQAGGSLRLSCAASGPAFSSSGMGWFRQA | 458 |
| | | PGKARDFVASIDWSGGSTYYGDSVKGRFTVSRDNAKNTVH | |
| | | LQMNSLRPEDTAVYYCARGTSGVAAVNLRGFFSWGPGTQV | |
| | | TVSS | |
| 148 | | QVQLVESGGGLVQAGGSLRLSCAASGPAFSSSGMGWFRQA | 459 |
| | | PGKARDFVASIDWSGKSTYYGDSVKGRFTVSRDNAKNTVH | |
| | | LQMNSLRPEDTAVYYCARGTSGVAAVNLRGFFSWGPGTQV | |
| | | TVSS | |
| 51 | | QVQLVESGGGLVQAGGSLRLSCAASGPAFSSSGMGWFRQA | 460 |
| | | PGKARDFVASINWSGGSAYYGDSVKGRFTVSRDNAKNTVH | |
| | | LQMNSLRPEDTAVYYCARGTSGVAAVNLRGFFSWGPGTQV | |
| | | TVSS | |
| 84 | | QVQLVESGGGLVQAGGSLRLSCAASGPAFSSSGMGWFRQA | 461 |
| | | PGKARDFVASMDWSGGSTYYGDSVKGRFTVSRDNAKNTVH | |
| | | LQMNSLRPEDTAVYYCARGTSGVAAVNLRGFFSWGPGTQV | |
| | | TVSS | |
| 157 | ABTX320 | QVQLVESGGGLVQAGGSLRLSCAASGPAFSSSGMGWFRQA | 462 |
| | | PGKAREFVASMDWTGGSTYYGDSVKGRFTVSRDNAKMTVH | |
| | | LQMNSLRPEDTAVYYCARGTSGVAAVNLRGFFSWGPGTQV | |
| | | TVSS | |
| HCDR3 63 | | | |
| 660 | ABTX322 | QLQLVESGGGLVQPGGSLRLSCAASGSDFLVDATTWFRQA | 463 |
| | | PGNQREFVAIMDIGGVTEYADSVKGRFTISRDHTKNTVYL | |
| | | QMNSLKVEDTAVYYCNTRGLWGQGTLVTVSS | |
| HCDR3 65 | | | |
| 155 | ABTX323 | QVQLQESGGGLVQAGGSLRLSCATSGITSSINVIGWYRQA | 464 |
| | | PGKQRELVALVNSGGQTHYADSVKGRFTISRDNAKNTVFL | |
| | | QMNSLKPEDTAEYYCHGRYGIDNYWGEGTQVTVSS | |
| HCDR3 66 | | | |
| 563 | ABTX330 | EVQLVESGGGLVQPGGSLRLSCAASGFPFSSSFMSWVRQA | 465 |
| | | PGKGLEWVSTIYSDGSTYYADSVKGRFTISRDNAKKTAYL | |
| | | QMNSLKAEDTAVYYCATVTGSIRGQGTQVTVSS | |
| 500 | | QVQLVESGGGLVQPGGSLRLSCAASGFNFSSSFMSWVRQA | 466 |
| | | PGKEVEWVSTIYSDGSTYYADSMKGRFTISRDNAKNTVYL | |
| | | QMSNLKAEDTAVYYCATVTGSIRGQGTQVTVSS | |
| HCDR3 67 | | | |
| 550 | ABTX324 | QVQLVESGGGLVQPGGSVRLSCATSGSIFSTNVMGWYRQA | 467 |
| | | PGKEREFVALIRGGGSTHYADSVKGRFIISRENAKTTVYL | |
| | | QMNGLKPEDTAVYYCVIWLGSPGAMSDYWGQGTQVTVSS | |
| HCDR3 68 | | | |
| 647 | | EVQLVESGGGLVQPGGSLRLSCAASGSVVSTNNMGWYRQA | 468 |
| | | PGKEREFVALIRTGGSTHVADSMKGRFTISRENAKNTVYL | |
| | | QMNSLKPEDTAVYYCVIWTGSPGALSDYWGQGTQVTVSS | |
| 420 | ABTX325 | QVQLVESGGGLVQPGGSLRLSCAASGSDASTNNMAWYRQA | 469 |
| | | PGKEREFVALIRTGGSTHVADSMKGRFTISRENAKNTVYL | |
| | | QMNSLKPEDTAVYYCVIWTGSPGALSDYWGQGTQVTVSS | |
| HCDR3 60 | | | |
| 1065 | | QLQLVESGGGLVQPGESLRLSCAASGFSFSRVAMNWYRQA | 470 |
| | | PGKERELVATISSDGSRTNYAHSVKGRFTISRENAKNMVY | |
| | | LQMNSLKLEDTAVYYCNVPGNSWGQGTQVTVSS | |
| 1091 | ABTX329 | QLQLVESGGGLVQPGESLRLSCAASGFSFSRVGMNWYRQA | 471 |
| | | PGKERELVASISSDGSRTNYAHFVKGRFTISRDNVKNMVY | |
| | | LQMNSLKLEDTAVYYCNVPGNSWGQGTQVTVSS | |
| HCDR3 57 | | | |
| 1043 | ABTX321 | QLQLVESGGGLVQPGESLRLSCVVSGDIFSFVGWGWYRQA | 472 |
| | | PGKQREVVAQISTGGLTNYADSVKGRFAISRDNAKRTVYL | |
| | | QMNSLKFEDTAVYYCNVPGHPWGQGTQVTVSS | |
| HCDR3 58 | | | |
| 1050 | ABTX328 | QVQLVESGGGLVQPGESLRLSCVVSGDIFSFIGWGWYRQA | 473 |
| | | PGKQRELVAQINTGGLTDYADSVKGRFTISRDNAKRTVYL | |
| | | QMNSLKFEDTAVYYCNFPGHSWGQGTQVTVSS | |
| HCDR3 43 | | | |
| 917 | ABTX319 | QVQLVESGGGLVQPGGSLRLSCAASGFTFSMYSMSWVRQA | 474 |
| | | PGKRLEWVSSISTGARDTAYADSVKGRFTISRDNADNTLY | |
| | | LHMNNLKPEDTAVYYCANGDLRYGPDGYDYRGQGTQVTVS | |
| | | S | |
| 928 | | QVQLVESGGGLVQPGGSLRLSCAASGFTFSMYSMSWVRQA | 475 |
| | | PGKRLEWVSSISTGARDTAYADSVKGRFTISRDNADNTLY | |
| | | LHMNNLKPEDTAVYYCANGDLRYGPDGYDYRGQGTQVTVV | |
| | | S | |
| 923 | | QVQLVESGGGLVQPGGSLRLSCAASGFTFSMYSMSWVRQA | 476 |
| | | PGKRLEWVSSISTGARDTSYADSVKGRFTISRDNADNTLY | |
| | | LHMNNLKPEDTAVYYCANGDLRYGPDGYDYRGQGTQVTVS | |
| | | S | |
| HCDR3 46 | | | |
| 834 | ABTX327 | QVQLVESGGGLVQPGGSLRLSCVASGGTFSTYGMGWFRQA | 477 |
| | | PGKEREFVAVITGSGVGTQYADSVKDRFTISRENAKNTVY | |
| | | LQMNTLKLEDTAVYYCVSGHRPGWAVIRADAYEYWGQGTQ | |
| | | VTVSS | |

Embodiments of complementarity determining regions (CDRs) are underlined in each sequence, where the CDRs, from N-terminus to C-terminus, are CDR1, CDR2, and CDR3. The non-underlined portions of each sequence correspond, in order from N-terminus to C-terminus, FR1, FR2, FR3, and FR4.

TABLE 1B
Anti-CD5 VHH antibody complementarity determining region
(CDR) amino acid sequences.

| Clone | VHH | CD | SEQ | | SEQ | | SEQ |
|---|---|---|---|---|---|---|---|
| Number | Name | R1 | ID NO | CDR2 | ID NO | CDR3 | ID NO |
| HCDR3 1 | | | | | | | |
| 199 | ABTX | NYA | 478 | RISRSGGRTDYADSV | 486 | ATVWEFTDGADQ | 498 |
| | 326 | AG | | KG | | YDY | |
| HCDR3 12 | | | | | | | |
| 662 | | SYT | 479 | AISWSAGRTYYADSM | 487 | DPWTSDSDYDRL | 499 |
| | | MG | | KG | | TMYDY | |
| 661 | | TYT | 480 | AISWSAGRTYYADSM | 487 | DPWTSDSDYDRL | 499 |
| | | MG | | KG | | TMYDY | |
| 641 | | SYT | 479 | AISWSAGRTYYADSM | 487 | DPWTSDSDYDRL | 499 |
| | | MG | | KG | | TMYDY | |
| 636 | | TYT | 480 | AISWSAGRTYYADSM | 487 | DPWTSDSDYDRL | 499 |
| | | MG | | KG | | TMYDY | |
| 739 | | TYT | 480 | AISWSAGRTYYADSM | 487 | DPWTSDSDYDRL | 499 |
| | | MG | | KG | | TMYDY | |
| 667 | | SYT | 479 | AISWSAGRTYYADSM | 487 | DPWTSDSDYDRL | 499 |
| | | MG | | KG | | TMYDY | |
| 657 | | TYT | 480 | AISWSAGRTYYADSM | 487 | DPWTSDSDYDRL | 499 |
| | | MG | | KG | | TMYDY | |
| 502 | ABTX | TYT | 480 | AISWSAGRTYYADSM | 487 | DPWTSDSDYDRL | 499 |
| | 315 | MG | | KG | | TMYDY | |
| HCDR3 13 | | | | | | | |
| 727 | | SYA | 481 | VISWSGGRTYYADSV | 488 | DPWTSDSDYERL | 500 |
| | | MG | | KG | | TMYDY | |
| 630 | | SYA | 481 | VISWSGGRTYYADSV | 488 | DPWTSDSDYERL | 500 |
| | | MG | | KG | | TMYDY | |
| 525 | ABTX | SYA | 481 | VISWSGGRTYYADSV | 488 | DPWTSDSDYERL | 500 |
| | 316 | MG | | KG | | TMYDY | |
| 728 | | SYA | 481 | VISWSGGRTYYADSV | 488 | DPWTSDSDYERL | 500 |
| | | MG | | KG | | TMYDY | |
| HCDR3 15 | | | | | | | |
| 133 | | TYN | 482 | AIDLYGRATRYANSV | 489 | DTSLPLGVLTES | 501 |
| | | MG | | KG | | QRLYGA | |
| 242 | | TYN | 482 | AINLEGYATRYANSV | 489 | DTSLPLGVLTES | 501 |
| | | MG | | KG | | QRLYGA | |
| 218 | | TYN | 482 | AIDLYGRATRYANSV | 489 | DTSLPLGVLTES | 501 |
| | | MG | | KG | | QRLYGA | |
| 309 | | TYN | 482 | AIDLYGRATRYANSV | 489 | DTSLPLGVLTES | 501 |
| | | MG | | RG | | QRLYGA | |
| 225 | ABTX | TYN | 482 | AIDLYGRATRYANSV | 489 | DTSLPLGVLTES | 501 |
| | 331 | MG | | KG | | QRLYGA | |
| HCDR3 17 | | | | | | | |
| 280 | ABTX | TYN | 482 | AIDLYGRATRYANSV | 489 | DTSLPLGVLTKS | 502 |
| | 317 | MG | | KG | | QRMYGA | |
| HCDR3 28 | | | | | | | |
| 294 | | AYA | 483 | AINWNGDTALRWNGF | 490 | DTVVSGSYYLAA | 503 |
| | | MG | | ATRYADSVKG | | RAEDYEY | |
| 333 | ABTX | AYA | 483 | AINWNGDTALRWNGF | 490 | DTVVSGSYYLAA | 503 |
| | 318 | MG | | ATRYADSVKG | | RAEDYEY | |
| 253 | | AYA | 483 | AINWNGDTALRWNGF | 490 | DTVVSGSYYLAA | 503 |
| | | MG | | ATRYADSVKG | | RAEDYEY | |
| 86 | | AYA | 483 | AINWNGDTALRWNGF | 490 | DTVVSGSYYLAA | 503 |
| | | MG | | ATRYADSVKG | | RAEDYEY | |
| HCDR3 50 | | | | | | | |
| 71 | | SSG | 484 | SMDWSGGSTYYGDSV | 491 | GTSGVAAVNLRG | 504 |
| | | MG | | KG | | FFS | |
| 3 | | SSG | 484 | SIDWSGGSTYYGDSV | 492 | GTSGVAAVNLRG | 504 |
| | | MG | | KG | | FFS | |
| 15 | | SSG | 484 | SMDWSGGSTYYGDSV | 491 | GTSGVAAVNLRG | 504 |
| | | MG | | KG | | FFS | |
| 43 | | SSG | 484 | SIDWGGGSTYYGDSV | 493 | GTSGVAAVNLRG | 504 |
| | | MG | | KG | | FFS | |
| 5 | | SSG | 484 | SIDWSGGSTYYGDSV | 492 | GTSGVAAVNLRG | 504 |
| | | MG | | KG | | FFS | |
| 148 | | SSG | 484 | SIDWSGKSTYYGDSV | 494 | GTSGVAAVNLRG | 504 |
| | | MG | | KG | | FFS | |
| 51 | | SSG | 484 | SINWSGGSAYYGDSV | 495 | GTSGVAAVNLRG | 504 |
| | | MG | | KG | | FFS | |
| 84 | | SSG | 484 | SMDWSGGSTYYGDSV | 491 | GTSGVAAVNLRG | 504 |
| | | MG | | KG | | FFS | |
| 157 | ABTX | SSG | 484 | SMDWTGGSTYYGDSV | 496 | GTSGVAAVNLRG | 504 |
| | 320 | MG | | KG | | FFS | |
| HCDR3 63 | | | | | | | |
| 660 | ABTX | VDA | 485 | IMDIGGVTEYADSVK | 497 | RGL | |
| | 322 | TT | | G | | | |
| HCDR3 65 | | | | | | | |
| 155 | ABTX | INV | 505 | LVNSGGQTHYADSVK | 516 | RYGIDNY | 528 |
| | 323 | IG | | G | | | |
| HCDR3 66 | | | | | | | |
| 563 | ABTX | SSF | 506 | TIYSDGSTYYADSVK | 517 | VTGSI | 529 |
| | 330 | MS | | G | | | |
| 500 | | SSF | 506 | TIYSDGSTYYADSMK | 518 | VTGSI | 529 |
| | | MS | | G | | | |
| HCDR3 67 | | | | | | | |
| 550 | ABTX | TNV | 507 | LIRGGGSTHYADSVK | 519 | WLGSPGAMSDY | 530 |
| | 324 | MG | | G | | | |
| HCDR3 68 | | | | | | | |
| 647 | | TNN | 508 | LIRTGGSTHVADSMK | 520 | WTGSPGALSDY | 531 |
| | | MG | | G | | | |
| 420 | ABTX | TNN | 509 | LIRTGGSTHVADSMK | 520 | WTGSPGALSDY | 531 |
| | 325 | MA | | G | | | |
| HCDR3 60 | | | | | | | |
| 1065 | | RVA | 510 | TISSDGSRTNYAHSV | 522 | PGNS | 532 |
| | | MN | | KG | | | |
| 1091 | ABTX | RVG | 511 | SISSDGSRTNYAHFV | 523 | PGNS | 532 |
| | 329 | MN | | KG | | | |
| HCDR3 57 | | | | | | | |
| 1043 | ABTX | FVG | 512 | QISTGGLTNYADSVK | 524 | PGHP | 533 |
| | 321 | WG | | G | | | |
| HCDR3 58 | | | | | | | |
| 1050 | ABTX | FIG | 513 | QINTGGLTDYADSVK | 525 | PGHS | 534 |
| | 328 | WG | | G | | | |
| HCDR3 43 | | | | | | | |
| 917 | ABTX | MYS | 514 | SISTGARDTAYADSV | 526 | GDLRYGPDGYDY | 535 |
| | 319 | MS | | KG | | | |
| 928 | | MYS | 514 | SISTGARDTAYADSV | 526 | GDLRYGPDGYDY | 535 |
| | | MS | | KG | | | |
| 923 | | MYS | 514 | SISTGARDTSYADSV | 526 | GDLRYGPDGYDY | 535 |
| | | MS | | KG | | | |
| HCDR3 46 | | | | | | | |
| 834 | ABTX | TYG | 515 | VITGSGVGTQYADSV | 527 | GHRPGWAVIRAD | 536 |
| | 327 | MG | | KD | | AYEY | |

TABLE 1C
Anti-CD5 VHH antibody framework region (FR) amino acid sequences.

| | | | SEQ | | SEQ | | SEQ | | SEQ |
|---|---|---|---|---|---|---|---|---|---|
| Clone | VHH | | ID | | ID | | ID | | ID |
| Number | Name | FR1 | NO | FR2 | NO | FR3 | NO | FR4 | NO |
| HCDR3 1 | | | | | | | | | |
| 199 | ABTX3 | QVQLVESG | 537 | WFRQAPG | 539 | RFTISRD | 540 | WGQG | 542 |
| | 26 | GGLVQPGG | | KEREFVA | | NAKSTVY | | TQVT | |
| | | SLRLSCAA | | | | LQMNSLR | | VSS | |
| | | SGRTFI | | | | PEDTAVY | | | |
| | | | | | | YCAE | | | |
| HCDR3 12 | | | | | | | | | |
| 662 | | EVQLVESG | 538 | WFRQAPG | 539 | RFTISRD | 541 | WGQG | 542 |
| | | GGLVQAGG | | KEREFVA | | NAKNTVY | | TQVT | |
| | | SLRLSCAA | | | | LQMNSLK | | VSS | |
| | | SGRTFG | | | | PEDTAVY | | | |
| | | | | | | YCAA | | | |
| 661 | | EVQLVESG | 538 | WFRQAPG | 539 | RFTISRD | 541 | WGQG | 542 |
| | | GGLVQAGG | | KEREFVA | | NAKNTVY | | TQVT | |
| | | SLRLSCAA | | | | LQMNSLK | | VSS | |
| | | SGRTFG | | | | PEDTAVY | | | |
| | | | | | | YCAA | | | |
| 641 | | QVQLQESG | 543 | WFRQAPG | 539 | RFTISRD | 541 | WGQG | 542 |
| | | GGLVQAGG | | KEREFVA | | NAKNTVY | | TQVT | |
| | | | | | | LQMNSLK | | VSS | |
| | | SLRLSCAA | | | | PEDTAVY | | | |
| | | SGRTFG | | | | YCAA | | | |
| 636 | | QVQLQESG | 543 | WFRQAPG | 539 | RFTISRD | 541 | WGQG | 542 |
| | | GGLVQAGG | | KEREFVA | | NAKNTVY | | TQVT | |
| | | SLRLSCAA | | | | LQMNSLK | | VSS | |
| | | SGRTFG | | | | PEDTAVY | | | |
| | | | | | | YCAA | | | |
| 739 | | QVQLQESG | 543 | WFRQAPG | 620 | RFTISRD | 541 | WGQG | 542 |
| | | GGLVQAGG | | KGREFVA | | NAKNTVY | | TQVT | |
| | | SLRLSCAA | | | | LQMNSLK | | VSS | |
| | | SGRTFG | | | | PEDTAVY | | | |
| | | | | | | YCAA | | | |
| 667 | | QVQLVESG | 619 | WFRQAPG | 539 | RFTISRD | 541 | WGQG | 542 |
| | | GGLVQAGG | | KEREFVA | | NAKNTVY | | TQVT | |
| | | SLRLSCAA | | | | LQMNSLK | | VSS | |
| | | SGRTFG | | | | PEDTAVY | | | |
| | | | | | | YCAA | | | |
| 657 | | QVQLVESG | 619 | WFRQAPG | 539 | RFTISRD | 541 | WGQG | 542 |
| | | GGLVQAGG | | KEREFVA | | NAKNTVY | | TQVT | |
| | | SLRLSCAA | | | | LQMNSLK | | VSS | |
| | | SGRTFG | | | | PEDTAVY | | | |
| | | | | | | YCAA | | | |
| 502 | ABTX3 | QVQLVESG | 619 | WFRQAPG | 621 | RFTISRD | 541 | WGQG | 542 |
| | 15 | GGLVQAGG | | REREFVA | | NAKNTVY | | TQVT | |
| | | SLRLSCAA | | | | LQMNSLK | | VSS | |
| | | SGRTFG | | | | PEDTAVY | | | |
| | | | | | | YCAA | | | |
| HCDR3 13 | | | | | | | | | |
| 727 | | EVQLVESG | 544 | WFRQAPG | 539 | RFTISRD | 547 | WGQG | 542 |
| | | GGLVQAGG | | KEREFVA | | NAKNTVN | | TQVT | |
| | | SLRLSCAA | | | | LQMNSLK | | VSS | |
| | | SGGTVS | | | | PEDTAVY | | | |
| | | | | | | YCAA | | | |
| 630 | | EVQLVESG | 545 | WFRQAPG | 539 | RFTISRD | 541 | WGQG | 542 |
| | | GGLVQAGG | | KEREFVA | | NAKNTVY | | TQVT | |
| | | SRRLSCAA | | | | LQMNSLK | | VSS | |
| | | SGGTVS | | | | PEDTAVY | | | |
| | | | | | | YCAA | | | |
| 525 | ABTX3 | QVQLVESG | 546 | WFRQAPG | 539 | RFTISRD | 547 | WGQG | 542 |
| | 16 | GGLVQAGG | | KEREFVA | | NAKNTVN | | TQVT | |
| | | SLRLSCAA | | | | LQMNSLK | | VSS | |
| | | SGGTVS | | | | PEDTAVY | | | |
| | | | | | | YCAA | | | |
| 728 | | QVQLVESG | 546 | WFRQAPG | 539 | RFTISRD | 541 | WGQG | 542 |
| | | GGLVQAGG | | KEREFVA | | NAKNTVY | | TQVT | |
| | | SLRLSCAA | | | | LQMNSLK | | VSS | |
| | | SGGTVS | | | | PEDTAVY | | | |
| | | | | | | YCAA | | | |
| HCDR3 15 | | | | | | | | | |
| 133 | | EVQLVESG | 548 | WFRHAPG | 552 | RFTISRD | 541 | WGQG | 542 |
| | | GGLVQAGA | | KDREFVA | | NAKNTVY | | TQVT | |
| | | SLRLSCAA | | | | LQMNSLK | | VSS | |
| | | SGRT | | | | | | | |
| | | | | | | PEDTAVY | | | |
| | | | | | | YCAA | | | |
| 242 | | QVQLQESG | 549 | WERHAPG | 553 | RFTISRD | 541 | WGQG | 542 |
| | | GGLVQAGA | | EDREFVA | | NAKNTVY | | TQVT | |
| | | SLRLSCAA | | | | LQMNSLK | | VSS | |
| | | SGRA | | | | PEDTAVY | | | |
| | | | | | | YCAA | | | |
| 218 | | QVQLQESG | 550 | WFRHAPG | 552 | RFTISRD | 541 | WGQG | 542 |
| | | GGLVQAGA | | KDREFVA | | NAKNTVY | | TQVT | |
| | | SLRLSCAA | | | | LQMNSLK | | VSS | |
| | | SGRT | | | | PEDTAVY | | | |
| | | | | | | YCAA | | | |
| 309 | | QVQLQESG | 550 | WFRHAPG | 552 | RFTISRD | 541 | WGQG | 542 |
| | | GGLVQAGA | | KDREFVA | | NAKNTVY | | TQVT | |
| | | SLRLSCAA | | | | LQMNSLK | | VSS | |
| | | SGRT | | | | PEDTAVY | | | |
| | | | | | | YCAA | | | |
| 225 | ABTX3 | QVQLVESG | 551 | WFRHAPG | 552 | RFTISRD | 541 | WGQG | 542 |
| | 31 | GGLVQAGA | | KDREFVA | | NAKNTVY | | TQVT | |
| | | SLRLSCAA | | | | LQMNSLK | | VSS | |
| | | SGRT | | | | PEDTAVY | | | |
| | | | | | | YCAA | | | |
| HCDR3 17 | | | | | | | | | |
| 280 | ABTX3 | QVQLVESG | 551 | WFRHAPG | 552 | RFTISRD | 541 | WGQG | 542 |
| | 17 | GGLVQAGA | | KDREFVA | | NAKNTVY | | TQVT | |
| | | SLRLSCAA | | | | LQMNSLK | | VSS | |
| | | SGRT | | | | PEDTAVY | | | |
| | | | | | | YCAA | | | |
| HCDR3 28 | | | | | | | | | |
| 294 | | QVQLQESG | 554 | WFRQAPG | 539 | RFIISRV | 555 | WGQG | 557 |
| | | GGSVQAGG | | KEREFVA | | NAKNTVN | | TQVS | |
| | | SLRLSCAA | | | | LQMNSLK | | VSS | |
| | | SGRAFS | | | | PEDTAVY | | | |
| | | | | | | YCAA | | | |
| 333 | ABTX3 | QVQLQESG | 554 | WFRQAPG | 539 | RFTISRV | 556 | WGQG | 542 |
| | 18 | GGSVQAGG | | KEREFVA | | IAKNTVN | | TQVT | |
| | | SLRLSCAA | | | | LQMNSLK | | VSS | |
| | | SGRAFS | | | | PEDTAVY | | | |
| | | | | | | YCAA | | | |
| 253 | | QVQLQESG | 554 | WFRQAPG | 539 | RFTISRV | 558 | WGQG | 557 |
| | | GGSVQAGG | | KEREFVA | | NAKNTVN | | TQVS | |
| | | SLRLSCAA | | | | LQMNSLK | | VSS | |
| | | SGRAFS | | | | PEDTAVY | | | |
| | | | | | | YCAA | | | |
| 86 | | QVQLQESG | 554 | WFRQAPG | 539 | RFTISRV | 558 | WGQG | 542 |
| | | GGSVQAGG | | KEREFVA | | NAKNTVN | | TQVT | |
| | | SLRLSCAA | | | | LQMNSLK | | VSS | |
| | | SGRAFS | | | | PEDTAVY | | | |
| | | | | | | YCAA | | | |
| HCDR3 50 | | | | | | | | | |
| 71 | | EVQLVESG | 559 | WFRQAPG | 563 | RFTVSRD | 564 | WGPG | 566 |
| | | GGLVQAGG | | KARDFVA | | NAKNTVH LQMNSLR | | TQVT | |
| | | | | | | | | VSS | |
| | | SLRLSCAA | | | | PEDTAVY | | | |
| | | SGPAFS | | | | YCAR | | | |
| 3 | | QVQLQESG | 560 | WFRQAPG | 563 | RFTVSRD | 564 | WGPG | 566 |
| | | GGLVQAGG | | KARDFVA | | NAKNTVH | | TQVT | |
| | | SLRLSCAA | | | | LQMNSLR | | VSS | |
| | | SGPAFS | | | | PEDTAVY | | | |
| | | | | | | YCAR | | | |
| 15 | | QVQLVESG | 561 | WFRQAPG | 563 | RFTVSRD | 564 | WGPG | 566 |
| | | GGLVQAGG | | KARDFVA | | NAKNTVH | | TQVT | |
| | | SLRLACAA | | | | LQMNSLR | | VSS | |
| | | SGAAFS | | | | PEDTAVY | | | |
| | | | | | | YCAR | | | |
| 43 | | QVQLVESG | 561 | WFRQAPG | 563 | RFTVSRD | 565 | WGPG | 566 |
| | | GGLVQAGG | | KARDFVA | | NAKNAVH | | TQVT | |
| | | SLRLSCAA | | | | LQMNSLR | | VSS | |
| | | SGPAFS | | | | PEDTAVY | | | |
| | | | | | | YCAR | | | |
| 5 | | QVQLVESG | 562 | WFRQAPG | 563 | RFTVSRD | 564 | WGPG | 566 |
| | | GGLVQAGG | | KARDFVA | | NAKNTVH | | TQVT | |
| | | SLRLSCAA | | | | LQMNSLR | | VSS | |
| | | SGPAFS | | | | PEDTAVY | | | |
| | | | | | | YCAR | | | |
| 148 | | QVQLVESG | 562 | WFRQAPG | 563 | RFTVSRD | 564 | WGPG | 566 |
| | | GGLVQAGG | | KARDFVA | | NAKNTVH | | TQVT | |
| | | SLRLSCAA | | | | LQMNSLR | | VSS | |
| | | SGPAFS | | | | PEDTAVY | | | |
| | | | | | | YCAR | | | |
| 51 | | QVQLVESG | 562 | WFRQAPG | 563 | RFTVSRD | 564 | WGPG | 566 |
| | | GGLVQAGG | | KARDFVA | | NAKNTVH | | TQVT | |
| | | SLRLSCAA | | | | LQMNSLR | | VSS | |
| | | SGPAFS | | | | PEDTAVY | | | |
| | | | | | | YCAR | | | |
| 84 | | QVQLVESG | 562 | WFRQAPG | 563 | RFTVSRD | 564 | WGPG | 566 |
| | | GGLVQAGG | | KARDFVA | | NAKNTVH | | TQVT | |
| | | SLRLSCAA | | | | LQMNSLR | | VSS | |
| | | SGPAFS | | | | PEDTAVY | | | |
| | | | | | | YCAR | | | |
| 157 | ABTX3 | QVQLVESG | 562 | WFRQAPG | 567 | RFTVSRD | 568 | WGPG | 566 |
| | 20 | GGLVQAGG | | KAREFVA | | NAKMTVH | | TQVT | |
| | | SLRLSCAA | | | | LQMNSLR | | VSS | |
| | | SGPAFS | | | | PEDTAVY | | | |
| | | | | | | YCAR | | | |
| HCDR3 63 | | | | | | | | | |
| 660 | ABTX3 | QLQLVESG | 569 | WFRQAPG | 570 | RFTISRD | 571 | WGQG | 572 |
| | 22 | GGLVQPGG | | NQREFVA | | HTKNTVY | | TLVT | |
| | | SLRLSCAA | | | | LQMNSLK | | VSS | |
| | | SGSDFL | | | | VEDTAVY | | | |
| | | | | | | YCNT | | | |
| HCDR3 65 | | | | | | | | | |
| 155 | ABTX3 | QVQLQESG | 573 | WYRQAPG | 574 | RFTISRD | 575 | WGEG | 576 |
| | 23 | GGLVQAGG | | KQRELVA | | NAKNTVF | | TQVT | |
| | | SLRLSCAT | | | | LQMNSLK | | VSS | |
| | | SGITSS | | | | | | | |
| | | | | | | PEDTAEY | | | |
| | | | | | | YCHG | | | |
| HCDR3 66 | | | | | | | | | |
| 563 | ABTX3 | EVQLVESG | 577 | WVRQAPG | 579 | RFTISRD | 581 | RGQG | 583 |
| | 30 | GGLVQPGG | | KGLEWVS | | NAKKTAY | | TQVT | |
| | | SLRLSCAA | | | | LQMNSLK | | VSS | |
| | | SGFPFS | | | | AEDTAVY | | | |
| | | | | | | YCAT | | | |
| 500 | | QVQLVESG | 578 | WVRQAPG | 580 | RFTISRD | 582 | RGQG | 583 |
| | | GGLVQPGG | | KEVEWVS | | NAKNTVY | | TQVT | |
| | | SLRLSCAA | | | | LQMSNLK | | VSS | |
| | | SGFNFS | | | | AEDTAVY | | | |
| | | | | | | YCAT | | | |
| HCDR3 67 | | | | | | | | | |
| 550 | ABTX3 | QVQLVESG | 584 | WYRQAPG | 585 | RFIISRE | 586 | WGQG | 542 |
| | 24 | GGLVQPGG | | KEREFVA | | NAKTTVY | | TQVT | |
| | | SVRLSCAT | | | | LQMNGLK | | VSS | |
| | | SGSIFS | | | | PEDTAVY | | | |
| | | | | | | YCVI | | | |
| HCDR3 68 | | | | | | | | | |
| 647 | | EVQLVESG | 587 | WYRQAPG | 585 | RFTISRE | 589 | WGQG | 542 |
| | | GGLVQPGG | | KEREFVA | | NAKNTVY | | TQVT | |
| | | SLRLSCAA | | | | LQMNSLK | | VSS | |
| | | SGSVVS | | | | PEDTAVY | | | |
| | | | | | | YCVI | | | |
| 420 | ABTX3 | QVQLVESG | 588 | WYRQAPG | 585 | RFTISRE | 589 | WGQG | 542 |
| | 25 | GGLVQPGG | | KEREFVA | | NAKNTVY | | TQVT | |
| | | SLRLSCAA | | | | LQMNSLK | | VSS | |
| | | SGSDAS | | | | PEDTAVY | | | |
| | | | | | | YCVI | | | |
| HCDR3 60 | | | | | | | | | |
| 1065 | | QLQLVESG | 590 | WYRQAPG | 591 | RFTISRE | 592 | WGQG | 542 |
| | | GGLVQPGE | | KERELVA | | NAKNMVY | | TQVT | |
| | | SLRLSCAA | | | | LQMNSLK | | VSS | |
| | | SGFSFS | | | | LEDTAVY | | | |
| | | | | | | YCNV | | | |
| 1091 | ABTX3 | QLQLVESG | 590 | WYRQAPG | 591 | RFTISRD | 593 | WGQG | 542 |
| | 29 | GGLVQPGE | | KERELVA | | NVKNMVY | | TQVT | |
| | | SLRLSCAA | | | | LQMNSLK | | VSS | |
| | | SGFSFS | | | | LEDTAVY | | | |
| | | | | | | YCNV | | | |
| HCDR3 57 | | | | | | | | | |
| 1043 | ABTX3 | QLQLVESG | 594 | WYRQAPG | 595 | RFAISRD | 596 | WGQG | 542 |
| | 21 | GGLVQPGE | | KQREVVA | | NAKRTVY | | TQVT | |
| | | SLRLSCVV | | | | LQMNSLK | | VSS | |
| | | SGDIFS | | | | FEDTAVY | | | |
| | | | | | | YCNV | | | |
| HCDR3 58 | | | | | | | | | |
| 1050 | ABTX3 | QVQLVESG | 597 | WYRQAPG | 574 | RFTISRD | 598 | WGQG | 542 |
| | 28 | GGLVQPGE | | KQRELVA | | NAKRTVY | | TQVT | |
| | | SLRLSCVV | | | | LQMNSLK | | VSS | |
| | | SGDIFS | | | | FEDTAVY | | | |
| | | | | | | YCNF | | | |
| HCDR3 43 | | | | | | | | | |
| 917 | ABTX3 | QVQLVESG | 599 | WVRQAPG | 600 | RFTISRD | 601 | RGQG | 583 |
| | 19 | GGLVQPGG | | KRLEWVS | | NADNTLY | | TQVT | |
| | | SLRLSCAA | | | | LHMNNLK | | VSS | |
| | | SGFTFS | | | | PEDTAVY | | | |
| | | | | | | YCAN | | | |
| 928 | | QVQLVESG | 599 | WVRQAPG | 600 | RFTISRD | 601 | RGQG | 622 |
| | | GGLVQPGG | | KRLEWVS | | NADNTLY | | TQVT | |
| | | SLRLSCAA | | | | LHMNNLK | | VVS | |
| | | SGFTFS | | | | PEDTAVY | | | |
| | | | | | | YCAN | | | |
| 923 | | QVQLVESG | 599 | WVRQAPG | 600 | RFTISRD | 601 | RGQG | 583 |
| | | GGLVQPGG | | KRLEWVS | | NADNTLY | | TQVT | |
| | | SLRLSCAA | | | | LHMNNLK | | VSS | |
| | | SGFTFS | | | | PEDTAVY | | | |
| | | | | | | YCAN | | | |
| HCDR3 46 | | | | | | | | | |
| 834 | ABTX3 | QVQLVESG | 602 | WFRQAPG | 539 | RFTISRE | 603 | WGQG | 542 |
| | 27 | GGLVQPGG | | KEREFVA | | NAKNTVY | | TQVT | |
| | | SLRLSCVA | | | | LQMNTLK | | VSS | |
| | | SGGTFS | | | | LEDTAVY | | | |
| | | | | | | YCVS | | | |

In various embodiments, the FR1 of a VHH antibody of the disclosure contains the following amino acid sequence:

(SEQ ID NO: 604)
X₁X₂QLX₃ESGGX₄VQX₅GX₆SX₇RLX₈CX₉X₁₀SGX₁₁X₁₂X₁₃X₁₄,

wherein
X₁ is E or Q;
X₂ is L or V;
X₃ is V or Q;
X₄ is L or S;
X₅ is P or A;
X₆ is A, E, or G;
X₇ is L, R, or V;
X₈ is A or S;
X₉ is A or V;
X₁₀ is A, T, or V;
X₁₁ is A, D, F, G, I, P, R, or S;
X₁₂ is A, D, I, N, P, S, T, or V;
X₁₃ is A, F, null, S, or V; and
X₁₄ is I, L, null, or S.

In various embodiments, the FR2 of a VHH antibody of the disclosure contains the following amino acid sequence:

WX₁₅RX₁₆APGX₁₇X₁₈X₁₉X₂₀X₂₁VX₂₂ (SEQ ID NO: 605), wherein
X₁₅ is F, V, or Y;
X₁₆ is H or Q;
X₁₇ is E or K;
X₁₈ A, D, E, G, R, or Q;
X₁₉ is L or R;
X₂₀ is D or E;
X₂₁ is F, L, V, or W; and
X₂₂ is A or S.

In various embodiments, the FR3 of a VHH antibody of the disclosure contains the following amino acid sequence:

RFX₂₃X₂₄SRX₂₅X₂₆X₂₇X₂₈X₂₉X₃₀X₃₁X₃₂LX₃₃MX₃₄X₃₅LX₃₆X₃₇EDTAX₃₈YYCX₃₉X₄₀ (SEQ ID NO: 606), wherein
X₂₃ is A, I, or T;
X₂₄ is I or V;
X₂₅ is D, E, or V;
X₂₆ is H, I, or N;
X₂₇ is A or T;
X₂₈ is D or K;
X₂₉ is K, M, N, R, S, or T;
X₃₀ is A, M, or T;
X₃₁ is A, L, or V;
X₃₂ is F, H, N, or Y;
X₃₃ is H or Q;
X₃₄ is N or S;
X₃₅ is G, N, S, or T;
X₃₆ is K or R;
X₃₇ is A, F, L, P, or V;
X₃₈ is V or E;
X₃₉ is A, H, N, or V; and
X₄₀ is A, E, F, G, I, N, R, T, or V.

In various embodiments, the FR4 of a VHH antibody of the disclosure contains the following amino acid sequence:

X₄₀GX₄₁GTX₄₂VX₄₃VX₄₄S (SEQ ID NO: 607), wherein
X₄₀ is R or W;
X₄₁ is Q, E, or P;
X₄₂ is L or Q;
X₄₃ is S or T; and
X₄₄ is S or V.

The CDRs of the anti-CD5 VHH polypeptides described herein may vary in amino acid sequence length. It will be appreciated by one skilled in the art that number of amino acids that constitute a CDR is not necessarily precise. In some cases, an amino acid residue, or 2 or 3 amino acid residues, at one end or both ends of a given CDR may be considered as part of the CDR or as part of the neighboring FR region. The CDR regions of representative anti-CD5 VHH antibody polypeptides described herein are presented in Tables 1A and 1B. The anti-CD5 VHH antibodies described herein demonstrate the CDR diversity that is selected during affinity maturation of CD5 binding polypeptides in the same animal. Despite such CDR diversity, the CD5 binding VHHs generated as described herein show detectable binding to CD5. The anti-CD5 VHH polypeptides demonstrate significant binding to the CD5 antigen, despite some variation among the CDR sequences in the context of their framework regions.

In view of the representative anti-CD5 VHH amino acid sequences listed in Tables 1A-1C, it will be appreciated by one skilled in the art that individual VHH polypeptides, (e.g., of from about 105 to about 140 amino acids in length and comprising 3 CDRs and 4 FR regions), which comprise at least about or equal to 85%, or 88%, or greater identity in amino acid sequence bind to CD5 antigen. In an embodiment, at least about or equal to 85%, 86%, 87%, 88%, 89%, 90%, 91%, 92%, 93%, 94%, 95%, 96%, 97%, 98%, 99%, or 100% amino acid sequence identity is tolerated among the anti-CD5 VHHs without adversely affecting or eliminating binding of the VHH polypeptides to the CD5 antigen. In an embodiment, such amino acid sequence variation among the anti-CD5 VHH polypeptides is tolerated in the CDRs of the VHH polypeptides without adversely affecting binding of the VHHs to CD5. In a particular embodiment, the amino acid sequence variations between or among anti-CD5 VHHs encompass one or more conservative amino acid substitutions or changes in a VHH amino acid sequence. In an embodiment, the one or more conservative amino acid substitutions or changes in a VHH amino acid sequence occur in one or more CDR sequences of the VHH, in one or more FR sequences of the VHH, or in CDR and FR sequences of the VHH.

The three CDRs of the anti-CD5 VHH polypeptides are arranged or positioned in the context of four FR regions as follows: FR1-CDR1-FR2-CDR2-FR3-CDR3-FR4, in which FR1 to FR4 refer to the framework regions 1-4, respectively, and in which CDR1 to CDR3 refer to the complementarity determining regions 1-3, respectively. An alignment of anti-CD5 VHHs, all of which specifically bind to CD5 protein antigen, demonstrates the extensive similarities among the sequences of each of the FRs (FR1, FR2, FR3 and FR4) found in the different CD5-binding VHH polypeptides. Similar to the FRs in conventional antibody polypeptides, the respective FRs (FR1, FR2, FR3 and FR4) of the anti-CD5 VHH polypeptides described herein are highly similar in sequence among different CD5-binding VHHs that were generated. Accordingly, provided are anti-CD5 VHH polypeptides comprising CDR1-3, in the structural context of FR1-4, that bind to CD5 protein, or to suitable fragments of the CD5 protein, as well as polypeptides that comprise or consist essentially of one or more of the anti-CD5 VHHs and/or CD5 binding fragments thereof (e.g., a chimeric antigen receptor (CAR) polypeptide).

In addition, the FRs of the CD5-binding VHHs described herein are highly or essentially similar in sequence to the FRs of VHHs produced in camelid animals, such as alpacas, camels, llamas, and the like. As they provide structural and conformational support for the CDRs of VHH polypeptides, the FRs and the FR1, FR2, FR3 and FR4 regions among camelid VHH polypeptides generally share significant sequence identity. See, e.g., A. M. Vattekatte et al., March 2020, PeerJ., 6(8): e8408. DOI: 10.7717/peerj.8408 and L. S. Mitchell and L. J. Colwell, 2018, Proteins, 86(7): 697-706).

Table 1C presents the amino acid sequences of the four framework regions, i.e., FR1, FR2, FR3 and FR4, respectively, of representative anti-CD5 VHH polypeptides described herein.

In embodiments, in cases in which a FR (or CDR) amino acid residue in a VHH polypeptide may be one of several alternative amino acid residues, the alternative amino acid residues will frequently share similar characteristics or properties, e.g., hydrophobicity, polarity, and/or charge. A conservative replacement (also called a conservative substitution) is an amino acid replacement or substitution in a polypeptide or region thereof that changes a given amino acid residue to a different amino acid residue with similar biochemical properties, such as charge, hydrophobicity, and/or size. By way of non-limiting example, the below Table 2 presents amino acids and their 1-letter codes categorized into six main classes based on their structure and the general chemical characteristics of their side chains (R groups).

TABLE 2
Classes of amino acids based on structural and chemical characteristics
of their side chains. In embodiments, an amino acid within an FR
or CDR of a VHH antibody of the disclosure is substituted with another
amino acid from the same class indicated in Table 2.

| Amino Acids | Class |
|---|---|
| Glycine (G), Alanine (A), Valine (V), | Aliphatic |
| Leucine (L), Isoleucine (I) | |
| Serine (S), Cysteine (C), Selenocysteine (U), | Hydroxyl or sulfur/ |
| Threonine (T), Methionine (M) | selenium containing |
| Proline (P) | Cyclic |
| Phenylalanine (F), Tyrosine (Y), | Aromatic |
| Tryptophan (W) | |
| Histidine (H), Lysine (K), Arginine (R) | Basic |
| Aspartate (D), Glutamate (E), Asparagine | Acidic and amides |
| (N), Glutamine (Q) | thereof |

In an embodiment, amino acid sequence substitutions or changes in an anti-CD5 VHH polypeptide relative to another anti-CD5 VHH polypeptide comprise conservative amino acid substitutions or changes such that a given amino acid residue is substituted with or replaced by a different amino acid residue with similar biochemical properties, such as charge, hydrophobicity, and/or size. In an embodiment, sequence variation between or among anti-CD5 VHH polypeptides results from one or more conservative amino acid changes and account for the percent sequence variation, e.g., 85%, 86%, 87%, 88%, 90%, 91%, 92%, 93%, 94%, 95%, 96%, 97%, 98%, or 99% sequence variation.

In some embodiments, the VHHs as described herein are humanized using methods and techniques practiced by those having skill in the art. (See, e.g., U.S. Pat. Nos. 8,975,382 and 10,550,174, the contents of which are incorporated by reference herein).

The anti-CD5 VHH antibodies described herein have widespread application (e.g., as antigen binding domains of chimeric antigen receptor polypeptides). In embodiments, the disclosure provides polynucleotides that encode operably linked modular components that constitute the described anti-CD5 VHHs. In embodiments, the anti-CD5 VHHs are recombinantly produced. In embodiments, the anti-CD5 VHHs encompass the proteins (polypeptides) encoded by the polynucleotides. In embodiments, the polynucleotide is DNA, cDNA, RNA, mRNA, or the like. In an embodiment, the anti-CD5 VHHs may be humanized or codon-optimized using methods practiced by those having skill in the art.

Suitable methods of producing or isolating antibody fragments having the requisite binding specificity and affinity for binding to an epitope tag include for example, methods which select recombinant antibody from a library or by PCR (e.g., U.S. Pat. Nos. 5,455,030 and 7,745,587 each of which is incorporated by reference herein in its entirety).

Functional fragments of antibodies, including fragments of chimeric, humanized, primatized, veneered, or single chain antibodies, can also be produced. Functional fragments or portions of the foregoing antibodies include those which are reactive with the CD5 protein. For example, antibody fragments capable of binding to CD5 or a portion thereof, include, but not limited to, scFvs, Fabs, VHHs, Fv, Fab, Fab′ and F(ab′)₂. Such fragments can be produced by enzymatic cleavage or by recombinant techniques. For instance, papain or pepsin cleavage are used generate Fab or F(ab′)₂ antibody fragments, respectively. Antibody fragments are produced in a variety of truncated forms using antibody-encoding genes in which one or more stop codons has been introduced upstream of the natural stop site. For example, a chimeric gene encoding a F(ab′)₂ heavy chain peptide portion can be designed to include DNA sequences encoding the CH₁ peptide domain and hinge region of an immunoglobulin heavy chain.

### Lipid Nanoparticles

Lipid nanoparticles are spherical vesicles made of ionizable lipids, which are positively charged at low pH (enabling RNA complexation) and neutral at physiological pH (reducing potential toxic effects, as compared with positively charged lipids, such as liposomes) (see Nature Reviews Materials 6:99 (2021)). Owing to their size and properties, lipid nanoparticles are taken up by cells via endocytosis, and the ionizability of the lipids at low pH (likely) enables endosomal escape, which allows release of polypeptides or polynucleotides contained within the lipid nanoparticle to be released into the cytoplasm. In addition, lipid nanoparticles may contain a helper lipid to promote cell binding, cholesterol to fill the gaps between the lipids, and/or a polyethylene glycol (PEG) to reduce opsonization by serum proteins and reticuloendothelial clearance. The relative amounts of ionizable lipid, helper lipid, cholesterol and PEG substantially affect the efficacy of lipid nanoparticles, and need to be optimized for a given application and administration route. Moreover, lipid type, size and surface charge impact the behavior of lipid nanoparticles in vivo. Lipid nanoparticles may be used for the delivery of a polynucleotide to a cell.

It can be advantageous to conjugate a lipid nanoparticle to an antigen-binding polypeptide (e.g., the CD5-binding polypeptides provided herein) where the antigen-binding polypeptide binds an antigen on a target cell so as to target the lipid nanoparticle to the garget cell. For example, given that T cells express CD5 on their surface, lipid nanoparticles conjugated to the CD5-binding polypeptides of the disclosure may be used for targeted delivery of a polynucleotide contained within the lipid nanoparticle to a T cell in a subject. Methods are available to the skilled practitioner for conjugating a lipid nanoparticle to an antigen-binding polypeptide (see, e.g., Yaozhong, et al. “Nanobody™-based delivery systems for diagnosis and targeted tumor therapy,” Front Immunol 8:1442 (2017)).

Lipid nanoparticles include any one or more lipids. In some embodiments, the lipid nanoparticles (LNPs) include one or more cationic/ionizable, PEGylated, structural, or other lipids, such as phospholipids. In some embodiments, an LNP comprises a cationic lipid, a helper lipid, and a PEG-modified lipid. In some embodiments, an LNP comprises a cationic lipid, a helper lipid, a PEG-modified lipid, and sterol. Cationic lipids include both permanently charged and ionizable lipids. The ionizable lipids, for example, comprise ionizable lipids including a central amine moiety and at least one biodegradable group. The lipids described herein may be advantageously used in lipid nanoparticles and lipid nanoparticle formulations for the delivery of a therapeutic and/or prophylactic agent, such as a nucleic acid molecule, to a mammalian cell, tissue, or organ.

Suitable LNPs include, for example, lipids familiar to a skilled practitioner or any novel inventive lipids that are generated in the future. Exemplary lipids are described, for example, in the following PCT patent application publications: WO 2015/095340, WO 2020/150320, WO 2020/219876, WO 2021/021634, WO 2021/113365, WO 2022/060871, WO 2017/075531, and WO 2021/141969, and the PCT Application No.: PCT/US2021/64339; the contents of each of which are incorporated herein by reference in their entirety for all purposes.

### Helper Lipids

In some embodiments, an LNP comprises one or more helper lipids. Helper lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-l-trans PE, l-stearoyl-2-oleoyl-phosphatidyethanolamine (SOPE), or a mixture thereof.

### PEG-Modified Lipids

In some embodiments, an LNP comprises one or more PEG lipids. PEG lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. In some embodiments, a PEG lipid can be PEG-c-DOMG, PEG-DMG, PEG-DLPE, PEG-DMPE, PEG-DPPC, or a PEG-DSPE lipid.

In some embodiments, the PEG lipid includes, but are not limited to, 1,2-dimyristoyl-sn-glycerol methoxypoly ethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1, 2-dimyristyloxlpropy 1-3-amine (PEG-c-DMA).

In some embodiments, the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG lipids includes those having lengths of from about C₁₄ to about C₂₂, e.g., from about C₁₄ to about C₁₆. In some embodiments, a PEG moiety, for example an mPEG-NEb, has a size of about 1000, 2000, 5000, 10,000, 15,000 or 20,000 Daltons. In some embodiments, the PEG lipid is PEG2k-DMG.

In some embodiments, the lipid nanoparticles described herein can comprise a PEG lipid which is a non-diffusible PEG. Non-limiting examples of non-diffusible PEGs include PEG-DSG and PEG-DSPE. PEG lipids are known in the art, such as those described in U.S. Pat. No. 8,158,601 and International Publ. No. WO 2015/130584 A2, the disclosures of which are incorporated herein by reference in their entirety for all purposes.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” the disclosure of which is incorporated herein by reference in its entirety for all purposes.

The lipid component of a lipid nanoparticle or lipid nanoparticle formulation may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. In some embodiments, a PEG lipid may be PEG-c-DOMG, PEG-DMG, PEG-DLPE, PEG-DMPE, PEG-DPPC, or a PEG-DSPE lipid.

### Sterols

In some embodiments, an LNP comprises one or more sterol-based lipids. In some embodiments, a sterol is a cholesterol, or a variant or derivative thereof. In some embodiments, a cholesterol is modified. In some embodiments, a cholesterol is an oxidized cholesterol. Exemplary sterols that are considered for use in the disclosed lipid nanoparticles include but are not limited to 25-hydroxycholesterol (25-OH), 20α-hydroxycholesterol (20α-OH), 27-hydroxycholesterol, 6-keto-5α-hydroxycholesterol, 7-ketocholesterol, 7β-hydroxycholesterol, 7α-hydroxycholesterol, 7β-25-dihydroxycholesterol, beta-sitosterol, stigmasterol, brassicasterol, campesterol, or combinations thereof. In some embodiments, a side-chain oxidized cholesterol can enhance cargo delivery relative to other cholesterol variants. In some embodiments, a cholesterol is an unmodified cholesterol. Other examples of suitable cholesterol-based lipids include, for example, DC-Choi (N,N-dimethyl-N-ethylcarboxamidocholesterol), 1,4-bis(3-N-oleylamino-propyl) piperazine (Gao, et al. Biochem. Biophys. Res. Comm. 179, 280 (1991); Wolf et al. BioTechniques 23, 139 (1997); U.S. Pat. No. 5,744,335, the disclosures of which are incorporated herein by reference in their entireties for all purposes), or ICE.

### Targeting Molecules

In some embodiments, an LNP comprises one or more targeting molecules. It should be understood that a targeting particle of the present disclosure may comprise at least one (e.g., two or more) targeting molecules that are the same as each other (e.g., targeting ligands) or different from each other (e.g., targeting ligands and targeting antibodies). In some embodiments, a targeting molecule is an anti-CD5 VHH antibody of the disclosure, or a CD5-binding fragment thereof.

### Targeting Particles

Compositions and methods of the disclosure involve targeting particles (also referred to as targeted particles). In various embodiments, a targeted particle is an LNPs (e.g., are capable of transporting molecules) of the disclosure, optionally with active agent encapsulated in or bound to (e.g., covalently or non-covalently conjugated to) the particle surface. Examples of particles of the present disclosure include, without limitation, liposomes and polymeric particles.

In various embodiments, a targeted particle of the disclosure (e.g., an LNP) contains an anti-CD5 VHH antibody of the disclosure. In some cases, the anti-CD5 VHH antibody is covalently linked to a PEG molecule of an LNP. In some cases the anti-CD5 VHH antibody is covalently linked to a PEG portion of a PEG-modified lipid of an LNP of the disclosure.

Non-limiting examples of cells that may be targeted by targeted particles of the disclosure include cells that express a CD5 polypeptide or a fragment thereof on their surface. In some cases, the cells targeted by the targeted particles of the disclosure are neoplastic cells (e.g., cells of a neoplasia, such as a B cell lymphoma or T cell lymphoma). The targeted cells may be T-cell acute lymphoblastic leukemia cells. In some embodiments, a targeted particle of the disclosure contains an anti-CD5 VHH antibody or CD5-binding fragment thereof conjugated to a surface thereof.

### Particle Conjugation

In some embodiments, particles comprise antibodies or antibody fragments on their surface (e.g., an anti-CD5 VHH antibody of the disclosure, or an antigen-binding fragment thereof). In some embodiments, the antibodies may be designed to bind to target cells without triggering their elimination by complement or other antibody effector mechanisms. This may be achieved either by using antibody fragments or antibodies with mutations that abrogate Fc receptor binding or other effector mechanisms.

These antibody and non-antibody based ligands may be conjugated (or attached or bound, as the terms are used interchangeably herein) to the particle surface covalently or non-covalently. The particles may be synthesized or modified post-synthesis to comprise one or more reactive groups on their exterior surface that can be used to conjugate the antibody and non-antibody based ligands. These particle reactive groups include without limitation thiol-reactive maleimide head groups, haloacetyl (e.g., iodoacetyl) groups, imidoester groups, N-hydroxysuccinimide esters, pyridyl disulfide groups, and the like. As an example, particles may be synthesized to include maleimide conjugated phospholipids such as, without limitation, DSPE-MaL-PEG2000. It will be understood that when surface modified in this manner, the particles are intended for use with ligands having complementary reactive groups (i.e., reactive groups that react with those of the particles).

Methods for conjugating ligands or receptors such as antibodies to particle surfaces are described by Kwong et al. Cancer Research, 2013, 73:1547-1558, the entire contents of which are incorporated by reference herein for all purposes. Other exemplary methods of conjugation can include a reversible conjugation, such that the delivery vehicle can be disassociated from the targeting domain upon exposure to certain conditions or chemical agents. In another embodiment, the conjugation is an irreversible conjugation, such that under normal conditions the delivery vehicle does not dissociate from the targeting domain.

In some embodiments, the conjugation comprises a covalent bond between an activated polymer conjugated lipid and the targeting domain. An activated polymer conjugated lipid is a molecule comprising a lipid portion and a polymer portion that has been activated via functionalization of a polymer conjugated lipid with a first coupling group. In one embodiment, the activated polymer conjugated lipid comprises a first coupling group capable of reacting with a second coupling group. In one embodiment, the activated polymer conjugated lipid is an activated pegylated lipid. In one embodiment, the first coupling group is bound to the lipid portion of the pegylated lipid. In another embodiment, the first coupling group is bound to the polyethylene glycol portion of the pegylated lipid. In one embodiment, the second functional group is covalently attached to the targeting domain.

The first coupling group and second coupling group can be any functional groups known to those of skill in the art to react together form a covalent bond, for example under mild reaction conditions or physiological conditions. In some embodiments, the first coupling group or second coupling group are selected from the group consisting of maleimides, N-hydroxysuccinimide (NHS) esters, carbodiimides, hydrazide, pentafluorophenyl (PFP) esters, phosphines, hydroxymethyl phosphines, psoralen, imidoesters, pyridyl disulfide, isocyanates, vinyl sulfones, alpha-haloacetyls, aryl azides, acyl azides, alkyl azides, diazirines, benzophenone, epoxides, carbonates, anhydrides, sulfonyl chlorides, cyclooctyne, aldehydes, and sulfhydryl groups. In some embodiments, the first coupling group or second coupling group is selected from the group consisting of free amines (—NH₂), free sulfhydryl groups (—SH), free hydroxide groups (—OH), carboxylates, hydrazides, and alkoxyamines. In some embodiments, the first coupling group is a functional group that is reactive toward sulfhydryl groups, such as maleimide, pyridyl disulfide, or a haloacetyl. In one embodiment, the first coupling group is a maleimide.

In one embodiment, the second coupling group is a sulfhydryl group. The sulfhydryl group can be installed on the targeting domain using any method known to those of skill in the art. In one embodiment, the sulfhydryl group is present on a free cysteine residue. In one embodiment, the sulfhydryl group is revealed via reduction of a disulfide on the targeting domain, such as through reaction with 2-mercaptoethylamine. In one embodiment, the sulfhydryl group is installed via a chemical reaction, such as the reaction between a free amine and 2-iminothilane or N-succinimidyl S-acetylthioacetate (SATA).

In some embodiments, the polymer conjugated lipid and targeting domain are functionalized with groups used in “click” chemistry. Bioorthogonal “click” chemistry comprises the reaction between a functional group with a 1,3-dipole, such as an azide, a nitrile oxide, a nitrone, an isocyanide, and the link, with an alkene or an alkyne dipolarophiles. Exemplary dipolarophiles include any strained cycloalkenes and cycloalkynes known to those of skill in the art, including, but not limited to, cyclooctynes, dibenzocyclooctynes, monofluorinated cyclcooctynes, difluorinated cyclooctynes, and biarylazacyclooctynone.

### Cargo

In some embodiments, a particle or LNP composition of the disclosure may contain a cargo, such as one or more nucleic acids (e.g., a polynucleotide encoding a chimeric antigen receptor, an mRNA molecule encoding a base editor of the disclosure, and/or a guide RNA molecule). In some embodiments, the cargo is or comprises one or more biologically active agents, such as an mRNA, guide RNA (gRNA), nucleic acid, RNA-guided DNA-binding agent, expression vector, template nucleic acid, antibody (e.g., monoclonal, chimeric, humanized, sdAb, and fragments thereof, etc.), cholesterol, hormone, peptide, protein, chemotherapeutic and other types of antineoplastic agent, low molecular weight drug, vitamin, co-factor, nucleoside, nucleotide, oligonucleotide, enzymatic nucleic acid, antisense nucleic acid, triplex forming oligonucleotide, antisense DNA or RNA composition, chimeric DNA: RNA composition, allozyme, aptamer, ribozyme, decoys and analogs thereof, plasmid and other types of vectors, and small nucleic acid molecule, RNAi agent, short interfering nucleic acid (siNA), short interfering RNA (siRNA), double-stranded RNA (dsRNA), micro-RNA (miRNA), short hairpin RNA (shRNA) and self-replicating RNA (e.g., an RNA molecule encoding a replicase enzyme activity and capable of directing its own replication or amplification in vivo) molecules, peptide nucleic acid (PNA), a locked nucleic acid ribonucleotide (LNA), morpholino nucleotide, threose nucleic acid (TNA), glycol nucleic acid (GNA), sisiRNA (small internally segmented interfering RNA), and iRNA (asymmetrical interfering RNA). The above list of biologically active agents is exemplary only, and is not intended to be limiting. Such compounds may be purified or partially purified, and may be naturally occurring or synthetic, and may be chemically modified.

Cargo delivered via an LNP composition may be an RNA, such as an mRNA molecule encoding a protein of interest. For example, in some embodiments, an mRNA for expressing a protein such as green fluorescent protein (GFP), an RNA-guided DNA-binding agent, or a Cas nuclease is described herein. LNP compositions that include a Cas nuclease mRNA, for example a Class 2 Cas nuclease mRNA that allows for expression in a cell of a Class 2 Cas nuclease such as a Cas9 or Cpfl protein are provided. Further, cargo may contain one or more guide RNAs or nucleic acids encoding guide RNAs. A template nucleic acid, e.g., for repair or recombination, may also be included in the composition or a template nucleic acid may be used in the methods described herein. In some embodiments, cargo comprises an mRNA that encodes a Streptococcus pyogenes Cas9, optionally and an S. pyogenes gRNA. In some embodiments, cargo comprises an mRNA that encodes a Neisseria meningitidis Cas9, optionally and an nme gRNA.

In some embodiments, the disclosed compositions, preparations, nanoparticles, and/or nanomaterials contain an mRNA encoding an RNA-guided DNA-binding agent, such as a Cas nuclease, and/or a base editor of the disclosure. In particular embodiments, the disclosed compositions, preparations, nanoparticles, and/or nanomaterials comprise an mRNA encoding a Class 2 Cas nuclease, such as S. pyogenes Cas9.

In some embodiments, cargo for an LNP composition includes at least one guide RNA containing a spacer sequence that mediates directing of a napDNAbp to a target DNA. gRNA may guide a Cas nuclease, Class 2 Cas nuclease, and/or base editor to a target sequence on a target nucleic acid molecule.

Target sequences for RNA-guided DNA binding proteins such as Cas proteins include both the positive and negative strands of genomic DNA (i.e., the sequence given and the sequence's reverse compliment), as a nucleic acid substrate for a Cas protein is a double stranded nucleic acid. Accordingly, where a gRNA spacer is said to be “complementary to a target sequence”, it is to be understood that the spacer may direct a guide RNA to bind to the reverse complement of a target sequence. Thus, in some embodiments, where the spacer binds the reverse complement of a target sequence, the spacer is identical to certain nucleotides of the target sequence (e.g., the target sequence not including the PAM) except for the substitution of U for T in the spacer sequence.

In some embodiments, an sgRNA is a “Cas9 sgRNA” capable of mediating RNA-guided DNA cleavage by a Cas9 protein. In some embodiments, a sgRNA is a “Cpfl sgRNA” capable of mediating RNA-guided DNA cleavage by a Cpfl protein. In some embodiments, a gRNA comprises a crRNA and tracr RNA sufficient for forming an active complex with a Cas9 protein and mediating RNA-guided DNA cleavage. In some embodiments, a gRNA comprises a crRNA sufficient for forming an active complex with a Cpfl protein and mediating RNA-guided DNA cleavage.

Certain embodiments of the disclosure also provide nucleic acids, e.g., expression cassettes, encoding a gRNA described herein.

Certain embodiments of the present disclosure also provide delivery of a base editor (e.g., an adenine base editors (“ABEs”), a cytidine base editor (“CBE”) or a cytidine adenine base editor (“CABE”)) using the LNPs compositions, preparations, nanoparticles, and/or nanomaterials described herein. Base editors and methods of their use are described herein and in, e.g., U.S. Pat. Nos. 10,113,163, 10,167,457 and 9,840,699, and U.S. Patent Publication No. 2021/0130805, the contents of each of which are hereby incorporated by reference in their entireties for all purposes.

### Editing of Target Genes

To edit a polynucleotide in a cell, cells within or collected from a subject are contacted with one or more guide RNAs and a nucleobase editor polypeptide comprising a nucleic acid programmable DNA binding protein (napDNAbp) and a cytidine deaminase or adenosine deaminase or comprising one or more deaminases with cytidine deaminase and/or adenosine deaminase activity (e.g., a “dual deaminase” which has cytidine and adenosine deaminase activity). Editing a polynucleotide in a cell may involve administering to a subject a lipid nanoparticle of the disclosure, where the lipid nanoparticle contains as a cargo a base editor system (e.g., an mRNA molecule encoding a base editor of the disclosure and a gRNA molecule). In some embodiments, cells to be edited are contacted with at least one nucleic acid, wherein the at least one nucleic acid encodes one or more guide RNAs and a nucleobase editor polypeptide comprising a nucleic acid programmable DNA binding protein (napDNAbp) and a cytidine deaminase. In some embodiments, the gRNA comprises nucleotide analogs. In some instances, the gRNA is added directly to a cell. These nucleotide analogs can inhibit degradation of the gRNA from cellular processes.

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase, cytidine deaminase, or a dual deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease.

Disclosed herein are base editors comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein (e.g., a Cas protein), also referred to as a “CRISPR protein-derived domain” of the base editor). A CRISPR protein-derived domain incorporated into a base editor can be modified compared to a wild-type or natural version of the CRISPR protein. A CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csnl or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2cl (e.g., SEQ ID NO: 232), Cas12c/C2c3, Cas12d/CasY, Cas12e/CasX, Cas12g, Cas12h, Cas12i, and Cas12j/CasΦ, CARF, DinG, Turbo Cas9 (i.e., an SpCas9 with the amino acid alterations Q844R, V842L, F846Y, L847M, and I852F), homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof. In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid.

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. In some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the napDNAbp is a circular permutant (e.g., SEQ ID NO: 238).

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). For example, where a polynucleotide programmable nucleotide binding domain comprises a nickase domain derived from Cas9, the Cas9-derived nickase domain can include a D10A mutation and a histidine at position 840. In another example, a Cas9-derived nickase domain comprises an H840A mutation, while the amino acid residue at position 10 remains a D.

In some embodiments, a Cas9 nuclease has an inactive (e.g., an inactivated) DNA cleavage domain, that is, the Cas9 is a nickase, referred to as an “nCas9” protein (for “nickase” Cas9; SEQ ID NO: 201). The Cas9 nickase may be a Cas9 protein that is capable of cleaving only one strand of a duplexed nucleic acid molecule (e.g., a duplexed DNA molecule). In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

Also provided herein are base editors comprising a polynucleotide programmable nucleotide binding domain which is catalytically dead (i.e., incapable of cleaving a target polynucleotide sequence). For example, in the case of a base editor comprising a Cas9 domain, the Cas9 can comprise both a D10A mutation and an H840A mutation. In further embodiments, a catalytically dead polynucleotide programmable nucleotide binding domain comprises a point mutation (e.g., D10A or H840A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152 (5): 1173-83, the entire contents of which are incorporated herein by reference.

The term “protospacer adjacent motif (PAM)” or PAM-like motif refers to a 2-6 base pair DNA sequence immediately following the DNA sequence targeted by a nucleic acid programmable DNA binding protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR (N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 3 below.

TABLE 3
Cas9 proteins and corresponding PAM sequences.

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-LRKIQK | NGTN |
| spCas9-LRVSQK | NGTN |
| spCas9-LRVSQL | NGTN |
| Cpf1 | 5′ (TTTV) |
| SpyMac | 5′-NAA-3′ |

N is A, C, T, or G; and V is A, C, or G.

In some embodiments, the PAM is NGC. In some embodiments, the NGC PAM is recognized by a Cas9 variant. In some embodiments, the Cas9 variant contains one or more amino acid substitutions selected from D1135V, G1218R, R1335Q, and T1337R (collectively termed VRQR) of spCas9 (SEQ ID No: 197), or a corresponding mutation in another Cas9. In some embodiments, the Cas9 variant contains one or more amino acid substitutions selected from D1135V, G1218R, R1335E, and T1337R (collectively termed VRER) of spCas9 (SEQ ID No: 197), or a corresponding mutation in another Cas9. In some embodiments, the Cas9 variant contains one or more amino acid substitutions selected from E782K, N968K, and R1015H (collectively termed KHH) of saCas9 (SEQ ID NO: 218).

In some cases, a Cas9 variant has specificity for the PAM 5′-NGC-3′. In some embodiments, a Cas9 variant includes one or more amino acid substitutions selected from D1135M, S1136Q, G1218K, E1219F, A1322R, D1332A, R1335E, and T1337K of spCas9 (SEQ ID No: 197), or a corresponding mutation in another Cas9. In some embodiments, a Cas9 variant includes one or more amino acid substitutions selected from D1135M, S1136Y, G1218K, E1219F, A1322R, D1332A, R1335E, and T1337K of spCas9 (SEQ ID No: 197), or a corresponding mutation in another Cas9. In some embodiments, the a Cas9 variant includes one or more amino acid substitutions selected from D1135L, S1136Y, G1218K, E1219F, A1322R, D1332A, R1335E, and T1337R of spCas9 (SEQ ID No: 197), or a corresponding mutation in another Cas9. In some embodiments, a Cas9 variant includes one or more amino acid substitutions selected from D1135M, S1136Y, G1218K, E1219F, A1322R, D1332A, R1335E, and T1337K of spCas9 (SEQ ID No: 197), or a corresponding mutation in another Cas9. In some embodiments, a Cas9 variant includes one or more amino acid substitutions selected from D1135L, S1136Y, G1218K, E1219F, A1283D, A1322R, D1332A, R1335E, and T1337K of spCas9 (SEQ ID No: 197), or a corresponding mutation in another Cas9. In some embodiments, a Cas9 variant includes one or more amino acid substitutions selected from D1135L, S1136Q, G1218K, E1219F, E1250K, A1283D, A1322R, D1332A, R1335E, and T1337K of spCas9 (SEQ ID No: 197), or a corresponding mutation in another Cas9. In some embodiments, a Cas9 variant includes one or more amino acid substitutions selected from D1135M, S1136Y, G1218K, E1219F, E1250K, A1283D, A1322R, D1332A, R1335E, and T1337R of spCas9 (SEQ ID No: 197), or a corresponding mutation in another Cas9. In some embodiments, a Cas9 variant includes one or more amino acid substitutions selected from R765A, Q768A, D1135L, S1136Y, G1218K, A1283D, E1219F, A1322R, D1332A, R1335E, and T1337K of spCas9 (SEQ ID No: 197), or a corresponding mutation in another Cas9. In some embodiments, any of the Cas9 proteins provided herein, including an SpCas9 comprises any one, two, three, four, five, six, seven, eight, nine, or ten of the following amino acid substitutions in a corresponding residue: R765A, Q768A, W1126R, R1359W, E1250K, A1239T, A1239V, A1283D, R1335D, D1135L, D1135M, D1135R, D1135W, S1136H, S1136Q, S1136Y, G1218D, G1218K, G1218R, G1218E, G1218L, E1219F, E1219K, E1219N, A1322A, A1322R, A1322K, D1332A, R1335V, T1337K, T1337T, D1332A, D1135V and T1337R.

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556 (7699), 57-63; Miller et al., “Continuous evolution of SpCas9 variants compatible with non-G PAMs” Nat. Biotechnol., 2020 April; 38 (4): 471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase, adenosine deaminase, or cytidine adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

Fusion Proteins or Complexes with Internal Insertions

Provided herein are fusion proteins or complexes comprising a heterologous polypeptide fused to a nucleic acid programmable nucleic acid binding protein, for example, a napDNAbp. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof. For example, a fusion protein can comprise a deaminase flanked by an N-terminal fragment and a C-terminal fragment of a Cas9 or Cas12 (e.g., Cas12b/C2cl), polypeptide.

The deaminase can be a circular permutant deaminase. In some embodiments, the deaminase is a circular permutant TadA, circularly permutated at amino acid residue 116, 136, or 65 as numbered in a TadA reference sequence.

The fusion protein or complexes can comprise more than one deaminase. The fusion protein or complex can comprise, for example, 1, 2, 3, 4, 5 or more deaminases. The deaminases in a fusion protein or complex can be adenosine deaminases, cytidine deaminases, or a combination thereof.

In some embodiments, the napDNAbp in the fusion protein or complex contains a Cas9 polypeptide or a fragment thereof. The Cas9 polypeptide can be a variant Cas9 polypeptide. The Cas9 polypeptide can be a circularly permuted Cas9 protein.

The heterologous polypeptide (e.g., deaminase) can be inserted in the napDNAbp (e.g., Cas9 or Cas12 (e.g., Cas12b/C2cl)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase (dual deaminase)) can be inserted into a napDNAbp without compromising function of the deaminase (e.g., base editing activity) or the napDNAbp (e.g., ability to bind to target nucleic acid and guide nucleic acid).

A fusion protein may comprise a linker between the deaminase and the napDNAbp polypeptide. The linker can be a peptide or a non-peptide linker. For example, the linker can be an XTEN, (GGGS)ₙ (SEQ ID NO: 246), SGGSSGGS (SEQ ID NO: 330), (GGGGS)ₙ (SEQ ID NO: 247), (G)ₙ, (EAAAK)n (SEQ ID NO: 248), (GGS)ₙ, SGSETPGTSESATPES (SEQ ID NO: 249). In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by

```
  (SEQ ID NO: 252)
GGAGGCTCTGGAGGAAGC
or
  (SEQ ID NO: 253)
GGCTCTTCTGGATCTGAAACACCTGGCACAAGCGAGAGCGCCACCCCT
GAGAGCTCTGGC.
```

In some embodiments, the napDNAbp in the fusion protein or complex is a Cas12 polypeptide, e.g., Cas12b/C2cl, or a functional fragment thereof capable of associating with a nucleic acid (e.g., a gRNA) that guides the Cas12 to a specific nucleic acid sequence.

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:

(SEQ ID NO: 262)
ATGGCCCCAAAGAAGAAGCGGAAGGTCGGTATCCACGGAGTCCCAGCA
GCC.

In other embodiments, the Cas12b polypeptide contains a mutation that silences the catalytic activity of a RuvC domain. In other embodiments, the Cas12b polypeptide contains D574A, D829A and/or D952A mutations.

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b.

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In an embodiment an adenosine deaminase domain of a base editor comprises all or a portion (e.g., a functional portion) of an ADAT comprising one or more mutations which permit the ADAT to deaminate a target A in DNA. For example, the base editor can comprise all or a portion (e.g., a functional portion) of an ADAT from Escherichia coli (EcTadA) comprising one or more of the following mutations: D108N, A106V, D147Y, E155V, L84F, H123Y, I156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises an alteration or set of alterations selected from those listed in Tables 5A-5E below:

TABLE 5A
Adenosine Deaminase Variants. Residue positions in the E. coli TadA variant (TadA*) are indicated.

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| TadA*0.1 | W | R | H | N | P | | R | N | L | S | A | D | H | G | A | S | D | R | E | I | K | K |
| TadA*0.2 | W | R | H | N | P | | R | N | L | S | A | D | H | G | A | S | D | R | E | I | K | K |
| TadA*1.1 | W | R | H | N | P | | R | N | L | S | A | N | H | G | A | S | D | R | E | I | K | K |
| TadA*1.2 | W | R | H | N | P | | R | N | L | S | V | N | H | G | A | S | D | R | E | I | K | K |
| TadA*2.1 | W | R | H | N | P | | R | N | L | S | V | N | H | G | A | S | Y | R | V | I | K | K |
| TadA*2.2 | W | R | H | N | P | | R | N | L | S | V | N | H | G | A | S | Y | R | V | I | K | K |
| TadA*2.3 | W | R | H | N | P | | R | N | L | S | V | N | H | G | A | S | Y | R | V | I | K | K |
| TadA*2.4 | W | R | H | N | P | | R | N | L | S | V | N | H | G | A | S | Y | R | V | I | K | K |
| TadA*2.5 | W | R | H | N | P | | R | N | L | S | V | N | H | G | A | S | Y | R | V | I | K | K |
| TadA*2.6 | W | R | H | N | P | | R | N | L | S | V | N | H | G | A | S | Y | R | V | I | K | K |
| TadA*2.7 | W | R | H | N | P | | R | N | L | S | V | N | H | G | A | S | Y | R | V | I | K | K |
| TadA*2.8 | W | R | H | N | P | | R | N | L | S | V | N | H | G | A | S | Y | R | V | I | K | K |
| TadA*2.9 | W | R | H | N | P | | R | N | L | S | V | N | H | G | A | S | Y | R | V | I | K | K |
| TadA*2.10 | W | R | H | N | P | | R | N | L | S | V | N | H | G | A | S | Y | R | V | I | K | K |
| TadA*2.11 | W | R | H | N | P | | R | N | L | S | V | N | H | G | A | S | Y | R | V | I | K | K |
| TadA*2.12 | W | R | H | N | P | | R | N | L | S | V | N | H | G | A | S | Y | R | V | I | K | K |
| TadA*3.1 | W | R | H | N | P | | R | N | F | S | V | N | Y | G | A | S | Y | R | V | F | K | K |
| TadA*3.2 | W | R | H | N | P | | R | N | F | S | V | N | Y | G | A | S | Y | R | V | F | K | K |
| TadA*3.3 | W | R | H | N | P | | R | N | F | S | V | N | Y | G | A | S | Y | R | V | F | K | K |
| TadA*3.4 | W | R | H | N | P | | R | N | F | S | V | N | Y | G | A | S | Y | R | V | F | K | K |
| TadA*3.5 | W | R | H | N | P | | R | N | F | S | V | N | Y | G | A | S | Y | R | V | F | K | K |
| TadA*3.6 | W | R | H | N | P | | R | N | F | S | V | N | Y | G | A | S | Y | R | V | F | K | K |
| TadA*3.7 | W | R | H | N | P | | R | N | F | S | V | N | Y | G | A | S | Y | R | V | F | K | K |
| TadA*3.8 | W | R | H | N | P | | R | N | F | S | V | N | Y | G | A | S | Y | R | V | F | K | K |
| TadA*4.1 | W | R | H | N | P | | R | N | L | S | V | N | H | G | N | S | Y | R | V | I | K | K |
| TadA*4.2 | W | G | H | N | P | | R | N | L | S | V | N | H | G | N | S | Y | R | V | I | K | K |
| TadA*4.3 | W | R | H | N | P | | R | N | F | S | V | N | Y | G | N | S | Y | R | V | F | K | K |
| TadA*5.1 | W | R | L | N | P | | L | N | F | S | V | N | Y | G | A | C | Y | R | V | F | N | K |
| TadA*5.2 | W | R | H | S | P | | R | N | F | S | V | N | Y | G | A | S | Y | R | V | F | K | T |
| TadA*5.3 | W | R | L | N | P | | L | N | I | S | V | N | Y | G | A | C | Y | R | V | F | N | K |
| TadA*5.4 | W | R | H | S | P | | R | N | F | S | V | N | Y | G | A | S | Y | R | V | F | K | T |
| TadA*5.5 | W | R | L | N | P | | L | N | F | S | V | N | Y | G | A | C | Y | R | V | F | N | K |
| TadA*5.6 | W | R | L | N | P | | L | N | F | S | V | N | Y | G | A | C | Y | R | V | F | N | K |
| TadA*5.7 | W | R | L | N | P | | L | N | F | S | V | N | Y | G | A | C | Y | R | V | F | N | K |
| TadA*5.8 | W | R | L | N | P | | L | N | F | S | V | N | Y | G | A | C | Y | R | V | F | N | K |
| TadA*5.9 | W | R | L | N | P | | L | N | F | S | V | N | Y | G | A | C | Y | R | V | F | N | K |
| TadA*5.10 | W | R | L | N | P | | L | N | F | S | V | N | Y | G | A | C | Y | R | V | F | N | K |
| TadA*5.11 | W | R | L | N | P | | L | N | F | S | V | N | Y | G | A | C | Y | R | V | F | N | K |
| TadA*5.12 | W | R | L | N | P | | L | N | F | S | V | N | Y | G | A | C | Y | R | V | F | N | K |
| TadA*5.13 | W | R | H | N | P | | L | D | F | S | V | N | Y | A | A | S | Y | R | V | F | K | K |
| TadA*5.14 | W | R | H | N | S | | L | N | F | C | V | N | Y | G | A | S | Y | R | V | F | K | K |
| TadA*6.1 | W | R | H | N | S | | L | N | F | S | V | N | Y | G | N | S | Y | R | V | F | K | K |
| TadA*6.2 | W | R | H | N | T | V | L | N | F | S | V | N | Y | G | N | S | Y | R | V | F | N | K |
| TadA*6.3 | W | R | L | N | S | | L | N | F | S | V | N | Y | G | A | C | Y | R | V | F | N | K |
| TadA*6.4 | W | R | L | N | S | | L | N | F | S | V | N | Y | G | N | C | Y | R | V | F | N | K |
| TadA*6.5 | W | R | L | N | T | V | L | N | F | S | V | N | Y | G | A | C | Y | R | V | F | N | K |
| TadA*6.6 | W | R | L | N | T | V | L | N | F | S | V | N | Y | G | N | C | Y | R | V | F | N | K |
| TadA*7.1 | W | R | L | N | A | | L | N | F | S | V | N | Y | G | A | C | Y | R | V | F | N | K |
| TadA*7.2 | W | R | L | N | A | | L | N | F | S | V | N | Y | G | N | C | Y | R | V | F | N | K |
| TadA*7.3 | L | R | L | N | A | | L | N | F | S | V | N | Y | G | A | C | Y | R | V | F | N | K |
| TadA*7.4 | R | R | L | N | A | | L | N | F | S | V | N | Y | G | A | C | Y | R | V | F | N | K |
| TadA*7.5 | W | R | L | N | A | | L | N | F | S | V | N | Y | G | A | C | Y | H | V | F | N | K |
| TadA*7.6 | W | R | L | N | A | | L | N | I | S | V | N | Y | G | A | C | Y | P | V | F | N | K |
| TadA*7.7 | L | R | L | N | A | | L | N | F | S | V | N | Y | G | A | C | Y | P | V | F | N | K |
| TadA*7.8 | L | R | L | N | A | | L | N | F | S | V | N | Y | G | N | C | Y | R | V | F | N | K |
| TadA*7.9 | L | R | L | N | A | | L | N | F | S | V | N | Y | G | N | C | Y | P | V | F | N | K |
| TadA*7.10 | R | R | L | N | A | | L | N | F | S | V | N | Y | G | A | C | Y | P | V | F | N | K |

TABLE 5B
TadA*8 Adenosine Deaminase Variants. Residue positions in the E. coli TadA variant
(TadA*) are indicated. Alterations are referenced to TadA*7.10 (first row).

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| TadA*7.10 | R | L | A | L | I | V | F | V | N | Y | C | Y | P | Q | V | F | N | T |
| TadA*8.1 | | | | | | | | | | | | T | | | | | | |
| TadA*8.2 | | | | | | | | | | | | R | | | | | | |
| TadA*8.3 | | | | | | | | | | | | | | S | | | | |
| TadA*8.4 | | | | | | | | | | H | | | | | | | | |
| TadA*8.5 | | | | | | S | | | | | | | | | | | | |
| TadA*8.6 | | | | | | | | | | | | | | | | | | R |
| TadA*8.7 | | | | | | | | | | | | | | R | | | | |
| TadA*8.8 | | | | | | | | | | H | | R | | R | | | | |
| TadA*8.9 | | | | | Y | | | | | | | R | | R | | | | |
| TadA*8.10 | | | | | | | | | | | | R | | R | | | | R |
| TadA*8.11 | | | | | | | | | | | | T | | R | | | | |
| TadA*8.12 | | | | | | | | | | | | T | | S | | | | |
| TadA*8.13 | | | | | Y | | | | | H | | R | | R | | | | |
| TadA*8.14 | | | | | Y | S | | | | | | | | | | | | |
| TadA*8.15 | | | | | | S | | | | | | R | | | | | | |
| TadA*8.16 | | | | | | S | | | | H | | R | | | | | | |
| TadA*8.17 | | | | | | S | | | | | | | | R | | | | |
| TadA*8.18 | | | | | | S | | | | H | | | | R | | | | |
| TadA*8.19 | | | | | | S | | | | H | | R | | R | | | | |
| TadA*8.20 | | | | | Y | S | | | | H | | R | | R | | | | |
| TadA*8.21 | | | | | | | | | | | | R | | S | | | | |
| TadA*8.22 | | | | | | S | | | | | | | | S | | | | |
| TadA*8.23 | | | | | | S | | | | H | | | | | | | | |
| TadA*8.24 | | | | | | S | | | | H | | T | | | | | | |

TABLE 5C
TadA*9 Adenosine Deaminase Variants. Alterations are referenced
to TadA*7.10. Additional details of TadA*9 adenosine
deaminases are described in International PCT Application
No. PCT/US2020/049975, which is incorporated herein by
reference in its entirety for all purposes.

| TadA*9 | |
|---|---|
| Description | Alterations |
| TadA*9.1 | E25F, V82S, Y123H, T133K, Y147R, Q154R |
| TadA*9.2 | E25F, V82S, Y123H, Y147R, Q154R |
| TadA*9.3 | V82S, Y123H, P124W, Y147R, Q154R |
| TadA*9.4 | L51W, V82S, Y123H, C146R, Y147R, Q154R |
| TadA*9.5 | P54C, V82S, Y123H, Y147R, Q154R |
| TadA*9.6 | Y73S, V82S, Y123H, Y147R, Q154R |
| TadA*9.7 | N38G, V82T, Y123H, Y147R, Q154R |
| TadA*9.8 | R23H, V82S, Y123H, Y147R, Q154R |
| TadA*9.9 | R21N, V82S, Y123H, Y147R, Q154R |
| TadA*9.10 | V82S, Y123H, Y147R, Q154R, A158K |
| TadA*9.11 | N72K, V82S, Y123H, D139L, Y147R, Q154R, |
| TadA*9.12 | E25F, V82S, Y123H, D139M, Y147R, Q154R |
| TadA*9.13 | M70V, V82S, M94V, Y123H, Y147R, Q154R |
| TadA*9.14 | Q71M, V82S, Y123H, Y147R, Q154R |
| TadA*9.15 | E25F, V82S, Y123H, T133K, Y147R, Q154R |
| TadA*9.16 | E25F, V82S, Y123H, Y147R, Q154R |
| TadA*9.17 | V82S, Y123H, P124W, Y147R, Q154R |
| TadA*9.18 | L51W, V82S, Y123H, C146R, Y147R, Q154R |
| TadA*9.19 | P54C, V82S, Y123H, Y147R, Q154R |
| TadA*9.2 | Y73S, V82S, Y123H, Y147R, Q154R |
| TadA*9.21 | N38G, V82T, Y123H, Y147R, Q154R |
| TadA*9.22 | R23H, V82S, Y123H, Y147R, Q154R |
| TadA*9.23 | R21N, V82S, Y123H, Y147R, Q154R |
| TadA*9.24 | V82S, Y123H, Y147R, Q154R, A158K |
| TadA*9.25 | N72K, V82S, Y123H, D139L, Y147R, Q154R, |
| TadA*9.26 | E25F, V82S, Y123H, D139M, Y147R, Q154R |
| TadA*9.27 | M70V, V82S, M94V, Y123H, Y147R, Q154R |
| TadA*9.28 | Q71M, V82S, Y123H, Y147R, Q154R |
| TadA*9.29 | E25F, I76Y, V82S, Y123H, Y147R, Q154R |
| TadA*9.30 | I76Y, V82T, Y123H, Y147R, Q154R |
| TadA*9.31 | N38G, I76Y, V82S, Y123H, Y147R, Q154R |
| TadA*9.32 | N38G, I76Y, V82T, Y123H, Y147R, Q154R |
| TadA*9.33 | R23H, I76Y, V82S, Y123H, Y147R, Q154R |
| TadA*9.34 | P54C, I76Y, V82S, Y123H, Y147R, Q154R |
| TadA*9.35 | R21N, I76Y, V82S, Y123H, Y147R, Q154R |
| TadA*9.36 | I76Y, V82S, Y123H, D138M, Y147R, Q154R |
| TadA*9.37 | Y72S, I76Y, V82S, Y123H, Y147R, Q154R |
| TadA*9.38 | E25F, I76Y, V82S, Y123H, Y147R, Q154R |
| TadA*9.39 | I76Y, V82T, Y123H, Y147R, Q154R |
| TadA*9.40 | N38G, I76Y, V82S, Y123H, Y147R, Q154R |
| TadA*9.41 | N38G, I76Y, V82T, Y123H, Y147R, Q154R |
| TadA*9.42 | R23H, I76Y, V82S, Y123H, Y147R, Q154R |
| TadA*9.43 | P54C, I76Y, V82S, Y123H, Y147R, Q154R |
| TadA*9.44 | R21N, I76Y, V82S, Y123H, Y147R, Q154R |
| TadA*9.45 | I76Y, V82S, Y123H, D138M, Y147R, Q154R |
| TadA*9.46 | Y72S, I76Y, V82S, Y123H, Y147R, Q154R |
| TadA*9.47 | N72K, V82S, Y123H, Y147R, Q154R |
| TadA*9.48 | Q71M, V82S, Y123H, Y147R, Q154R |
| TadA*9.49 | M70V, V82S, M94V, Y123H, Y147R, Q154R |
| TadA*9.50 | V82S, Y123H, T133K, Y147R, Q154R |
| TadA*9.51 | V82S, Y123H, T133K, Y147R, Q154R, A158K |
| TadA*9.52 | M70V, Q71M, N72K, V82S, Y123H, Y147R, Q154R |
| TadA*9.53 | N72K, V82S, Y123H, Y147R, Q154R |
| TadA*9.54 | Q71M, V82S, Y123H, Y147R, Q154R |
| TadA*9.55 | M70V, V82S, M94V, Y123H, Y147R, Q154R |
| TadA*9.56 | V82S, Y123H, T133K, Y147R, Q154R |
| TadA*9.57 | V82S, Y123H, T133K, Y147R, Q154R, A158K |
| TadA*9.58 | M70V, Q71M, N72K, V82S, Y123H, Y147R, Q154R |

In some embodiments, the adenosine deaminase comprises a TadA*8.20 adenosine deaminase variant further comprising an F149Y amino acid alteration. In some embodiments, the adenosine deaminase comprises a TadA*8.20 adenosine deaminase variant further comprising the amino acid alterations R147D, F149Y, T166I, and D167N (TadA*8.10+). In some embodiments, the adenosine deaminase comprises a TadA*8.20 adenosine deaminase variant further comprising the amino acid alterations S82T and F149Y (TadA*9v1). In some embodiments, the adenosine deaminase comprises a TadA*8.20 adenosine deaminase variant further comprising the amino acid alterations Y147D, F149Y, T166I, D167N and S82T (TadA*9v2).

In some embodiments, the adenosine deaminase comprises one or more of M1I, M1S, S2A, S2E, S2H, S2R, S2L, E3L, V4D, V4E, V4M, V4K, V4S, V4T, V4A, E5K, F6S, F6G, F6H, F6Y, F6I, F6E, S7K, H8E, H8Y, H8H, H8Q, H8E, H8G, H8S, E9Y, E9K, E9V, E9E, Y10F, Y10W, Y10Y, M12S, M12L, M12R, M12W, R13H, R13I, R13Y, R13R, R13G, R13S, H14N, A15D, A15V, A15L, A15H, T17T, T17A, T17W, T17L, T17F, T17R, T17S, L18A, L18E, L18N, L18L, L18S, A19N, A19H, A19K, A19A, A19D, A19G, A19M, R21N, K20K, K20A, K20R, K20E, K20G, K20C, K20Q R21A, R21R, R21N, R21Y, R21C G22P, A22W, A22R, W23D, R23H, W23G, W23Q, W23L, W23R, W23H W23D W23M, W23W, W23I, D24E, D24G, D24W, D24D, D24R, E25F, E25M, E25D, E25A, E25G, E25R, E25E, E25H E25V, E25S, E25Y, R26D, R26E, R26G, R26N, R26Q, R26C, R26L, R26K, R26W, R26C, R26P, R26R, R26A, R26H, E27E, E27Q, E27H, E27C, E27G, E27K, E27S, E27P, E27R, E27L, E27V, E27D, V28V, V28A, V28C, V28G, V28P, V28S, V28T, P29V, P29P, P29A, P29G, P29K, P29L, V30V, V30I, V30L, V30F, V30G, V30A, V30M, L34S, L34V, L34L, L34M, L34W, L34G, H36E, H36V, L36H, H36L, H36N, N37N, N37H, N37R, N37T, N37S, N38G, N38R, N38N, N38E, V40I, W45A, W45W, W45R, W45L, W45N, N46N, N46M, N46P, N46G, N46L, N46R, N46V, R46W, R46F, R46Q, R46M, R47A, R47Q, R47F, R47K, R47P, R47W, R47M, R47R, R47G, R47S, R47V, R47H, P48T, P48L, P48A, P48I, P48S, P48R, P48K, P48D, P48E, P48H, P48G, P48P, P48N, I49G, I49H, I49V, I49F, I49H, I49I, I49M, I49N, I49K, I49Q, I49T, G50L, G50S, G50R, G50G, R51H, R51L, R51N, L51W, R51Y, R51G, R51V, R51R, H52D, H52Y, H52I, H52H, D53D, D53E, D53G, D53P, P54C, P54T, P54P, P54E, A55H, T55A, T55I, T55V, T55G, T55T, A56A, A56H, A56W, A56E, A56S, H57P, H57A, H57H, H57N, A58G, A58E, A58A, A58R, E59A, E59G, E59I, E59Q, E59W, E59E, E59T, E59H, E59P, M61A, M61I, M61L, M61V, M61P, M61G, M61I, L63S, L63V, L63T, L63R, L63H, L63A, R64A, R64Q, R64R, R64D, Q65V, Q65H, Q65G, Q65P, Q65F, Q65Q, Q65R, G66V, G66E, G66T, G66G, G66C, G67G, G67W, G67I, G67A, G67D, G67L, G67V, L68Q, L68M, L68V, L68H, L68L, L68G, V69A, V69M, V69V, M70V, M70L, E70A, M70A, M70M, M70E, M70T, M70v, Q71M, Q71N, Q71L, Q71R, Q71Q, Q71I, N72A, N72K, N72S, N72D, N72Y, N72N, N72H, N72G, N72M, Y73G, Y73I, Y73K, Y73R, Y73S, Y73Y, Y73H, Y73A, R74A, R74Q, R74G, R74K, R74L, R74N, R74G, R74K, R74R, I76H, I76R, I76W, I76Y, I76V, I76Q, I76L, I76D, I76F, I76I, I76N, I76T, I76Y, D77G, D77D, D77A, D77Q, A78Y, A78T, A78G, A78A, A78I, T79M, T79R, T79L, T79T, L80M, L80Y, L80I, L80V, L80L, Y81D, Y81V, Y81Y, Y81M, V82A, V82S, V82G, V82T, V82V, V82Q, V82Y, T83L, T83F, T83T, T83N, L84E, L84F, L84Y, L84I, L84L, L84M, L84A, L84T, L84S, E85K, E85G, E85P, E85S, E85E, E85F, E85V, E85R, P86T, P86C, P86P, P86L, P86N, P86K, P86H, C87M, C87I, C87S, C87N, C87P, S87C, S87L, S87V, V88A, V88M, V88V, V88T, V88E, V88D, V88S, C90S, C90P, C90A, C90T, C90M, A91A, A91G, A91S, A91V, A91T, A91C, A91L, G92T, G92M, G92A, G92Y, G92G, A93I, A93C, A93M, A93V, A93A, M94M, M94T, M94A, M94V, M94L, M94I, M94H, I95S, I95G, I95L, I95H, I95V, H96A, H96L, H96R, H96S, H96H, H96N, H96E, S97C, S97G, S97I, S97M, S97R, S97S, S97P, R98K, R98I, R98N, R98Q, R98G, R98H, R98C, R98L, R98R, G100R, G100V, G100K, G100A, G100S, G100M, G100I, R101V, R101R, R101S, R101C, V102A, V102F, V102I, V102V, D103A, V103A, V103G, V103F, V103V, F104G, D104N, F104V, F104I, F104L, F104A, F104F, F104R, G105V, G105W, G105G, G105M, G105A, A106T, V106Q, V106F, V106W, V106M, A106A, A106Q, A106F, A106G, A106W, A106M, A106V, A106R, A106L, A106S, A106B, A106I, R107C, R107G, R107P, R107K, R107A, R107N, R107W, R107H, R107S, R107R, R107F, D108N, D108F, D108G, D108V, D108A, D108Y, D108H, D108I, D108K, D108L, D108M, D108Q, N108Q, N108F, N108W, N108M, N108K, D108K, D108F, D108M, D108Q, D108R, D108W, D108S, D108E, D108T, D108R, D108D, A109H, A109K, A109R, A109S, A109T, A109V, A109A, A109D, K110G, K110H, K110I, K110R, K110T, K110K, K110A, K110I, T111A, T111G, T111H, T111R, T111T, T111K, G112A, G112G, G112H, G112T, G112R, A113N, A114G, A114H, A114V, A114C, A114S, A114A, G115S, G115G, G115M, G115L, G115A, G115F, L117M, L117L, L117W, L117A, L117S, L117N, L117V, M118D, M118G, M118K, M118N, M118V, M118M, M118L, M118R, D119L, D119N, D119S, D119V, D119D, V120H, V120L, V120V, V120T, V120A, V120E, V120G, V120D, L121D, L121M, L121N, L121K, L121L, H122H, H122N, H122P, H122R, H122S, H122Y, H122G, H122T, H122L, H123C, H123G, H123P, H123V, H123Y, Y123H, H123Y, H123H, P124P, P124H, P124A, P124Y, P124D, P124G, P124I, P124L, P124W, G125H, G125I, G125A, G125M, G125K, G125G, G125P, M126D, M126H, M126K, M126I, M126N, M126O, M126S, M126Y, M126M, M126G, N127H, N127S, N127D, N127K, N127R, N127N, N127I, N127P, N127M, H128R, H128N, H128L, H128H, R129H, R129Q, R129V, R129I, R129E, R129V, R129R, R129M, R129P, V130R, V130V, V130E, V130D, E131E, E131I, E131V, E131K, I132I, I132F, I132T, I132L, I132V, I132E, T133V, T133E, T133G, T133K, T133T, T133A, T133H, T133F, T133I, E134A, E134E, E134G, E134I, E134H, E134K, E134T, G135G, G135V, G135I, G135P, G135E, I136G, I136L, I136T, I136I, I137A, I137D, I137E, L137M, I137S, L137L, L137I, A138D, A138E, A138G, S138A, A138N, A138S, A138T, A138V, A138Y, A138A, A138M, A138L, D139E, D139I, D139C, D139L, D139M, D139D, D139G, D139H, D139A, E140A, E140C, E140L, E140R, E140K, E140E, E140D, C141S, C141A, C141C, C141V, C141E, A142N, A142D, A142G, A142A, A142L, A142S, A142T, A142N, A142S, A142V, A142E, A142C, A143D, A143E, A143G, A143D, A143G, A143E, A143L, A143W, A143M, A143S, A143Q, A143R, A143A, A143I, L144S, L144L, L144T, L144A, L145A, L145F, L145G, L145D, L145L, L145C, L145E, L145s, C146R, S146A, S146C, S146D, S146F, S146R, S146T, S146D, S146G, S146S, S146L, D147D, D147L, D147F, D147G, D147Y, Y147T, Y147R, Y147D, D147R, D147Y, D147A, D147T, D147H, D147F, D147U, D147V, D147I, D147C, F148L, F148F, F148R, F148Y, F148A, F148T, F149C, F149M, F149R, F149Y, F149N, F149F, F149A, F149T, F149V R150R, R150M, R150D, R150F, M151F, M151P, M151R, M151V, M151M, M151E, R152C, R152F, R152H, R152P, R152R, R152P, R152Q, R152M, R152O, R153C, R153Q, R153R, R153V, R153E, R153A, R153P, Q154E, Q154H, Q154M, Q154R, Q154L, Q154S, Q154V, Q154Q, Q154F, Q154I, Q154A, Q154K, E155F, E155G, E155I, E155K, E155P, E155V, E155D, E155E, E155L, E155Q, I156V, I156A, I156I, I156L, I156F, I156D, I156K, I156N, I156R, I156Y, E157A, E157F, E157I, E157P, E157T, E157V, N157K, K157N, K157V, K157P, K157I, K157F, K157F, K157T, K157A, K157S, K157R, A158Q, A158K, A158V, A158A, A158D, A158S, A158T, A158N, Q159S, Q159Q, Q159A, Q159F, Q159K, Q159L, Q159N, K160A, K160S, K160E, K160K, K160N, K160F, K160Q, K161T, K161K, K161R, K161I, K161A, K161N, K161Q, K161S, K161T, A162D, A162Q, R162H, R162P, A162S, A162A, A162N, A162M, A162K, Q163G, Q163S, Q163Q, Q163A, Q163H, Q163N, Q163R, S164F, S164S, S164Q, S164I, S164R, S164Y, S165S, S165P, S165Q, S165A, S165D, S165I, S165T, S165Y, T166T, T166Q, T166E, T166S, T166D, T166K, T166I, T166N, T166P, T166R, D167S D167D, D167I, D167G, D167T, D167A and/or D167N mutation in a TadA reference sequence (e.g., TadA*7.10,ecTadA, or TadA8e), and any alternative mutation at the corresponding position, or one or more corresponding mutations in another adenosine deaminase. Additional mutations are described in U.S. Patent Application Publication No. 2022/0307003 A1 U.S. Pat. No. 11,155,803, and International Patent Application Publications No. WO 2023/288304 A2, PCT/CN2022/143408, WO 2018/027078 A1, WO 2021/158921 A1 and WO 2023/034959 A2, the disclosures of which are incorporated herein by reference in their entirety for all purposes.

In various embodiments, an adenosine deaminase of the disclosure lacks an N-terminal methionine.

In some embodiments, the disclosure provides TadA variants comprising an alteration at an amino acid selected from one or more of L36, I76, V82, Y147, Q154, and N157 compared to TadA*7.10. In some embodiments, the disclosure provides TadA variants comprising one or more of the following alterations relative to TadA*7.10: L36H, I76Y, V82T, Y147T, Q154S, and N157K. In some embodiments, the disclosure provides TadA variants comprising the following alterations relative to TadA*7.10: L36H, I76Y, V82T, Y147T, Q154S, and N157K. In some embodiments, the disclosure provides TadA variants comprising the following alterations relative to TadA*7.10: F84Y, A109L, A109V, A109I, A109F, A109S, A109T, A109N, V155S, V155T, V155N, F156Y, F156W, F156R, F156N, and F156Q. In some embodiments, the disclosure provides TadA variants comprising the following alterations relative to TadA*7.10: E3N, E3K, E3G, F6A, H14D, L18A, W23I, W23R, P29T, P29Y, P29Q, V35Q, L36S, N38D, G42M, N46Y, P48A, G50A, H52L, A62V, L63R, L63F, Q65R, G67N, L68V, M70I, N72Y, T79H, Y81V, V82S, M94R, G100V, V102E, V102S, R107A, A114C, G115E, M118L, D119L, H122T, P124H, P124K, P124Q, H128R, V130F, I132K, I132T, E140L, A142N, A142S, L144Q, L145R, L145N, Y147A, F149A, R152P, F156N, and K160E.

In some embodiments, the disclosure provides TadA variants comprising a V82T, Y147T, and/or a Q154S mutation. In some embodiments, the disclosure provides TadA variants comprising a V82T, Y147T, and/or a Q154S mutation. In some embodiments, the disclosure provides TadA*8.8 further comprising a V82T mutation. In some embodiments, the disclosure provides TadA*8.8 further comprising a V82T, a Y147T, and a Q154S mutation. In some embodiments, the disclosure provides TadA*8.17 further comprising a V82T mutation. In some embodiments, the disclosure provides TadA*8.17 further comprising a V82T, a Y147T, and a Q154S mutation. In some embodiments, the disclosure provides TadA*8.20 further comprising a V82T mutation. In some embodiments, the disclosure provides TadA*8.20 further comprising a V82T, a Y147T, and a Q154S mutation.

In embodiments, a variant of TadA*7.10 comprises one or more alterations selected from any of those alterations provided herein.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, the TadA*8 is a variant as shown in Table 5D. Table 5D shows certain amino acid position numbers in the TadA amino acid sequence and the amino acids present in those positions in the TadA-7.10 adenosine deaminase. Table 5D also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e. In one embodiment, an adenosine deaminase is a TadA*8 that comprises or consists essentially of SEQ ID NO: 316 or a fragment thereof having adenosine deaminase activity.

TABLE 5D
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA-7.10 | R | V | A | T | D | H | Y | F | T | D |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 5E. Table 5E shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5E
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In particular embodiments, the fusion proteins or complexes comprise a single (e.g., provided as a monomer) TadA* (e.g., TadA*8 or TadA*9). Throughout the present disclosure, an adenosine deaminase base editor that comprises a single TadA* domain is indicates using the terminology ABEm or ABE #m, where “#” is an identifying number (e.g., ABE8.20m), where “m” indicates “monomer.” In some embodiments, the TadA* is linked to a Cas9 nickase. In some embodiments, the fusion proteins or complexes of the disclosure comprise as a heterodimer of a wild-type TadA (TadA(wt)) linked to a TadA*. Throughout the present disclosure, an adenosine deaminase base editor that comprises a single TadA* domain and a TadA (wt) domain is indicates using the terminology ABEd or ABE #d, where “#” is an identifying number (e.g., ABE8.20d), where “d” indicates “dimer.” In other embodiments, the fusion proteins or complexes of the disclosure comprise as a heterodimer of a TadA*7.10 linked to a TadA*. In some embodiments, the base editor is ABE8 comprising a TadA* variant monomer. In some embodiments, the base editor is ABE comprising a heterodimer of a TadA* and a TadA (wt). In some embodiments, the base editor is ABE comprising a heterodimer of a TadA* and TadA*7.10. In some embodiments, the base editor is ABE comprising a heterodimer of a TadA*. In some embodiments, the TadA* is selected from Tables 5A-5E.

In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N. M., et al., “Programmable base editing of A·T to G·C in genomic DNA without DNA cleavage” Nature, 551, 464-471 (2017), the entire contents of which are hereby incorporated by reference.

### C to T Editing

In some embodiments, a base editor disclosed herein comprises a fusion protein or complex comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T:A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, the base editor can comprise a uracil stabilizing protein as described herein. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids.

In some embodiments, a cytidine deaminase of a base editor comprises all or a portion (e.g., a functional portion) of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins or complexes described herein, for example by making point mutations in the deaminase domain, affect the processivity of the fusion proteins (e.g., base editors) or complexes. For example, mutations that reduce, but do not eliminate, the catalytic activity of a deaminase domain within a base editing fusion protein or complexes can make it less likely that the deaminase domain will catalyze the deamination of a residue adjacent to a target residue, thereby narrowing the deamination window. The ability to narrow the deamination window can prevent unwanted deamination of residues adjacent to specific target residues, which can reduce or prevent off-target effects.

In some embodiments, an APOBEC deaminase incorporated into a base editor can comprise one or more mutations selected from the group consisting of H121R, H122R, R126A, R126E, R118A, W90A, W90Y, and R132E of rAPOBEC1; D316R, D317R, R320A, R320E, R313A, W285A, W285Y, and R326E of hAPOBEC3G; and any alternative mutation at the corresponding position, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion (e.g., a functional portion) of an APOBEC1 deaminase.

In some embodiments, the fusion proteins or complexes of the disclosure comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

In embodiments, a fusion protein of the disclosure comprises two or more nucleic acid editing domains.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Cytidine Adenosine Base Editors (CABEs)

In some embodiments, a base editor described herein comprises an adenosine deaminase variant that has increased cytidine deaminase activity. Such base editors may be referred to as “cytidine adenosine base editors (CABEs)” or “cytosine base editors derived from TadA* (CBE-Ts),” and their corresponding deaminase domains may be referred to as “TadA* acting on DNA cytosine (TADC)” domains or TadA-derived cytidine deaminases (TadA-CD). Base editors containing adenosine deaminase variants having both cytidine deaminase and adenosine deaminase activity (i.e., TadA-Dual deaminases) may be referred to as TadA-based dual editors (TadDE). In some instances, an adenosine deaminase variant has both adenine and cytosine deaminase activity (i.e., is a dual deaminase). In some embodiments, the adenosine deaminase variants deaminate adenine and cytosine in DNA. In some embodiments, the adenosine deaminase variants deaminate adenine and cytosine in single-stranded DNA. In some embodiments, the adenosine deaminase variants deaminate adenine and cytosine in RNA. In some embodiments, the adenosine deaminase variant predominantly deaminates cytosine in DNA and/or RNA (e.g., greater than 30%, 40%, 50%, 60%, 70%, 80%, 90%, 95%, or 99% of all deaminations catalyzed by the adenosine deaminase variant, or the number of cytosine deaminations catalyzed by the variant is about or at least about 2-fold, 3-fold, 4-fold, 5-fold, 6-fold, 7-fold, 8-fold, 9-fold, 10-fold, 25-fold, 50-fold, 75-fold, 100-fold, 500-fold, or 1,000-fold greater than the number adenine deaminations catalyzed by the variant). In some embodiments, the adenosine deaminase variant has approximately equal cytosine and adenosine deaminase activity (e.g., the two activities are within about 10% or 20% of each other). In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. In some embodiments, the target polynucleotide is present in a cell in vitro or in vivo. In some embodiments, the cell is a bacteria, yeast, fungi, insect, plant, or mammalian cell. Examples of adenosine deaminase variants having increased cytidine deaminase activity include those described in International Patent Application Publications No. WO 2024/040083 and WO 2022/204574, the disclosures of which are hereby incorporated by reference in their entireties for all purposes.

In some embodiments, the CABE comprises a bacterial TadA deaminase variant (e.g., ecTadA). In some embodiments, the CABE comprises a truncated TadA deaminase variant. In some embodiments, the CABE comprises a fragment of a TadA deaminase variant. In some embodiments, the CABE comprises a TadA*8.20 variant.

In some embodiments, an adenosine deaminase variant of the disclosure is a TadA adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity (e.g., at least about 30%, 40%, 50% or more of the activity of a reference adenosine deaminase (e.g., TadA*8.20 or TadA*8.19)). In some instances, the adenosine deaminase variant comprises one or more alterations that increase cytosine deaminase activity (e.g., at least about 10-fold, 20-fold, 30-fold, 40-fold, 50-fold, 60-fold, 70-fold or more increase) relative to the activity of a reference adenosine deaminase and comprise undetectable adenosine deaminase activity or adenosine deaminase activity that is less than 30%, 20%, 10%, or 5% of that of a reference adenosine deaminase. In some embodiments, the reference adenosine deaminase is TadA*8.20 or TadA*8.19.

In some embodiments, the adenosine deaminase variant is an adenosine deaminase comprising two or more alterations at an amino acid position selected from the group consisting of 2, 4, 6, 8, 13, 17, 23, 27, 29, 30, 47, 48, 49, 67, 76, 77, 82, 84, 96, 100, 107, 112, 114, 115, 118, 119, 122, 127, 142, 143, 147, 149, 158, 159, 162 165, 166, and 167, of an amino acid sequence having at least about 70%, 75%, 80%, 85%, 90%, 95%, 98%, 99% or greater identity to SEQ ID NO: 1, or a corresponding alteration in another deaminase. I

In some embodiments, the adenosine deaminase variant is an adenosine deaminase comprising one or more alterations selected from the group consisting of S2H, V4K, V4S, V4T, V4Y, F6G, F6H, F6Y, H8Q, R13G, T17A, T17W, R23Q, E27C, E27G, E27H, E27K, E27Q, E27S, E27G, P29A, P29G, P29K, V30F, V30I, R47G, R47S, A48G, I49K, I49M, I49N, I49Q, I49T, G67W, I76H, I76R, I76W, Y76H, Y76R, Y76W, F84A, F84M, H96N, G100A, G100K, T111H, G112H, A114C, G115M, M118L, H122G, H122R, H122T, N127I, N127K, N127P, A142E, R147H, A158V, Q159S, A162C, A162N, A162Q, and S165P of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising an amino acid alteration or combination of amino acid alterations selected from those listed in any of Tables 6A-6F.

The residue identity of exemplary adenosine deaminase variants that are capable of deaminating adenine and/or cytidine in a target polynucleotide (e.g., DNA) is provided in Tables 6A-6F below. Further examples of adenosine deaminase variants include the following variants of 1.17 (see Table 6A): 1.17+E27H; 1.17+E27K; 1.17+E27S; 1.17+E27S+I49K; 1.17+E27G; 1.17+149N; 1.17+E27G+I49N; and 1.17+E27Q. In some embodiments, any of the amino acid alterations provided herein are substituted with a conservative amino acid. Additional mutations known in the art can be further added to any of the adenosine deaminase variants provided herein.

In some embodiments, the base editor systems comprising a CABE provided herein have at least about a 30%, 40%, 50%, 60%, 70% or more C to T editing activity in a target polynucleotide (e.g., DNA). In some embodiments, a base editor system comprising a CABE as provided herein has an increased C to T base editing activity (e.g., increased at least about 30-fold, 40-fold, 50-fold, 60-fold, 70-fold or more) relative to a reference base editor system comprising a reference adenosine deaminase (e.g., TadA*8.20 or TadA*8.19).

TABLE 6A
Adenosine Deaminase Variants. Mutations are indicated with reference to TadA*8.20.
“S” indicates “Surface,” and “NAS” indicates “Near Active Site.”

| | location in structure | | | | | | | | | | |
|---|---|---|---|---|---|---|---|---|---|---|---|
| | N/A | Sh1 | Sh1 | Sh1 | NAS | NAS | NAS | NAS | | S | |
| | Amino Acid No. (*START Met is AA#1) | | | | | | | | | | |
| | 2 | 8 | 13 | 17 | 27 | 47 | 48 | 49 | 67 | 76 | 77 |
| TadA*8.20 | S | H | R | T | E | R | A | I | G | Y | D |
| TadA*8.19 | | | | | | | | | | I | |
| 1.1 | | | | | H | | | | | I | |
| 1.2 | | | | | H | | | K | | I | |
| 1.3 | | | | | S | | | K | | I | |
| 1.4 | | | | | S | | | K | | I | |
| 1.5 | | | | | K | | | | | | |
| 1.6 | | | | | K | | | | | | |
| 1.7 | | | | | H | | | | | I | |
| 1.8 | | | | | S | | | K | W | | |
| 1.9 | | | | | | | | T | W | | |
| 1.10 | | | | | C | | | | | I | |
| 1.11 | | | G | | Q | | | | | | |
| 1.12 | | | | A | H | | | M | | I | |
| 1.13 | | | | | | | | Q | | I | |
| 1.14 | H | | | | | | | K | | I | |
| TadA*8.20 | S | H | R | T | E | R | A | I | G | Y | D |
| TadA*8.19 | | | | | | | | | | I | |
| 1.15 | | | | | | S | | | | | |
| 1.16 | | Q | | | | | | Q | | I | |
| 1.17 | | | | A | | | G | | | | |
| 1.18 | | | | | G | | | | | | |
| 1.19 | | | | | G | | | N | | | |
| 1.20 | | | | | G | | | | | | G |

| | location in structure | | | | | | | | | | | |
|---|---|---|---|---|---|---|---|---|---|---|---|---|
| | I | NAS | | NAS | S | | S | S | S | | S | |
| | Amino Acid No. (*START Met is AA#1) | | | | | | | | | | | |
| | 82 | 84 | 96 | 107 | 112 | 115 | 118 | 119 | 127 | 142 | 162 | 165 |
| TadA*8.20 | S | F | H | R | G | G | M | D | N | A | A | S |
| TadA*8.19 | | | | | | | | | | | | |
| 1.1 | | M | | | | | | | | | | |
| 1.2 | | | | | | | | | | | | |
| 1.3 | | | | | | | | | | | | |
| 1.4 | | | | | | | | | | | N | |
| 1.5 | | | | | | | | | | | | |
| 1.6 | | | | | | | | N | | | | |
| 1.7 | | | | | | | | | | | | |
| 1.8 | | | | | | | | | | | | |
| 1.9 | | | N | | | | | | | | | |
| 1.10 | | | | | | | | N | | | | |
| 1.11 | | | | | | | | | K | | | |
| 1.12 | | | | | | | L | | | | | |
| 1.13 | | | | | | M | | | | | | |
| 1.14 | | | | | H | | | | | | | |
| 1.15 | | | | C | | | | | | | | |
| 1.16 | | | | | | | | | | | | |
| 1.17 | T | | | | | | | | | E | | |
| 1.18 | | | | | | | | | | | | |
| 1.19 | | | | | | | | | | | | |
| 1.20 | | | | | | | | | | | | P |

TABLE 6B
Adenosine deaminase variants. Mutations are
indicated with reference to TadA*8.20.

| | Position No. | | | | | | | | | |
|---|---|---|---|---|---|---|---|---|---|---|
| | 27 | 29 | 30 | 49 | 82 | 84 | 107 | 112 | 115 | 142 |
| | TadA*8.20 | | | | | | | | | |
| | E | P | V | I | S | F | R | G | G | A |
| | Alterations Evaluated | | | | | | | | | |
| | G/S/H | G/A/K | I/L/F | K | T | L/A | C | H | M | E |
| S1.1 | S | | | K | T | | | | | |
| S1.2 | S | | | K | T | | C | | | |
| S1.3 | S | | | K | T | | | H | | |
| S1.4 | S | | | K | T | | | | M | |
| S1.5 | S | | | K | T | | | | | E |
| S1.6 | S | | | K | T | | C | H | | |
| S1.7 | S | | | K | T | | C | | M | |
| S1.8 | S | | | K | T | | C | | | E |
| S1.9 | S | | | K | T | | | H | | E |
| S1.10 | S | | | K | T | | | | M | E |
| S1.11 | S | | | K | T | | C | H | M | E |
| S1.12 | S | | I | K | T | | | | | |
| S1.13 | S | | I | K | T | | C | | | |
| S1.14 | S | | I | K | T | | | H | | |
| S1.15 | S | | I | K | T | | | | M | |
| S1.16 | S | | I | K | T | | | | | E |
| S1.17 | S | | I | K | T | | C | H | | |
| S1.18 | S | | I | K | T | | C | | M | |
| S1.19 | S | | I | K | T | | C | | | E |
| S1.20 | S | | I | K | T | | | H | | E |
| S1.21 | S | | I | K | T | | | | M | E |
| S1.22 | S | | I | K | T | | C | H | M | E |
| S1.23 | S | | L | K | T | | | | | |
| S1.24 | S | | L | K | T | | C | | | |
| S1.25 | S | | L | K | T | | | H | | |
| S1.26 | S | | L | K | T | | | | M | |
| S1.27 | S | | L | K | T | | | | | E |
| S1.28 | S | | L | K | T | | C | H | | |
| S1.29 | S | | L | K | T | | C | | M | |
| S1.30 | S | | L | K | T | | C | | | E |
| S1.31 | S | | L | K | T | | | H | | E |
| S1.32 | S | | L | K | T | | | | M | E |
| S1.33 | S | | L | K | T | | C | H | M | E |
| S1.34 | S | | F | K | T | A | | | | |
| S1.35 | S | | F | K | T | A | C | | | |
| S1.36 | S | | F | K | T | A | | H | | |
| S1.37 | S | | F | K | T | A | | | M | |
| S1.38 | S | | F | K | T | A | | | | E |
| S1.39 | S | | F | K | T | A | C | H | | |
| S1.40 | S | | F | K | T | A | C | | M | |
| S1.41 | S | | F | K | T | A | C | | | E |
| S1.42 | S | | F | K | T | A | | H | | E |
| S1.43 | S | | F | K | T | A | | | M | E |
| S1.44 | S | | F | K | T | A | C | H | M | E |
| S1.45 | S | | | K | T | L | | | | |
| S1.46 | S | | | K | T | L | C | | | |
| S1.47 | S | | | K | T | L | | H | | |
| S1.48 | S | | | K | T | L | | | M | |
| S1.49 | S | | | K | T | L | | | | E |
| S1.50 | S | | | K | T | L | C | H | | |
| S1.51 | S | | | K | T | L | C | | M | |
| S1.52 | S | | | K | T | L | C | | | E |
| S1.53 | S | | | K | T | L | | H | | E |
| S1.54 | S | | | K | T | L | | | M | E |
| S1.55 | S | | | K | T | L | C | H | M | E |
| S1.56 | S | | I | K | T | L | | | | |
| S1.57 | S | | I | K | T | L | C | | | |
| S1.58 | S | | I | K | T | L | | H | | |
| S1.59 | S | | I | K | T | L | | | M | |
| S1.60 | S | | I | K | T | L | | | | E |
| S1.61 | S | | I | K | T | L | C | H | | |
| S1.62 | S | | I | K | T | L | C | | M | |
| S1.63 | S | | I | K | T | L | C | | | E |
| S1.64 | S | | I | K | T | L | | H | | E |
| S1.65 | S | | I | K | T | L | | | M | E |
| S1.66 | S | | I | K | T | L | C | H | M | E |
| S1.67 | S | G | | K | T | | | | | |
| S1.68 | S | G | | K | T | | C | | | |
| S1.69 | S | G | | K | T | | | H | | |
| S1.70 | S | G | | K | T | | | | M | |
| S1.71 | S | G | | K | T | | | | | E |
| S1.72 | S | G | | K | T | | C | H | | |
| S1.73 | S | G | | K | T | | C | | M | |
| S1.74 | S | G | | K | T | | C | | | E |
| S1.75 | S | G | | K | T | | | H | | E |
| S1.76 | S | G | | K | T | | | | M | E |
| S1.77 | S | G | | K | T | | C | H | M | E |
| S1.78 | | G | | K | T | | | | | |
| S1.79 | | G | | K | T | | C | | | |
| S1.80 | | G | | K | T | | | H | | |
| S1.81 | | G | | K | T | | | | M | |
| S1.82 | | G | | K | T | | | | | E |
| S1.83 | | G | | K | T | | C | H | | |
| S1.84 | | G | | K | T | | C | | M | |
| S1.85 | | G | | K | T | | C | | | E |
| S1.86 | | G | | K | T | | | H | | |
| S1.87 | | G | | K | T | | | | M | E |
| S1.88 | | G | | K | T | | C | H | M | E |
| S1.89 | | K | | K | T | | | | | |
| S1.90 | | K | | K | T | | C | | | |
| S1.91 | | K | | K | T | | | H | | |
| S1.92 | | K | | K | T | | | | M | |
| S1.93 | | K | | K | T | | | | | E |
| S1.94 | | K | | K | T | | C | H | | |
| S1.95 | | K | | K | T | | C | | M | |
| S1.96 | | K | | K | T | | C | | | E |
| S1.97 | | K | | K | T | | | H | | E |
| S1.98 | | K | | K | T | | | | M | E |
| S1.99 | | K | | K | T | | C | H | M | E |
| S1.100 | | K | | K | T | | | | | |
| S1.101 | | K | I | K | T | | C | | | |
| S1.102 | | K | I | K | T | | | H | | |
| S1.103 | | K | I | K | T | | | | M | |
| S1.104 | | K | I | K | T | | | | | E |
| S1.105 | | K | I | K | T | | C | H | | |
| S1.106 | | K | I | K | T | | C | | M | |
| S1.107 | | K | I | K | T | | C | | | E |
| S1.108 | | K | I | K | T | | | H | | E |
| S1.109 | | K | I | K | T | | | | M | E |
| S1.110 | | K | I | K | T | | C | H | M | E |
| S1.111 | | K | | K | T | L | | | | |
| S1.112 | | K | | K | T | L | C | | | |
| S1.113 | | K | | K | T | L | | H | | |
| S1.114 | | K | | K | T | L | | | M | |
| S1.115 | | K | | K | T | L | | | | E |
| S1.116 | | K | | K | T | L | C | H | | |
| S1.117 | | K | | K | T | L | C | | M | |
| S1.118 | | K | | K | T | L | C | | | E |
| S1.119 | | K | | K | T | L | | H | | E |
| S1.120 | | K | | K | T | L | | | M | E |
| S1.121 | | K | | K | T | L | C | H | M | E |
| S1.122 | | K | I | K | T | L | | | | |
| S1.123 | | K | I | K | T | L | C | | | |
| S1.124 | | K | I | K | T | L | | H | | |
| S1.125 | | K | I | K | T | L | | | M | |
| S1.126 | | K | I | K | T | L | | | | E |
| S1.127 | | K | I | K | T | L | C | H | | |
| S1.128 | | K | I | K | T | L | C | | M | |
| S1.129 | | K | I | K | T | L | C | | | E |
| S1.130 | | K | I | K | T | L | | H | | E |
| S1.131 | | K | I | K | T | L | | | M | E |
| S1.132 | | K | I | K | T | L | C | H | M | E |
| S1.133 | G | | | K | T | | | | | |
| S1.134 | G | | | K | T | | C | | | |
| S1.135 | G | | | K | T | | | H | | |
| S1.136 | G | | | K | T | | | | M | |
| S1.137 | G | | | K | T | | | | | E |
| S1.138 | G | | | K | T | | C | H | | |
| S1.139 | G | | | K | T | | C | | M | |
| S1.140 | G | | | K | T | | C | | | E |
| S1.141 | G | | | K | T | | | H | | E |
| S1.142 | G | | | K | T | | | | M | E |
| S1.143 | G | | | K | T | | C | H | M | E |
| S1.144 | H | | | K | T | | | | | |
| S1.145 | H | | | K | T | | C | | | |
| S1.146 | H | | | K | T | | | H | | |
| S1.147 | H | | | K | T | | | | M | |
| S1.148 | H | | | K | T | | | | | E |
| S1.149 | H | | | K | T | | C | H | | |
| S1.150 | H | | | K | T | | C | | M | |
| S1.151 | H | | | K | T | | C | | | E |
| S1.152 | H | | | K | T | | | H | | E |
| S1.153 | H | | | K | T | | | | M | E |
| S1.154 | H | | | K | T | | C | H | M | E |
| S1.155 | S | | | | T | | | | | |
| S1.156 | S | | | | T | | C | | | |
| S1.157 | S | | | | T | | | H | | |
| S1.158 | S | | | | T | | | | M | |
| S1.159 | S | | | | T | | | | | E |
| S1.160 | S | | | | T | | C | H | | |
| S1.161 | S | | | | T | | C | | M | |
| S1.162 | S | | | | T | | C | | | E |
| S1.163 | S | | | | T | | | H | | E |
| S1.164 | S | | | | T | | | | M | E |
| S1.165 | S | | | | T | | C | H | M | E |
| S1.166 | | A | | | T | | | | | |
| S1.167 | | A | | | T | | C | | | |
| S1.168 | | A | | | T | | | H | | |
| S1.169 | | A | | | T | | | | M | |
| S1.170 | | A | | | T | | | | | E |
| S1.171 | | A | | | T | | C | H | | |
| S1.172 | | A | | | T | | C | | M | |
| S1.173 | | A | | | T | | C | | | E |
| S1.174 | | A | | | T | | | H | | E |
| S1.175 | | A | | | T | | | | M | E |
| S1.176 | | A | | | T | | C | H | M | E |
| S1.177 | S | | I | | T | | | | | |
| S1.178 | S | | I | | T | | C | | | |
| S1.179 | S | | I | | T | | | H | | |
| S1.180 | S | | I | | T | | | | M | |
| S1.181 | S | | I | | T | | | | | E |
| S1.182 | S | | I | | T | | C | H | | |
| S1.183 | S | | I | | T | | C | | M | |
| S1.184 | S | | I | | T | | C | | | E |
| S1.185 | S | | I | | T | | | H | | E |
| S1.186 | S | | I | | T | | | | M | E |
| S1.187 | S | | I | | T | | C | H | M | E |
| S1.188 | | A | I | | T | L | | | | |
| S1.189 | | A | I | | T | L | C | | | |
| S1.190 | | A | I | | T | L | | H | | |
| S1.191 | | A | I | | T | L | | | M | |
| S1.192 | | A | I | | T | L | | | | E |
| S1.193 | | A | I | | T | L | C | H | | |
| S1.194 | | A | I | | T | L | C | | M | |
| S1.195 | | A | I | | T | L | C | | | E |
| S1.196 | | A | I | | T | L | | H | | E |
| S1.197 | | A | I | | T | L | | | M | E |
| S1.198 | | A | I | | T | L | C | H | M | E |
| S1.199 | S | A | L | K | T | L | C | H | M | E |

TABLE 6C
Adenosine deaminase variants. Mutations are indicated with reference to variant 1.2 (Table 6A).

| | | Residue identity (START Met | | | | | | | | |
|---|---|---|---|---|---|---|---|---|---|---|
| Variant | Alternative | is amino acid #1) | | | | | | | | |
| Name | Variant Names | 4 | 6 | 17 | 23 | 76 | 77 | 100 | 111 | 114 |
| Reference | 1.2 (see Table 6A) | V | F | T | R | I | D | G | T | A |
| TadAC2.1 | pDKL-135; 2.1 | K | | | | | | | | C |
| TadAC2.2 | pDKL-136; 2.2 | K | | | | | G | | | |
| Reference | 1.2 (see Table 6A) | V | F | T | R | I | D | G | T | A |
| TadAC2.3 | pDKL-137; 2.3 | | Y | | | | | A | | |
| TadAC2.4 | pDKL-138; 2.4 | T | | | | R | | | | |
| TadAC2.5 | pDKL-139; 2.5 | | Y | | | W | | | | |
| TadAC2.6 | pDKL-140; 2.6 | | Y | | | | | | | |
| TadAC2.7 | pDKL-141; 2.7 | | Y | | | | | | | C |
| TadAC2.8 | pDKL-142; 2.8 | | Y | | | | | | | |
| TadAC2.9 | pDKL-143; 2.9 | K | | | | M | | | | |
| TadAC2.10 | pDKL-144; 2.10 | | G | | | R | | K | | |
| TadAC2.11 | pDKL-145; 2.11 | | H | | | | | | | |
| TadAC2.12 | pDKL-146; 2.12 | | | | | | | | | C |
| TadAC2.13 | pDKL-147; 2.13 | | Y | | | H | | | | |
| TadAC2.14 | pDKL-148; 2.14 | | | | | | | | | |
| TadAC2.15 | pDKL-149; 2.15 | | | | Q | R | | | | |
| TadAC2.16 | pDKL-150; 2.16 | | | | | H | | | | |
| TadAC2.17 | pDKL-151; 2.17 | | Y | | | | | | H | |
| TadAC2.18 | pDKL-152; 2.18 | | | | | W | | | | |
| TadAC2.19 | pDKL-153; 2.19 | | | | | | | | H | |
| TadAC2.20 | pDKL-154; 2.20 | | | | | | | | | |
| TadAC2.21 | pDKL-155; 2.21 | | Y | | | R | | | | |
| TadAC2.22 | pDKL-156; 2.22 | | | W | | H | | | | |
| TadAC2.23 | pDKL-157; 2.23 | S | | | | Y | | | | |
| TadAC2.24 | pDKL-158; 2.24 | | | | | | | | | |

| | | Residue identity (START Met is | | | | | | | | |
|---|---|---|---|---|---|---|---|---|---|---|
| | Alternative | amino acid #1) | | | | | | | | |
| Variant Name | Variant Names | 119 | 122 | 127 | 143 | 147 | 158 | 159 | 162 | 166 |
| Reference | 1.2 (see Table 6A) | D | H | N | A | R | A | Q | A | T |
| TadAC2.1 | pDKL-135; 2.1 | | | | | | | | | |
| TadAC2.2 | pDKL-136; 2.2 | | | | | | | | | |
| TadAC2.3 | pDKL-137; 2.3 | | R | | | | | | | |
| TadAC2.4 | pDKL-138; 2.4 | | G | | | | | | | |
| TadAC2.5 | pDKL-139; 2.5 | | | | | | | | | |
| TadAC2.6 | pDKL-140; 2.6 | N | | | | | | | | |
| TadAC2.7 | pDKL-141; 2.7 | | | | | | | | | |
| TadAC2.8 | pDKL-142; 2.8 | | | | | | | | | |
| Reference | 1.2 (see Table 6A) | D | H | N | A | R | A | Q | A | T |
| TadAC2.9 | pDKL-143; 2.9 | | T | | | | | | | |
| TadAC2.10 | pDKL-144; 2.10 | | | | | | | | | |
| TadAC2.11 | pDKL-145; 2.11 | | N | | | | | | | |
| TadAC2.12 | pDKL-146; 2.12 | | | | | | | | | |
| TadAC2.13 | pDKL-147; 2.13 | | R | | | | | | | I |
| TadAC2.14 | pDKL-148; 2.14 | | | P | | | | | | |
| TadAC2.15 | pDKL-149; 2.15 | | | | | | | | | |
| TadAC2.16 | pDKL-150; 2.16 | | R | | | | V | | | |
| TadAC2.17 | pDKL-151; 2.17 | | | | | | | | | |
| TadAC2.18 | pDKL-152; 2.18 | | | | | | | | | |
| TadAC2.19 | pDKL-153; 2.19 | | G | | | | | | C | |
| TadAC2.20 | pDKL-154; 2.20 | | | | E | | | | | |
| TadAC2.21 | pDKL-155; 2.21 | | | | | | | | | |
| TadAC2.22 | pDKL-156; 2.22 | | G | | | | V | | | |
| TadAC2.23 | pDKL-157; 2.23 | | | | E | | | S | | |
| TadAC2.24 | pDKL-158; 2.24 | | | I | | | | | Q | |

TABLE 6D
Adenosine deaminase variants. Mutations are indicated with reference to TadA*8.20.

| | AA Positions | | | | | | | | | | | | | | |
|---|---|---|---|---|---|---|---|---|---|---|---|---|---|---|---|
| | 6 | 27 | 49 | 76 | 77 | 82 | 107 | 112 | 114 | 115 | 119 | 122 | 127 | 142 | 143 |
| TadA*8.20 | F | E | I | Y | D | S | R | G | A | G | D | H | N | A | A |
| S1.154 | F | H | K | Y | D | T | C | H | | M | | | | E | |
| Alterations | Y | | | W | G | | | | C | | N | G | P | | E |
| from Table | | | | | | | | | | | | | | | |
| 6C | | | | | | | | | | | | | | | |
| S2.1 | Y | H | K | W | | T | C | H | | M | | | | E | |
| S2.2 | Y | H | K | | G | T | C | H | | M | | | | E | |
| S2.3 | Y | H | K | | | T | C | H | C | M | | | | E | |
| S2.4 | Y | H | K | | | T | C | H | | M | N | | | E | |
| S2.5 | Y | H | K | | | T | C | H | | M | | G | | E | |
| S2.6 | Y | H | K | | | T | C | H | | M | | | P | E | |
| S2.7 | Y | H | K | | | T | C | H | | M | | | | E | E |
| S2.8 | Y | H | K | | | T | C | H | | M | | | | A | E |
| S2.9 | Y | H | K | W | G | T | C | H | | M | | | | E | |
| S2.10 | Y | H | K | W | | T | C | H | C | M | | | | E | |
| S2.11 | Y | H | K | W | | T | C | H | | M | N | | | E | |
| S2.12 | Y | H | K | W | | T | C | H | | M | | G | | E | |
| S2.13 | Y | H | K | W | | T | C | H | | M | | | P | E | |
| S2.14 | Y | H | K | W | | T | C | H | | M | | | | E | E |
| S2.15 | Y | H | K | W | | T | C | H | | M | | | | A | E |
| S2.16 | Y | H | K | | G | T | C | H | C | M | | | | E | |
| S2.17 | Y | H | K | | G | T | C | H | | M | N | | | E | |
| S2.18 | Y | H | K | | G | T | C | H | | M | | G | | E | |
| S2.19 | Y | H | K | | G | T | C | H | | M | | | P | E | |
| S2.20 | Y | H | K | | G | T | C | H | | M | | | | E | E |
| S2.21 | Y | H | K | | G | T | C | H | | M | | | | A | E |
| S2.22 | Y | H | K | | | T | C | H | C | M | N | | | E | |
| S2.23 | Y | H | K | | | T | C | H | C | M | | G | | E | |
| S2.24 | Y | H | K | | | T | C | H | C | M | | | P | E | |
| S2.25 | Y | H | K | | | T | C | H | | M | N | G | | E | |
| S2.26 | Y | H | K | | | T | C | H | | M | N | | P | E | |
| S2.27 | Y | H | K | | | T | C | H | | M | | G | P | E | |
| S2.28 | Y | H | K | W | G | T | C | H | C | M | | | | E | |
| S2.29 | Y | H | K | W | G | T | C | H | | M | N | | | E | |
| S2.30 | Y | H | K | W | G | T | C | H | | M | | G | | E | |
| S2.31 | Y | H | K | W | G | T | C | H | | M | | | P | E | |
| S2.32 | Y | H | K | W | G | T | C | H | | M | | | | E | E |
| S2.33 | Y | H | K | W | G | T | C | H | | M | | | | A | E |
| S2.34 | Y | H | K | W | | T | C | H | C | M | N | | | E | |
| S2.35 | Y | H | K | W | | T | C | H | C | M | | G | | E | |
| S2.36 | Y | H | K | W | | T | C | H | C | M | | | P | E | |
| S2.37 | Y | H | K | W | | T | C | H | C | M | | | | E | E |
| S2.38 | Y | H | K | W | | T | C | H | C | M | | | | A | E |
| S2.39 | Y | H | K | W | | T | C | H | | M | N | G | | E | |
| S2.40 | Y | H | K | W | | T | C | H | | M | N | | P | E | |
| S2.41 | Y | H | K | W | | T | C | H | | M | | G | P | E | |
| S2.42 | Y | H | K | W | | T | C | H | C | M | N | G | | E | |
| S2.43 | Y | H | K | W | | T | C | H | C | M | N | | P | E | |
| S2.44 | Y | H | K | W | | T | C | H | C | M | | G | P | E | |
| S2.45 | Y | H | K | W | G | T | C | H | C | M | N | | | E | |
| S2.46 | Y | H | K | W | G | T | C | H | C | M | | G | | E | |
| S2.47 | Y | H | K | W | G | T | C | H | C | M | | | P | E | |
| S2.48 | Y | H | K | W | G | T | C | H | C | M | | | | E | E |
| S2.49 | Y | H | K | W | G | T | C | H | C | M | | | | A | E |
| S2.50 | Y | H | K | W | G | T | C | H | C | M | N | G | | E | |
| S2.51 | Y | H | K | W | G | T | C | H | C | M | N | | P | E | |
| S2.52 | Y | H | K | W | G | T | C | H | C | M | | G | P | E | |
| S2.53 | Y | H | K | W | | T | C | H | C | M | N | G | P | E | E |
| S2.54 | Y | H | K | W | | T | C | H | C | M | N | G | P | A | E |
| S2.55 | Y | H | K | W | G | T | C | H | C | M | N | G | P | E | E |
| S2.56 | Y | H | K | W | G | T | C | H | C | M | N | G | P | A | E |

TABLE 6E
Hybrid constructs. Mutations are indicated with reference to TadA*7.10.

| | TadA amino acid subsitutions | | | | | | | | | | | |
|---|---|---|---|---|---|---|---|---|---|---|---|---|
| | 76 | 82 | 109 | 111 | 119 | 122 | 123 | 147 | 149 | 154 | 166 | 167 |
| TadA*7.10 | I | V | A | T | D | H | Y | Y | F | Q | T | D |
| TadA*8e | | | S | R | N | N | | D | Y | | I | N |
| TadA*8.20 | Y | S | | | | | H | R | | R | | |
| TadA*8.17 | | S | | | | | | | | R | | |
| pNMG-B878 | Y | S | | | | | H | D | | R | | |
| pNMG-B879 | Y | S | | | | | H | R | Y | R | | |
| pNMG-B880 | Y | S | | | | | H | R | | R | I | |
| pNMG-B881 | Y | S | | | | | H | R | | R | | N |
| pNMG-B882 | Y | S | | | | | H | D | Y | R | I | N |
| pNMG-B883 | Y | S | | R | N | | H | R | | R | | |
| pNMG-B884 | Y | S | S | R | N | N | H | R | | R | | |
| pNMG-B885 | Y | S | S | | | | H | R | | R | | |
| pNMG-B886 | Y | S | | R | | | H | R | | R | | |
| pNMG-B887 | Y | S | | | N | | H | R | | R | | |
| pNMG-B888 | Y | S | | | | N | H | R | | R | | |
| pNMG-B889 | Y | S | S | R | | | H | R | | R | | |
| pNMG-B890 | Y | S | | | N | N | H | R | | R | | |
| pNMG-B891 | Y | S | S | R | N | N | H | D | Y | R | I | N |

TABLE 6F
Base editor variants. Mutations are indicated with reference to TadA*8.19/8.20.

| | AA positions: | | | | | | | | | | | | |
|---|---|---|---|---|---|---|---|---|---|---|---|---|---|
| | 17 | 27 | 48 | 49 | 76 | 82 | 84 | 118 | 142 | 147 | 149 | 166 | 167 |
| | ABE8.19m/8.20m | | | | | | | | | | | | |
| | T | E | A | I | Y/I | S | F | M | A | Y | F | T | D |
| 1.1 + 8e(B879) | | H | | | I | | M | | | | Y | | |
| 1.2 + 8e(B879) | | H | | K | I | | | | | | Y | | |
| 1.12 + 8e(B879) | A | H | | M | I | | | L | | | Y | | |
| 1.17 + 8e(B879) | A | | G | | | T | | | E | | Y | | |
| 1.18 + 8e(B879) | | G | | | | | | | | | Y | | |
| 1.19 + 8e(B879) | | G | | N | | | | | | | Y | | |
| 1.1 + 8e(B882) | | H | | | I | | M | | | D | Y | I | N |
| 1.2 + 8e(B882) | | H | | K | I | | | | | D | Y | I | N |
| 1.12 + 8e(B882) | A | H | | M | I | | | L | | D | Y | I | N |
| 1.17 + 8e(B882) | A | | G | | | T | | | E | D | Y | I | N |
| 1.18 + 8e(B882) | | G | | | | | | | | D | Y | I | N |
| 1.19 + 8e(B882) | | G | | N | | | | | | D | Y | I | N |

A TadA-derived cytidine deaminase (e.g., TadA-CD), according to certain embodiments, comprises an amino acid sequence that is at least 80% identical, at least 85% identical, at least 90% identical, at least 95% identical, at least 98% identical, at least 99% identical, and at least 99.5% identical to the amino acid sequence of SEQ ID NO: 652, wherein residue 27 of SEQ ID NO: 652 is any amino acid expect for E (glutamic acid). TadA-CDs with other sequence homologies are also possible. For example, in certain embodiments, the TadA-derived cytidine deaminase (e.g., TadA-CD) comprises an amino acid sequence that is at least 80% identical, at least 85% identical, at least 90% identical, at least 95% identical, at least 98% identical, at least 99% identical, and at least 99.5% identical to the amino acid sequence of SEQ ID NO: 652, wherein residue 28 of SEQ ID NO: 652 is any amino acid expect for V (valine). In another exemplary embodiment, the TadA-derived cytidine deaminase is at least 80% identical, at least 85% identical, at least 90% identical, at least 95% identical, at least 98% identical, at least 99% identical, and at least 99.5% identical to the amino acid sequence of SEQ ID NO: 652, wherein residue 96 of SEQ ID NO: 652 is any amino acid expect for H (histidine). In another exemplary embodiment, the TadA-derived cytidine deaminase is at least 80% identical, at least 85% identical, at least 90% identical, at least 95% identical, at least 98% identical, at least 99% identical, and at least 99.5% identical to the amino acid sequence of SEQ ID NO: 652, wherein residue 26 of SEQ ID NO: 652 is any amino acid expect for R (arginine). In various embodiments, the TadA-derived cytidine deaminase comprises an alteration at one or more of positions 26, 27, 28, 48, 73, or 96 compared to SEQ ID NO: 652.

As will be appreciated by those of skill in the art, TadA-derived cytidine deaminases (e.g., TadA-CD) may comprise a plurality of mutations relative to the parent adenosine deaminase (e.g., TadA-8e). In some embodiments, the deaminase of the instant application (e.g., TadA-CD) comprises mutations at residues E27, V28, and H96. In some embodiments, the disclosed deaminase further comprises at least one mutation at a residue selected from R26, M61, Y73, I76, M151, Q154, and A158, in the amino acid sequence of SEQ ID NO: 652, or corresponding mutations in a homologous adenosine deaminase.

In some embodiments, the deaminase comprises at least one mutation selected from E27A, E27K, V28G, V28A, and H96N, and further comprises at least one mutation at a residue selected from R26G, M61I, Y73H, Y73S, Y73C, I76F, M151I, Q154R, Q154H, and A158S, in the amino acid sequence of SEQ ID NO: 652, or a corresponding mutation in a homologous adenosine deaminase. Other mutations are also possible. For example, in certain embodiments, the TadA-CD enzyme comprises mutations selected from E27A, V28G, and H96N, and further comprises at least one mutation selected from R26G, M61I, Y73H, Y73S, Y73C, I76F, M151I, Q154R, Q154H, and A158S, in the amino acid sequence of SEQ ID NO: 652, or corresponding mutations in a homologous adenosine deaminase.

Other exemplary embodiments may include (1) deaminases comprising mutations E27K, V28G, and H96N, and further comprising at least one mutation selected from R26G, M61I, Y73H, Y73S, Y73C, I76F, M151I, Q154R, Q154H, and A158S, in the amino acid sequence of SEQ ID NO: 652 or corresponding mutations in a homologous adenosine deaminase; (2) deaminases comprising mutations E27A, V28A, and H96N, and further comprising at least one mutation selected from R26G, M61I, Y73H, Y73S, Y73C, I76F, M151I, Q154R, Q154H, and A158S, in the amino acid sequence of SEQ ID NO: 652, or corresponding mutations in a homologous adenosine deaminase; (3) deaminases comprising mutations E27K, V28A, and H96N, and further comprising at least one mutation selected from R26G, M61I, Y73H, Y73S, Y73C, I76F, M151I, Q154R, Q154H, and A158S, in the amino acid sequence of SEQ ID NO: 652, or corresponding mutations in a homologous adenosine deaminase.

In some embodiments, the TadA-derived cytidine deaminases (TadA-CD) comprise at least two mutations at residues selected from R26, M61, Y73, I76, M151, Q154, and A158 (relative to a reference adenosine deaminase). In other embodiments, the TadA-CD comprises at least two mutations at residues selected from R26G, M61I, Y73H, I76F, M151I, Q154H, Q154R, and A158S.

In some embodiments, the addition of a V106W mutation improves the selectivity by suppressing A deamination to a greater extent than C deamination.

In some embodiments, a TadA-based dual editor comprises an adenosine deaminase variant comprising one, two, three, four, or five mutations selected from R26G, V28A, A48R, Y73S, and H96N (e.g., SEQ ID NO: 658).

As such, in some embodiments, provided herein are deaminases that comprise mutations at residues R26, V28, A48, and Y73 in the amino acid sequence of SEQ ID NO: 652, or corresponding mutations in a homologous adenosine deaminase. Further provided herein are deaminases that comprise mutations at residues R26, E27, V28, A48, and Y73 (e.g., further comprise a mutation at E27) in the amino acid sequence of SEQ ID NO: 652. In particular embodiments, these deaminases comprise the mutations R26G, V28A, A48R, Y73S, and H96N. In some embodiments, these deaminases comprise the mutations R26G, V28G, A48R, and Y73C.

TadA-CD variants may comprise at least one mutation selected from R26G, E27A, V28G, I76F, H96N, and M151I (e.g, TadA-CDa, SEQ ID NO: 653); R26G, E27A, V28G, I76F, H96N, and A158S (e.g, TadA-CDb, SEQ ID NO: 654); R26G, E27A, V28G, I76F, H96N, Q154R, and A158S (e.g, TadA-CDc, SEQ ID NO: 655); E27A, V28G, Y73H, H96N, Q154H, and A158S (e.g., TadA-CDd, SEQ ID NO: 656); R26G, V28A, A48R, Y73S, and H96N (e.g., TadA-CDe, SEQ ID NO: 657); V28A, A48R, and Y73S (e.g, TadA-CDf, SEQ ID NO: 658), and R26G, V28G, A48R, and Y73C (e.g, TadA-CDg, SEQ ID NO: 659).

In some preferred embodiments, the deaminase comprises the mutations R26G, E27A, V28G, I76F, H96N, and A158S (e.g., TadA-CDa, SEQ ID NO: 653), R26G, E27A, V28G, I76F, H96N, Q154R, and A158S (e.g., TadA-CDb, SEQ ID NO: 654), R26G, E27A, V28G, I76F, H96N, and M151I (e.g., TadA-CDc, SEQ ID NO: 655), E27K, V28A, M61I, and H96N (e.g., TadA-CDd, SEQ ID NO: 656), E27A, V28G, Y73H, H96N, Q154H, and A158S (e.g., TadA-CDe, SEQ ID NO: 657), R26G, V28A, A48R, Y73S, and H96N (e.g., TadA-CDf, SEQ ID NO: 658), and R26G, V28G, A48R, and Y73C (e.g., TadA-CDg, SEQ ID NO: 659).

In some embodiments, the TadA-CD variants described above and herein may also comprises a V106W mutation.

In some embodiments, the TadA-CD variants comprise at least 80%, at least 85%, at least 90%, at least 95%, at least 98%, at least 99%, or at least 99.5% to any of the amino acid sequences of SEQ ID NOs: 652-659.

In some embodiments, the evolved TadA-Dual deaminase comprises the mutations R26G, V28A, N46I, A48R, Y73P, and H96N (TadA-CD-1, SEQ ID NO: 660) relative to the amino acid sequence of SEQ ID NO: 652. In some embodiments, the evolved TadA-Dual deaminase comprises the mutations R26G, V28A, N46T, A48R, Y73P, and H96N (TadA-CD-2, SEQ ID NO: 661) relative to the amino acid sequence of SEQ ID NO: 652. In some embodiments, the evolved TadA-Dual deaminase comprises the mutations R26G, V28A, N46T, A48R, Y73S, and H96N (TadA-CD-3, SEQ ID NO: 662) relative to the amino acid sequence of SEQ ID NO: 652. In some embodiments, the evolved TadA-Dual deaminase comprises the mutations R26G, V28A, N46V, A48R, Y73S, and H96N (TadA-CD-4, SEQ ID NO:663) relative to the amino acid sequence of SEQ ID NO: 652. In some embodiments, the evolved TadA-Dual deaminase comprises the mutations R26G, V28A, N46V, A48R, Y73P, and H96N (TadA-CD-5, SEQ ID NO: 664) relative to the amino acid sequence of SEQ ID NO: 652. In some embodiments, the evolved TadA-Dual deaminase comprises the mutations R26G, V28A, N46L, A48R, Y73P, and H96N (TadA-CD-6, SEQ ID NO: 665) relative to the amino acid sequence of SEQ ID NO: 652. In some embodiments, the evolved TadA-Dual deaminase comprises the mutations V28A, N46L, A48P, and Y73P (TadA-CD-7, SEQ ID NO: 666) relative to the amino acid sequence of SEQ ID NO: 652. In some embodiments, the evolved TadA-Dual deaminase comprises the mutations V28A, N46C, A48P, and Y73P (TadA-CD-8, SEQ ID NO: 667) relative to the amino acid sequence of SEQ ID NO: 652. In some embodiments, the evolved TadA-Dual deaminase comprises the mutations R26G, V28A, N46V, A48R, Y73P, and H96N (TadA-CD-9, SEQ ID NO: 668) relative to the amino acid sequence of SEQ ID NO: 652. In some embodiments, the evolved TadA-Dual deaminase comprises the mutations R26G, V28A, N46V, A48R, Q71H, Y73P, and H96N (TadA-CD-10, SEQ ID NO: 669) relative to the amino acid sequence of SEQ ID NO: 652. In some embodiments, the evolved TadA-Dual deaminase comprises the mutations R26G, V28A, N46L, A48R, Y73P, and H96N (TadA-CD-11, SEQ ID NO: 670) relative to the amino acid sequence of SEQ ID NO: 652. In some embodiments, the evolved TadA-Dual deaminase comprises the mutations R26G, V28A, N46C, A48R, Y73P, and H96N (TadA-CD-12, SEQ ID NO: 671) relative to the amino acid sequence of SEQ ID NO: 652. In some embodiments, the evolved TadA-Dual deaminase comprises the mutations R26G, V28A, N46C, A48R, Y73P, H96N, and A162V (TadA-CD-13, SEQ ID NO: 672) relative to the amino acid sequence of SEQ ID NO: 652.

In some embodiments, the evolved TadA-Dual deaminase comprises the mutations R26G, V28A, N46I, A48R, Y73S, and H96N (TadA-CD-14, SEQ ID NO: 673) relative to the amino acid sequence of SEQ ID NO: 652. In some embodiments, the evolved TadA-Dual deaminase comprises the mutations R26G, V28A, A48R, Q71S, Y73S, and H96N (TadA-CD-15, SEQ ID NO: 674) relative to the amino acid sequence of SEQ ID NO: 652. In some embodiments, the evolved TadA-Dual deaminase comprises the mutations R26G, V28A, N46L, A48R, and Y73P (TadA-CD-16, SEQ ID NO: 675) relative to the amino acid sequence of SEQ ID NO: 652. In some embodiments, the evolved TadA-Dual deaminase comprises the mutations R26G, V28A, N46L, A48R, Y73P, and H96N (TadA-CD-17, SEQ ID NO: 676) relative to the amino acid sequence of SEQ ID NO: 652. In some embodiments, the evolved TadA-Dual deaminase comprises the mutations R26G, V28A, Y73P, and H96N (TadA-CD-18, SEQ ID NO: 677) relative to the amino acid sequence of SEQ ID NO: 652. In some embodiments, the evolved TadA-Dual deaminase comprises the mutations R26G, V28A, N46V, A48R, Y73S, and H96N (TadA-CD-19, SEQ ID NO: 678) relative to the amino acid sequence of SEQ ID NO: 652. In some embodiments, the evolved TadA-Dual deaminase comprises the mutations R26G, V28A, N46V, A48R, Y73P, and H96N (TadA-CD-20, SEQ ID NO: 679) relative to the amino acid sequence of SEQ ID NO: 652. In some embodiments, the evolved TadA-Dual deaminase comprises the mutations R26G and N46L (TadA-CD-21, SEQ ID NO: 680) relative to the amino acid sequence of SEQ ID NO: 652. In some embodiments, the evolved TadA-Dual deaminase comprises the mutations R26G, V28A, N46I, A48R, Y73P, and H96N (TadA-CD-22, SEQ ID NO: 681) relative to the amino acid sequence of SEQ ID NO: 652. In some embodiments, the evolved TadA-Dual deaminase comprises the mutations R26G, V28A, N46V, A48R, Y73P, and H96N (TadA-CD-23, SEQ ID NO: 682) relative to the amino acid sequence of SEQ ID NO: 652. In some embodiments, the evolved TadA-Dual deaminase comprises the mutations R26G, V28A, A48P, Y73H, T79P, and H96N (TadA-CD-24, SEQ ID NO: 683) relative to the amino acid sequence of SEQ ID NO: 652. In some embodiments, the evolved TadA-Dual deaminase comprises the mutations R26G, N46I, and H96N (TadA-CD-25, SEQ ID NO: 684) relative to the amino acid sequence of SEQ ID NO: 652.

In some embodiments, the evolved TadA-Dual deaminase comprises the mutations R26G, V28A, N46V, A48R, Y73P, and H96N (TadA-CD-26, SEQ ID NO: 685) relative to the amino acid sequence of SEQ ID NO: 652. In some embodiments, the evolved TadA-Dual deaminase comprises the mutations R26G, V28A, N46L, A48R, Y73S, and H96N (TadA-CD-27, SEQ ID NO: 686) relative to the amino acid sequence of SEQ ID NO: 652. In some embodiments, the evolved TadA-Dual deaminase comprises the mutations R26G, V28A, N46C, A48R, H96N, and A162V (TadA-CD-28, SEQ ID NO: 687) relative to the amino acid sequence of SEQ ID NO: 652. In some embodiments, the evolved TadA-Dual deaminase comprises the mutations R26G, V28A, N46V, A48R, Q71H, Y73P, and H96N (TadA-CD-29, SEQ ID NO: 688) relative to the amino acid sequence of SEQ ID NO: 652. In some embodiments, the evolved TadA-Dual deaminase comprises the mutations R26G, V28A, N46C, A48R, Y73P, and H96N (TadA-CD-30, SEQ ID NO: 689) relative to the amino acid sequence of SEQ ID NO: 652. In some embodiments, the evolved TadA-Dual deaminase comprises the mutations R26G, V28A, N46C, A48R, Y73P, H96N, and A162V (TadA-CD-31, SEQ ID NO: 690) relative to the amino acid sequence of SEQ ID NO: 652.

In some embodiments, the evolved TadA-Dual deaminase comprises the mutations R26G, V28A, N46V, A48R, Y73P, and H96N (TadA-CD-32, SEQ ID NO: 691) relative to the amino acid sequence of SEQ ID NO: 652. In some embodiments, the evolved TadA-Dual deaminase comprises the mutations R26G, V28A, N46V, A48R, Y73S, and H96N (TadA-CD-33, SEQ ID NO: 692) relative to the amino acid sequence of SEQ ID NO: 652. In some embodiments, the evolved TadA-Dual deaminase comprises the mutations R26G, V28A, N46V, A48P, Y73S, and H96N (TadA-CD-34, SEQ ID NO: 693) relative to the amino acid sequence of SEQ ID NO: 652. In some embodiments, the evolved TadA-Dual deaminase comprises the mutations R26G, V28A, N46C, A48R, Y73P, and H96N (TadA-CD-35, SEQ ID NO: 694) relative to the amino acid sequence of SEQ ID NO: 652. In some embodiments, the evolved TadA-Dual deaminase comprises the mutations R26G, V28A, L34M, N46L, A48R, Y73P, and H96N (TadA-CD-36, SEQ ID NO: 695) relative to the amino acid sequence of SEQ ID NO: 652.

In some embodiments, the evolved TadA-Dual deaminase comprises the mutations R26G, V28A, N46L, A48R, Y73P, and H96N (TadA-CD-37, SEQ ID NO: 696) relative to the amino acid sequence of SEQ ID NO: 652. In some embodiments, the evolved TadA-Dual deaminase comprises the mutations R26G, V28A, N46L, A48P, R64K, Y73P, and H96N (TadA-CD-38, SEQ ID NO: 697) relative to the amino acid sequence of SEQ ID NO: 652.

In some embodiments, the evolved TadA-Dual deaminase comprises the mutations N46I, S73P, and H154Q (TadA-CD-1, SEQ ID NO: 660) relative to the amino acid sequence of SEQ ID NO: 658. In some embodiments, the evolved TadA-Dual deaminase comprises the mutations N46T (TadA-CD-2, SEQ ID NO: 661) relative to the amino acid sequence of SEQ ID NO: 658. In some embodiments, the evolved TadA-Dual deaminase comprises the mutations N46T and H154Q (TadA-CD-3, SEQ ID NO: 662) relative to the amino acid sequence of SEQ ID NO: 658. In some embodiments, the evolved TadA-Dual deaminase comprises the mutations N46V and H154Q (TadA-CD-4, SEQ ID NO: 663) relative to the amino acid sequence of SEQ ID NO: 658. In some embodiments, the evolved TadA-Dual deaminase comprises the mutations N46V, S73P, G105S, and H154Q (TadA-CD-5, SEQ ID NO: 664) relative to the amino acid sequence of SEQ ID NO: 658. In some embodiments, the evolved TadA-Dual deaminase comprises the mutations N46L, S73P, and H154Q (TadA-CD-6, SEQ ID NO: 665) relative to the amino acid sequence of SEQ ID NO: 658. In some embodiments, the evolved TadA-Dual deaminase comprises the mutations G26R N46L, R48P, S73P, N96H, and H154Q (TadA-CD-7, SEQ ID NO: 666) relative to the amino acid sequence of SEQ ID NO: 658. In some embodiments, the evolved TadA-Dual deaminase comprises the mutations N46C, N96H, and H154Q (TadA-CD-8, SEQ ID NO: 667) relative to the amino acid sequence of SEQ ID NO: 658. In some embodiments, the evolved TadA-Dual deaminase comprises the mutations N46V, S73P, and H154Q (TadA-CD-9, SEQ ID NO: 668) relative to the amino acid sequence of SEQ ID NO: 658. In some embodiments, the evolved TadA-Dual deaminase comprises the mutations N46V, Q71H, S73P, and H154Q (TadA-CD-10, SEQ ID NO: 669) relative to the amino acid sequence of SEQ ID NO: 658. In some embodiments, the evolved TadA-Dual deaminase comprises the mutations N46L and H154Q (TadA-CD-11, SEQ ID NO: 670) relative to the amino acid sequence of SEQ ID NO: 658. In some embodiments, the evolved TadA-Dual deaminase comprises the mutations N46C, S73P, and H154Q (TadA-CD-12, SEQ ID NO: 671) relative to the amino acid sequence of SEQ ID NO: 658. In some embodiments, the evolved TadA-Dual deaminase comprises the mutations N46C, S73P, H154Q, and A162V (TadA-CD-13, SEQ ID NO: 672) relative to the amino acid sequence of SEQ ID NO: 658.

In some embodiments, the evolved TadA-Dual deaminase comprises the mutations N46I and H154Q (TadA-CD-14, SEQ ID NO: 673) relative to the amino acid sequence of SEQ ID NO: 658. In some embodiments, the evolved TadA-Dual deaminase comprises the mutations Q71S and H154Q (TadA-CD-15, SEQ ID NO: 674) relative to the amino acid sequence of SEQ ID NO: 652. In some embodiments, the evolved TadA-Dual deaminase comprises the mutations N46L, S73P, N79T, and N96H (TadA-CD-16, SEQ ID NO: 675) relative to the amino acid sequence of SEQ ID NO: 658. In some embodiments, the evolved TadA-Dual deaminase comprises the mutations N46L, S73P, N79T (TadA-CD-17, SEQ ID NO: 676) relative to the amino acid sequence of SEQ ID NO: 658. In some embodiments, the evolved TadA-Dual deaminase comprises the mutations R48A, S73P, and N79T (TadA-CD-18, SEQ ID NO: 677) relative to the amino acid sequence of SEQ ID NO: 658. In some embodiments, the evolved TadA-Dual deaminase comprises the mutations N46V and N79T (TadA-CD-19, SEQ ID NO: 678) relative to the amino acid sequence of SEQ ID NO: 658. In some embodiments, the evolved TadA-Dual deaminase comprises the mutations N46V, S73P, and N79T (TadA-CD-20, SEQ ID NO: 679) relative to the amino acid sequence of SEQ ID NO: 658. In some embodiments, the evolved TadA-Dual deaminase comprises the mutations A28V, N46L, R48A, S73Y, N79T, and N96H (TadA-CD-21, SEQ ID NO: 680) relative to the amino acid sequence of SEQ ID NO: 658. In some embodiments, the evolved TadA-Dual deaminase comprises the mutations N46I, S73P, and N79T (TadA-CD-22, SEQ ID NO: 681) relative to the amino acid sequence of SEQ ID NO: 658. In some embodiments, the evolved TadA-Dual deaminase comprises the mutations N46V, S73P, N79T, and G106S (TadA-CD-23, SEQ ID NO: 682) relative to the amino acid sequence of SEQ ID NO: 658. In some embodiments, the evolved TadA-Dual deaminase comprises the mutations R48P, S73H, and N79P (TadA-CD-24, SEQ ID NO: 683) relative to the amino acid sequence of SEQ ID NO: 658. In some embodiments, the evolved TadA-Dual deaminase comprises the mutations A28V, N46I, R48A, S73Y, and N79T (TadA-CD-25, SEQ ID NO: 684) relative to the amino acid sequence of SEQ ID NO: 658.

In some embodiments, the evolved TadA-Dual deaminase comprises the mutations N46V and S73P (TadA-CD-26, SEQ ID NO: 685) relative to the amino acid sequence of SEQ ID NO: 658. In some embodiments, the evolved TadA-Dual deaminase comprises the mutation N46L (TadA-CD-27, SEQ ID NO: 686) relative to the amino acid sequence of SEQ ID NO: 658. In some embodiments, the evolved TadA-Dual deaminase comprises the mutations N46C, S73Y, and A162V (TadA-CD-28, SEQ ID NO: 687) relative to the amino acid sequence of SEQ ID NO: 658. In some embodiments, the evolved TadA-Dual deaminase comprises the mutations N46V, Q71H, and S73P (TadA-CD-29, SEQ ID NO: 688) relative to the amino acid sequence of SEQ ID NO: 658. In some embodiments, the evolved TadA-Dual deaminase comprises the mutations N46C and S73P (TadA-CD-30, SEQ ID NO: 689) relative to the amino acid sequence of SEQ ID NO: 658. In some embodiments, the evolved TadA-Dual deaminase comprises the mutations N46C, S73P, and A162V (TadA-CD-31, SEQ ID NO: 690) relative to the amino acid sequence of SEQ ID NO: 658. In some embodiments, the evolved TadA-Dual deaminase comprises the mutations N46V and S73P (TadA-CD-32, SEQ ID NO: 691) relative to the amino acid sequence of SEQ ID NO: 658. In some embodiments, the evolved TadA-Dual deaminase comprises the mutation N46V (TadA-CD-33, SEQ ID NO: 692) relative to the amino acid sequence of SEQ ID NO: 658. In some embodiments, the evolved TadA-Dual deaminase comprises the mutations N46V and R48P (TadA-CD-34, SEQ ID NO: 693) relative to the amino acid sequence of SEQ ID NO: 658. In some embodiments, the evolved TadA-Dual deaminase comprises the mutations N46CV and S73P (TadA-CD-35, SEQ ID NO: 694) relative to the amino acid sequence of SEQ ID NO: 658. In some embodiments, the evolved TadA-Dual deaminase comprises the mutations L34M, N46L and S73P (TadA-CD-36, SEQ ID NO: 695) relative to the amino acid sequence of SEQ ID NO: 658. In some embodiments, the evolved TadA-Dual deaminase comprises the mutations N46L and S73P (TadA-CD-37, SEQ ID NO: 696) relative to the amino acid sequence of SEQ ID NO: 658. In some embodiments, the evolved TadA-Dual deaminase comprises the mutations N46L, r48P, R64K and S73P (TadA-CD-38, SEQ ID NO: 697) relative to the amino acid sequence of SEQ ID NO: 658.

In some embodiments, the TadA-CDs evolved from TadA-dual comprise at least 80%, 85%, 90%, 95%, 98%, 99%, or 99.5% identical to any of the amino acid sequences of SEQ ID NOs: 39, 41-54, and 359-383.

Exemplary TadA-derived cytosine base editor amino acid sequences include: TadA-CDa base editor (SpCas9n napDNAbp domain) (TadCBEa) (SEQ ID NO: 698), TadA-CDb base editor (SpCas9n napDNAbp domain) (TadCBEb) (SEQ ID NO: 699), TadA-CDe base editor (SpCas9n napDNAbp domain) (TadCBEc) (SEQ ID NO: 700), TadA-CDd base editor (SpCas9n napDNAbp domain) (TadCBEd) (SEQ ID NO: 701), TadA-CDe base editor (SpCas9n napDNAbp domain) (TadCBEe) (SEQ ID NO: 702), TadA-CDa (V106W) base editor (SpCas9n napDNAbp domain) (TadCBEa (V106W)) (SEQ ID NO: 703), TadA-CDd (V106W) base editor (SpCas9n napDNAbp domain) (TadCBEd (V106W)) (SEQ ID NO: 704), TadA-CDf base editor (SpCas9n napDNAbp domain) (TadCBEf) (SEQ ID NO: 705), TadA-CDg base editor (SpCas9n napDNAbp domain) (TadCBEg) (SEQ ID NO: 706), TadA-CDa: eNme2Cas9 base editor (SEQ ID NO: 707), TadA-CDa: SaCas9 base editor (SEQ ID NO: 708), TadA-CDa: SpCas9-NG base editor (SEQ ID NO: 709), TadA-CDa: enCjCas9 base editor (SEQ ID NO: 710).

Exemplary polynucleotides encoding TadA-derived cytosine base editors of the disclosure include: TadCBEa-eNme2-C-BE4max vector (SEQ ID NO: 711), TadCBEa-enCjCas9-BE4max vector (SEQ ID NO: 712), TadCBEa-SpCas9-BE4max vector (SEQ ID NO: 713), TadCBEa-SaCas9-BE4max vector (SEQ ID NO: 714), TadCBEa-SpCas9-NG-BE4max vector (SEQ ID NO: 715).

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, a guide polynucleotide described herein can be RNA or DNA. In one embodiment, the guide polynucleotide is a gRNA.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

A guide polynucleotide may include natural or non-natural (or unnatural) nucleotides (e.g., peptide nucleic acid or nucleotide analogs). In some cases, the targeting region of a guide nucleic acid sequence (e.g., a spacer) can be at least 15, 16, 17, 18, 19, 20, 21, 22, 23, 24, 25, 26, 27, 28, 29, or 30 nucleotides in length.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 317-327 and 425. Thus, a skilled artisan can change the genomic target of the Cas protein specificity is partially determined by how specific the gRNA targeting sequence is for the genomic target compared to the rest of the genome. In embodiments, the spacer is about 10, 11, 12, 13, 14, 15, 16, 17, 18, 19, 20, 22, 23, 24, 25, or more nucleotides in length. The spacer of a gRNA can be or can be about 19, 20, or 21 nucleotides in length.

A gRNA or a guide polynucleotide can target any exon or intron of a gene target. In some embodiments, a composition comprises multiple gRNAs that all target the same exon or multiple gRNAs that target different exons. An exon and/or an intron of a gene can be targeted. A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

The guide polynucleotides can comprise standard ribonucleotides, modified ribonucleotides (e.g., pseudouridine), ribonucleotide isomers, and/or ribonucleotide analogs.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and may be separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications. Such modifications can increase base editing ˜2 fold in vivo or in vitro. In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the NLS is present in a linker or the NLS is flanked by linkers, for example described herein. A bipartite NLS comprises two basic amino acid clusters, which are separated by a relatively short spacer sequence (hence bipartite-2 parts, while monopartite NLSs are not). The NLS of nucleoplasmin, KR[PAATKKAGQA]KKK (SEQ ID NO: 191), is the prototype of the ubiquitous bipartite signal: two clusters of basic amino acids, separated by a spacer of about 10 amino acids. The sequence of an exemplary bipartite NLS follows:

(SEQ ID NO: 328)
PKKKRKVEGADKRTADGSEFESPKKKRKV.

In some embodiments, any of the fusion proteins or complexes provided herein comprise an NLS comprising the amino acid sequence EGADKRTADGSEFESPKKKRKV (amino acids 8 to 29 of SEQ ID NO 328). In some embodiments, any of the adenosine base editors provided herein comprise an NLS comprising the amino acid sequence EGADKRTADGSEFESPKKKRKV (amino acids 8 to 29 of SEQ ID NO: 328). In some embodiments, the NLS is at a C-terminal portion of the adenosine base editor. In some embodiments, the NLS is at the C-terminus of the adenosine base editor.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises an uracil glycosylase inhibitor (UGI) domain. In some cases, a base editor is expressed in a cell in trans with a UGI polypeptide. In some embodiments, cellular DNA repair response to the presence of U:G heteroduplex DNA can be responsible for a reduction in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleotide (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH₂) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fe domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g., an MS2, an MS2 C-5 mutant, or an MS2 F-5 mutant), a non-natural RNA motif, a PP7 operator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH₂) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self-complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and Voß, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., cytidine deaminase and/or adenosine deaminase), a uracil glycosylase inhibitor (UGI) domain, epitope tags, and reporter gene sequences.

In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises an evolved TadA variant. In some embodiments, the base editor is ABE8.1, which comprises or consists essentially of the following sequence or a fragment thereof having adenosine deaminase activity: SEQ ID NO: 331. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 7 refers to a monomeric form of TadA*7.10 comprising the alterations described. The term “heterodimer” as used in Table 7 refers to the specified wild-type E. coli TadA adenosine deaminase fused to a TadA*7.10 comprising the alterations as described.

TABLE 7
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + N157K |
| ABE-824m | MSP824 | monomer_TadA*7.10 + V82G + Y147D + F149Y + Q154S + D167N |
| ABE-825m | MSP825 | monomer_TadA*7.10 + L36H + V82G + Y147D + F149Y + Q154S + N157K + D167N |
| ABE-827m | MSP827 | monomer_TadA*7.10 + L36H + I76Y + V82G + Y147T + Q154S + N157K |
| ABE-828m | MSP828 | monomer_TadA*7.10 + I76Y + V82G + Y147D + F149Y + Q154S + D167N |
| ABE-829m | MSP829 | monomer_TadA*7.10 + L36H + I76Y + V82G + Y147D + F149Y + Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + I76Y + V82G + Y147T + Q154S) |
| ABE-823d | MSP823 | heterodimer_(WT) + (TadA*7.10 + L36H + V82G + Y147T + Q154S + N157K) |
| ABE-824d | MSP824 | heterodimer_(WT) + (TadA*7.10 + V82G + Y147D + F149Y + Q154S + D167N) |
| ABE-825d | MSP825 | heterodimer_(WT) + (TadA*7.10 + L36H + V82G + Y147D + F149Y + Q154S + N157K + D167N) |
| ABE-827d | MSP827 | heterodimer_(WT) + (TadA*7.10 + L36H + I76Y + V82G + Y147T + Q154S + N157K) |
| ABE-828d | MSP828 | heterodimer_(WT) + (TadA*7.10 + I76Y + V82G + Y147D + F149Y + Q154S + D167N) |
| ABE-829d | MSP829 | heterodimer_(WT) + (TadA*7.10 + L36H + I76Y + V82G + Y147D + F149Y + Q154S + N157K + D167N) |

In some embodiments, the base editor comprises a domain comprising all or a portion (e.g., a functional portion) of a uracil glycosylase inhibitor (UGI) or a uracil stabilizing protein (USP) domain.

### Linkers

In certain embodiments, linkers may be used to link any of the peptides or peptide domains of the disclosure. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.).

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS)ₙ (SEQ ID NO: 246), (GGGGS)ₙ (SEQ ID NO: 247), and (G)n to more rigid linkers of the form (EAAAK)ₙ (SEQ ID NO: 248), (SGGS)ₙ (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32 (6): 577-82; the entire contents are incorporated herein by reference) and (XP)n) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

```
  (SEQ ID NO: 356)
SGGSSGSETPGTSESATPESSGGS,
  (SEQ ID NO: 357)
SGGSSGGSSGSETPGTSESATPESSGGSSGGS,
  (SEQ ID NO: 358)
GGSGGSPGSPAGSPTSTEEGTSESATPESGPGTSTEPSEGSAPGSPAGS
PTSTEEGTSTEPSEGSAPGTSTEPSEGSAPGTSESATPESGPGSEPATS
GGSGGS,
  (SEQ ID NO: 716)
EGGSEEEEESGS,
or
(SEQ ID NO: 717)
KGPKPKKEESEK.
```

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS (SEQ ID NO: 355). In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence: SGGSSGGSSGSETPGTSESATPESSGGSSGGSSGGSSGGS (SEQ ID NO: 360). In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSGGS (SEQ ID NO: 361). In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTEE
GTSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P(AP)4 (SEQ ID NO: 367), P(AP)7 (SEQ ID NO: 368), P(AP)10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10 (1): 439; the entire contents are incorporated herein by reference). Such proline-rich linkers are also termed “rigid” linkers.

Nucleic Acid Programmable DNA Binding Proteins with Guide RNAs

Provided herein are compositions and methods for base editing in cells. Further provided herein are compositions comprising a guide polynucleotide sequence, e.g., a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g., a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 3 or 5′-NAA-3′). In some embodiments, the guide nucleic acid (e.g., guide RNA) is complementary to a sequence in a gene of interest (e.g., a gene associated with a disease or disorder). Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence.

The domains of the base editor disclosed herein can be arranged in any order.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence.

### CAR-T Cell Therapies

Modification of immune cells to express a chimeric antigen receptor can enhance an immune cell's immunoreactive activity, wherein the chimeric antigen receptor has an affinity for an epitope on an antigen, wherein the antigen is associated with an altered fitness of an organism. For example, the chimeric antigen receptor can have an affinity for an epitope on a protein expressed in a diseased cell. Because the CAR-T cells can act independently of major histocompatibility complex (MHC), activated CAR-T cells can kill the diseased cell expressing the antigen. The direct action of the CAR-T cell evades defensive mechanisms that have evolved in response to MHC presentation of antigens to immune cells.

In embodiments, the immune cells contain a kill switch (e.g., RQR8 or an antibody-drug conjugate target). In some cases, a chimeric antigen receptor expressed by the cell contains the kill switch

The modified immune cells and methods provided herein address known limitations of CAR-T therapy and is a promising development towards the next generation of precision cell-based therapies.

In embodiments, one or more genes are modified in an allogeneic immune cell so that the modified allogeneic immune cell has a reduced level of, lacks, or have virtually undetectable levels of beta-2-microglobulin.

Immune cells and/or immune effector cells can be isolated or purified from a sample collected from a subject or a donor using standard techniques known in the art. For example, immune effector cells can be isolated or purified from a whole blood sample by lysing red blood cells and removing peripheral mononuclear blood cells by centrifugation. The immune effector cells can be further isolated or purified using a selective purification method that isolates the immune effector cells based on cell-specific markers such as CD25, CD3, CD4, CD8, CD28, CD45RA, or CD45RO. In one embodiment, CD4⁺ is used as a marker to select T cells. In one embodiment, CD8⁺ is used as a marker to select T cells. In one embodiment, CD4⁺ and CD8⁺ are used as a marker to select regulatory T cells.

In another embodiment, the present disclosure provides T cells that have targeted gene knock-outs at the TCR constant region (TRAC), which is responsible for TCRαβ surface expression. TCRαβ-deficient CAR-T cells are compatible with allogeneic immunotherapy (Qasim et al., Sci. Transl. Med. 9, eaaj2013 (2017); Valton et al., Mol Ther. 2015 September; 23 (9): 1507-1518). If desired, residual TCRαβ T cells are removed using CliniMACS magnetic bead depletion to minimize the risk of GVHD. In another embodiment, the present disclosure provides donor T cells selected ex vivo to recognize minor histocompatibility antigens expressed on recipient hematopoietic cells, thereby minimizing the risk of graft-versus-host disease (GVHD), which is the main cause of morbidity and mortality after transplantation (Warren et al., Blood 2010; 115 (19): 3869-3878).

A technique for isolating or purifying immune effector cells is flow cytometry. In fluorescence activated cell sorting a fluorescently labelled antibody with affinity for an immune effector cell marker is used to label immune effector cells in a sample. A gating strategy appropriate for the cells expressing the marker is used to segregate the cells.

In embodiments, the immune effector cells contemplated in the present disclosure are effector T cells. In some embodiments, the effector T cell is a naïve CD8⁺ T cell, a cytotoxic T cell, a natural killer T (NKT) cell, a natural killer (NK) cell, or a regulatory T (Treg) cell. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell). In some embodiments, immune effector cells are effector NK cells. In some embodiments, the immune effector cell is any other subset of T cells. The modified immune effector cell may express, in addition to the chimeric antigen receptor (CAR), an exogenous cytokine, a different chimeric receptor, or any other agent that would enhance immune effector cell signaling or function. For example, co-expression of the chimeric antigen receptor and a cytokine may enhance the CAR-T cell's ability to lyse a target cell.

Provided herein are also polynucleotides that encode the chimeric antigen receptors (CARs) described herein. In some embodiments, the nucleic acid molecule is isolated or purified. Delivery of the nucleic acid molecules ex vivo or in vivo can be accomplished using methods known in the art or according to the methods of the disclosure. For example, a polynucleotide encoding a chimeric antigen receptor can be delivered to a cell in vivo using a lipid nanoparticle conjugated to a CD5-binding polypeptide of the disclosure, and containing a polynucleotide encoding the chimeric antigen receptor. Alternatively, immune cells obtained from a subject may be transformed with a nucleic acid vector encoding the chimeric antigen receptor. The vector may then be used to transform recipient immune cells so that these cells will then express the chimeric antigen receptor. Efficient means of transforming immune cells include transfection and transduction. Such methods are well known in the art. For example, applicable methods for delivery of the nucleic acid molecule encoding the chimeric antigen receptor (and the nucleic acid(s) encoding the base editor) can be found in International Application No. PCT/US2009/040040 and U.S. Pat. Nos. 8,450,112; 9,132,153; and 9,669,058, each of which is incorporated herein in its entirety. Additionally, those methods and vectors described herein for delivering the nucleic acid encoding the base editor are applicable to delivering the nucleic acid encoding the chimeric antigen receptor.

### Chimeric Antigen Receptors and CAR-T Cells

The present disclosure provides immune cells modified (e.g., in vivo) to express chimeric antigen receptors (CARs). Modification of immune cells to express a chimeric antigen receptor can enhance an immune cell's immunoreactive activity, wherein the chimeric antigen receptor has an affinity for an epitope on an antigen, wherein the antigen is associated with an altered fitness of an organism. For example, the chimeric antigen receptor can have an affinity for an epitope on a protein expressed in a neoplastic cell. Because the CAR-T cells can act independently of major histocompatibility complex (MHC), activated CAR-T cells can kill the neoplastic cell expressing the antigen. The direct action of the CAR-T cell evades neoplastic cell defensive mechanisms that have evolved in response to MHC presentation of antigens to immune cells. Exemplary chimeric antigen receptors, modified immune cells, and methods for preparing the same are described in PCT Applications No. PCT/US2020/013964, PCT/US2020/052822, PCT/US2020/018178, PCT/US2021/52035, and PCT/US2022/075021, or in Hardke-Wolenski, et al., Biomedicines 10:1493 (2022), the disclosures of which are incorporated herein by reference in their entirety for all purposes. In some embodiments, the modified immune cells of the disclosure express a CAR containing an antigen binding domain containing a CD5-binding polypeptide of the disclosure.

However, target antigens associated with neoplastic cells may also be expressed on healthy immune cells. Accordingly, activated CAR-T cells not only kill neoplastic cells expressing the target antigen but also healthy immune cells that also express the target antigen. To prevent this fratricide or self-killing of immune cells, the disclosure provides a CAR-T that has been modified using nucleobase editors to reduce or eliminate the expression of a target antigen (e.g., CD5) to provide fratricide resistance. In some embodiments, the disclosure provides a fratricide resistant modified immune effector cell that expresses a chimeric antigen receptor to target a neoplastic cell.

Some embodiments comprise autologous immune cell immunotherapy, wherein T cells within a subject in need of CAR-T cell therapy are modified in vivo to express a chimeric antigen receptor. In some embodiments, the T cells are modified by administering to the subject a lipid nanoparticle conjugated to an anti-CD5 polypeptide of the disclosure and containing a polynucleotide encoding a chimeric antigen receptor. The modified immune cells express the chimeric antigen receptor and are effectively redirected against specific antigens. The immune cells modified to express the chimeric antigen receptor are effective in treating a neoplasia (e.g., T- or NK-cell malignancy) in the subject.

Some embodiments comprise autologous immune cell immunotherapy, wherein immune cells are obtained from a subject having a disease or altered fitness characterized by cancerous or otherwise altered cells expressing a surface marker. The obtained immune cells are genetically modified to express a chimeric antigen receptor and are effectively redirected against specific antigens. Thus, in some embodiments, immune cells are obtained from a subject in need of CAR-T immunotherapy. In some embodiments, these autologous immune cells are cultured and modified shortly after they are obtained from the subject. In other embodiments, the autologous cells are obtained and then stored for future use. This practice may be advisable for individuals who may be undergoing parallel treatment that will diminish immune cell counts in the future. In allogeneic immune cell immunotherapy, immune cells can be obtained from a donor other than the subject who will be receiving treatment. In some embodiments, immune cells are obtained from a healthy subject or donor and are genetically modified to express a chimeric antigen receptor and are effectively redirected against specific antigens. The immune cells, after modification to express a chimeric antigen receptor, are administered to a subject for treating a neoplasia (e.g., T- or NK-cell malignancy). In some embodiments, immune cells to be modified to express a chimeric antigen receptor can be obtained from pre-existing stock cultures of immune cells.

Immune cells and/or immune effector cells can be isolated or purified from a sample collected from a subject or a donor using standard techniques known in the art. For example, immune effector cells can be isolated or purified from a whole blood sample by lysing red blood cells and removing peripheral mononuclear blood cells by centrifugation. The immune effector cells can be further isolated or purified using a selective purification method that isolates the immune effector cells based on cell-specific markers such as CD25, CD3, CD4, CD8, CD28, CD45RA, or CD45RO. In one embodiment, CD4⁺ is used as a marker to select T cells. In one embodiment, CD8⁺ is used as a marker to select T cells. In one embodiment, CD4⁺ and CD8⁺ are used as a marker to select regulatory T cells.

Provided herein are also nucleic acids that encode the chimeric antigen receptors described herein. In some embodiments, the nucleic acid is isolated or purified. Delivery of the nucleic acids ex vivo or in vitro can be accomplished using methods known in the art. For example, immune cells obtained from a subject may be transformed with a nucleic acid vector encoding the chimeric antigen receptor. The vector may then be used to transform recipient immune cells so that these cells will then express the chimeric antigen receptor. Efficient means of transforming immune cells include transfection and transduction. Such methods are well known in the art. For example, applicable methods for delivery the nucleic acid molecule encoding the chimeric antigen receptor can be found in International Application No. PCT/US2009/040040 and U.S. Pat. Nos. 8,450,112; 9,132,153; and 9,669,058, each of which is incorporated herein in its entirety. Additionally, those methods and vectors described herein for delivering a polynucleotide are applicable to delivering the polynucleotide encoding the chimeric antigen receptor.

### Extracellular Binding Domain

The chimeric antigen receptors of the disclosure include an extracellular binding domain. The extracellular binding domain of a chimeric antigen receptor contemplated herein comprises an amino acid sequence of an antibody (e.g., a CD5-binding polypeptide of the disclosure), or an antigen binding fragment thereof, that has an affinity for a specific antigen. In some embodiments, the antigen is a cluster of differentiation 5 (CD5) polypeptide, or a fragment thereof.

In some embodiments the chimeric antigen receptor comprises an amino acid sequence of an antibody. In some embodiments, the chimeric antigen receptor comprises the amino acid sequence of an antigen binding fragment of an antibody (e.g., a VHH antibody of the disclosure). The antibody (or fragment thereof) portion of the extracellular binding domain recognizes and binds to an epitope of an antigen (e.g., CD5). In some embodiments, the antibody fragment portion of a chimeric antigen receptor is a VHH antibody. In other embodiments, the antibody fragment portion of a chimeric antigen receptor is a multichain variable fragment, which comprises more than one extracellular binding domains and therefore bind to more than one antigen simultaneously. In a multiple chain variable fragment embodiment, a hinge region may separate the different variable fragments, providing necessary spatial arrangement and flexibility.

In some embodiments, the extracellular binding domain is a CD5 binding polypeptide of the disclosure.

In other embodiments, the antibody portion of a chimeric antigen receptor comprises at least one heavy chain and at least one light chain. In some embodiments, the antibody portion of a chimeric antigen receptor comprises two heavy chains, joined by disulfide bridges and two light chains, wherein the light chains are each joined to one of the heavy chains by disulfide bridges. In some embodiments, the light chain comprises a constant region and a variable region. Complementarity determining regions residing in the variable region of an antibody are responsible for the antibody's affinity for a particular antigen. Thus, antibodies that recognize different antigens comprise different complementarity determining regions. Complementarity determining regions reside in the variable domains of the extracellular binding domain, and variable domains (i.e., the variable heavy and variable light) can be linked with a linker or, in some embodiments, with disulfide bridges. In some embodiments, the variable heavy chain and variable light chain are linked by a (GGGGS)ₙ linker (SEQ ID NO: 247), wherein the n is an integer from 1 to 10. In some embodiments, the linker is a (GGGGS)₃ linker (SEQ ID NO: 624).

In some embodiments, the antigen recognized and bound by the extracellular domain is a protein or peptide, a nucleic acid, a lipid, or a polysaccharide. Antigens can be heterologous, such as those expressed in a pathogenic bacteria or virus. Antigens can also be synthetic; for example, some individuals have extreme allergies to synthetic latex and exposure to this antigen can result in an extreme immune reaction. In some embodiments, the antigen is autologous, and is expressed on a diseased or otherwise altered cell.

For example, in some embodiments, the antigen is expressed in a neoplastic cell. In some embodiments, the neoplastic cell is a malignant T- or NK-cell. In some embodiments, the malignant T- or NK-cell is a malignant precursor T- or NK-cell. In some embodiments, the malignant T- or NK-cell is a malignant mature T- or NK-cell. Nonlimiting examples of neoplasia include T-cell acute lymphoblastic leukemia (T-ALL), mycosis fungoides (MF), Sézary syndrome (SS), Peripheral T/NK-cell lymphoma, Anaplastic large cell lymphoma ALK+, Primary cutaneous T-cell lymphoma, T-cell large granular lymphocytic leukemia, Angioimmunoblastic T/NK-cell lymphoma, Hepatosplenic T-cell lymphoma, Primary cutaneous CD30+lymphoproliferative disorders, Extranodal NK/T-cell lymphoma, Adult T-cell leukemia/lymphoma, T-cell prolymphocytic leukemia, Subcutaneous panniculitis-like T-cell lymphoma, Primary cutaneous gamma-delta T-cell lymphoma, Aggressive NK-cell leukemia, and Enteropathy-associated T-cell lymphoma.

Antibody-antigen interactions are noncovalent interactions resulting from hydrogen bonding, electrostatic or hydrophobic interactions, or from van der Waals forces. The affinity of extracellular binding domain of the chimeric antigen receptor for an antigen can be calculated with the following formula:

$${K}_{A} = [ Antibody - Antigen ] / [ Antibody ] [ Antigen ] , wherein$$

$$[ Ab ] = molar concentration of unoccupied binding sites on the antibody ;$$

$$[ Ab ] = molar concentration of unoccupied binding sites on the antigen ;$$

$$and$$

$$[ Ab - Ag ] = molar concentration of the antibody - antigen complex .$$

The antibody-antigen interaction can also be characterized based on the dissociation of the antigen from the antibody. The dissociation constant (K_{D}) is the ratio of the association rate to the dissociation rate and is inversely proportional to the affinity constant. Thus, K_{D}=1/KA. Those skilled in the art will be familiar with these concepts and will know that traditional methods, such as ELISA assays, can be used to calculate these constants.

### Transmembrane Domain

The chimeric antigen receptors of the disclosure include a transmembrane domain. The transmembrane domain of the chimeric antigen receptors described herein spans the CAR-T cell's lipid bilayer cellular membrane and separates the extracellular binding domain and the intracellular signaling domain. In some embodiments, this domain is derived from other receptors having a transmembrane domain, while in other embodiments, this domain is synthetic. In some embodiments, the transmembrane domain may be derived from a non-human transmembrane domain and, in some embodiments, humanized. By “humanized” is meant having the sequence of the nucleic acid encoding the transmembrane domain optimized such that it is more reliably or efficiently expressed in a human subject. In some embodiments, the transmembrane domain is derived from another transmembrane protein expressed in a human immune effector cell. Examples of such proteins include, but are not limited to, subunits of the T cell receptor (TCR) complex, PD1, or any of the Cluster of Differentiation proteins, or other proteins, that are expressed in the immune effector cell and that have a transmembrane domain. In some embodiments, the transmembrane domain will be synthetic, and such sequences will comprise many hydrophobic residues.

Transmembrane domains for use in the disclosed CARs can include at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some embodiments, the transmembrane domain is derived from CD4, CD8α, CD28 and CD3ζ.

The chimeric antigen receptor is designed, in some embodiments, to comprise a spacer between the transmembrane domain and the extracellular domain, the intracellular domain, or both. Such spacers can be 1, 2, 3, 4, 5, 6, 7, 8, 9, 10, 11, 12, 13, 14, 15, 16, 17, 18, 19, or 20 amino acids in length. In some embodiments, the spacer can be 20, 30, 40, 50, 60, 70, 80, 90, or 100 amino acids in length. In still other embodiments the spacer can be between 100 and 500 amino acids in length. The spacer can be any polypeptide that links one domain to another and are used to position such linked domains to enhance or optimize chimeric antigen receptor function.

### Intracellular Signaling Domain

The chimeric antigen receptors of the disclosure include an intracellular signaling domain. The intracellular signaling domain is the intracellular portion of a protein expressed in a T cell that transduces a T cell effector function signal (e.g., an activation signal) and directs the T cell to perform a specialized function. T cell activation can be induced by a number of factors, including binding of cognate antigen to the T cell receptor on the surface of T cells and binding of cognate ligand to costimulatory molecules on the surface of the T cell. A T cell co-stimulatory molecule is a cognate binding partner on a T cell that specifically binds with a co-stimulatory ligand, thereby mediating a co-stimulatory response by the T cell, such as, but not limited to, proliferation. Co-stimulatory molecules include but are not limited to an MHC class I molecule. Activation of a T cell leads to immune response, Such as T cell proliferation and differentiation (see, e.g., Smith-Garvin et al., Annu. Rev. Immunol., 27:591-619, 2009). Exemplary T cell signaling domains are known in the art. Non-limiting examples include the CD3ζ, CD8, CD28, CD27, CD154, GITR (TNFRSF18), CD134 (OX40), and CD137 (4-1BB) signaling domains.

The intracellular signaling domain of the chimeric antigen receptor contemplated herein comprises a primary signaling domain. In some embodiments, the chimeric antigen receptor comprises the primary signaling domain and a secondary, or co-stimulatory, signaling domain.

In some embodiments, the primary signaling domain comprises one or more immunoreceptor tyrosine-based activation motifs, or ITAMs. In some embodiments, the primary signaling domain comprises more than one ITAM. ITAMs incorporated into the chimeric antigen receptor may be derived from ITAMs from other cellular receptors. In some embodiments, the primary signaling domain comprising an ITAM may be derived from subunits of the TCR complex, such as CD3γ, CD3ε, CD3ζ, or CD3δ. In some embodiments, the primary signaling domain comprising an ITAM may be derived from FcRγ, FcRβ, CD5, CD22, CD79a, CD79b, or CD66d.

In some embodiments, the primary signaling domain is selected from the group consisting of CD8, CD28, CD134 (OX40), CD137 (4-1BB), and CD3ζ.

In some embodiments, the secondary, or co-stimulatory, signaling domain is derived from CD2, CD4, CD5, CD8α, CD28, CD83, CD134, CD137 (4-1BB), ICOS, or CD154, or a combination thereof. In some embodiments, the co-signaling domain is a cytoplasmic domain.

In some embodiments, the CAR comprises one or more signaling domains.

### Molecular Switches

In various embodiments, an immune cell (e.g., a CAR-T cell) of the disclosure expresses a molecular switch alternatively referred to as a “kill switch,” “suicide switch,” or “safety switch.” In some cases, a CAR of the disclosure contains a molecular switch. A kill switch is activated by a pharmaceutical agent (e.g., an antibody). When a kill switch is activated, the kill switch mediates killing of the cell expressing the kill switch. For example, in an embodiment, a kill switch expressed on the surface of a cell mediates the induction of complement-mediated killing of the cell in the presence of a monoclonal antibody (e.g., Rituximab). In some cases, a kill switch binds Rituximab.

### Immunoconjugates

In some embodiments, an anti-CD5 VHH antibody of the disclosure is or is part of an immunoconjugate (“anti-CD5 VHH antibody immunoconjugate”), in which the anti-CD5 VHH antibody is conjugated to one or more heterologous molecule(s), such as, but not limited to, a cytotoxic or an imaging agent. The fusion of the cytotoxic agent with the anti-CD5 VHH antibody may have therapeutic value. Cytotoxic agents include, but are not limited to, radioactive isotopes (e.g., At²¹¹, I¹³¹, I¹²⁵, Y⁹⁰, Rel⁸⁶, Rel⁸⁸, Sml⁵³, Bi²¹², P³², Pb²¹² and radioactive isotopes of Lu); chemotherapeutic agents (e.g., maytansinoids, taxanes, methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins. In some embodiments, the antibody is conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

Among the anti-CD5 VHH antibody immunoconjugates are antibody-drug conjugates (ADCs), in which an anti-CD5 VHH antibody is conjugated to one or more drugs, including but not limited to a maytansinoid (see U.S. Pat. Nos. 5,208,020, 5,416,064 and European Patent EP 0 425 235 B 1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53: 3336-3342 (1993); and Lode et al, Cancer Res. 58: 2925-2928 (1998)); an anthracycline such as daunomycin or doxorubicin (see Kratz et al., Current Med. Chem. 13: 477-523 (2006); Jeffrey et al., Bioorganic & Med. Chem. Letters 16: 358-362 (2006); Torgov et al., Bioconj. Chem. 16: 717-721 (2005); Nagy et al, Proc. Natl. Acad. Sci. USA 97: 829-834 (2000); Dubowchik et al, Bioorg. & Med. Chem. Letters 12: 1529-1532 (2002); King et al, J. Med. Chem. 45: 4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

Also among the anti-CD5 VHH antibody immunoconjugates are those in which the antibody is conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some embodiments, the anti-CD5 VHH antibody is conjugated to a protein degrader, such as those described in Frere, G., et al. Methods in Cell Biology, 167:1-26 (2022) and Sasso, J., et al. Biochemistry, 62:601-623 (2023), or in International Patent Applications No. WO 2021/053555, WO 2021/249517, WO 2020/006264, WO 2008/115516, WO 2021/126805, WO 2021/178920, WO 2021/127080, WO 2014/094138, WO 2015/200795, WO 2017/117118, and WO 2020/079103 the disclosures of which is incorporated herein by reference in their entireties for all purposes. In some embodiments, the degrader is CC-122, CC-220, CC-99282, CFT7455, DKY709, CR8, Glue01, HQ005, FPFT-2216, TMX-4116, Eragidomide, BTX-1188, MG-277, ZHX-1-161, Indisulam, E7820, dCeMM1, CQS, NRX-252114, NRX-252262, BI-3802, CCT369260, Cyclosporin A, Lupkynis, Sanglifehrin A, Auxin, Jasmonate, lenalidomide (Revlimid), lenalidomide, pomalidomide (Pomalyst), or thalidomide. Non-limiting examples of types of protein degraders suitable for use in compositions, conjugates, and/or methods of the disclosure include heterobifunctional degraders and molecular glue degraders.

Also among the anti-CD5 VHH antibody immunoconjugates are those in which the anti-CD5 VHH antibody is conjugated to a radioactive atom to form a radioconjugate. Exemplary radioactive isotopes include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu.

Conjugates of an anti-CD5 VHH antibody and cytotoxic agent may be made using any of a number of known protein coupling agents, e.g., linkers, (see Vitetta et al., Science 238:1098 (1987)). The linker may be a “cleavable linker” facilitating release of a cytotoxic drug in the cell, such as acid-labile linkers, peptidase-sensitive linkers, photolabile linkers, dimethyl linkers, and disulfide-containing linkers (Chari et al., Cancer Res. 52: 127-131 (1992); U.S. Pat. No. 5,208,020).

### Modified Polynucleotides

To enhance expression, stability, and/or genomic/base editing efficiency, and/or reduce possible toxicity, a polynucleotide of the disclosure can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

### Expression of Polypeptides in a Host Cell

Polypeptides of the present disclosure may be expressed in virtually any host cell of interest, including mammalian cells (e.g., human cells). In some embodiments, the host cell is an immune cell (e.g., T- or NK-cell). In some embodiments, the host cell is an immune cell (e.g., T- or NK-cell). In some embodiments, the host cell is a T cell.

An expression vector containing a DNA encoding a polypeptide can be produced, for example, by linking the DNA to the downstream of a promoter in a suitable expression vector.

In some embodiments, the nucleic acid sequence is inserted into the genome of the cell (e.g., T cell or NK cell) by introducing a vector, for example, a viral or non-viral vector, comprising the nucleic acid. Examples of viral vectors include, but are not limited to, adeno-associated viral (AAV) vectors, retroviral vectors or lentiviral vectors. In some embodiments, the lentiviral vector is an integrase-deficient lentiviral vector. In some embodiments, the nucleic acid sequence is inserted into the genome of the cell (e.g., T cell) via non-viral delivery. In non-viral delivery methods, the nucleic acid can be naked DNA, or in a non-viral plasmid or vector.

Regarding the promoter to be used, any promoter appropriate for a host to be used for gene expression can be used. For example, when the host is an animal cell, an SRα promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK), MND (a synthetic promoter that contains the U3 region of a modified MoMuLV LTR with myeloproliferative sarcoma virus enhancer) promoter, and the like can be used. In some embodiments, the promoter is a CMV promoter or an SR.alpha. promoter, or the like.

### Polynucleotides and Vectors

In some cases, more than one anti-CD5-binding VHH antibody (i.e., anti-CD5 VHH) is coupled or linked (e.g., covalently linked) to other sequences, e.g., a leader amino acid sequence, domains of a chimeric antigen receptor, one or more spacer or linker (flexible spacer or linker) amino acid sequences, or one or more epitope tag amino acid sequences. In an embodiment, a polynucleotide molecule, such as a recombinant or isolated polynucleotide molecule, encodes a polypeptide containing an anti-CD5 VHH polypeptide (e.g., a VHH antibody or a chimeric antigen receptor). In an embodiment, the polynucleotide encodes a fragment or portion of the anti-CD5 VHH, where the fragment or portion maintains CD5 binding activity.

In an embodiment, an anti-CD5 VHH can be humanized, i.e., modified to increase its similarity to antibodies or antibody variants produced naturally in humans, using techniques known and practiced in the art. Briefly and by way of nonlimiting example, a humanized antibody can be generated by inserting the appropriate CDR coding sequences (e.g., ‘donor’ sequences that are responsible for the desired binding properties) into a human antibody “scaffold” (e.g., ‘acceptor’ sequences) comprising essentially invariant framework region (FR) sequences (FRs). In embodiments, the CDRs of the anti-CD5 VHH antibodies described herein may be inserted into FRs, which provide the structural scaffold that allows the CDRs to bind to CD5. Recombinant DNA methods using an appropriate vector and expression in mammalian cells are employed and routinely practiced in the art to achieve the production of recombinant humanized antibodies.

In an embodiment, the polynucleotide encodes a CD5-binding VHH molecule having binding function, or a functional binding portion thereof. In embodiments, antibody fragments, microproteins, darpins, anticalins, peptide mimetic molecules, aptamers, synthetic molecules, etc. can be linked to the anti-CD5 VHH binding molecule.

In an embodiment, an anti-CD5 VHH can be modified, for example, by attachment (e.g., either directly or indirectly via a linker or spacer) to another agent (e.g., a detectable label, a cytotoxic drug, and/or another polypeptide). Accordingly, a polynucleotide (e.g., DNA) that encodes one anti CD5 VHH is joined (in reading frame) with a polynucleotide encoding a second polypeptide, and so on. In certain embodiments, additional amino acids are encoded within the polynucleotide between the anti-CD5 VHH and other polypeptides so as to produce an unstructured region (e.g., a flexible spacer) that separates the anti-CD5 VHH from the other polypeptides to better promote independent folding of each polypeptide into its active or functional conformation or shape. Commercially available techniques for fusing proteins (or their encoding polynucleotides) may be employed to recombinantly join or couple polypeptide sequences to one another.

The compositions and methods described herein in various embodiments include an isolated polynucleotide sequence or an isolated polynucleotide molecule that encodes a polypeptide (e.g., anti-CD5 VHH or a chimeric antigen receptor containing an anti-CD5 VHH domain of the disclosure). Accordingly, in some embodiments, the isolated polynucleotide sequence or isolated polynucleotide molecule comprises or consists of a polynucleotide sequence that encodes a polypeptide molecule (anti-CD5 VHH) having an amino acid sequence listed in any one of Tables 1A-1C, or a functional portion thereof, as described herein. In an embodiment, a composition comprises a combination of the isolated polynucleotide sequences or isolated polynucleotide molecules.

Also encompassed by the present disclosure are polynucleotide sequences, DNA or RNA, which are substantially complementary to the DNA sequences encoding the polypeptides described herein, and which specifically hybridize with these DNA sequences under conditions of stringency known to those of skill in the art. As referred to herein, substantially complementary means that the nucleotide sequence of the polynucleotide need not reflect the exact sequence of the original encoding sequences, but must be sufficiently similar in sequence to permit hybridization with a nucleic acid sequence under high stringency conditions. For example, non-complementary bases can be interspersed in a nucleotide sequence, or the sequences can be longer or shorter than the polynucleotide sequence, provided that the sequence has a sufficient number of bases complementary to the sequence to allow hybridization thereto. Conditions for stringency are described, e.g., in Ausubel, F. M., et al., Current Protocols in Molecular Biology, (Current Protocol, 1994), and Brown, et al., Nature, 366:575 (1993); and further defined in conjunction with certain assays.

Vectors and plasmids containing one or more of the polynucleotide molecules encoding the anti-CD5 VHH amino acid sequences of any one of Tables 1A-1C, or a functional portion thereof, are provided. Suitable vectors for use in eukaryotic and prokaryotic cells are known in the art and are commercially available or readily prepared by the skilled practitioner in the art. Additional vectors can also be found, for example, in Ausubel, F. M., et al., Ibid, and in Sambrook et al., “Molecular Cloning: A Laboratory Manual,” 2nd ED. (1989), and other editions.

Uses of plasmids, vectors or viruses (viral vectors) containing polynucleotides encoding the anti-CD5 VHHs as described herein include generation of mRNA or protein in vitro or in vivo. In related embodiments, host cells transformed with the plasmids, vectors, or virus vectors are provided, as described above. Nucleic acid molecules can be inserted into a construct (such as a prokaryotic expression plasmid, a eukaryotic expression vector, or a viral vector construct, which can, optionally, replicate and/or integrate into a recombinant host cell by known methods. The host cell can be a eukaryote or prokaryote and can include, for example and without limitation, yeast (such as Pichia pastoris or Saccharomyces cerevisiae), bacteria (such as E. coli, or Bacillus subtilis), animal cells or tissue (CHO or COS cells), insect Sf9 cells (such as baculoviruses infected SF9 cells), or mammalian cells (somatic or embryonic cells, Human Embryonic Kidney (HEK) cells, Chinese hamster ovary (CHO) cells, HeLa cells, human 293 cells (Expi293F), and monkey COS-7 cells). Suitable host cells also include a mammalian cell, a bacterial cell, a yeast cell, an insect cell, a plant cell, or an algal cell.

In another aspect, an RNA polynucleotide, in particular, mRNA, encodes a polypeptide as described herein. mRNA encoding the polypeptides may contain a 5′ cap structure, a 5′ UTR, an open reading frame, a 3′ UTR and poly-A sequence followed by a C30 stretch and a histone stem loop sequence (Thess, A. et al., 2015, Mol Ther, 23 (9): 1456-1464; Thran, M. et al., 2017, EMBO Molecular Medicine, DOI: 10.15252/emmm.201707678). Sequences may be codon-optimized for human use using techniques and protocols known and used by those skilled in the art. In an embodiment, the mRNA sequences do not include chemically modified bases. mRNAs encoding the anti-CD5 VHHs thereof as described herein may be capped enzymatically or further polyadenylated for in vivo studies/use. In an embodiment, a polypeptide of the disclosure is encoded by a mRNA molecule. In an embodiment, the mRNA may be delivered to or introduced into a cell.

Expression of proteins, which normally have a shortened serum half-life, by encoding mRNA, particularly sequence optimized, unmodified mRNA, advantageously prolongs the bioavailability of these proteins for in vivo activity. (see, e.g., K. Kariko et al, 2012, Mol. Ther., 20:948-953; Thess, A. et al., 2015, Mol Ther, 23 (9): 1456-1464).

### Recombinant Polypeptide Expression

In general, polypeptides of the disclosure (e.g., VHH antibodies) may be produced by transformation of a suitable host cell with all or part of a polypeptide-encoding nucleic acid molecule or fragment thereof in a suitable expression vehicle.

Those skilled in the field of molecular biology will understand that any of a wide variety of systems may be used to express a recombinant protein. The precise host cell used is not critical to the various aspects of the disclosure. A polypeptide of the disclosure may be produced in a prokaryotic host (e.g., E. coli) or in a eukaryotic host (e.g., an immune cell, such an immune effector cell (e.g., a T cell) Saccharomyces cerevisiae, insect cells, e.g., Sf21 cells, or mammalian cells, e.g., NIH 3T3, HeLa, or COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides (e.g., VHH antibodies or chimeric antigen receptors) of the disclosure. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

Once the recombinant polypeptide of the disclosure is expressed, it can be isolated, e.g., using affinity chromatography. In one example, an antibody (e.g., produced as described herein) raised against an antigen of the disclosure may be attached to a column and used to isolate the recombinant polypeptide. Lysis and fractionation of polypeptide-harboring cells prior to affinity chromatography may be performed by standard methods.

Once isolated, a recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the disclosure, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

### Delivery Systems

### Nucleic Acid-Based Delivery and Conjugated Lipid Nanoparticles

Nucleic acid molecules encoding a polypeptide according to the present disclosure can be administered to subjects or delivered into cells in vitro or in vivo by art-known methods or as described herein. For example, a polypeptide of the disclosure can be delivered by vectors (e.g., viral or non-viral vectors), or by naked DNA, DNA complexes, lipid nanoparticles, or a combination of the aforementioned compositions. A polypeptide may be delivered to a cell using any methods available in the art including, but not limited to, physical methods (e.g., electroporation, particle gun, calcium phosphate transfection), viral methods, non-viral methods (e.g., liposomes, cationic methods, lipid nanoparticles, polymeric nanoparticles), or biological non-viral methods (e.g., attenuated bacterial, engineered bacteriophages, mammalian virus-like particles, biological liposomes, erythrocyte ghosts, exosomes). In embodiments, the lipid nanoparticle is conjugated to a CD5-binding polypeptide of the disclosure. Such lipid nanoparticles can be useful in delivering a polynucleotide contained within the lipid nanoparticle (e.g., a polynucleotide encoding a chimeric antigen receptor) to a T cell of a subject in vivo. Methods for conjugating a polypeptide, such as a VHH antibody, to a lipid nanoparticle are known in the art (see, e.g., Yaozhong, et al. “Nanobody™-based delivery systems for diagnosis and targeted tumor therapy,” Front Immunol 8:1442 (2017)).

Nanoparticles, which can be organic or inorganic, are useful for delivering a polynucleotide or polypeptide to a cell. Nanoparticles are well known in the art and any suitable nanoparticle can be used to deliver a polypeptide or a polynucleotide encoding the same to a cell. In one example, organic (e.g., lipid and/or polymer) nanoparticles are suitable for use as delivery vehicles in certain embodiments of this disclosure. Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, WO2024019936, and WO2021141969, the disclosures of each of which is incorporated herein by reference in its entirety for all purposes.

### Viral Vectors

A polypeptide or polynucleotide can be delivered with a viral vector. In some embodiments, a polypeptide disclosed herein can be encoded on a polypeptide that is contained in a viral vector. In some embodiments, a polypeptide can be encoded on one or more viral vectors. Non-limiting examples of viral vectors include lentivirus (e.g., HIV and FIV-based vectors), Adenovirus (e.g., AD100), Retrovirus (e.g., Maloney murine leukemia virus, MML-V), herpesvirus vectors (e.g., HSV-2), rabies virus (see, e.g., U.S. Patent Application No. US 2022/0290164 A1, the disclosure of which is incorporated by reference in its entirety for all purposes), and Adeno-associated viruses (AAVs), or other plasmid or viral vector types.

### Non-Viral Platforms for Gene Transfer

Non-viral platforms for introducing a heterologous polynucleotide into a cell of interest are known in the art.

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas9 nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In other embodiments, a single-stranded DNA (ssDNA) can produce efficient HDR with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (IssDNA) donors.

### Pharmaceutical Compositions

In some aspects, the present disclosure provides a pharmaceutical composition comprising any of the polypeptides, polynucleotides, or lipid nanoparticles described herein.

The pharmaceutical compositions of the present disclosure can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, any of the polypeptides or lipid nanoparticles described herein are provided as part of a pharmaceutical composition. In some embodiments, the pharmaceutical composition comprises any of the CD5-binding polypeptides described herein. In some embodiments pharmaceutical composition comprises a lipid nanoparticle conjugated to a CD5-binding polypeptide of the disclosure and containing a polynucleotide encoding a chimeric antigen receptor, and a pharmaceutically acceptable excipient. Pharmaceutical compositions can optionally comprise one or more additional therapeutically active substances.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present disclosure provide methods of treating a subject in need, the method comprising administering to a subject in need an effective therapeutic amount of a pharmaceutical composition as described herein. More specifically, the methods of treatment include administering to a subject in need thereof one or more pharmaceutical compositions comprising a CD5-binding polypeptide of the disclosure, such as a composition comprising a lipid nanoparticle conjugated to the CD5-binding polypeptide.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

### Substantially Identical Amino Acid and Nucleotide Sequences for VHHS

There is a large body of information in the literature supporting the fact that closely related antibody (Ab) sequences are capable of performing the same binding and therapeutic functions such that this is now generally accepted by those with ordinary skill in the art of immunological sciences. The creation of Abs with small numbers of amino acid sequence variations occurs naturally within mammals and some other animal species during the process of ‘affinity maturation’ in which Ab-producing cells that bind a newly encountered antigen (Ag) are expanded, and their progeny cells contain random mutations within portions of the Ab coding DNA that results in new, related Ab sequences. The cells expressing Abs that have gained improved binding properties for the new Ag are then selected and expanded, thereby increasing the amount of the improved antibody in the animal. This process continues through multiple generations of mutation and selection until Abs with greatly improved antigen binding properties result. The process of Ab affinity maturation demonstrates that related, yet not identical, Ab amino acid sequences can possess similar target binding properties and perform similar therapeutic functions in vivo.

The present disclosure provides anti-CD5 VHH antibodies having related sequences that are capable of binding CD5. The Abs described herein are heavy-chain only, single domain VHH antibodies, which are generated in camelid alpacas, which have been reported to be convenient sources of camelid VHH antibodies (See, e.g., Maass, D. R. et al., 2007, J. Immunol. Methods, 324:13-25). Briefly, an animal capable of producing VHH antibodies in response to an antigen are immunized with a selected CD5 antigen (CD5 Ag) one or multiple times to permit the animal to undergo affinity maturation of the anti-CD5 VHHs that are produced. Anti-CD5 VHHs are then isolated and the encoding DNA selected for expression of soluble VHHs that bind CD5 Ag and have potential therapeutic or diagnostic properties. During this process, many examples of closely related anti-CD5 binding VHHs are isolated, which are distinctive, and which are presumably intermediates that result from the affinity maturation process which occurs during anti-CD5 VHH production in alpaca lymphocytes. These related anti-CD5 VHHs are screened for binding to CD5 Ag, and the most promising members of homology groups of CD5-binding VHHs are identified and become lead candidates for further development.

Similar to all mammalian antibodies, VHHs consist of four, well-conserved ‘framework’ regions (FRs) which are important in forming the antibody structure. Between the FRs (FR1, FR2, FR3 and FR4) are three much less well-conserved CDRs or hypervariable regions (CDR1, CDR2 and CDR3) which principally interact with and bind to antigenic determinants or epitopes on antigens (Ags), such as CD5. The CDR sequences vary widely so as to interact and bind to epitopes of Ags. The third CDR, CDR3, is generally the longest in sequence and is most diverse of the CDRs within VHHs, both in size and sequence. By way of nonlimiting example, CDR3 in VHHs can range in size from about 5 to about 30 amino acid residues. Without intending to be bound by theory, VHHs and CDR3 regions that bind to the same CD5 target Ag are considered to have resulted from affinity maturation of a common precursor VHH within the animal and are classified as a ‘homology group.’ Individual VHHs within a homology group are classified by their binding to the target Ag, and the members of the VHH homology group are able to ‘compete’ with each other for binding to the Ag, thus demonstrating that they bind to the same region on the target Ag. In VHH molecules, the CDRs (CDR1, CDR2 and CD3) play a role in the ability of a VHH to bind to the target Ag, e.g., CD5, in conjunction with CDR1 and CDR2.

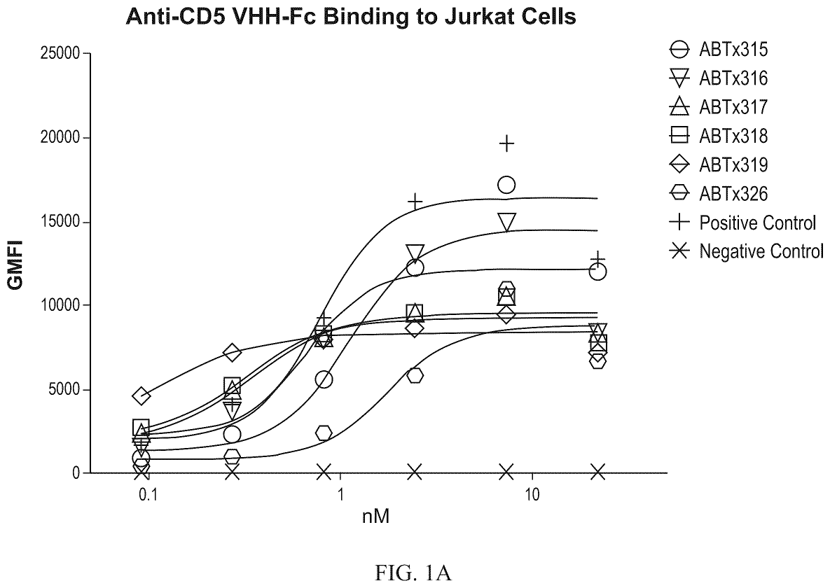

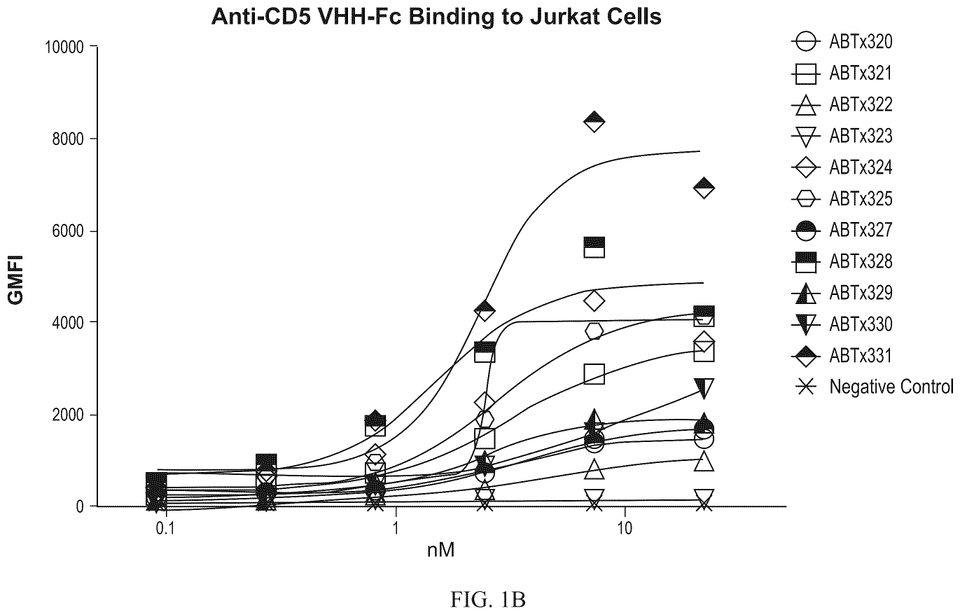

Since the FRs maintain the structure of a VHH and the positioning of the CDRs for binding to the target Ag, the FRs of VHHs typically do not vary extensively in sequence (FIG. 1). However, some VHH FR amino acid sequence variation is permissible, particularly in cases in which an amino acid substitution involves the replacement or substitution of one amino acid with another amino acid having similar properties (e.g., similarity in being charged or uncharged), i.e., a conservative substitution. Such conservative changes in FRs can often be found naturally within VHHs that have undergone affinity maturation in an animal. Similar to the case with FRs, VHH CDRs also typically do not vary extensively in amino acid sequence or type so as not to compromise their ability to specifically bind to Ag. As would be appreciated by one skilled in the art, an estimation of the extent of amino acid sequence variation that can be tolerated within VHHs without compromising their Ag binding ability can be made by observing the variation that occurs naturally within affinity-matured homology groups of VHHs isolated from the same types of animals and which bind to the same Ag.

In an embodiment, sequence variation is particularly acceptable in the CDR regions, e.g., CDR1, CDR2, and/or CDR3, while the feature of VHH binding to antigen CD5 is maintained. In an embodiment, amino acid sequence variation results from conservative amino acid substitutions in a VHH sequence. In an embodiment, the conservative amino acid substitutions are in one or more CDR sequences of the VHH polypeptide. In an embodiment, the conservative amino acid substitutions are in one or more FR sequences of the VHH polypeptide. In an embodiment, the conservative amino acid substitutions are in one or more CDR sequences and in one or more FR sequences of the VHH polypeptide.

An example evidencing that VHH sequence variation is acceptable within related VHHs having the same Ag binding characteristics is described in Tremblay et al., 2013, Infect Immun 81:4592-4603. In this report, 11 VHH sequences comprise a large homology group with closely related CDR3 sequences, and the unusual property of cross-specific binding to two different Shiga toxins, Stx1 and Stx2. Two of the more distantly related VHH members of this homology group are characterized as having common Ag binding characteristics. These two related VHHs were found to have 32 amino acid changes in the total VHH sequence of 120 or 121 residues. Thus, a 26% variation in amino acid sequence did not adversely affect the common Ag binding properties of the VHH proteins.

### KITS

The disclosure provides kits for the treatment of a disease or disorder (e.g., a neoplasia such as a lymphoma) in a subject, where the kit contains a CD5-binding polypeptide of the disclosure, or a polynucleotide encoding the same, and a suitable carrier or excipient. In some embodiments, the kit contains a lipid nanoparticle containing a CD5-binding polypeptide of the disclosure, where the CD5-binding polypeptide is conjugated to the lipid nanoparticle. In some cases, the lipid nanoparticle containing the CD5-binding polypeptide further contains a polynucleotide encoding a chimeric antigen receptor.

The kits may further comprise written instructions for using a CD5-binding polypeptide or lipid nanoparticle conjugated to a CD5-binding polypeptide as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit comprises instructions in the form of a label or separate insert (package insert) for suitable operational parameters. In yet another embodiment, the kit comprises one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of the various aspects and embodiments of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the various aspects and embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Discovery and Characterization of Anti-Cluster of Differentiation 5 (CD5) VHH Antibodies

Experiments were undertaken to identify and characterize VHH antibodies capable of binding to human and/or cynomolgus CD5 polypeptides by llama and alpaca immunization and phage display panning. Amino acid sequences for antibodies identified are provided in Tables 1A-1C.

Tables 8A-8C list data obtained using periplasmic extracts (P.E.) from individual clones following phage display panning that expressed VHH antibodies capable of binding to HEK293T cells surface-expressing human or cynomolgus CD5 polypeptides. Table 8A lists the HCDR3 variant families to which antibodies evaluated belonged (see Tables 1A-1C).

The data of Table 8B was prepared by transiently transfecting HEK293T cells with polynucleotides encoding human CD5 (huCD5) or rhesus macaque (Macaca mulatta) CD5 (rhCD5) DNA using lipofectamine. The cells were resuspended to a final concentration of 1.0E+06 cells/ml in flow cytometry (FACS) buffer and aliquoted in a V-bottom 96-well plate. Periplasmic extract (P.E.) samples and mouse anti-c-myc 9E10 antibody (Roche, Cat nr. 11667203001) were pre-mixed. Cells were incubated with Phapgemid-anti-c-myc mix followed by incubation with goat anti-mouse IgG-APC (Thermo, Cat nr. A-865) in FACS buffer. Analysis was then performed using iQUE3 screener equipment.

The data of Table 8C was prepared by loading biotinylated human CD5-HIS and biotinylated cynomolgus CD5-HIS onto streptavidin (SA) Octet pins. The SA-human CD5 and the SA-Cyno CD5 bound pins were then dipped into the PE from individual clones so that PE specific for human or cyno CD5 would bind in solution. The off-rates were then calculated by placing the sensor into Octet buffer and PE dissociation was measured versus time.

TABLE 8A
VHH antibodies evaluated and their corresponding
HCDR3 variant families.

| VHH Antibody name | HCDR3 variant family |
|---|---|
| ABTx315 | 1 |
| ABTx316 | 12 |
| ABTx317 | 13 |
| ABTx318 | 15 |
| ABTx319 | 17 |
| ABTx320 | 28 |
| ABTx321 | 43 |
| ABTx322 | 46 |
| ABTx323 | 50 |
| ABTx324 | 57 |
| ABTx325 | 58 |
| ABTx326 | 60 |
| ABTx327 | 63 |
| ABTx328 | 65 |
| ABTx329 | 66 |
| ABTx330 | 67 |
| ABTx331 | 68 |

TABLE 8B
Percent of cells expressing human CD5 or cynomolgus
CD5 that showed binding to the indicated antibodies
present in periplasmic extract.

| VHH Antibody name | Human CD5 Cells | Cynomolgus CD5 Cells |
|---|---|---|
| ABTx315 | 86.41 | 79.06 |
| ABTx316 | 85.80 | 81.74 |
| ABTx317 | 82.83 | 79.49 |
| ABTx318 | 84.90 | 67.14 |
| ABTx319 | 74.76 | 62.65 |
| ABTx320 | 80.30 | 77.73 |
| ABTx321 | 84.87 | 87.65 |
| ABTx322 | 70.12 | 82.33 |
| ABTx323 | 64.13 | 54.56 |
| ABTx324 | 73.33 | 73.15 |
| ABTx325 | 77.43 | 80.03 |
| ABTx326 | 41.58 | 58.43 |
| ABTx327 | 68.03 | 71.70 |
| ABTx328 | 45.39 | 40.66 |
| ABTx329 | 72.02 | 66.48 |
| ABTx330 | 78.60 | 72.02 |
| ABTx331 | 89.20 | 71.85 |

TABLE 8C
Dissociation constants for binding of the indicated
VHH antibodies to human CD5 or cynolmogus CD5.

| | Human | Cynomolgus CD5 |
|---|---|---|
| VHH Antibody name | CD5 kd (s − 1) | kd (s − 1) |
| ABTx315 | 5.66E−04 | 3.45E−04 |
| ABTx316 | 5.40E−03 | 3.43E−02 |
| ABTx317 | 1.31E−02 | 1.18E−01 |
| ABTx318 | 1.38E−02 | 5.46E−02 |
| ABTx319 | 1.16E−02 | 5.31E−02 |
| ABTx320 | 1.37E−03 | 7.57E−04 |
| ABTx321 | 7.70E−03 | 1.36E−02 |
| ABTx322 | 3.31E−04 | 2.80E−03 |
| ABTx323 | 1.38E−04 | 3.47E−04 |
| ABTx324 | 2.71E−03 | 1.77E−02 |
| ABTx325 | 2.70E−03 | 1.43E−02 |
| ABTx326 | 8.25E−04 | 4.15E−03 |
| ABTx327 | 1.95E−02 | 3.29E−02 |
| ABTx328 | 7.04E−05 | 6.34E−05 |
| ABTx329 | 3.72E−04 | 4.88E−04 |
| ABTx330 | 2.31E−03 | 1.02E−02 |
| ABTx331 | 1.45E−02 | 5.57E−02 |

Binding constants for the anti-CD3 VHH antibodies were measured and are provided in Table 9. Biotinylated human CD5-HIS and biotinylated cynomolgus CD5-HIS were loaded to streptavidin (SA) Octet pins. The SA-human CD5 and the SA-Cyno CD5 bound pins were then dipped into the periplasmic extract (PE) from individual clones so that PE specific for human or cyno CD5 would bind in solution. The off-rates were then calculated by placing the sensor into Octet buffer and PE dissociation was measured versus time. Human/Cynomolgus binding ratios were calculated by dividing the K_{D}(M) of the VHH to the human CD5 with the K_{D}(M) of the VHH binding with cynomolgus CD5 protein.

TABLE 9
Binding constants for the indicated antibodies.

| | huCD5 | | | CynoCD5 | | | |
|---|---|---|---|---|---|---|---|
| Entity | K_{D} (M) | kₐ (1/Ms) | k_{dis} (1/s) | K_{D} (M) | Kₐ (1/Ms) | k_{dis} (1/s) | Hu/Cyno K_{D} |
| ABTx315 | 4.00E−10 | 1.60E+05 | 6.30E−05 | 2.20E−10 | 2.60E+05 | 5.80E−05 | 1.8 |
| ABTx316 | 1.40E−09 | 1.10E+05 | 1.50E−04 | 1.60E−09 | 1.30E+05 | 2.10E−04 | 0.9 |
| ABTx317 | 2.10E−09 | 7.10E+04 | 1.50E−04 | 2.40E−09 | 8.40E+04 | 2.00E−04 | 0.9 |
| ABTx318 | 2.50E−09 | 6.50E+04 | 1.60E−04 | 2.40E−09 | 9.10E+04 | 2.20E−04 | 1 |
| ABTx319 | 1.80E−09 | 7.20E+04 | 1.30E−04 | 2.20E−09 | 9.40E+04 | 2.00E−04 | 0.8 |
| ABTx320 | 2.80E−09 | 6.90E+04 | 1.90E−04 | 2.60E−09 | 7.60E+04 | 2.00E−04 | 1.1 |
| ABTx321 | 2.30E−10 | 2.20E+05 | 5.20E−05 | 3.70E−10 | 2.40E+05 | 8.80E−05 | 0.6 |
| ABTx322 | 1.00E−09 | 9.50E+04 | 1.00E−04 | 1.10E−09 | 9.70E+04 | 1.10E−04 | 0.9 |
| ABTx323 | 1.20E−09 | 9.30E+04 | 1.10E−04 | 1.30E−09 | 8.90E+04 | 1.20E−04 | 0.9 |
| ABTx324 | 1.70E−09 | 9.90E+04 | 1.70E−04 | 2.30E−09 | 7.10E+04 | 1.60E−04 | 0.7 |
| ABTx325 | 1.50E−09 | 9.00E+04 | 1.30E−04 | 1.80E−09 | 6.80E+04 | 1.30E−04 | 0.8 |
| ABTx326 | 2.00E−09 | 7.40E+04 | 1.50E−04 | 2.20E−09 | 6.80E+04 | 1.50E−04 | 0.9 |
| ABTx327 | 2.20E−10 | 1.60E+05 | 3.60E−05 | 7.80E−10 | 1.40E+05 | 1.10E−04 | 0.3 |
| ABTx328 | 3.60E−10 | 2.60E+05 | 9.20E−05 | 9.30E−10 | 1.90E+05 | 1.80E−04 | 0.4 |
| ABTx329 | 6.90E−10 | 1.30E+05 | 9.30E−05 | 1.60E−09 | 1.10E+05 | 1.70E−04 | 0.4 |
| ABTx330 | 6.50E−10 | 1.30E+05 | 8.30E−05 | 1.40E−09 | 1.10E+05 | 1.40E−04 | 0.5 |
| ABTx331 | 1.30E−09 | 9.40E+04 | 1.20E−04 | 2.20E−09 | 7.10E+04 | 1.60E−04 | 0.6 |

Experiments were undertaken demonstrating that the anti-CD5 VHH antibodies as Fc fusion proteins bound in a dose-dependent manner to Jurkat cells surface-expressing CD5 (FIGS. 1A and 1B). VHH-Fc were serially diluted in FACS buffer and mixed with Jurkat cells for 15 minutes. Cells were washed twice in FACS buffer and then mixed with anti-Human Fc Dylite650 diluted 1:500 and incubated for 10 minutes. Cells were washed twice in FACS buffer and then run on Attune cytometer measuring GMFI on each cell in the RL1 channel. Calculated EC50 values are provided in Tables 10 and 11.

TABLE 10
EC50 values for the indicated anti-CD5 VHH antibodies.

| | ABTx315 | ABTx316 | ABTx317 | ABTx318 | ABTx319 | ABTx326 | 5CAR scFV |
|---|---|---|---|---|---|---|---|
| EC50 | 1.123 | 0.6534 | 0.3411 | 0.3171 | 0.1160 | 1.720 | 0.7960 |

TABLE 11
EC50 values for the indicated anti-CD5 VHH antibodies.

| | ABTx320 | ABTx321 | ABTx322 | ABTx323 | ABTx324 | ABTx325 | ABTx327 | ABTx328 | ABTx329 | ABTx330 | ABTx331 |
|---|---|---|---|---|---|---|---|---|---|---|---|
| EC50 | 2.855 | 3.380 | 4.057 | 3.150 | 2.468 | 2.935 | 3.303 | 1.545 | 2.361 | 22.11 | 2.305 |

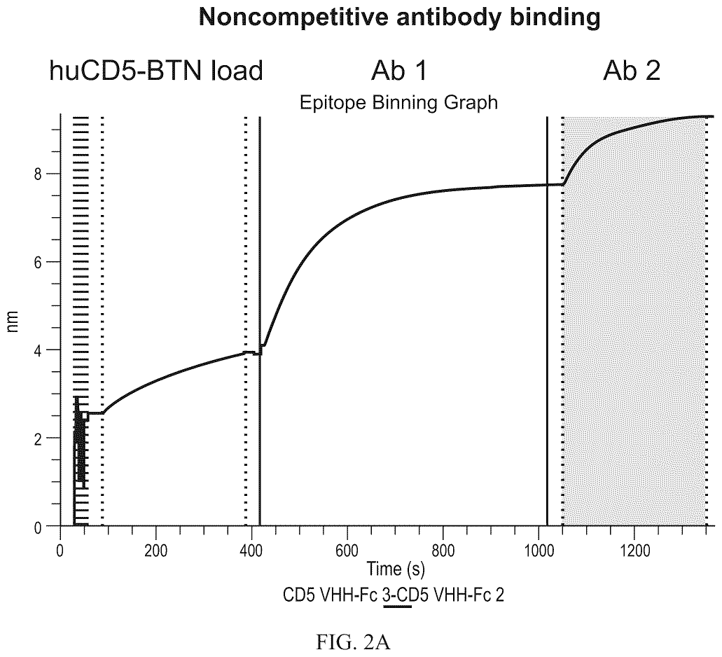

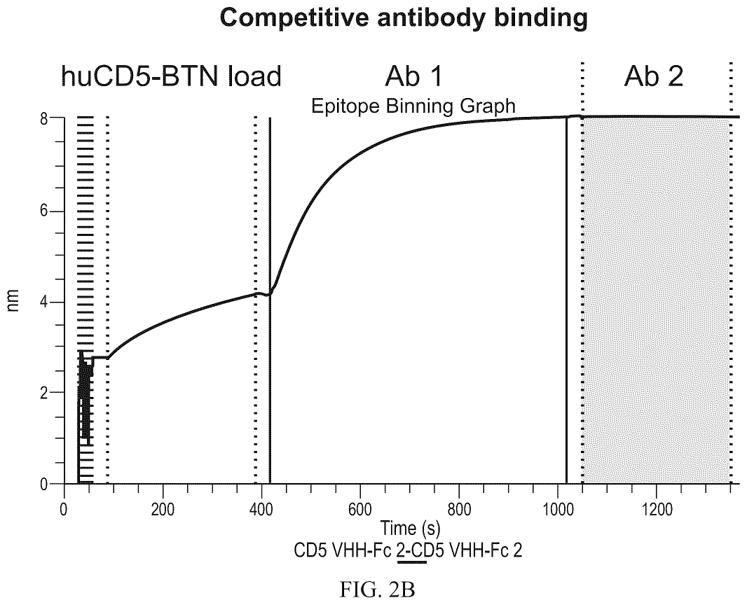

Experiments were undertaken to demonstrate that the anti-CD5 VHH antibodies bound different epitopes on CD5. As shown in FIGS. 2A and 2B, assays were conducted to determine whether or not the anti-CD5 VHH antibodies competed with the antibody UCHT2 for binding to CD5. Using a 32 well high-throughput experiment setting, 18 streptavidin biosensors were loaded with huCD5-BTN, and then all 18 biosensors were contacted in a first association step (see Ab 1 regions of FIGS. 2A and 2B) with UCHT2 antibody for an extended association time to allow binding of the UCHT2 antibody to the human CD5 antibody to reach saturation. For the second association step (see Ab 2 regions of FIGS. 2A and 2B), each biosensor was assigned to one of 17 anti-CD5 VHH-Fcs or UCHT2 to evaluate binding competition. Table 12 provides response units for the anti-CD5 VHH antibodies when competitively binding to CD5 with UCHT2. The lower the response unit, the more UCHT2 interfered with the binding of the VHH, which suggested overlapping epitopes.

TABLE 12
Response units for simultaneous binding of the indicated antibodies
to CD5 together with UCHT2 and relative magnitudes of UCHT2
competitive binding, where “−” indicates undetectable
or minimal competitive binding and increasing numbers of “+”
symbols correspond to increases in competitive binding.

| | | UCHT2 Competitive |
|---|---|---|
| Antibody Name | Response Units | Binding |
| UCHT2 | 0 | +++ |
| ABTx315 | 0.4982 | + |
| ABTx316 | 0.3942 | + |
| ABTx317 | 1.2383 | − |
| ABTx318 | 1.1547 | − |
| ABTx319 | 1.1729 | − |
| ABTx320 | 0.325 | + |
| ABTx321 | 0.0713 | +++ |
| ABTx322 | −0.1367 | +++ |
| ABTx323 | 0.3171 | + |
| ABTx324 | 0.6632 | − |
| ABTx325 | 0.3955 | + |
| ABTx326 | 0.0571 | +++ |
| ABTx327 | 0.025 | +++ |
| ABTx328 | 0.1529 | ++ |
| ABTx329 | 0.2398 | ++ |
| ABTx330 | 0.3673 | + |
| ABTx331 | 0.2321 | ++ |

### OTHER EMBODIMENTS

From the foregoing description, it will be apparent that variations and modifications may be made to the various aspects and embodiments described herein to adopt them to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The disclosure may be related to International Patent Applications No. PCT/US22/75021, PCT/US20/13964, PCT/US20/52822, PCT/US20/18178, PCT/US21/52035, PCT/US22/81241, PCT/US23/67780, PCT/US23/68543, PCT/US23/72911, PCT/US24/18668, PCT/US24/39693, and/or PCT/US2024/020699, the disclosures of which is incorporated herein by reference in their entirety for all purposes.

What is claimed:

1. A VHH antibody or an antigen binding fragment thereof that specifically binds to a cluster of differentiation 5 (CD5) polypeptide, wherein the VHH antibody comprises three Complementarity Determining Regions (CDRs): CDR1, CDR2 and CDR3, that are structurally positioned between four camelid VHH framework (FR) regions (FR1-FR4) as follows: FR1-CDR1-FR2-CDR2-FR3-CDR3-FR4; wherein
a) CDR1 is selected from the group consisting of: NYAAG (SEQ ID NO: 478), SYTMG (SEQ ID NO: 479), TYTMG (SEQ ID NO: 480), SYAMG (SEQ ID NO: 481), TYNMG (SEQ ID NO: 482), AYAMG (SEQ ID NO: 483), SSGMG (SEQ ID NO: 484), VDATT (SEQ ID NO: 485), INVIG (SEQ ID NO: 505), SSFMS (SEQ ID NO: 506), TNVMG (SEQ ID NO: 507), TNNMG (SEQ ID NO: 508), TNNMA (SEQ ID NO: 509), RVAMN (SEQ ID NO: 510), RVGMN (SEQ ID NO: 511), FVGWG (SEQ ID NO: 512), FIGWG (SEQ ID NO: 513), MYSMS (SEQ ID NO: 514), and TYGMG (SEQ ID NO: 515);
b) CDR2 is selected from the group consisting of RISRSGGRTDYADSVKG (SEQ ID NO: 486), AISWSAGRTYYADSMKG (SEQ ID NO: 487), VISWSGGRTYYADSVKG (SEQ ID NO: 488), AIDLYGRATRYANSVKG (SEQ ID NO: 489), AINLEGYATRYANSVKG (SEQ ID NO: 615), AIDLYGRATRYANSVRG (SEQ ID NO: 616), AINWNGDTALRWNGFATRYADSVKG (SEQ ID NO: 490), SMDWSGGSTYYGDSVKG (SEQ ID NO: 491), SIDWSGGSTYYGDSVKG (SEQ ID NO: 492), SIDWGGGSTYYGDSVKG (SEQ ID NO: 493), SIDWSGKSTYYGDSVKG (SEQ ID NO: 494), SINWSGGSAYYGDSVKG (SEQ ID NO: 495), SMDWTGGSTYYGDSVKG (SEQ ID NO: 496), IMDIGGVTEYADSVKG (SEQ ID NO: 497), LVNSGGQTHYADSVKG (SEQ ID NO: 516), TIYSDGSTYYADSVKG (SEQ ID NO: 517), TIYSDGSTYYADSMKG (SEQ ID NO: 518), LIRGGGSTHYADSVKG (SEQ ID NO: 519), LIRTGGSTHVADSMKG (SEQ ID NO: 520), TISSDGSRTNYAHSVKG (SEQ ID NO: 522), SISSDGSRTNYAHFVKG (SEQ ID NO: 523), QISTGGLTNYADSVKG (SEQ ID NO: 524), QINTGGLTDVYADSVKG (SEQ ID NO: 617), SISTGARDTAYADSVKG (SEQ ID NO: 526), SISTGARDTSYADSVKG (SEQ ID NO: 618), and VITGSGVGTQYADSVKD (SEQ ID NO: 527); and
c) CDR3 is selected from the group consisting of: ATVWEFTDGADQYDY (SEQ ID NO: 498), DPWTSDSDYDRLTMYDY (SEQ ID NO: 499), DPWTSDSDYERLTMYDY (SEQ ID NO: 500), DTSLPLGVLTESQRLYGA (SEQ ID NO: 501), DTSLPLGVLTKSQRMYGA (SEQ ID NO: 502), DTVVSGSYYLAARAEDYEY (SEQ ID NO: 503), GTSGVAAVNLRGFFS (SEQ ID NO: 504), RGL, RYGIDNY (SEQ ID NO: 528), VTGSI (SEQ ID NO: 529), WLGSPGAMSDY (SEQ ID NO: 530), WTGSPGALSDY (SEQ ID NO: 531), PGNS (SEQ ID NO: 532), PGHP (SEQ ID NO: 533), PGHS (SEQ ID NO: 534), GDLRYGPDGYDY (SEQ ID NO: 535), and GHRPGWAVIRADAYEY (SEQ ID NO: 536).

2. The method of claim 1, wherein:
a) CDR1 comprises the amino acid sequence NYAAG (SEQ ID NO: 478), CDR2 comprises the amino acid sequence RISRSGGRTDYADSVKG (SEQ ID NO: 486), and CDR3 comprises the amino acid sequence ATVWEFTDGADQYDY (SEQ ID NO: 498);
b) CDR1 comprises the amino acid sequence SYTMG (SEQ ID NO: 479), CDR2 comprises the amino acid sequence AISWSAGRTYYADSMKG (SEQ ID NO: 487), and CDR3 comprises the amino acid sequence DPWTSDSDYDRLTMYDY (SEQ ID NO: 499);
c) CDR1 comprises the amino acid sequence TYTMG (SEQ ID NO: 480), CDR2 comprises the amino acid sequence AISWSAGRTYYADSMKG (SEQ ID NO: 487), and CDR3 comprises the amino acid sequence DPWTSDSDYDRLTMYDY (SEQ ID NO: 499);
d) CDR1 comprises the amino acid sequence SYTMG (SEQ ID NO: 479), CDR2 comprises the amino acid sequence AISWSAGRTYYADSMKG (SEQ ID NO: 487), and CDR3 comprises the amino acid sequence DPWTSDSDYDRLTMYDY (SEQ ID NO: 499);
e) CDR1 comprises the amino acid sequence TYTMG (SEQ ID NO: 480), CDR2 comprises the amino acid sequence AISWSAGRTYYADSMKG (SEQ ID NO: 487), and CDR3 comprises the amino acid sequence DPWTSDSDYDRLTMYDY (SEQ ID NO: 499);
f) CDR1 comprises the amino acid sequence TYTMG (SEQ ID NO: 480), CDR2 comprises the amino acid sequence AISWSAGRTYYADSMKG (SEQ ID NO: 487), and CDR3 comprises the amino acid sequence DPWTSDSDYDRLTMYDY (SEQ ID NO: 499);
g) CDR1 comprises the amino acid sequence SYTMG (SEQ ID NO: 479), CDR2 comprises the amino acid sequence AISWSAGRTYYADSMKG (SEQ ID NO: 487), and CDR3 comprises the amino acid sequence DPWTSDSDYDRLTMYDY (SEQ ID NO: 499);
h) CDR1 comprises the amino acid sequence TYTMG (SEQ ID NO: 480), CDR2 comprises the amino acid sequence AISWSAGRTYYADSMKG (SEQ ID NO: 487), and CDR3 comprises the amino acid sequence DPWTSDSDYDRLTMYDY (SEQ ID NO: 499);
i) CDR1 comprises the amino acid sequence TYTMG (SEQ ID NO: 480), CDR2 comprises the amino acid sequence AISWSAGRTYYADSMKG (SEQ ID NO: 487), and CDR3 comprises the amino acid sequence DPWTSDSDYDRLTMYDY (SEQ ID NO: 499);
j) CDR1 comprises the amino acid sequence SYAMG (SEQ ID NO: 481), CDR2 comprises the amino acid sequence VISWSGGRTYYADSVKG (SEQ ID NO: 488), and CDR3 comprises the amino acid sequence DPWTSDSDYERLTMYDY (SEQ ID NO: 500);
k) CDR1 comprises the amino acid sequence SYAMG (SEQ ID NO: 481), CDR2 comprises the amino acid sequence VISWSGGRTYYADSVKG (SEQ ID NO: 488), and CDR3 comprises the amino acid sequence DPWTSDSDYERLTMYDY (SEQ ID NO: 500);
l) CDR1 comprises the amino acid sequence SYAMG, CDR2 comprises the amino acid sequence VISWSGGRTYYADSVKG (SEQ ID NO: 488), and CDR3 comprises the amino acid sequence DPWTSDSDYERLTMYDY (SEQ ID NO: 500);
m) CDR1 comprises the amino acid sequence SYAMG, CDR2 comprises the amino acid sequence VISWSGGRTYYADSVKG (SEQ ID NO: 488), and CDR3 comprises the amino acid sequence DPWTSDSDYERLTMYDY (SEQ ID NO: 500);
n) CDR1 comprises the amino acid sequence TYNMG (SEQ ID NO: 482), CDR2 comprises the amino acid sequence AIDLYGRATRYANSVKG (SEQ ID NO: 489), and CDR3 comprises the amino acid sequence DTSLPLGVLTESQRLYGA (SEQ ID NO: 501);
o) CDR1 comprises the amino acid sequence TYNMG (SEQ ID NO: 482), CDR2 comprises the amino acid sequence AINLEGYATRYANSVKG (SEQ ID NO: 615), and CDR3 comprises the amino acid sequence DTSLPLGVLTESQRLYGA (SEQ ID NO: 501);
p) CDR1 comprises the amino acid sequence TYNMG (SEQ ID NO: 482), CDR2 comprises the amino acid sequence AIDLYGRATRYANSVKG (SEQ ID NO: 489), and CDR3 comprises the amino acid sequence DTSLPLGVLTESQRLYGA (SEQ ID NO: 501);
q) CDR1 comprises the amino acid sequence TYNMG (SEQ ID NO: 482), CDR2 comprises the amino acid sequence AIDLYGRATRYANSVRG (SEQ ID NO: 616), and CDR3 comprises the amino acid sequence DTSLPLGVLTESQRLYGA (SEQ ID NO: 501);
r) CDR1 comprises the amino acid sequence TYNMG (SEQ ID NO: 482), CDR2 comprises the amino acid sequence AIDLYGRATRYANSVKG (SEQ ID NO: 489), and CDR3 comprises the amino acid sequence DTSLPLGVLTESQRLYGA (SEQ ID NO: 501);
s) CDR1 comprises the amino acid sequence TYNMG (SEQ ID NO: 482), CDR2 comprises the amino acid sequence AIDLYGRATRYANSVKG (SEQ ID NO: 489), and CDR3 comprises the amino acid sequence DTSLPLGVLTKSQRMYGA (SEQ ID NO: 502);
t) CDR1 comprises the amino acid sequence AYAMG, CDR2 comprises the amino acid sequence AINWNGDTALRWNGFATRYADSVKG (SEQ ID NO: 490), and CDR3 comprises the amino acid sequence DTVVSGSYYLAARAEDYEY (SEQ ID NO: 503);
u) CDR1 comprises the amino acid sequence AYAMG, CDR2 comprises the amino acid sequence AINWNGDTALRWNGFATRYADSVKG (SEQ ID NO: 490), and CDR3 comprises the amino acid sequence DTVVSGSYYLAARAEDYEY (SEQ ID NO: 503);
v) CDR1 comprises the amino acid sequence AYAMG, CDR2 comprises the amino acid sequence AINWNGDTALRWNGFATRYADSVKG (SEQ ID NO: 490), and CDR3 comprises the amino acid sequence DTVVSGSYYLAARAEDYEY (SEQ ID NO: 503);
w) CDR1 comprises the amino acid sequence AYAMG, CDR2 comprises the amino acid sequence AINWNGDTALRWNGFATRYADSVKG (SEQ ID NO: 490), and CDR3 comprises the amino acid sequence DTVVSGSYYLAARAEDYEY (SEQ ID NO: 503);
x) CDR1 comprises the amino acid sequence SSGMG (SEQ ID NO: 484), CDR2 comprises the amino acid sequence SMDWSGGSTYYGDSVKG (SEQ ID NO: 491), and CDR3 comprises the amino acid sequence GTSGVAAVNLRGFFS (SEQ ID NO: 504);
y) CDR1 comprises the amino acid sequence SSGMG (SEQ ID NO: 484), CDR2 comprises the amino acid sequence SIDWSGGSTYYGDSVKG (SEQ ID NO: 492), and CDR3 comprises the amino acid sequence GTSGVAAVNLRGFFS (SEQ ID NO: 504);
z) CDR1 comprises the amino acid sequence SSGMG (SEQ ID NO: 484), CDR2 comprises the amino acid sequence SMDWSGGSTYYGDSVKG (SEQ ID NO: 491), and CDR3 comprises the amino acid sequence GTSGVAAVNLRGFFS (SEQ ID NO: 504);
aa) CDR1 comprises the amino acid sequence SSGMG (SEQ ID NO: 484), CDR2 comprises the amino acid sequence SIDWGGGSTYYGDSVKG (SEQ ID NO: 493), and CDR3 comprises the amino acid sequence GTSGVAAVNLRGFFS (SEQ ID NO: 504);
ab) CDR1 comprises the amino acid sequence SSGMG (SEQ ID NO: 484), CDR2 comprises the amino acid sequence SIDWSGGSTYYGDSVKG (SEQ ID NO: 492), and CDR3 comprises the amino acid sequence GTSGVAAVNLRGFFS (SEQ ID NO: 504);
ac) CDR1 comprises the amino acid sequence SSGMG (SEQ ID NO: 484), CDR2 comprises the amino acid sequence SIDWSGKSTYYGDSVKG (SEQ ID NO: 494), and CDR3 comprises the amino acid sequence GTSGVAAVNLRGFFS (SEQ ID NO: 504);
ad) CDR1 comprises the amino acid sequence SSGMG (SEQ ID NO: 484), CDR2 comprises the amino acid sequence SINWSGGSAYYGDSVKG (SEQ ID NO: 495), and CDR3 comprises the amino acid sequence GTSGVAAVNLRGFFS (SEQ ID NO: 504);
ae) CDR1 comprises the amino acid sequence SSGMG (SEQ ID NO: 484), CDR2 comprises the amino acid sequence SMDWSGGSTYYGDSVKG (SEQ ID NO: 491), and CDR3 comprises the amino acid sequence GTSGVAAVNLRGFFS (SEQ ID NO: 504);
af) CDR1 comprises the amino acid sequence SSGMG (SEQ ID NO: 484), CDR2 comprises the amino acid sequence SMDWTGGSTYYGDSVKG (SEQ ID NO: 496), and CDR3 comprises the amino acid sequence GTSGVAAVNLRGFFS (SEQ ID NO: 504);
ag) CDR1 comprises the amino acid sequence VDATT (SEQ ID NO: 485), CDR2 comprises the amino acid sequence IMDIGGVTEYADSVKG (SEQ ID NO): 497), and CDR3 comprises the amino acid sequence RGL;
ah) CDR1 comprises the amino acid sequence INVIG (SEQ ID NO: 505), CDR2 comprises the amino acid sequence LVNSGGQTHYADSVKG (SEQ ID NO: 516), and CDR3 comprises the amino acid sequence RYGIDNY (SEQ ID NO: 528);
ai) CDR1 comprises the amino acid sequence SSFMS (SEQ ID NO: 506), CDR2 comprises the amino acid sequence TIYSDGSTYYADSVKG (SEQ ID NO: 517), and CDR3 comprises the amino acid sequence VTGSI (SEQ ID NO: 529);
aj) CDR1 comprises the amino acid sequence SSFMS (SEQ ID NO: 506), CDR2 comprises the amino acid sequence TIYSDGSTYYADSMKG (SEQ ID NO: 518), and CDR3 comprises the amino acid sequence VTGSI (SEQ ID NO: 529);
ak) CDR1 comprises the amino acid sequence TNVMG (SEQ ID NO: 507), CDR2 comprises the amino acid sequence LIRGGGSTHYADSVKG (SEQ ID NO: 519), and CDR3 comprises the amino acid sequence WLGSPGAMSDY (SEQ ID NO: 530);
al) CDR1 comprises the amino acid sequence TNNMG (SEQ ID NO: 508), CDR2 comprises the amino acid sequence LIRTGGSTHVADSMKG (SEQ ID NO: 520), and CDR3 comprises the amino acid sequence WTGSPGALSDY (SEQ ID NO: 531);
am) CDR1 comprises the amino acid sequence TNNMA (SEQ ID NO: 509), CDR2 comprises the amino acid sequence LIRTGGSTHVADSMKG (SEQ ID NO: 520), and CDR3 comprises the amino acid sequence WTGSPGALSDY (SEQ ID NO: 531);
an) CDR1 comprises the amino acid sequence RVAMN (SEQ ID NO: 510), CDR2 comprises the amino acid sequence TISSDGSRTNYAHSVKG (SEQ ID NO: 522), and CDR3 comprises the amino acid sequence PGNS (SEQ ID NO: 532);
ao) CDR1 comprises the amino acid sequence RVGMN (SEQ ID NO: 511), CDR2 comprises the amino acid sequence SISSDGSRTNYAHFVKG (SEQ ID NO: 523), and CDR3 comprises the amino acid sequence PGNS (SEQ ID NO: 532);
ap) CDR1 comprises the amino acid sequence FVGWG (SEQ ID NO: 512), CDR2 comprises the amino acid sequence QISTGGLTNYADSVKG (SEQ ID NO: 524), and CDR3 comprises the amino acid sequence PGHP (SEQ ID NO: 533);
aq) CDR1 comprises the amino acid sequence FIGWG (SEQ ID NO: 513), CDR2 comprises the amino acid sequence QINTGGLTDYADSVKG (SEQ ID NO: 525), and CDR3 comprises the amino acid sequence PGHS (SEQ ID NO: 534);
ar) CDR1 comprises the amino acid sequence MYSMS (SEQ ID NO: 514), CDR2 comprises the amino acid sequence SISTGARDTAYADSVKG (SEQ ID NO: 526), and CDR3 comprises the amino acid sequence GDLRYGPDGYDY (SEQ ID NO: 535);
as) CDR1 comprises the amino acid sequence MYSMS (SEQ ID NO: 514), CDR2 comprises the amino acid sequence SISTGARDTAYADSVKG (SEQ ID NO: 526), and CDR3 comprises the amino acid sequence GDLRYGPDGYDY (SEQ ID NO: 535);
at) CDR1 comprises the amino acid sequence MYSMS (SEQ ID NO: 514), CDR2 comprises the amino acid sequence SISTGARDTSYADSVKG (SEQ ID NO: 618), and CDR3 comprises the amino acid sequence GDLRYGPDGYDY (SEQ ID NO: 535); or
au) CDR1 comprises the amino acid sequence TYGMG (SEQ ID NO: 515), CDR2 comprises the amino acid sequence VITGSGVGTQYADSVKD (SEQ ID NO: 527), and CDR3 comprises the amino acid sequence GHRPGWAVIRADAYEY (SEQ ID NO: 536).

3. The VHH antibody of claim 1, wherein:
a) FR1 comprises the following amino acid sequence:
X₁X₂QLX₃ESGGX₄VQX₅GX₆SX₇RLX₈CX₉X₁₀SGX₁₁X₁₂X₁₃X₁₄ (SEQ ID NO: 604), wherein
X₁ is E or Q;
X₂ is L or V;
X₃ is V or Q;
X₄ is L or S;
X₅ is P or A;
X₆ is A, E, or G;
X₇ is L, R, or V;
X₈ is A or S;
X₉ is A or V;
X₁₀ is A, T, or V;
X₁₁ is A, D, F, G, I, P, R, or S;
X₁₂ is A, D, I, N, P, S, T, or V;
X₁₃ is A, F, null, S, or V; and
X₁₄ is I, L, null, or S;
b) FR2 comprises the amino acid sequence:
WX₁₅RX₁₆APGX₁₇X₁₈X₁₉X₂₀X₂₁VX₂₂ (SEQ ID NO: 605), wherein
X₁₅ is F, V, or Y;
X₁₆ is H or Q;
X₁₇ is E or K;
X₁₈ A, D, E, G, R, or Q;
X₁₉ is L or R;
X₂₀ is D or E;
X₂₁ is F, L, V, or W; and
X₂₂ is A or S;
c) FR3 comprises the amino acid sequence:
RFX₂₃X₂₄SRX₂₅X₂₆X₂₇X₂₈X₂₉X₃₀X₃₁X₃₂LX₃₃MX₃₄X₃₅LX₃₆X₃₇EDTAX₃₈YYCX₃₉X₄₀ (SEQ ID NO: 606), wherein
X₂₃ is A, I, or T;
X₂₄ is I or V;
X₂₅ is D, E, or V;
X₂₆ is H, I, or N;
X₂₇ is A or T;
X₂₈ is D or K;
X₂₉ is K, M, N, R, S, or T;
X₃₀ is A, M, or T;
X₃₁ is A, L, or V;
X₃₂ is F, H, N, or Y;
X₃₃ is H or Q;
X₃₄ is N or S;
X₃₅ is G, N, S, or T;
X₃₆ is K or R;
X₃₇ is A, F, L, P, or V;
X₃₈ is V or E;
X₃₉ is A, H, N, or V; and
X₄₀ is A, E, F, G, I, N, R, T, or V; and/or
d) FR4 comprises the amino acid sequence:
X₄₀GX₄₁GTX₄₂VX₄₃VX₄₄S (SEQ ID NO: 607), wherein
X₄₀ is R or W;
X₄₁ is Q, E, or P;
X₄₂ is L or Q;
X₄₃ is S or T; and
X₄₄ is S or V.

4. The VHH antibody of claim 1, wherein:
a) FR1 comprises an amino acid sequence selected from the group consisting of:
(SEQ ID NO: 537)
QVQLVESGGGLVQPGGSLRLSCAASGRTFI,
(SEQ ID NO: 538)
EVQLVESGGGLVQAGGSLRLSCAASGRTFG,
(SEQ ID NO: 543)
QVQLQESGGGLVQAGGSLRLSCAASGRTFG,
(SEQ ID NO: 619)
QVQLVESGGGLVQAGGSLRLSCAASGRTFG,
(SEQ ID NO: 544)
EVQLVESGGGLVQAGGSLRLSCAASGGTVS,
(SEQ ID NO: 545)
EVQLVESGGGLVQAGGSRRLSCAASGGTVS,
(SEQ ID NO: 546)
QVQLVESGGGLVQAGGSLRLSCAASGGTVS,
(SEQ ID NO: 548)
EVQLVESGGGLVQAGASLRLSCAASGRT,
(SEQ ID NO: 549)
QVQLQESGGGLVQAGASLRLSCAASGRA,
(SEQ ID NO: 550)
QVQLQESGGGLVQAGASLRLSCAASGRT,
(SEQ ID NO: 551)
QVQLVESGGGLVQAGASLRLSCAASGRT,
(SEQ ID NO: 554)
QVQLQESGGGSVQAGGSLRLSCAASGRAFS,
(SEQ ID NO: 559)
EVQLVESGGGLVQAGGSLRLSCAASGPAFS,
(SEQ ID NO: 560)
QVQLQESGGGLVQAGGSLRLSCAASGPAFS,
(SEQ ID NO: 561)
QVQLVESGGGLVQAGGSLRLACAASGAAFS,
(SEQ ID NO: 562)
QVQLVESGGGLVQAGGSLRLSCAASGPAFS,
(SEQ ID NO: 569)
QLQLVESGGGLVQPGGSLRLSCAASGSDFL,
(SEQ ID NO: 573)
QVQLQESGGGLVQAGGSLRLSCATSGITSS,
(SEQ ID NO: 577)
EVQLVESGGGLVQPGGSLRLSCAASGFPFS,
(SEQ ID NO: 578)
QVQLVESGGGLVQPGGSLRLSCAASGFNFS,
(SEQ ID NO: 584)
QVQLVESGGGLVQPGGSVRLSCATSGSIFS,
(SEQ ID NO: 587)
EVQLVESGGGLVQPGGSLRLSCAASGSVVS,
(SEQ ID NO: 588)
QVQLVESGGGLVQPGGSLRLSCAASGSDAS,
(SEQ ID NO: 590)
QLQLVESGGGLVQPGESLRLSCAASGFSFS,
(SEQ ID NO: 594)
QLQLVESGGGLVQPGESLRLSCVVSGDIFS,
(SEQ ID NO: 597)
QVQLVESGGGLVQPGESLRLSCVVSGDIFS,
(SEQ ID NO: 599)
QVQLVESGGGLVQPGGSLRLSCAASGFTFS,
and
(SEQ ID NO: 602)
QVQLVESGGGLVQPGGSLRLSCVASGGTFS;
b) FR2 comprises an amino acid sequence selected from the group consisting of:
```
  (SEQ ID NO: 539)
WFRQAPGKEREFVA,
  (SEQ ID NO: 620)
WFRQAPGKGREFVA,
  (SEQ ID NO: 621)
WFRQAPGREREFVA,
(SEQ ID NO: 552)
WFRHAPGKDREFVA,
  (SEQ ID NO: 553)
WFRHAPGEDREFVA,
  (SEQ ID NO: 563)
WFRQAPGKARDFVA,
  (SEQ ID NO: 567)
WFRQAPGKAREFVA,
  (SEQ ID NO: 570)
WFRQAPGNQREFVA,
  (SEQ ID NO: 574)
WYRQAPGKQRELVA,
  (SEQ ID NO: 579)
WVRQAPGKGLEWVS,
  (SEQ ID NO: 580)
WVRQAPGKEVEWVS,
  (SEQ ID NO: 585)
WYRQAPGKEREFVA,
  (SEQ ID NO: 591)
WYRQAPGKERELVA,
  (SEQ ID NO: 595)
WYRQAPGKQREVVA,
and
  (SEQ ID NO: 600)
WVRQAPGKRLEWVS;
```
c) FR3 comprises an amino acid sequence selected from the group consisting of:
```
(SEQ ID NO: 540)
RFTISRDNAKSTVYLQMNSLRPEDTAVYYCAE,
(SEQ ID NO: 541)
RFTISRDNAKNTVYLQMNSLKPEDTAVYYCAA,
(SEQ ID NO: 547)
RFTISRDNAKNTVNLQMNSLKPEDTAVYYCAA,
(SEQ ID NO: 555)
RFIISRVNAKNTVNLQMNSLKPEDTAVYYCAA,
(SEQ ID NO: 556)
RFTISRVIAKNTVNLQMNSLKPEDTAVYYCAA,
(SEQ ID NO: 558)
RFTISRVNAKNTVNLQMNSLKPEDTAVYYCAA,
(SEQ ID NO: 564)
RFTVSRDNAKNTVHLQMNSLRPEDTAVYYCAR,
(SEQ ID NO: 565)
RFTVSRDNAKNAVHLQMNSLRPEDTAVYYCAR,
(SEQ ID NO: 568)
RFTVSRDNAKMTVHLQMNSLRPEDTAVYYCAR,
(SEQ ID NO: 571)
RFTISRDHTKNTVYLQMNSLKVEDTAVYYCNT,
(SEQ ID NO: 575)
RFTISRDNAKNTVFLQMNSLKPEDTAEYYCHG,
(SEQ ID NO: 581)
RFTISRDNAKKTAYLQMNSLKAEDTAVYYCAT,
(SEQ ID NO: 582)
RFTISRDNAKNTVYLQMSNLKAEDTAVYYCAT,
(SEQ ID NO: 586)
RFIISRENAKTTVYLQMNGLKPEDTAVYYCVI,
(SEQ ID NO: 589)
RFTISRENAKNTVYLQMNSLKPEDTAVYYCVI,
(SEQ ID NO: 592)
RFTISRENAKNMVYLQMNSLKLEDTAVYYCNV,
(SEQ ID NO: 593)
RFTISRDNVKNMVYLQMNSLKLEDTAVYYCNV,
(SEQ ID NO: 596)
RFAISRDNAKRTVYLQMNSLKFEDTAVYYCNV,
(SEQ ID NO: 598)
RFTISRDNAKRTVYLQMNSLKFEDTAVYYCNF,
  (SEQ ID NO: 601)
RFTISRDNADNTLYLHMNNLKPEDTAVYYCAN,
and
  (SEQ ID NO: 603)
RFTISRENAKNTVYLQMNTLKLEDTAVYYCVS;
```
and/or
d) FR4 comprises an amino acid sequence selected from the group consisting of:
```
  (SEQ ID NO: 542)
WGQGTQVTVSS,
  (SEQ ID NO: 557)
WGOGTQVSVSS,
(SEQ ID NO: 566)
WGPGTQVTVSS,
  (SEQ ID NO: 572)
WGQGTLVTVSS,
  (SEQ ID NO: 576)
WGEGTQVTVSS,
  (SEQ ID NO: 583)
RGQGTQVTVSS,
and
  (SEQ ID NO: 622)
RGQGTQVTVVS.
```

5. The VHH antibody of claim 1, wherein:
a) FR1 comprises the amino acid sequence QVQLVESGGGLVQPGGSLRLSCAASGRTFI (SEQ ID NO: 537), FR2 comprises the amino acid sequence WFRQAPGKEREFVA (SEQ ID NO: 539), FR3 comprises the amino acid sequence RFTISRDNAKSTVYLQMNSLRPEDTAVYYCAE (SEQ ID NO: 540), and FR4 comprises the amino acid sequence WGQGTQVTVSS (SEQ ID NO: 542);
b) FR1 comprises the amino acid sequence EVOLVESGGGLVQAGGSLRLSCAASGRTFG (SEQ ID NO: 538), FR2 comprises the amino acid sequence WFRQAPGKEREFVA (SEQ ID NO: 539), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
c) FR1 comprises the amino acid sequence EVOLVESGGGLVQAGGSLRLSCAASGRTFG (SEQ ID NO: 538), FR2 comprises the amino acid sequence WFRQAPGKEREFVA (SEQ ID NO: 539), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
d) FR1 comprises the amino acid sequence QVQLQESGGGLVQAGGSLRLSCAASGRTFG (SEQ ID NO: 543), FR2 comprises the amino acid sequence WFRQAPGKEREFVA (SEQ ID NO: 539), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
e) FR1 comprises the amino acid sequence QVQLQESGGGLVQAGGSLRLSCAASGRTFG (SEQ ID NO: 543), FR2 comprises the amino acid sequence WFRQAPGKEREFVA (SEQ ID NO: 539), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
f) FR1 comprises the amino acid sequence QVQLQESGGGLVQAGGSLRLSCAASGRTFG (SEQ ID NO: 543), FR2 comprises the amino acid sequence WFRQAPGKGREFVA (SEQ ID NO: 620), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
g) FR1 comprises the amino acid sequence QVQLVESGGGLVQAGGSLRLSCAASGRTFG (SEQ ID NO: 619), FR2 comprises the amino acid sequence WFRQAPGKEREFVA (SEQ ID NO: 539), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
h) FR1 comprises the amino acid sequence QVQLVESGGGLVQAGGSLRLSCAASGRTFG (SEQ ID NO: 619), FR2 comprises the amino acid sequence WFRQAPGKEREFVA (SEQ ID NO: 539), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
i) FR1 comprises the amino acid sequence QVQLVESGGGLVQAGGSLRLSCAASGRTFG (SEQ ID NO: 619), FR2 comprises the amino acid sequence WFRQAPGREREFVA (SEQ ID NO: 621), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
j) FR1 comprises the amino acid sequence EVOLVESGGGLVQAGGSLRLSCAASGGTVS (SEQ ID NO: 544), FR2 comprises the amino acid sequence WFRQAPGKEREFVA (SEQ ID NO: 539), FR3 comprises the amino acid sequence RFTISRDNAKNTVNLQMNSLKPEDTAVYYCAA (SEQ ID NO: 547), and FR4 comprises the amino acid sequence WGQGTQVTVSS (SEQ ID NO: 542);
k) FR1 comprises the amino acid sequence EVOLVESGGGLVQAGGSRRLSCAASGGTVS (SEQ ID NO: 545), FR2 comprises the amino acid sequence WFRQAPGKEREFVA (SEQ ID NO: 539), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
l) FR1 comprises the amino acid sequence QVQLVESGGGLVQAGGSLRLSCAASGGTVS (SEQ ID NO: 546), FR2 comprises the amino acid sequence WFRQAPGKEREFVA (SEQ ID NO: 539), FR3 comprises the amino acid sequence RFTISRDNAKNTVNLQMNSLKPEDTAVYYCAA (SEQ ID NO: 547), and FR4 comprises the amino acid sequence WGQGTQVTVSS (SEQ ID NO: 542);
m) FR1 comprises the amino acid sequence QVQLVESGGGLVQAGGSLRLSCAASGGTVS (SEQ ID NO: 546), FR2 comprises the amino acid sequence WFRQAPGKEREFVA (SEQ ID NO: 539), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
n) FR1 comprises the amino acid sequence EVOLVESGGGLVQAGASLRLSCAASGRT (SEQ ID NO: 548), FR2 comprises the amino acid sequence WFRHAPGKDREFVA (SEQ ID NO: 552), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
o) FR1 comprises the amino acid sequence QVQLQESGGGLVQAGASLRLSCAASGRA (SEQ ID NO: 549), FR2 comprises the amino acid sequence WFRHAPGEDREFVA (SEQ ID NO: 553), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
p) FR1 comprises the amino acid sequence QVQLQESGGGLVQAGASLRLSCAASGRT (SEQ ID NO: 550), FR2 comprises the amino acid sequence WFRHAPGKDREFVA (SEQ ID NO: 552), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
q) FR1 comprises the amino acid sequence QVQLQESGGGLVQAGASLRLSCAASGRT (SEQ ID NO: 550), FR2 comprises the amino acid sequence WFRHAPGKDREFVA (SEQ ID NO: 552), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
r) FR1 comprises the amino acid sequence QVQLVESGGGLVQAGASLRLSCAASGRT (SEQ ID NO: 551), FR2 comprises the amino acid sequence WFRHAPGKDREFVA (SEQ ID NO: 552), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
s) FR1 comprises the amino acid sequence QVQLVESGGGLVQAGASLRLSCAASGRT (SEQ ID NO: 551), FR2 comprises the amino acid sequence WFRHAPGKDREFVA (SEQ ID NO: 552), FR3 comprises the amino acid sequence RFTISRDNAKNTVYLQMNSLKPEDTAVYYCAA (SEQ ID NO: 541), and FR4 comprises the amino acid sequence WGQGTQVTVSS (SEQ ID NO: 542);
t) FR1 comprises the amino acid sequence QVQLQESGGGSVQAGGSLRLSCAASGRAFS (SEQ ID NO: 554), FR2 comprises the amino acid sequence WFRQAPGKEREFVA (SEQ ID NO: 539), FR3 comprises the amino acid sequence RFIISRVNAKNTVNLQMNSLKPEDTAVYYCAA (SEQ ID NO: 555), and FR4 comprises the amino acid sequence WGQGTQVSVSS (SEQ ID NO: 557);
u) FR1 comprises the amino acid sequence QVQLQESGGGSVQAGGSLRLSCAASGRAFS (SEQ ID NO: 554), FR2 comprises the amino acid sequence WFRQAPGKEREFVA (SEQ ID NO: 539), FR3 comprises the amino acid sequence RFTISRVIAKNTVNLQMNSLKPEDTAVYYCAA (SEQ ID NO: 556), and FR4 comprises the amino acid sequence WGQGTQVTVSS (SEQ ID NO: 542);
v) FR1 comprises the amino acid sequence QVQLQESGGGSVQAGGSLRLSCAASGRAFS (SEQ ID NO: 554), FR2 comprises the amino acid sequence WFRQAPGKEREFVA (SEQ ID NO: 539), FR3 comprises the amino acid sequence RFTISRVNAKNTVNLQMNSLKPEDTAVYYCAA (SEQ ID NO: 558), and FR4 comprises the amino acid sequence WGQGTQVSVSS (SEQ ID NO: 557);
w) FR1 comprises the amino acid sequence QVQLQESGGGSVQAGGSLRLSCAASGRAFS (SEQ ID NO: 554), FR2 comprises the amino acid sequence WFRQAPGKEREFVA (SEQ ID NO: 539), FR3 comprises the amino acid sequence RFTISRVNAKNTVNLQMNSLKPEDTAVYYCAA (SEQ ID NO: 558), and FR4 comprises the amino acid sequence WGQGTQVTVSS (SEQ ID NO: 542);
x) FR1 comprises the amino acid sequence EVOLVESGGGLVQAGGSLRLSCAASGPAFS (SEQ ID NO: 559), FR2 comprises the amino acid sequence WFRQAPGKARDFVA (SEQ ID NO: 563), FR3 comprises the amino acid sequence RFTVSRDNAKNTVHLQMNSLRPEDTAVYYCAR (SEQ ID NO: 564), and FR4 comprises the amino acid sequence WGPGTQVTVSS (SEQ ID NO: 566);
y) FR1 comprises the amino acid sequence QVQLQESGGGLVQAGGSLRLSCAASGPAFS (SEQ ID NO: 560), FR2 comprises the amino acid sequence WFRQAPGKARDFVA (SEQ ID NO: 563), FR3 comprises the amino acid sequence RFTVSRDNAKNTVHLQMNSLRPEDTAVYYCAR (SEQ ID NO: 564), and FR4 comprises the amino acid sequence WGPGTQVTVSS (SEQ ID NO: 566);
z) FR1 comprises the amino acid sequence QVQLVESGGGLVQAGGSLRLACAASGAAFS (SEQ ID NO: 561), FR2 comprises the amino acid sequence WFRQAPGKARDEVA (SEQ ID NO: 563), FR3 comprises the amino acid sequence RFTVSRDNAKNTVHLQMNSLRPEDTAVYYCAR (SEQ ID NO: 564), and FR4 comprises the amino acid sequence WGPGTQVTVSS (SEQ ID NO: 566);
aa) FR1 comprises the amino acid sequence QVQLVESGGGLVQAGGSLRLSCAASGPAFS (SEQ ID NO: 562), FR2 comprises the amino acid sequence WFRQAPGKARDFVA (SEQ ID NO: 563), FR3 comprises the amino acid sequence RFTVSRDNAKNAVHLQMNSLRPEDTAVYYCAR (SEQ ID NO: 565), and FR4 comprises the amino acid sequence WGPGTQVTVSS (SEQ ID NO: 566);
ab) FR1 comprises the amino acid sequence QVQLVESGGGLVQAGGSLRLSCAASGPAFS (SEQ ID NO: 562), FR2 comprises the amino acid sequence WFRQAPGKARDFVA (SEQ ID NO: 563), FR3 comprises the amino acid sequence RFTVSRDNAKNTVHLQMNSLRPEDTAVYYCAR (SEQ ID NO: 564), and FR4 comprises the amino acid sequence WGPGTQVTVSS (SEQ ID NO: 566);
ac) FR1 comprises the amino acid sequence QVQLVESGGGLVQAGGSLRLSCAASGPAFS (SEQ ID NO: 562), FR2 comprises the amino acid sequence WFRQAPGKARDFVA (SEQ ID NO: 563), FR3 comprises the amino acid sequence RFTVSRDNAKNTVHLQMNSLRPEDTAVYYCAR (SEQ ID NO: 564), and FR4 comprises the amino acid sequence WGPGTQVTVSS (SEQ ID NO: 566);
ad) FR1 comprises the amino acid sequence QVQLVESGGGLVQAGGSLRLSCAASGPAFS (SEQ ID NO: 562), FR2 comprises the amino acid sequence WFRQAPGKARDFVA (SEQ ID NO: 563), FR3 comprises the amino acid sequence RFTVSRDNAKNTVHLQMNSLRPEDTAVYYCAR (SEQ ID NO: 564), and FR4 comprises the amino acid sequence WGPGTQVTVSS (SEQ ID NO: 566);
ae) FR1 comprises the amino acid sequence QVQLVESGGGLVQAGGSLRLSCAASGPAFS (SEQ ID NO: 562), FR2 comprises the amino acid sequence WFRQAPGKARDEVA (SEQ ID NO: 563), FR3 comprises the amino acid sequence RFTVSRDNAKNTVHLQMNSLRPEDTAVYYCAR (SEQ ID NO: 564), and FR4 comprises the amino acid sequence WGPGTQVTVSS (SEQ ID NO: 566);
af) FR1 comprises the amino acid sequence QVQLVESGGGLVQAGGSLRLSCAASGPAFS (SEQ ID NO: 562), FR2 comprises the amino acid sequence WFRQAPGKAREFVA (SEQ ID NO: 567), FR3 comprises the amino acid sequence RFTVSRDNAKMTVHLQMNSLRPEDTAVYYCAR (SEQ ID NO: 568), and FR4 comprises the amino acid sequence WGPGTQVTVSS (SEQ ID NO: 566);
ag) FR1 comprises the amino acid sequence QLQLVESGGGLVQPGGSLRLSCAASGSDFL (SEQ ID NO: 569), FR2 comprises the amino acid sequence WFRQAPGNQREFVA (SEQ ID NO: 570), FR3 comprises the amino acid sequence RFTISRDHTKNTVYLQMNSLKVEDTAVYYCNT (SEQ ID NO: 571), and FR4 comprises the amino acid sequence WGQGTLVTVSS (SEQ ID NO: 572);
ah) FR1 comprises the amino acid sequence QVQLQESGGGLVQAGGSLRLSCATSGITSS (SEQ ID NO: 573), FR2 comprises the amino acid sequence WYRQAPGKQRELVA (SEQ ID NO: 574), FR3 comprises the amino acid sequence RFTISRDNAKNTVFLQMNSLKPEDTAEYYCHG (SEQ ID NO: 575), and FR4 comprises the amino acid sequence WGEGTQVTVSS (SEQ ID NO: 576);
ai) FR1 comprises the amino acid sequence EVQLVESGGGLVQPGGSLRLSCAASGFPFS (SEQ ID NO: 577), FR2 comprises the amino acid sequence WVRQAPGKGLEWVS (SEQ ID NO: 579), FR3 comprises the amino acid sequence RFTISRDNAKKTAYLQMNSLKAEDTAVYYCAT (SEQ ID NO: 581), and FR4 comprises the amino acid sequence RGQGTQVTVSS (SEQ ID NO: 583);
aj) FR1 comprises the amino acid sequence QVQLVESGGGLVQPGGSLRLSCAASGENES (SEQ ID NO: 578), FR2 comprises the amino acid sequence WVRQAPGKEVEWVS (SEQ ID NO: 580), FR3 comprises the amino acid sequence RFTISRDNAKNTVYLQMSNLKAEDTAVYYCAT (SEQ ID NO: 582), and FR4 comprises the amino acid sequence RGQGTQVTVSS (SEQ ID NO: 583);
ak) FR1 comprises the amino acid sequence QVQLVESGGGLVQPGGSVRLSCATSGSIFS (SEQ ID NO: 584), FR2 comprises the amino acid sequence WYRQAPGKEREFVA (SEQ ID NO: 585), FR3 comprises the amino acid sequence RFIISRENAKTTVYLQMNGLKPEDTAVYYCVI (SEQ ID NO: 586), and FR4 comprises the amino acid sequence WGQGTQVTVSS (SEQ ID NO: 542);
al) FR1 comprises the amino acid sequence EVOLVESGGGLVQPGGSLRLSCAASGSVVS (SEQ ID NO: 587), FR2 comprises the amino acid sequence WYRQAPGKEREFVA (SEQ ID NO: 585), FR3 comprises the amino acid sequence RFTISRENAKNTVYLQMNSLKPEDTAVYYCVI (SEQ ID NO: 589), and FR4 comprises the amino acid sequence WGQGTQVTVSS (SEQ ID NO: 542);
am) FR1 comprises the amino acid sequence QVQLVESGGGLVQPGGSLRLSCAASGSDAS (SEQ ID NO: 588), FR2 comprises the amino acid sequence WYRQAPGKEREFVA (SEQ ID NO: 585), FR3 comprises the amino acid sequence RFTISRENAKNTVYLQMNSLKPEDTAVYYCVI (SEQ ID NO: 589), and FR4 comprises the amino acid sequence WGQGTQVTVSS (SEQ ID NO: 542);
an) FR1 comprises the amino acid sequence QLQLVESGGGLVQPGESLRLSCAASGFSFS (SEQ ID NO: 590), FR2 comprises the amino acid sequence WYRQAPGKERELVA (SEQ ID NO: 591), FR3 comprises the amino acid sequence RFTISRENAKNMVYLQMNSLKLEDTAVYYCNV (SEQ ID NO: 592), and FR4 comprises the amino acid sequence WGQGTQVTVSS (SEQ ID NO: 542);
ao) FR1 comprises the amino acid sequence QLQLVESGGGLVQPGESLRLSCAASGFSFS (SEQ ID NO: 590), FR2 comprises the amino acid sequence WYRQAPGKERELVA (SEQ ID NO: 591), FR3 comprises the amino acid sequence RFTISRDNVKNMVYLQMNSLKLEDTAVYYCNV (SEQ ID NO: 593), and FR4 comprises the amino acid sequence WGQGTQVTVSS (SEQ ID NO: 542);
ap) FR1 comprises the amino acid sequence QLQLVESGGGLVQPGESLRLSCVVSGDIFS (SEQ ID NO: 594), FR2 comprises the amino acid sequence WYRQAPGKQREVVA (SEQ ID NO: 595), FR3 comprises the amino acid sequence RFAISRDNAKRTVYLQMNSLKFEDTAVYYCNV (SEQ ID NO: 596), and FR4 comprises the amino acid sequence WGQGTQVTVSS (SEQ ID NO: 542);
aq) FR1 comprises the amino acid sequence QVQLVESGGGLVQPGESLRLSCVVSGDIFS (SEQ ID NO: 597), FR2 comprises the amino acid sequence WYRQAPGKQRELVA (SEQ ID NO: 574), FR3 comprises the amino acid sequence RFTISRDNAKRTVYLQMNSLKFEDTAVYYCNF (SEQ ID NO: 598), and FR4 comprises the amino acid sequence WGQGTQVTVSS (SEQ ID NO: 542);
ar) FR1 comprises the amino acid sequence QVQLVESGGGLVQPGGSLRLSCAASGFTFS (SEQ ID NO: 599), FR2 comprises the amino acid sequence WVRQAPGKRLEWVS (SEQ ID NO: 600), FR3 comprises the amino acid sequence RFTISRDNADNTLYLHMNNLKPEDTAVYYCAN (SEQ ID NO: 601), and FR4 comprises the amino acid sequence RGQGTQVTVSS (SEQ ID NO: 583);
as) FR1 comprises the amino acid sequence QVQLVESGGGLVQPGGSLRLSCAASGFTFS (SEQ ID NO: 599), FR2 comprises the amino acid sequence WVRQAPGKRLEWVS (SEQ ID NO: 600), FR3 comprises the amino acid sequence RFTISRDNADNTLYLHMNNLKPEDTAVYYCAN (SEQ ID NO: 601), and FR4 comprises the amino acid sequence RGQGTQVTVVS (SEQ ID NO: 622);
at) FR1 comprises the amino acid sequence QVQLVESGGGLVQPGGSLRLSCAASGFTFS (SEQ ID NO: 599), FR2 comprises the amino acid sequence WVRQAPGKRLEWVS (SEQ ID NO: 600), FR3 comprises the amino acid sequence RFTISRDNADNTLYLHMNNLKPEDTAVYYCAN (SEQ ID NO: 601), and FR4 comprises the amino acid sequence RGQGTQVTVSS (SEQ ID NO: 583); or
au) FR1 comprises the amino acid sequence QVQLVESGGGLVQPGGSLRLSCVASGGTFS, FR2 comprises the amino acid sequence WFRQAPGKEREFVA (SEQ ID NO: 539), FR3 comprises the amino acid sequence RFTISRENAKNTVYLQMNTLKLEDTAVYYCVS (SEQ ID NO: 603), and FR4 comprises the amino acid sequence WGQGTQVTVSS (SEQ ID NO: 542).

6. The VHH antibody of claim 1, wherein the VHH antibody comprises an amino acid sequence having at least 85% sequence identity to an amino acid sequence selected from the group consisting of:
```
  (SEQ ID NO: 431)
QVQLVESGGGLVQPGGSLRLSCAASGRTFINYAAGWFRQAPGKEREFVARISRSGGRTDYADSV
KGRFTISRDNAKSTVYLQMNSLRPEDTAVYYCAEATVWEFTDGADQYDYWGQGTQVTVSS;
(SEQ ID NO: 432)
EVQLVESGGGLVQAGGSLRLSCAASGRTFGSYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 433)
EVQLVESGGGLVQAGGSLRLSCAASGRTFGTYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 434)
QVQLQESGGGLVQAGGSLRLSCAASGRTFGSYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 435)
QVQLQESGGGLVQAGGSLRLSCAASGRTFGTYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 436)
QVQLQESGGGLVQAGGSLRLSCAASGRTFGTYTMGWFRQAPGKGREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 437)
QVQLVESGGGLVQAGGSLRLSCAASGRTFGSYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 438)
QVQLVESGGGLVQAGGSLRLSCAASGRTFGTYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 439)
QVQLVESGGGLVQAGGSLRLSCAASGRTFGTYTMGWFRQAPGREREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 440)
EVQLVESGGGLVQAGGSLRLSCAASGGTVSSYAMGWFRQAPGKEREFVAVISWSGGRTYYADSV
KGRFTISRDNAKNTVNLQMNSLKPEDTAVYYCAADPWTSDSDYERLTMYDYWGQGTQVTVSS;
(SEQ ID NO: 441)
EVQLVESGGGLVQAGGSRRLSCAASGGTVSSYAMGWFRQAPGKEREFVAVISWSGGRTYYADSV
KGRFTISRDNAKNTVYLQMNSLKPEDTAVYYCAADPWTSDSDYERLTMYDYWGQGTQVTVSS;
(SEQ ID NO: 442)
QVQLVESGGGLVQAGGSLRLSCAASGGTVSSYAMGWFRQAPGKEREFVAVISWSGGRTYYADSV
KGRFTISRDNAKNTVNLQMNSLKPEDTAVYYCAADPWTSDSDYERLTMYDYWGQGTQVTVSS;
(SEQ ID NO: 443)
QVQLVESGGGLVQAGGSLRLSCAASGGTVSSYAMGWFRQAPGKEREFVAVISWSGGRTYYADSV
KGRFTISRDNAKNTVYLQMNSLKPEDTAVYYCAADPWTSDSDYERLTMYDYWGQGTQVTVSS;
(SEQ ID NO: 444)
EVQLVESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTESQRLYGAWGQGTQVTVSS;
(SEQ ID NO: 445)
QVQLQESGGGLVQAGASLRLSCAASGRATYNMGWFRHAPGEDREFVAAINLEGYATRYANSVKG
RFTISRDNAKNTVYLQMNSLKPEDTAVYYCAADTSLPLGVLTESQRLYGAWGQGTQVTVSS;
(SEQ ID NO: 446)
QVQLQESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTESQRLYGAWGQGTQVTVSS;
(SEQ ID NO: 447)
QVQLQESGGGLVQAGASLRLSCAASGRTTYNMGWFRHAPGKDREFVAAIDLYGRATRYANSVRG
RFTISRDNAKNTVYLQMNSLKPEDTAVYYCAADTSLPLGVLTESQRLYGAWGQGTQVTVSS;
(SEQ ID NO: 448)
QVQLVESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTESQRLYGAWGQGTQVTVSS;
(SEQ ID NO: 449)
QVQLVESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTKSQRMYGAWGQGTQVTVSS;
  (SEQ ID NO: 450)
QVQLQESGGGSVQAGGSLRLSCAASGRAFSAYAMGWFRQAPGKEREFVAAINWNGDTALRWNGF
ATRYADSVKGRFIISRVNAKNTVNLQMNSLKPEDTAVYYCAADTVVSGSYYLAARAEDYEYWGQ
GTQVSVSS;
  (SEQ ID NO: 451)
QVQLQESGGGSVQAGGSLRLSCAASGRAFSAYAMGWFRQAPGKEREFVAAINWNGDTALRWNGF
ATRYADSVKGRFTISRVIAKNTVNLQMNSLKPEDTAVYYCAADTVVSGSYYLAARAEDYEYWGQ
GTQVTVSS;
  (SEQ ID NO: 452)
QVQLQESGGGSVQAGGSLRLSCAASGRAFSAYAMGWFRQAPGKEREFVAAINWNGDTALRWNGF
ATRYADSVKGRFTISRVNAKNTVNLQMNSLKPEDTAVYYCAADTVVSGSYYLAARAEDYEYWGQ
GTQVSVSS;
  (SEQ ID NO: 453)
QVQLQESGGGSVQAGGSLRLSCAASGRAFSAYAMGWFRQAPGKEREFVAAINWNGDTALRWNGF
ATRYADSVKGRFTISRVNAKNTVNLQMNSLKPEDTAVYYCAADTVVSGSYYLAARAEDYEYWGQ
GTQVTVSS;
  (SEQ ID NO: 454)
EVQLVESGGGLVQAGGSLRLSCAASGPAFSSSGMGWFRQAPGKARDFVASMDWSGGSTYYGDSV
KGRFTVSRDNAKNTVHLQMNSLRPEDTAVYYCARGTSGVAAVNLRGFFSWGPGTQVTVSS;
(SEQ ID NO: 455)
QVQLQESGGGLVQAGGSLRLSCAASGPAFSSSGMGWFRQAPGKARDFVASIDWSGGSTYYGDSV
KGRFTVSRDNAKNTVHLQMNSLRPEDTAVYYCARGTSGVAAVNLRGFFSWGPGTQVTVSS;
  (SEQ ID NO: 456)
QVQLVESGGGLVQAGGSLRLACAASGAAFSSSGMGWFRQAPGKARDFVASMDWSGGSTYYGDSV
KGRFTVSRDNAKNTVHLQMNSLRPEDTAVYYCARGTSGVAAVNLRGFFSWGPGTQVTVSS;
(SEQ ID NO: 457)
QVQLVESGGGLVQAGGSLRLSCAASGPAFSSSGMGWFRQAPGKARDFVASIDWGGGSTYYGDSV
KGRFTVSRDNAKNAVHLQMNSLRPEDTAVYYCARGTSGVAAVNLRGFFSWGPGTQVTVSS;
  (SEQ ID NO: 458)
QVQLVESGGGLVQAGGSLRLSCAASGPAFSSSGMGWFRQAPGKARDFVASIDWSGGSTYYGDSV
KGRFTVSRDNAKNTVHLQMNSLRPEDTAVYYCARGTSGVAAVNLRGFFSWGPGTQVTVSS;
  (SEQ ID NO: 459)
QVQLVESGGGLVQAGGSLRLSCAASGPAFSSSGMGWFRQAPGKARDFVASIDWSGKSTYYGDSV
KGRFTVSRDNAKNTVHLQMNSLRPEDTAVYYCARGTSGVAAVNLRGFFSWGPGTQVTVSS;
  (SEQ ID NO: 460)
QVQLVESGGGLVQAGGSLRLSCAASGPAFSSSGMGWFRQAPGKARDFVASINWSGGSAYYGDSV
KGRFTVSRDNAKNTVHLQMNSLRPEDTAVYYCARGTSGVAAVNLRGFFSWGPGTQVTVSS;
  (SEQ ID NO: 461)
QVQLVESGGGLVQAGGSLRLSCAASGPAFSSSGMGWFRQAPGKARDFVASMDWSGGSTYYGDSV
KGRFTVSRDNAKNTVHLQMNSLRPEDTAVYYCARGTSGVAAVNLRGFFSWGPGTQVTVSS;
  (SEQ ID NO: 462)
QVQLVESGGGLVQAGGSLRLSCAASGPAFSSSGMGWFRQAPGKAREFVASMDWTGGSTYYGDSV
KGRFTVSRDNAKMTVHLQMNSLRPEDTAVYYCARGTSGVAAVNLRGFFSWGPGTQVTVSS;
(SEQ ID NO: 463)
QLQLVESGGGLVQPGGSLRLSCAASGSDFLVDATTWFRQAPGNQREFVAIMDIGGVTEYADSVK
GRFTISRDHTKNTVYLQMNSLKVEDTAVYYCNTRGLWGQGTLVTVSS;
  (SEQ ID NO: 464)
QVQLQESGGGLVQAGGSLRLSCATSGITSSINVIGWYRQAPGKQRELVALVNSGGQTHYADSVK
GRFTISRDNAKNTVFLQMNSLKPEDTAEYYCHGRYGIDNYWGEGTQVTVSS;
  (SEQ ID NO: 465)
EVQLVESGGGLVQPGGSLRLSCAASGFPFSSSFMSWVRQAPGKGLEWVSTIYSDGSTYYADSVK
GRFTISRDNAKKTAYLQMNSLKAEDTAVYYCATVTGSIRGQGTQVTVSS;
  (SEQ ID NO: 466)
QVQLVESGGGLVQPGGSLRLSCAASGFNFSSSFMSWVRQAPGKEVEWVSTIYSDGSTYYADSMK
GRFTISRDNAKNTVYLQMSNLKAEDTAVYYCATVTGSIRGQGTQVTVSS;
  (SEQ ID NO: 467)
QVQLVESGGGLVQPGGSVRLSCATSGSIFSTNVMGWYRQAPGKEREFVALIRGGGSTHYADSVK
GRFIISRENAKTTVYLQMNGLKPEDTAVYYCVIWLGSPGAMSDYWGQGTQVTVSS;
  (SEQ ID NO: 468)
EVQLVESGGGLVQPGGSLRLSCAASGSVVSTNNMGWYRQAPGKEREFVALIRTGGSTHVADSMK
GRFTISRENAKNTVYLQMNSLKPEDTAVYYCVIWTGSPGALSDYWGQGTQVTVSS;
  (SEQ ID NO: 469)
QVQLVESGGGLVQPGGSLRLSCAASGSDASTNNMAWYRQAPGKEREFVALIRTGGSTHVADSMK
GRFTISRENAKNTVYLQMNSLKPEDTAVYYCVIWTGSPGALSDYWGQGTQVTVSS;
(SEQ ID NO: 470
QLQLVESGGGLVQPGESLRLSCAASGFSFSRVAMNWYRQAPGKERELVATISSDGSRTNYAHSV
KGRFTISRENAKNMVYLQMNSLKLEDTAVYYCNVPGNSWGQGTQVTVSS);
  (SEQ ID NO: 471)
QLQLVESGGGLVQPGESLRLSCAASGFSFSRVGMNWYRQAPGKERELVASISSDGSRTNYAHFV
KGRFTISRDNVKNMVYLQMNSLKLEDTAVYYCNVPGNSWGQGTQVTVSS;
  (SEQ ID NO: 472)
QLQLVESGGGLVQPGESLRLSCVVSGDIFSFVGWGWYRQAPGKQREVVAQISTGGLTNYADSVK
GRFAISRDNAKRTVYLQMNSLKFEDTAVYYCNVPGHPWGQGTQVTVSS;
  (SEQ ID NO: 473)
QVQLVESGGGLVQPGESLRLSCVVSGDIFSFIGWGWYRQAPGKQRELVAQINTGGLTDYADSVK
GRFTISRDNAKRTVYLQMNSLKFEDTAVYYCNFPGHSWGQGTQVTVSS;
  (SEQ ID NO: 474)
QVQLVESGGGLVQPGGSLRLSCAASGFTFSMYSMSWVRQAPGKRLEWVSSISTGARDTAYADSV
KGRFTISRDNADNTLYLHMNNLKPEDTAVYYCANGDLRYGPDGYDYRGQGTQVTVSS;
  (SEQ ID NO: 475)
QVQLVESGGGLVQPGGSLRLSCAASGFTFSMYSMSWVRQAPGKRLEWVSSISTGARDTAYADSV
KGRFTISRDNADNTLYLHMNNLKPEDTAVYYCANGDLRYGPDGYDYRGQGTQVTVVS;
  (SEQ ID NO: 476)
QVQLVESGGGLVQPGGSLRLSCAASGFTFSMYSMSWVRQAPGKRLEWVSSISTGARDTSYADSV
KGRFTISRDNADNTLYLHMNNLKPEDTAVYYCANGDLRYGPDGYDYRGQGTQVTVSS;
and
(SEQ ID NO: 477)
QVQLVESGGGLVQPGGSLRLSCVASGGTFSTYGMGWFRQAPGKEREFVAVITGSGVGTQYADSV
KDRFTISRENAKNTVYLQMNTLKLEDTAVYYCVSGHRPGWAVIRADAYEYWGQGTQVTVSS.
```

7. The VHH antibody of claim 1, wherein the VHH antibody comprises an amino acid sequence selected from the group consisting of:
```
  (SEQ ID NO: 431)
QVQLVESGGGLVQPGGSLRLSCAASGRTFINYAAGWFRQAPGKEREFVARISRSGGRTDYADSV
KGRFTISRDNAKSTVYLQMNSLRPEDTAVYYCAEATVWEFTDGADQYDYWGQGTQVTVSS;
(SEQ ID NO: 432)
EVQLVESGGGLVQAGGSLRLSCAASGRTFGSYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 433)
EVQLVESGGGLVQAGGSLRLSCAASGRTFGTYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 434)
QVQLQESGGGLVQAGGSLRLSCAASGRTFGSYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 435)
QVQLQESGGGLVQAGGSLRLSCAASGRTFGTYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 436)
QVQLQESGGGLVQAGGSLRLSCAASGRTFGTYTMGWFRQAPGKGREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 437)
QVQLVESGGGLVQAGGSLRLSCAASGRTFGSYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 438)
QVQLVESGGGLVQAGGSLRLSCAASGRTFGTYTMGWFRQAPGKEREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 439)
QVQLVESGGGLVQAGGSLRLSCAASGRTFGTYTMGWFRQAPGREREFVAAISWSAGRTYYADSM
KGRFTISRDNAKNTVYLQMNSLKPEDTAVYYCAADPWTSDSDYDRLTMYDYWGQGTQVTVSS;
(SEQ ID NO: 440)
EVQLVESGGGLVQAGGSLRLSCAASGGTVSSYAMGWFRQAPGKEREFVAVISWSGGRTYYADSV
KGRFTISRDNAKNTVNLQMNSLKPEDTAVYYCAADPWTSDSDYERLTMYDYWGQGTQVTVSS;
(SEQ ID NO: 441)
EVQLVESGGGLVQAGGSRRLSCAASGGTVSSYAMGWFRQAPGKEREFVAVISWSGGRTYYADSV
KGRFTISRDNAKNTVYLQMNSLKPEDTAVYYCAADPWTSDSDYERLTMYDYWGQGTQVTVSS;
(SEQ ID NO: 442)
QVQLVESGGGLVQAGGSLRLSCAASGGTVSSYAMGWFRQAPGKEREFVAVISWSGGRTYYADSV
KGRFTISRDNAKNTVNLQMNSLKPEDTAVYYCAADPWTSDSDYERLTMYDYWGQGTQVTVSS;
(SEQ ID NO: 443)
QVQLVESGGGLVQAGGSLRLSCAASGGTVSSYAMGWFRQAPGKEREFVAVISWSGGRTYYADSV
KGRFTISRDNAKNTVYLQMNSLKPEDTAVYYCAADPWTSDSDYERLTMYDYWGQGTQVTVSS;
(SEQ ID NO: 444)
EVQLVESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTESQRLYGAWGQGTQVTVSS;
(SEQ ID NO: 445)
QVQLQESGGGLVQAGASLRLSCAASGRATYNMGWFRHAPGEDREFVAAINLEGYATRYANSVKG
RFTISRDNAKNTVYLQMNSLKPEDTAVYYCAADTSLPLGVLTESQRLYGAWGQGTQVTVSS;
(SEQ ID NO: 446)
QVQLQESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTESQRLYGAWGQGTQVTVSS;
(SEQ ID NO: 447)
QVQLQESGGGLVQAGASLRLSCAASGRTTYNMGWFRHAPGKDREFVAAIDLYGRATRYANSVRG
RFTISRDNAKNTVYLQMNSLKPEDTAVYYCAADTSLPLGVLTESQRLYGAWGQGTQVTVSS;
(SEQ ID NO: 448)
QVQLVESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTESQRLYGAWGQGTQVTVSS;
(SEQ ID NO: 449)
QVQLVESGGGLVQAGASLRLSCAASGRTTYNMGWFRHAPGKDREFVAAIDLYGRATRYANSVKG
RFTISRDNAKNTVYLQMNSLKPEDTAVYYCAADTSLPLGVLTKSQRMYGAWGQGTQVTVSS;
  (SEQ ID NO: 450)
QVQLQESGGGSVQAGGSLRLSCAASGRAFSAYAMGWFRQAPGKEREFVAAINWNGDTALRWNGF
ATRYADSVKGRFIISRVNAKNTVNLQMNSLKPEDTAVYYCAADTVVSGSYYLAARAEDYEYWGQ
GTQVSVSS;
  (SEQ ID NO: 451)
QVQLQESGGGSVQAGGSLRLSCAASGRAFSAYAMGWFRQAPGKEREFVAAINWNGDTALRWNGF
ATRYADSVKGRFTISRVIAKNTVNLQMNSLKPEDTAVYYCAADTVVSGSYYLAARAEDYEYWGQ
GTQVTVSS;
  (SEQ ID NO: 452)
QVQLQESGGGSVQAGGSLRLSCAASGRAFSAYAMGWFRQAPGKEREFVAAINWNGDTALRWNGF
ATRYADSVKGRFTISRVNAKNTVNLQMNSLKPEDTAVYYCAADTVVSGSYYLAARAEDYEYWGQ
GTQVSVSS;
  (SEQ ID NO: 453)
QVQLQESGGGSVQAGGSLRLSCAASGRAFSAYAMGWFRQAPGKEREFVAAINWNGDTALRWNGF
ATRYADSVKGRFTISRVNAKNTVNLQMNSLKPEDTAVYYCAADTVVSGSYYLAARAEDYEYWGQ
GTQVTVSS;
  (SEQ ID NO: 454)
EVQLVESGGGLVQAGGSLRLSCAASGPAFSSSGMGWFRQAPGKARDFVASMDWSGGSTYYGDSV
KGRFTVSRDNAKNTVHLQMNSLRPEDTAVYYCARGTSGVAAVNLRGFFSWGPGTQVTVSS;
  (SEQ ID NO: 455)
QVQLQESGGGLVQAGGSLRLSCAASGPAFSSSGMGWFRQAPGKARDFVASIDWSGGSTYYGDSV
KGRFTVSRDNAKNTVHLQMNSLRPEDTAVYYCARGTSGVAAVNLRGFFSWGPGTQVTVSS;
  (SEQ ID NO: 456)
QVQLVESGGGLVQAGGSLRLACAASGAAFSSSGMGWFRQAPGKARDFVASMDWSGGSTYYGDSV
KGRFTVSRDNAKNTVHLQMNSLRPEDTAVYYCARGTSGVAAVNLRGFFSWGPGTQVTVSS;
  (SEQ ID NO: 457)
QVQLVESGGGLVQAGGSLRLSCAASGPAFSSSGMGWFRQAPGKARDFVASIDWGGGSTYYGDSV
KGRFTVSRDNAKNAVHLQMNSLRPEDTAVYYCARGTSGVAAVNLRGFFSWGPGTQVTVSS;
  (SEQ ID NO: 458)
QVQLVESGGGLVQAGGSLRLSCAASGPAFSSSGMGWFRQAPGKARDFVASIDWSGGSTYYGDSV
KGRFTVSRDNAKNTVHLQMNSLRPEDTAVYYCARGTSGVAAVNLRGFFSWGPGTQVTVSS;
  (SEQ ID NO: 459)
QVQLVESGGGLVQAGGSLRLSCAASGPAFSSSGMGWFRQAPGKARDFVASIDWSGKSTYYGDSV
KGRFTVSRDNAKNTVHLQMNSLRPEDTAVYYCARGTSGVAAVNLRGFFSWGPGTQVTVSS;
  (SEQ ID NO: 460)
QVQLVESGGGLVQAGGSLRLSCAASGPAFSSSGMGWFRQAPGKARDFVASINWSGGSAYYGDSV
KGRFTVSRDNAKNTVHLQMNSLRPEDTAVYYCARGTSGVAAVNLRGFFSWGPGTQVTVSS;
  (SEQ ID NO: 461)
QVQLVESGGGLVQAGGSLRLSCAASGPAFSSSGMGWFRQAPGKARDFVASMDWSGGSTYYGDSV
KGRFTVSRDNAKNTVHLQMNSLRPEDTAVYYCARGTSGVAAVNLRGFFSWGPGTQVTVSS;
  (SEQ ID NO: 462)
QVQLVESGGGLVQAGGSLRLSCAASGPAFSSSGMGWFRQAPGKAREFVASMDWTGGSTYYGDSV
KGRFTVSRDNAKMTVHLQMNSLRPEDTAVYYCARGTSGVAAVNLRGFFSWGPGTQVTVSS;
  (SEQ ID NO: 463)
QLQLVESGGGLVQPGGSLRLSCAASGSDFLVDATTWFRQAPGNQREFVAIMDIGGVTEYADSVK
GRFTISRDHTKNTVYLQMNSLKVEDTAVYYCNTRGLWGQGTLVTVSS;
  (SEQ ID NO: 464)
QVQLQESGGGLVQAGGSLRLSCATSGITSSINVIGWYRQAPGKQRELVALVNSGGQTHYADSVK
GRFTISRDNAKNTVFLQMNSLKPEDTAEYYCHGRYGIDNYWGEGTQVTVSS;
  (SEQ ID NO: 465)
EVQLVESGGGLVQPGGSLRLSCAASGFPFSSSFMSWVRQAPGKGLEWVSTIYSDGSTYYADSVK
GRFTISRDNAKKTAYLQMNSLKAEDTAVYYCATVTGSIRGQGTQVTVSS;
  (SEQ ID NO: 466)
QVQLVESGGGLVQPGGSLRLSCAASGFNFSSSFMSWVRQAPGKEVEWVSTIYSDGSTYYADSMK
GRFTISRDNAKNTVYLQMSNLKAEDTAVYYCATVTGSIRGQGTQVTVSS;
  (SEQ ID NO: 467)
QVQLVESGGGLVQPGGSVRLSCATSGSIFSTNVMGWYRQAPGKEREFVALIRGGGSTHYADSVK
GRFIISRENAKTTVYLQMNGLKPEDTAVYYCVIWLGSPGAMSDYWGQGTQVTVSS;
  (SEQ ID NO: 468)
EVQLVESGGGLVQPGGSLRLSCAASGSVVSTNNMGWYRQAPGKEREFVALIRTGGSTHVADSMK
GRFTISRENAKNTVYLQMNSLKPEDTAVYYCVIWTGSPGALSDYWGQGTQVTVSS;
  (SEQ ID NO: 469)
QVQLVESGGGLVQPGGSLRLSCAASGSDASTNNMAWYRQAPGKEREFVALIRTGGSTHVADSMK
GRFTISRENAKNTVYLQMNSLKPEDTAVYYCVIWTGSPGALSDYWGQGTQVTVSS;
  (SEQ ID NO: 470)
QLQLVESGGGLVQPGESLRLSCAASGFSFSRVAMNWYRQAPGKERELVATISSDGSRTNYAHSV
KGRFTISRENAKNMVYLQMNSLKLEDTAVYYCNVPGNSWGQGTQVTVSS;
  (SEQ ID NO: 471)
QLQLVESGGGLVQPGESLRLSCAASGFSFSRVGMNWYRQAPGKERELVASISSDGSRTNYAHFV
KGRFTISRDNVKNMVYLQMNSLKLEDTAVYYCNVPGNSWGQGTQVTVSS;
  (SEQ ID NO: 472)
QLQLVESGGGLVQPGESLRLSCVVSGDIFSFVGWGWYRQAPGKQREVVAQISTGGLTNYADSVK
GRFAISRDNAKRTVYLQMNSLKFEDTAVYYCNVPGHPWGQGTQVTVSS;
  (SEQ ID NO: 473)
QVQLVESGGGLVQPGESLRLSCVVSGDIFSFIGWGWYRQAPGKQRELVAQINTGGLTDYADSVK
GRFTISRDNAKRTVYLQMNSLKFEDTAVYYCNFPGHSWGQGTQVTVSS;
  (SEQ ID NO: 474)
QVQLVESGGGLVQPGGSLRLSCAASGFTFSMYSMSWVRQAPGKRLEWVSSISTGARDTAYADSV
KGRFTISRDNADNTLYLHMNNLKPEDTAVYYCANGDLRYGPDGYDYRGQGTQVTVSS;
  (SEQ ID NO: 475)
QVQLVESGGGLVQPGGSLRLSCAASGFTFSMYSMSWVRQAPGKRLEWVSSISTGARDTAYADSV
KGRFTISRDNADNTLYLHMNNLKPEDTAVYYCANGDLRYGPDGYDYRGQGTQVTVVS;
  (SEQ ID NO: 476)
QVQLVESGGGLVQPGGSLRLSCAASGFTFSMYSMSWVRQAPGKRLEWVSSISTGARDTSYADSV
KGRFTISRDNADNTLYLHMNNLKPEDTAVYYCANGDLRYGPDGYDYRGQGTQVTVSS;
and
(SEQ ID NO: 477)
QVQLVESGGGLVQPGGSLRLSCVASGGTFSTYGMGWFRQAPGKEREFVAVITGSGVGTQYADSV
KDRFTISRENAKNTVYLQMNTLKLEDTAVYYCVSGHRPGWAVIRADAYEYWGQGTQVTVSS.
```

8. A chimeric antigen receptor polypeptide comprising the VHH antibody of claim 1, or an antigen-binding fragment thereof.

9. An immunoconjugate comprising the VHH antibody of claim 1.

10. A polynucleotide encoding the VHH antibody of claim 1.

11. A vector comprising the polynucleotide of claim 10.

12. A cell expressing the VHH antibody of claim 1.

13. A lipid nanoparticle comprising the VHH antibody of claim 1.

14. The lipid nanoparticle of claim 13, wherein the lipid nanoparticle is conjugated to the VHH antibody.

15. The lipid nanoparticle of claim 14, wherein the VHH antibody is covalently bound to a polyethylene glycol (PEG) molecule of the lipid nanoparticle.

16. The lipid nanoparticle of claim 14, wherein the VHH antibody is covalently bound to a PEG portion of a PEG-modified lipid of the lipid nanoparticle.

17. The lipid nanoparticle of claim 13, wherein the lipid nanoparticle comprises a polynucleotide encoding a chimeric antigen receptor.

18. The lipid nanoparticle of claim 13, wherein the chimeric antigen receptor comprises an antigen binding domain capable of binding a marker associated with a neoplasia.

19. A composition comprising the VHH antibody of claim 1, and a carrier or excipient.

20. A method for treating a neoplasia in a subject in need thereof, the method comprising administering to the subject the lipid nanoparticle of claim 17.

21. The method of claim 20, wherein the neoplasia is a B cell lymphoma or a T cell lymphoma.
